(19) United States
(12) Patent Application Publication
(10) Pub. No.: US 2015/0344888 A1
(43) Pub. Date: Dec. 3, 2015

(54) USP30 INHIBITORS AND METHODS OF USE

(71) Applicant: GENENTECH, INC., South San Francisco, CA (US)

(72) Inventors: Yingnan Zhang, South San Francisco, CA (US); Baris Bingol, South San Francisco, CA (US); Jacob Corn, South San Francisco, CA (US)

(73) Assignee: GENENTECH, INC., South San Francisco, CA (US)

(21) Appl. No.: 14/659,204

(22) Filed: Mar. 16, 2015

Related U.S. Application Data
(63) Continuation of application No. PCT/EP2013/006898, filed on Sep. 13, 2013.
(60) Provisional application No. 61/809,927, filed on Apr. 9, 2013.
(60) Provisional application No. 61/701,963, filed on Sep. 17, 2012.

(51) Int. Cl. C12N 15/113; A61K 38/10; C07K 7/08; A61K 31/7088; A61K 45/06

(52) U.S. Cl. CPC C12N 15/1137; A61K 31/7088; A61K 45/06; C07K 7/08; A61K 38/10; C12N 2310/11; C12N 2310/14

(57) ABSTRACT

Inhibitors of USP30 and methods of using inhibitors of USP30 are provided. In some embodiments, methods of treating conditions involving mitochondrial defects are provided.

### RELATED APPLICATIONS

1. Field

Inhibitors of USP30 and methods of using inhibitors of USP30 are provided. In some embodiments, methods of treating conditions involving mitochondrial defects are provided.

2. Background

Mitophagy is a specialized autophagy pathway that eliminates mitochondria through degradation by lysosomes. As such, it removes mitochondria during normal cellular turnover of organelles, during maturation of erythrocytes, and following fertilization to eliminate sperm-derived mitochondria. Mitophagy also mediates the clearance of damaged mitochondria, an important aspect of mitochondria quality control. Defective or excess mitochondria, if left uncleared, may become an aberrant source of oxidative stress and compromise healthy mitochondria through mitochondrial fusion. In yeast, selective blockade of mitophagy causes increased production of reactive oxygen species (ROS) by excess mitochondria and loss of mitochondrial DNA (mt-DNA). Impaired mitochondria quality control could also affect key biosynthetic pathways, ATP production, and Ca2+ buffering, and disturb overall cellular homeostasis.

Parkinson's disease (PD), the second most common neurodegenerative disorder after Alzheimer's disease (AD), is characterized most prominently by loss of dopaminergic neurons in the substantia nigra. Although the pathogenic mechanisms of PD are not clear, several lines of evidence suggest that mitochondrial dysfunction is central to PD. MPTP, a mitochondrial toxin, damages dopamine neurons and produces clinical parkinsonism in humans. Epidemiologic evidence links PD with exposure to pesticides such as rotenone (a complex I inhibitor) and paraquat (an oxidative stressor). Consistent with mitochondrial impairment, reduced complex I activity and high levels of mt-DNA mutations have been found in substantia nigra from PD patients. Similarly, functional and morphological changes in mitochondria are present in genetic models of PD. Perhaps most compellingly, early-onset familial PD can be caused by mutations in Parkin ubiquitin-ligase and PINK1 serine/threonine protein kinase, both of which function to maintain healthy mitochondria through regulating mitochondrial dynamics and quality control.

Genetic studies in flies established that PINK1 acts upstream of Parkin to maintain proper mitochondria morphology and function. PINK1 recruits Parkin from the cytoplasm to the surface of damaged mitochondria, leading to Parkin-mediated ubiquitination of mitochondrial outer membrane proteins and removal of damaged mitochondria by mitophagy. PD-associated mutations in either PINK1 or Parkin impair Parkin recruitment, mitochondrial ubiquitination and mitophagy. Parkin regulates multiple aspects of mitochondrial function such as mitochondrial dynamics and trafficking, and may also influence mitochondria biogenesis. The degradation of a broad range of outer mitochondrial membrane proteins on damaged mitochondria appears to be affected by Parkin. Among these mitochondria associated proteins, MIRO, a component of the mitochondria-kinesin motor adaptor complex, may be a shared substrate of both Parkin and PINK-1.

Parkin expression and/or activity can be impaired through genetic mutations in familial PD or by phosphorylation in sporadic PD. In the context of the inherently high mitochondrial oxidative stress in substantia nigra dopamine neurons, loss of Parkin-mediated mitochondrial quality control could explain the greater susceptibility of substantia nigra neurons to neurodegeneration. Promoting clearance of damaged mitochondria and enhancing mitochondrial quality control could be beneficial in PD.

### SUMMARY

In some embodiments, methods of increasing mitophagy in a cell are provided. In some embodiments, the method comprises contacting the cell with an inhibitor of USP30.

In some embodiments, methods of increasing mitochondrial ubiquitination in a cell are provided. In some embodiments, methods of increasing ubiquitination of at least one, at least two, at least three, at least four, at least five, at least six, at least seven, at least eight, at least nine, at least ten, at least eleven, at least twelve, at least thirteen, or fourteen proteins selected from Tom20, MIRO, MUL1, ASNS, FKBP8, TOM70, MAT2B, PRDX3, IDE, VDAC1, VDAC2, VDAC3, IP05, PSD13, UBP13, and PTH2 in a cell are provided. In some embodiments, the method comprises contacting the cell with an inhibitor of USP30.

In some embodiments, the method comprises increasing ubiquitination of at least one, at least two, or three amino acids selected from K56, K61, and K68 of Tom 20. In some embodiments, the method comprises increasing ubiquitination of at least one, at least two, at least three, at least four, at least five, at least six, at least seven, or eight amino acids selected from K153, K187, K330, K427, K512, K535, K567, and K572 of MIRO. In some embodiments, the method comprises increasing ubiquitination of at least one, at least two, or three amino acids selected from K273, K299, and K52 of MUL1. In some embodiments, the method comprises increasing ubiquitination of at least one, at least two, at least three, at least four, at least five, at least six, at least seven, at least eight, or nine amino acids selected from K147, K168, K176, K221, K244, K275, K478, K504, and K556 of ASNS. In some embodiments, the method comprises increasing ubiquitination of at least one, at least two, at least three, at least four, at least five, at least six, at least seven, or eight amino acids selected from K249, K271, K273, K284, K307, K317, K334, and K340 of FKBP8. In some embodiments, the method comprises increasing ubiquitination of at least one, at least two, at least three, at least four, at least five, at least six, at least seven, at least eight, at least nine, or at least ten amino acids selected from K78, K120, K123, K126, K129, K148, K168, K170, K178, K185, K204, K230, K233, K245, K275, K278, K312, K326, K349, K359, K441, K463, K470, K471, K494, K501, K524, K536, K563, K570, K599, K600, and K604 of TOM70. In some embodiments, the method comprises increasing ubiquitination of at least one, at least two, at least three, or four amino acids selected from K209, K245, K316, and K326 of MAT2B. In some embodiments, the method comprises increasing ubiquitination of at least one, at least two, at least three, at least four, or five amino acids selected from K83, K91, K166, K241, and K253 of PRDX3. In some embodiments, the method comprises increasing ubiquitination of at least one, at least two, at least three, at least four, at least five, or six amino acids selected from K558, K657, K854, K884, K929, and K933 of IDE. In some embodiments, the method comprises increasing ubiquitination of at least one, at least two, at least three, at least four, at least five, at least six, or seven amino acids selected from K20, K53, K61, K109, K110, K266, and K274 of VDAC1. In some embodiments, the method comprises increasing ubiquitination of at least one, at least two, at least three, at least four, at least five, or six amino acids selected from K31, K64, K120, K121, K277, and K285 of VDAC2. In some embodiments, the method comprises increasing ubiquitination of at least one, at least two, at least three, at least four, at least five, at least six, at least seven, or eight amino acids selected from K20, K53, K61, K109, K110, K163, K266, and K274 of VDAC3. In some embodiments, the method comprises increasing ubiquitination of at at least one, at least two, at least three, at least four, at least five, at least six, at least seven, at least eight, at least nine, or at least ten amino acids selected from K238, K353, K436, K437, K548, K556, K613, K678, K690, K705, K775, and K806 of IP05. In some embodiments, the method comprises increasing ubiquitination of at least one, at least two, at least three, at least four, at least five, at least six, at least seven, at least eight, at least nine, or at least ten amino acids selected from K2, K32, K99, K115, K122, K132, K161, K186, K313, K321, K347, K350, and K361 of PSD13. In some embodiments, the method comprises increasing ubiquitination of at least one, at least two, at least three, at least four, at least five, at least six, at least seven, at least eight, at least nine, or at least ten amino acids selected from K18, K190, K259, K326, K328, K401, K405, K414, K418, K435, K586, K587, and K640 of UBP13. In some embodiments, the method comprises increasing ubiquitination of at least one, at least two, at least three, at least four, at least five, at least six, at least seven, at least eight, or nine amino acids selected from 47, 76, 81, 95, 106, 119, 134, 171, 177 of PTH2.

In some embodiments, the cell is under oxidative stress. In some embodiments, methods of reducing oxidative stress in a cell are provided. In some embodiments, a method comprises contacting the cell with an inhibitor of USP30.

In some embodiments, the cell comprises a pathogenic mutation in Parkin, a pathogenic mutation in PINK1, or a pathogenic mutation in Parkin and a pathogenic mutation in PINK1. Nonlimiting exemplary pathogenic mutations in Parkin are shown in Table 1. Thus, in some embodiments, the pathogenic mutation in Parkin is selected from the mutations in Table 1. Nonlimiting exemplary pathogenic mutations in PINK1 are shown in Table 2. In some embodiments, the pathogenic mutation in PINK1 selected from the mutations in Table 2.

In various embodiments, the cell is selected from a neuron, a cardiac cell, and a muscle cell. In some such embodiments, the cell is ex vivo or in vitro. Alternatively, in some such embodiments, the cell is comprised in a subject.

In some embodiments, methods of treating conditions involving mitochondrial defects in a subject are provided. In some embodiments, the method comprises administering to the subject an effective amount of an inhibitor of USP30. In some embodiments, the condition involving a mitochondrial defect is selected from a condition involving a mitophagy defect, a condition involving a mutation in mitochondrial DNA, a condition involving mitochondrial oxidative stress, a condition involving a defect in mitochondrial shape or morphology, a condition involving a defect in mitochondrial membrane potential, and a condition involving a lysosomal storage defect.

In some embodiments, the condition involving a mitochondrial defect is selected from a neurodegenerative disease; mitochondrial myopathy, encephalopathy, lactic acidosis, and stroke-like episodes (MELAS) syndrome; Leber's hereditary optic neuropathy (LHON); neuropathy, ataxia, retinitis pigmentosa-maternally inherited Leigh syndrome (NARP-MILS); Danon disease; ischemic heart disease leading to myocardial infarction; multiple sulfatase deficiency (MSD); mucolipidosis II (ML II); mucolipidosis III (ML III); mucolipidosis IV (ML IV); GM1-gangliosidosis (GM1); neuronal ceroid-lipofuscinoses (NCL1); Alpers disease; Barth syndrome; Beta-oxidation defects; carnitine-acyl-carnitine deficiency; carnitine deficiency; creatine deficiency syndromes; co-enzyme Q10 deficiency; complex I deficiency; complex II deficiency; complex III deficiency; complex IV deficiency; complex V deficiency; COX deficiency; chronic progressive external ophthalmoplegia syndrome (CPEO); CPT I deficiency; CPT II deficiency; glutaric aciduria type II; Kearns-Sayre syndrome; lactic acidosis; long-chain acyl-CoA dehydrongenase deficiency (LCHAD); Leigh disease or syndrome; lethal infantile cardiomyopathy (LIC); Luft disease; glutaric aciduria type II; medium-chain acyl-CoA dehydrongenase deficiency (MCAD); myoclonic epilepsy and ragged-red fiber (MERRF) syndrome; mitochondrial recessive ataxia syndrome; mitochondrial cytopathy; mitochondrial DNA depletion syndrome; myoneurogastointestinal disorder and encephalopathy; Pearson syndrome; pyruvate carboxylase deficiency; pyruvate dehydrogenase deficiency; POLG mutations; medium/short-chain 3-hydroxyacyl-CoA dehydrogenase (M/SCHAD) deficiency; and very long-chain acyl-CoA dehydrongenase (VLCAD) deficiency.

In some embodiments, methods of treating neurodegenerative diseases are provided. In some embodiments, the method comprises administering to a subject an effective amount of an inhibitor of USP30.

In some embodiments, the neurodegenerative disease is selected from Alzheimer's disease, Parkinson's disease, amyotrophic lateral sclerosis (ALS), Huntington's disease, ischemia, stroke, dementia with Lewy bodies, and frontotemporal dementia.

In some embodiments, methods of treating Parkinson's disease are provided. In some embodiments, the method comprises administering to a subject an effective amount of an inhibitor of USP30.

In some embodiments, methods of treating conditions involving cells undergoing oxidative stress are provided. In some embodiments, the method comprises administering to a subject an effective amount of an inhibitor of USP30.

In some embodiments involving treatment of a subject, the subject comprises a pathogenic mutation in Parkin, a pathogenic mutation in PINK1, or a pathogenic mutation in Parkin and a pathogenic mutation in PINK1 in at least a portion of the subject's cells. In some embodiments, the pathogenic mutation in Parkin is selected from the mutations in Table 1. In some embodiments, the pathogenic mutation in PINK1 is selected from the mutations in Table 2.

In some embodiments, the inhibitor of USP30 is administered orally, intramuscularly, intravenously, intraarterially, intraperitoneally, or subcutaneously. In some embodiments, the method comprises administering at least one additional therapeutic agent. In some embodiments, the at least one additional therapeutic agent is selected from levodopa, a dopamine agonist, a monoamino oxygenase (MAO) B inhibitor, a catechol O-methyltransferase (COMT) inhibitor, an anticholinergic, amantadine, riluzole, a cholinesterase inhibitor, memantine, tetrabenazine, an antipsychotic, clonazepam, diazepam, an antidepressant, and an anti-convulsant.

In any of the methods described herein, the inhibitor of USP30 may be an inhibitor of USP30 expression. Nonlimiting exemplary inhibitors of USP30 expression include antisense oligonucleotides and short interfering RNAs (siRNAs). In any of the methods described herein, the inhibitor of USP30 may be an inhibitor of USP30 activity. Nonlimiting exemplary inhibitors of USP30 activity include antibodies, peptides, peptibodies, aptamers, and small molecules.

In some embodiments, a peptide inhibitor of USP30 comprises the amino acid sequence:

| | |
|---|---|
| X₁X₂CX₃X₄X₅X₆X₇X₈X₉X₁₀X₁₁CX₁₂ | (SEQ ID NO: 48) |

wherein:

X₁ is selected from L, M, A, S, and V;

X₂ is selected from Y, D, E, I, L, N, and S;

X₃ is selected from F, I, and Y;

X₄ is selected from F, I, and Y;

X₅ is selected from D and E;

X₆ is selected from L, M, V, and P;

X₇ is selected from S, N, D, A, and T;

X₈ is selected from Y, D, F, N, and W;

X₉ is selected from G, D, and E;

X₁₀ is selected from Y and F;

X₁₀ is selected from L, V, M, Q, and W; and

X₁₂ is selected from F, L, C, V, and Y.

In some embodiments, a peptide inhibitor of USP30 peptide comprises an amino acid sequence that is at least 80%, at least 85%, at least 90%, at least 95%, or 100% identical to an amino acid sequence selected from SEQ ID NOs: 1 to 22. In some embodiments, the peptide inhibits USP30 with an IC50 of less than 10 μM. In some embodiments, the IC50 of a peptide inhibitor of USP30 for at least one, at least two, or at least three peptidases selected from USP7, USP5, UCHL3, and USP2 is greater than 20 μM, greater than 30 μM, greater than 40 μM, or greater than 50 μM.

In some embodiments, an antisense oligonucleotide comprises a nucleotide sequence that is at least at least 80%, at least 85%, at least 90%, at least 95%, or 100% complementary to a region of USP30 mRNA and/or a region of USP30 pre-mRNA. In some embodiments, the region of USP30 mRNA or region of USP30 pre-mRNA is at least at least 10, at least 15, at least 20, at least 25, at least 30, at least 40, at least 50, at least 60, at least 70, at least 80, at least 90, or at least 100 nucleotides long. In some embodiments, the antisense oligonucleotide is 10 to 500 nucleotides long, or 10 to 400 nucleotides long, or 10 to 300 nucleotides long, or 10 to 200 nucleotides long, or 10 to 100 nucleotides long, or 15 to 100 nucleotides long, or 10 to 50 nucleotides long, or 15 to 50 nucleotides long. An antisense oligonucleotide may comprise one or more non-nucleotide components.

In some embodiments, an siRNA comprises a nucleotide sequence that is at least at least 80%, at least 85%, at least 90%, at least 95%, or 100% identical to a region of USP30 mRNA and/or a region of USP30 pre-mRNA. In some embodiments, the region of USP30 mRNA or region of USP30 pre-mRNA is at least at least 10, at least 15, at least 20, or at least 25 nucleotides long. In some embodiments, the siRNA is 10 to 200 nucleotides long, or 10 to 100 nucleotides long, or 15 to 100 nucleotides long, or 10 to 60 nucleotides long, or 15 to 60 nucleotides long, or 10 to 50 nucleotides long, or 15 to 50 nucleotides long, or 10 to 30 nucleotides long, or 15 to 30 nucleotides long. In some embodiments, an siRNA is an shRNA.

An embodiment of the present invention is an inhibitor of USP30 for the treatment of a condition involving a mitochondrial defect in a subject. In a particular embodiment the condition involving a mitochondrial defect is selected from a condition involving a mitophagy defect, a condition involving a mutation in mitochondrial DNA, a condition involving mitochondrial oxidative stress, a condition involving a defect in mitochondrial shape or morphology, a condition involving a defect in mitochondrial membrane potential, and a condition involving a lysosomal storage defect. in another particular embodiment the condition involving a mitochondrial defect is selected from a neurodegenerative disease; mitochondrial myopathy, encephalopathy, lactic acidosis, and stroke-like episodes (MELAS) syndrome; Leber's hereditary optic neuropathy (LHON); neuropathy, ataxia, retinitis pigmentosa-maternally inherited Leigh syndrome (NARP-MILS); Danon disease; ischemic heart disease leading to myocardial infarction; multiple sulfatase deficiency (MSD); mucolipidosis II (ML II); mucolipidosis III (ML III); mucolipidosis IV (ML IV); GM1-gangliosidosis (GM1); neuronal ceroid-lipofuscinoses (NCL1); Alpers disease; Barth syndrome; Beta-oxidation defects; carnitine-acyl-carnitine deficiency; carnitine deficiency; creatine deficiency syndromes; co-enzyme Q10 deficiency; complex I deficiency; complex II deficiency; complex III deficiency; complex IV deficiency; complex V deficiency; COX deficiency; chronic progressive external ophthalmoplegia syndrome (CPEO); CPT I deficiency; CPT II deficiency; glutaric aciduria type II; Kearns-Sayre syndrome; lactic acidosis; long-chain acyl-CoA dehydrongenase deficiency (LCHAD); Leigh disease or syndrome; lethal infantile cardiomyopathy (LIC); Luft disease; glutaric aciduria type II; medium-chain acyl-CoA dehydrongenase deficiency (MCAD); myoclonic epilepsy and ragged-red fiber (MERRF) syndrome; mitochondrial recessive ataxia syndrome; mitochondrial cytopathy; mitochondrial DNA depletion syndrome; myoneurogastointestinal disorder and encephalopathy; Pearson syndrome; pyruvate carboxylase deficiency; pyruvate dehydrogenase deficiency; POLG mutations; medium/short-chain 3-hydroxyacyl-CoA dehydrogenase (M/SCHAD) deficiency; and very long-chain acyl-CoA dehydrongenase (VLCAD) deficiency. In a more particular embodiment the neurodegenerative disease is selected from Alzheimer's disease, Parkinson's disease, amyotrophic lateral sclerosis (ALS), Huntington's disease, ischemia, stroke, dementia with Lewy bodies, and frontotemporal dementia.

Another embodiment of the present invention is an inhibitor of USP30 for the treatment of a neurodegenerative disease in a subject comprising administering to the subject. In a particular embodiment, the neurodegenerative disease is selected from Parkinson's disease, Alzheimer's disease, Huntington's disease, amyotrophic lateral sclerosis (ALS), ischemia, stroke, dementia with Lewy bodies, and frontotemporal dementia.

Also an embodiment of the present invention is an inhibitor of USP30 for the treatment of Parkinson's disease in a subject.

In another embodiment of the present invention, the inhibitor of USP30 is administered orally, intramuscularly, intravenously, intraarterially, intraperitoneally, or subcutaneously.

In a particular embodiment of the present invention. the inhibitor of USP30 for the use in a treatment as described herein is combined with at least one additional therapeutic agent. in a further particular embodiment, the at least one additional therapeutic agent is selected from levodopa, a dopamine agonist, a monoamino oxygenase (MAO) B inhibitor, a catechol O-methyltransferase (COMT) inhibitor, an anticholinergic, amantadine, riluzole, a cholinesterase inhibitor, memantine, tetrabenazine, an antipsychotic, clonazepam, diazepam, an antidepressant, and an anti-convulsant.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows immunostaining of HeLa cells cotransfected with GFP-Parkin, and individual FLAG-tagged DUBs. Following 24 hours of expression, cells were treated with CCP (20 μM, 24 h) and immunostained for GFP, FLAG, and endogenous Tom20. Representative images are shown for FLAG-tagged USP30, DUBA2, UCH-L1, USP15 and ATXN3; other DUBs are not shown. Scale bar, 10 μm. FIG. 1B shows immunostaining of SH-SY5Y cells cotransfected with GFP-Parkin and the indicated control (β-Gal) and USP30 constructs. Following 24 hours of expression, cells were treated with CCCP (20 μM, 24 h) and immunostained for myc, FLAG, and endogenous Tom20 and HSP60 (Scale bar, 5 μm). FIG. 1C shows quantification of percent of cells with Tom20 or HSP60 staining from FIG. 1B (***p<0.001 by One-way ANOVA-Dunnett's Multiple Comparison test. N=3 experiments. Error bars represent SEM). FIG. 1D shows quantification of total Tom20 and HSP60 fluorescence intensity per cell from FIG. 1B (**p<0.01 by One-way ANOVA-Dunnett's Multiple Comparison test. n=63, 67 and 54 cells for control (β-Gal), USP30-FLAG and USP30-C77S-FLAG groups, respectively. N=3 experiments. Error bars represent SEM). Figure l E shows quantification of percent of cells containing Parkin clusters from FIG. 1B (***p<0.001 by One-way ANOVA-Dunnett's Multiple Comparison test. N=3 experiments. Error bars represent SEM).

FIG. 2A shows immunostaining of transfected USP30-FLAG (red) and mitochondria-targeted GFP (green) in cultured rat hippocampal neurons. Merge is shown in color; individual channels in gray-scale. Scale bar, 5 μm. FIG. 2B shows immunostaining of SH-Sy5Y cells transfected with control or USP30 siRNA. Following 3 days of knockdown, cells were fixed and immunostained for endogenous USP30 and HSP60. USP30 siRNA primarily decreased mitochondrial USP30 antibody staining (Scale bar, 5 μm). Higher magnification images of the boxed regions are shown on the right panel (Scale bar, 2 μm). FIG. 2C shows immunoblots of cytoplasm- and mitochondria-enriched fractions from rat brain with USP30, HSP60, and GAPDH antibodies. FIG. 2D shows immunostaining of SH-SY5Y cells cotransfected with GFP-Parkin and the indicated control (β-Gal) and USP30 constructs. Following 24 hours of expression, cells were treated with CCCP (20 μM, 4 h) and immunostained for GFP, FLAG, and endogenous Tom20 and polyubiquitin chains (detected with the FK2 antibody) (Scale bar, 5 μm). FIG. 2E is a plot showing the quantification of mitochondria-associated polyubiquitin staining intensity normalized by mitochondrial area from FIG. 2D (integrated fluorescence intensity of mitochondrial FK2 staining/area of Tom20 staining) (***p<0.001 by One-way ANOVA-Dunnett's Multiple Comparison test. n=61, 45 and 59 cells for β-Gal, USP30-FLAG and USP30-C77S-FLAG groups, respectively. Error bars represent SEM). FIG. 2F shows immunoblots of cell lysates from GFP-Parkin expressing stable HEK-293 cells transfected with the indicated control (β-Gal) and USP30 constructs. Following 24 hours of expression, cells were treated with CCCP (5 μM, 2 hours) and lysed. FIG. 2G is a plot showing the quantification of immunoblot signal for GFP-Parkin normalized to actin from FIG. 2F (***p<0.001 by One-way ANOVA-Dunnett's Multiple Comparison test. N=6 experiments. Error bars represent SEM).

FIG. 3A shows that mt-Keima differentially highlights cytoplasmic (green) and lysosomal (red) mitochondria. Cultured hippocampal neurons were transfected with mt-Keima and GFP. Following 2 days of expression, cells were imaged with 458 nm (shown in green) or 543 nm (shown in red) light excitation. GFP signal was used to outline the cell (shown in white). Scale bar, 5 μm. FIG. 3B shows mt-Keima imaging in neurons transfected with Parkin shRNA knockdown constructs. Scale bar, 5 μm. FIG. 3C is a plot showing the quantification of mitophagy index from FIG. 3B (**p<0.01 and ***p<0.001 by One-way ANOVA-Dunnett's Multiple Comparison test. n=52-109 cells per group. N=3-6 experiments. Error bars represent SEM). FIG. 3D shows mt-Keima imaging in neurons transfected with PINK1 shRNA knockdown constructs. Scale bar, 5 μm. FIG. 3E is a plot showing the quantification of mitophagy index from FIG. 3D (**p<0.01 and ***p<0.001 by One-way ANOVA-Dunnett's Multiple Comparison test. n=52-109 cells per group. N=3-6 experiments. Error bars represent SEM). FIG. 3F shows mt-Keima imaging in neurons transfected with PINK1-GFP and Parkin-shRNA#1 (luciferase shRNA and β-Gal as controls). Scale bar, 5 μm. FIG. 3G is a plot showing the quantification of mitophagy index from FIG. 3F (***p<0.001 by One-way ANOVA-Dunnett's Multiple Comparison test. n=55-77 cells. N=3 experiments. Error bars represent SEM).

FIG. 4A shows mt-Keima imaging in cultured hippocampal neurons before and after NH₄Cl treatment (50 mM, 2 minutes). mt-Keima signal collected with 543 nm or 458 nm laser excitation sources are shown in red and green, respectively. Scale bar, 5 μm. FIG. 4B shows imaging of mt-Keima and Lysotracker (shown in gray scale) in hippocampal neurons. Scale bar, 5 μm. FIG. 4C shows post-hoc immunostaining for endogenous LAMP-1 in neurons imaged for mt-Keima signal. Immediately following mt-Keima imaging, cells were fixed and stained with anti-LAMP1 antibody (shown in gray scale). Scale bar, 5 μm. FIG. 4D is a plot showing quantification of mitophagy index following 1, 3 and 6-7 days of mt-Keima expression in cultured hippocampal neurons (*p<0.05 and ***p<0.001 using One-way ANOVA-Bonferroni's Multiple Comparison test. n=56-146 cells. N=6 experiments. Error bars represent SEM). FIG. 4E is an immunoblot of HEK-293 cell lysates transfected with FLAG-Parkin cDNA and Parkin shRNA expression constructs. PSD-95-FLAG was co-transfected as control. FIG. 4F is an immunoblot of HEK-293 cell lysates transfected with PINK1-GFP cDNA and PINK shRNA constructs. PSD-95-FLAG was co-transfected as control. FIG. 4G shows an immunoblot of endogenous Parkin in cultured hippocampal neurons infected with Adeno-associated virus expressing the indicated shRNAs. FIG. 4H shows an immunoblot of endogenous PINK1 in cultured hippocampal neurons infected with Adeno-associated virus expressing the indicated shRNAs. FIG. 4I shows mt-Keima imaging in neurons transfected with GFP-Parkin (or GFP as control). Scale bar, 5 μm. FIG. 4J is a plot showing quantification of mitophagy index from FIG. 4I (p=0.52 by Student's t-test. n=61-67 cells. N=3 experiments. Error bars represent SEM).

FIG. 5A shows mt-Keima imaging in neurons transfected with USP30-FLAG or USP30-C77S-FLAG. Scale bar, 5 μm. FIG. 5B shows immunoblots of HEK-293 cell lysates transfected with the indicated cDNA and shRNA constructs. PSD-95-FLAG was co-transfected as control. FIG. 5C shows an immunoblot of endogenous USP30 in cultured hippocampal neurons infected with Adeno-associated virus particles expressing the USP30 shRNA. FIG. 5D shows mt-Keima imaging in neurons transfected with rat USP30 shRNA and human USP30 cDNA expression constructs (luciferase shRNA and β-Gal as controls). Scale bar, 5 μm. FIG. 5E is a plot showing quantification of mitophagy index from FIG. 5A (***p<0.001 by One-way ANOVA-Bonferroni's Multiple Comparison test. 43-122 cells. N=6 experiments. Error bars represent SEM). FIG. 5F is a plot showing the quantification of mitophagy index from FIG. 5B (**p<0.01 and ***p<0.001 by One-way ANOVA-Dunnett's Multiple Comparison test. n=96-101 cells. N=4 experiments. Error bars represent SEM).

FIG. 6A shows immunoblots of anti-HA-immunoprecipitates for endogenous MIRO and Tom20 in a parental HEK-293 cell line (that lacks GFP-Parkin) transfected with HA-ubiquitin and the indicated constructs. Following 24 hours of expression, cells were treated with CCCP (5 μM, 2 hours) and ubiquitinated proteins were immunoprecipitated with anti-HA beads. Immunoprecipitates and inputs were blotted with the indicated antibodies. FIG. 6B shows immunoblots of anti-HA-immunoprecipitates for endogenous MIRO and Tom20 with USP30 knockdown. GFP-Parkin expressing stable HEK-293 cells were transfected with HA-ubiquitin and the indicated shRNA and cDNA expression constructs. Following 6 days of expression, cells were processed as in FIG. 6A. FIGS. 6C and E show immunoblots of anti-HA-immunoprecipitates for endogenous Miro and Tom20 from cells transfected with the indicated HA-tagged ubiquitin mutants and treated with CCCP (20 μM, 2 hours). FIGS. 6D and F show quantification of immunoblot signals from (C) and (E). Amount of ubiquitination afforded by the ubiquitin mutants are reported relative to wild-type ubiquitin (**p<0.01 and ***p<0.001 compared to ‘wild-type HA-ubiquitin+CCCP’ group, using one-way ANOVA with Dunnett's Multiple Comparison test. 6 denotes ***p<0.001). FIG. 6G shows immunoblots of GFP-Parkin HEK-293 stable cell lysates that were transfected with the indicated FLAG-tagged USP30 constructs and treated with CCCP (5 μM, 1-6 hours). FIG. 6H is a plot showing quantification of immunoblot signals normalized to actin shown in FIG. 6G (*p<0.05, **p<0.01, ***p<0.001 compared to β-Gal control, using Two-way ANOVA with Bonferroni's Multiple Comparison test. Immunoblot signals for all other proteins (VDAC, Mfn-1, Tom70, Hsp60) did not reach significance. N=3-5 experiments).

FIG. 7A shows immunoblots of anti-HA-immunoprecipitates for endogenous MIRO and Tom20 with USP30 overexpression. HEK-293 cells stably expressing GFP-Parkin were transfected with HA-ubiquitin and the indicated constructs. Following 24 hours of expression, cells were treated with CCCP (5 μM, 2 hours) and ubiquitinated proteins were immunoprecipitated with anti-HA beads. Immunoprecipitates and inputs were blotted with the indicated antibodies. FIG. 7B is a plot showing quantification of the immunoblot signal for co-IP′ed MIRO from FIG. 7A. FIG. 7C is a plot showing quantification of the immunoblot signal for co-IP′ed Tom20 from FIG. 7A. Protein levels co-precipitated with anti-HA beads are normalized to ‘β-Gal+CCCP’ group (*p<0.05, **p<0.01 and ***p<0.001 by One-way ANOVA-Dunnett's Multiple Comparison test, compared to β-Gal+CCCP. N=3-5 experiments. Error bars represent SEM). FIG. 7D shows immunoblots of anti-HA immunoprecipitates for endogenous MIRO and Tom20 with USP30 knockdown. GFP-Parkin expressing stable HEK-293 cells were transfected with HA-ubiquitin and the indicated shRNA plasmids. Following 6 days of expression, cells were processed as in FIG. 7A. FIG. 7E is a plot showing quantification of the immunoblot signal for co-IP′ed MIRO from FIG. 7D. FIG. 7F is a plot showing quantification of the immunoblot signal for co-IP′ed Tom20 from FIG. 7D. Protein levels co-precipitated with anti-HA beads is normalized to luciferase shRNA+CCCP′ group (*p<0.05, **p<0.01 and ***p<0.001 by One-way ANOVA-Dunnett's Multiple Comparison test, compared to ‘luciferase shRNA+CCCP’. N=4-6 experiments. Error bars represent SEM).

FIG. 8A shows immunoblots of HA-ubiquitin precipitates from GFP-Parkin HEK-293 cells transfected with the indicated constructs. Following transfection and treatment with CCCP (5 μM, 2 hours), ubiquitinated proteins were immunoprecipitated with anti-HA beads, and precipitates and inputs were immunoblotted with the indicated antibodies. FIG. 8B shows mt-Keima imaging in neurons transfected with Tom20-myc and USP30 constructs (β-Gal as control). Scale bar, 5 μm. FIG. 8C shows mt-Keima imaging in neurons transfected with USP30 shRNA and MIRO cDNA constructs (luciferase RNAi and β-Gal as controls). Scale bar, 5 μm. FIG. 8D is a plot showing the quantification of mitophagy index from FIG. 8B (***p<0.001 by One-way ANOVA-Dunnett's Multiple Comparison test. n=67-80 cells for all groups. N=3 experiments. Error bars represent SEM). FIG. 8E is a plot showing quantification of mitophagy index from FIG. 8C (*p<0.05 and ***p<0.001 by One-way ANOVA-Bonferroni's Multiple Comparison test. n=72-75 cells for all groups. N=3 experiments. Error bars represent SEM).

FIG. 9A shows extracted ion chromatograms corresponding to K-GG peptides identified from Tom20 in the USP30 knockdown experiment. Relative abundance of each ubiquitinated peptide is shown on the y-axis relative to the most abundant analysis, which precursor ion m/z indicated above each peak. The sequence of each K-GG peptide is shown below in green. Asterisks denote modified lysine residues. FIG. 9B shows extracted ion chromatograms corresponding to K-GG peptides identified from USP30 in the Parkin overexpression experiment. The data are presented in a similar manner as in (A). FIG. 9C shows immunoblots of anti-HA-immunoprecipitates for endogenous USP30 from cells transfected with wild-type, K161N and G430D GFP-Parkin constructs. After 24 hours of expression, cells were treated with CCCP (20 μM, 2 hours) and ubiquitinated proteins were immunoprecipitated with anti-HA beads. Immunoprecipitates and inputs were blotted with the indicated antibodies. FIG. 9D shows quantification of immunoblot signal for co-IP′ed USP30 from (C). Protein levels co-precipitating with anti-HA beads are normalized to the ‘wild-type GFP-Parkin+CCCP’ group. (***p<0.001 by One-way ANOVA-Dunnett's Multiple Comparison test, compared to ‘wild-type GFP-Parkin+CCCP’. N=4 experiments. Error bars represent S.E.M.) FIG. 9E shows immunoblots of lysates prepared from HEK-293 cells transfected with the indicated GFP and GFP-Parkin constructs and treated with CCCP (20 μM). FIG. 9F shows quantification of immunoblot signal for USP30 normalized to actin from (E). (**p<0.01, ***p<0.001 compared to wild-type GFP-Parkin, using Two-way ANOVA with Bonferroni's Multiple Comparison test. N=4 experiments. Error bars represent S.E.M.)

FIG. 10A shows immunostaining in GFP-Parkin-G430D expressing stable SH-SY5Y cells transfected with the indicated siRNAs and cDNA expression constructs. Following 3 days of expression, cells were treated with CCCP (20 μM, 24 hours), and fixed and stained for GFP, FLAG, and endogenous Tom20. Scale bar, 5 μm. FIG. 10B is a plot showing quantification of Tom20 fluorescence intensity from FIG. 10A (***p<0.001 by One-way ANOVA-Dunnett's Multiple Comparison test, Error bars represent SEM). FIG. 10C is a plot showing quantification of GFP-Parkin-G430D puncta area from FIG. 10A (***p<0.001 by One-way ANOVA-Dunnett's Multiple Comparison test, Error bars represent SEM). FIG. 10D shows mt-Keima imaging in neurons transfected with Parkin shRNA and USP30-C77A-FLAG. Scale bar, 5 μm. FIG. 10E is a plot showing quantification of mitophagy index from FIG. 10D (***p<0.001 by One-way ANOVA-Dunnett's Multiple Comparison test. N=71-77 cells. N=3 experiments. Error bars represent SEM).

FIG. 11A shows an immunoblot for endogenous USP30 in SH-SY5Y cells transfected with USP30 siRNA for 3 days. FIGS. 11B and 11C show immunostaining in GFP-Parkin-G430D expressing stable SH-SY5Y cells transfected with the indicated siRNAs. Following 3 days of knockdown, cells were treated with CCCP (20 μM, 24 hours), and fixed and stained for GFP and endogenous Tom20. Scale bar, 5 μm. FIG. 11D is a plot showing quantification of fold change in Tom20 staining intensity from FIGS. 11B and 11C normalized to control siRNA (***p<0.001 by One-way ANOVA-Dunnett's Multiple Comparison test. Error bars represent SEM). FIGS. 11E and 11F show immunostaining in GFP-Parkin-G430D (E) and GFP-Parkin-K161N (F) expressing SH-SY5Y cells transfected with USP30 siRNA. Following 3 days of knockdown, cells were treated with CCCP (20 μM, 24 hours), and fixed and stained for GFP and endogenous Tom20 and HSP60. Scale bar, 5 μm. FIGS. 11G and 11H are plots showing quantification of fold change in Tom20 (G) and HSP60 (H) staining intensity from FIGS. 11E and 11F normalized to control siRNA. (*p<0.05, **p<0.01 and ***<0.001 by Student's t-test. N=2-3 experiments. Error bars represent S.E.M.)

FIG. 12A shows transverse sections of indirect flight muscles (IFMs) from wild-type, parkin mutant (park²⁵) and “parkin mutant; dUSP30 knockdown” (park²⁵; Actin-GAL4>UAS-dUSP30^{RNAi}) flies. Electron-dense mitochondria are marked with arrowheads. Mitochondria with reduced and disorganized cristae (hence pale in appearance) are outlined with dashed lines (top panel—Scale bar, 1 μm). Higher magnification images are shown in the lower panels (Scale bar, 0.2 μm). FIGS. 12B and C show quantification of mitochondrial integrity from (A). Percent area of mitochondria containing disorganized cristae over total mitochondrial area (B), and percent of muscle area containing disorganized mitochondria (C) are blindly quantified. (*p<0.05, **p<0.01 and ***p<0.001, compared to wild-type by Two-way ANOVA-Bonferroni's Multiple Comparison test. ***p<0.001 for park²⁵ versus park²⁵; Actin-GAL4>UAS-dUSP30^{RNAi}. 34-55 imaging fields per fly, N=3-4 flies. Error bars represent S.E.M.) FIGS. 12D and E show effect of dUSP30 knockdown and paraquat on climbing assay in Drosophila. Percent of flies climbing >15 cm in 30 seconds, treated with vehicle (5% sucrose) or paraquat (10 mM, 48 hours), for the indicated genotypes. (**p<0.01 and ***p<0.001 by One-Way ANOVA with Bonferroni's multiple comparisons test. N=4-10 experiments. Error bars represent S.E.M.) FIG. 12F shows dopamine neurotransmitter levels per Drosophila head for the indicated genotypes, as determined by ELISA. (*p<0.05 and ***p<0.001 by One-way ANOVA-Bonferroni's Multiple Comparison test. n=28 heads per genotype. N=4 experiments. Error bars represent S.E.M.). FIGS. 12G and H show effect of dUSP30 knockdown and paraquat on survival in Drosophila. Percent of flies still alive, treated with vehicle or paraquat (10 mM, up to 96 hours), for the indicated genotypes. (**p<0.01 and ***p<0.001 using Two-Way ANOVA with Bonferroni's multiple comparisons test. N=3 (G) and 4 (H) experiments. Error bars represent S.E.M.)

FIG. 13A and FIG. 13B shows asymmetric “volcano plot” demonstrating the subset of 41 proteins whose ubiquitination significantly increased (p<0.05) for the “Combo” treatment versus CCCP-treatment alone in both USP30 knockdown (left side) and GFP-Parkin overexpression (right side) experiments. “Combo” refers to cells treated with CCCP and expressing USP30-shRNA, or treated with CCCP and expressing GFP-Parkin, in the two experiments, respectively. For this subset of proteins, fold-increase in ubiquitination (x-axis) and the p-value (y-axis) are reported. Mitochondrial proteins (identified based on the Human MitoCarta database) are shown in red.

FIG. 14 shows inhibition of various peptidases, including USP30, by inhibitory peptides USP30_{—}3 (“pep3”; SEQ ID NO: 1) and USP30_{—}8 (“pep8”; SEQ ID NO: 2), as described in Example 10.

FIG. 15A and FIG. 15B shows a graph of residue probability by peptide position for USP30_{—}3 and certain affinity-matured peptides, along with the signal to noise ratio (“S/N”), ELISA signal (“signal”), number of clones for each sequence (“n”), total number of clones (“total”), and the number of unique sequences (“Uniq”), as described in Example 10.

FIG. 16 shows a graph of signal to noise ratio for USP30_{—}3 and three affinity matured peptides, as described in Example 10. For each peptide, the targets tested were, from left to right, USP2, USP7, USP14, USP30, UCHL1, UCHL3, and UCHL5. The sequences for each peptide are shown below.

FIG. 17A shows ratiometric mito-roGFP imaging in hippocampal neurons transfected with USP30 shRNA. The “relative oxidation index” was shown in a ‘color scale’ from 0 (mito-roGFP ratio after DTT treatment, 1 mM, shown in black) to 1 (mito-roGFP ratio after aldrithiol treatment, 100 μM, shown in red). FIG. 17B is a plot showing quantification of relative oxidation from FIG. 17A (***p<0.001 by Student's t-test. n=24 cells for luciferase shRNA and 36 cells for USP30 shRNA. N=3 experiments. Error bars represent SEM). FIG. 17C shows quantitative RT-PCR of dUSP30 mRNA. qRT-PCR in Actin-GALA UAS-dUSP30^{RNAi} and Actin-GAL4>UAS-dUSP30^{RNAi} flies, expressed relative to Actin-GAL4. dUSP30 mRNA levels were normalized to Drosophila RpII140 mRNA levels in each group. N=7 experiments. ***p<0.001 by One-Way ANOVA with Bonferroni's multiple comparisons test. FIG. 17D shows climbing assay in control flies (Actin-GAL4). Flies were treated with vehicle control (5% sucrose) or paraquat (10 mM, 48 hours). L-DOPA (1 mM, 48 hours) was administered simultaneously with paraquat, as indicated. (***p<0.001 by One-Way ANOVA-Dunnett's Multiple Comparison test. N=6 experiments. Error bars represent S.E.M.). FIG. 17E shows serotonin levels per fly head, as assessed by ELISA. Flies were treated with paraquat (10 mM, 48 hours) or vehicle control (5% sucrose). (p-values calculated by One-Way ANOVA-Bonferroni's Multiple Comparison test. n=8 heads, N=2 experiments. Error bars represent S.E.M.). FIGS. 17F and G show quantitative RT-PCR measurement of (F) dUSP47 and (G) dYOD1 m RNA levels in flies of the indicated genotypes, expressed as relative to Actin-GAL4 genotype. TaqMan assays Dm01795269_g1 (Drosophila CG5486 (USP47)) and Dm01840115_s1 (Drosophila CG4603 (YOD1)) were used. Dm02134593_g1 (RpII140) was used for normalization. (p**<0.01 and p***<0.001 using One-Way ANOVA-Dunnett's Multiple Comparison test. N=3 replicates. Error bars represent S.E.M.) FIGS. 17H and I show survival curves of flies of the indicated genotype, treated with vehicle or paraquat (10 mM). Graph shows percent flies alive at indicated times after feeding with paraquat. (*p<0.05, p**<0.01, and p***<0.001 using Two-Way ANOVA with Bonferroni's Multiple Comparisons test. N=5 (H) and 4 (1) experiments. Error bars represent S.E.M.)

### DETAILED DESCRIPTION

The present inventors have identified USP30, a mitochondria-localized deubiquitinase (DUB) as an atagonist of Parkin-mediated mitophagy. USP30, through its deubiquitinase activity, counteracts ubiquitination and degradation of damaged mitochondria, and inhibition of USP30 rescues mitophagy defects caused by mutant Parkin. Further, USP30 inhibition of USP30 decreases oxidative stress and provides protection against the mitochondrial toxin, rotenone. Since damaged mitochondria are more likely to accumulate Parkin, USP30 inhibition should preferentially clear unhealthy mitochondria. In addition to neurons (such as substantia nigra neurons, which are especially vulnerable to mitochondria dysfunction in Parkinson's disease), long-lived metabolically active cells such as cardiomyocytes also rely on an efficient mitochondria quality control system. In this context, Parkin has been shown to protect cardiomyocytes against ischemia/reperfusion injury through activating mitophagy and clearing damaged mitochondria in response to ischemic stress. Thus, inhibitors of USP30 are provided for us in treating a conditions involving mitochondrial defects, including neurological conditions, cardiac conditions, and systemic conditions.

### I. DEFINITIONS

An “inhibitor” refers to an agent capable of blocking, neutralizing, inhibiting, abrogating, reducing and/or interfering with one or more of the activities of a target and/or reducing the expression of the target protein (or the expression of nucleic acids encoding the target protein) Inhibitors include, but are not limited to, antibodies, polypeptides, peptides, nucleic acid molecules, short interfering RNAs (siRNAs) and other inhibitory RNAs, small molecules (e.g., small inorganic molecules), polysaccharides, polynucleotides, antisense oligonucleotides, aptamers, and peptibodies. An inhibitor may decrease the activity and/or expression of a target protein by at least 10% (e.g., by at least 15%, 20%, 25%, 30%, 35%, 40%, 45%, 50%, 55%, 60%, 65%, 70%, 75%, 80%, 85%, 90%, or even 100% decrease) as compared to the expression and/or activity of the target protein that is untreated with the inhibitor.

An “inhibitor of USP30” refers to an agent capable of blocking, neutralizing, inhibiting, abrogating, reducing and/or interfering with one or more of the activities of USP30 and/or reducing the expression of USP30 (or the expression of nucleic acids encoding USP30). In some embodiments, an inhibitor of USP30 reduces the deubiquitinase activity of USP30. In some embodiments, an inhibitor of USP30 reduces deubiquitinase activity by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100%. Deubiquitinase activity may be reduced by an inhibitor by any mechanism, including, but not limited to, interfering with the active site of USP30, interfering with target recognition, altering the conformation of USP30, interfering with proper subcellular localization of USP30, etc. In some embodiments, an inhibitor of USP30 inhibits USP30 expression, which may be expression as the mRNA (e.g., it inhibits transcription of the USP30 gene to produce USP30 mRNA) and/or protein level (e.g., it inhibits translation of the USP30 mRNA to produce USP30 protein). In some embodiments, an inhibitor of USP30 expression reduces the level of USP30 protein by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or 100%.

The terms “mitophagy” and “mitochondrial degradation” are used interchangeably to refer to the regulated degradation of mitochondria through the lysosomal machinery of a cell.

A “condition involving a mitochondrial defect” refers to a condition involving a defect or defects in mitochondrial function, mitochondrial shape/morphology, mitochondrial membrane potential, and/or mitophagy in a cell population. Conditions involving a mitochondrial defect include, but are not limited to, conditions involving a defect in mitophagy, such that mitophagy occurs in the cell population at a slower rate or to a lesser extent than in a normal cell population. In some embodiments, the defect in mitophagy is accompanied by other mitochondrial defects such that the decreased mitophagy results in the increased presence of defective mitochondria. Conditions involving a mitochondrial defect also include, but are not limited to, conditions involving mutations in mitochondrial DNA that result in altered mitochondrial function. Conditions involving a mitochondrial defect also include conditions involving mitochondrial oxidative stress, in which increased levels of reactive oxygen species (ROS) and/or reactive nitrogen species (RNS) in a cell are associated with protein aggregation and/or mitochondrial dysfunction. Mitochondrial oxidative stress may result in mitochondrial dysfunction, or mitochondrial dysfunction may result in oxidative stress. Conditions involving a mitochondrial defect also include, but are not limited to, conditions involving defects in mitochondrial shape/morphology and conditions involving defects in mitochondrial membrane potential. Exemplary conditions involving mitochondrial defects include, but are not limited to, neurodegenerative diseases (such as Parkinson's disease, Huntington's disease, amyotrophic lateral sclerosis (ALS), Alzheimer's disease, ischemia, stroke, dementia with Lewy bodies, and frontotemporal dementia); mitochondrial myopathy, encephalopathy, lactic acidosis, and stroke-like episodes (MELAS) syndrome; Leber's hereditary optic neuropathy (LHON); neuropathy, ataxia, retinitis pigmentosa-maternally inherited Leigh syndrome (NARP-MILS); Danon disease; myoclonic epilepsy with ragged red fibers (MERFF) syndrome; ischemic heart disease leading to myocardial infarction; multiple sulfatase deficiency (MSD); mucolipidosis II (ML II); mucolipidosis III (ML III); mucolipidosis IV (ML IV); GM1-gangliosidosis (GM1); neuronal ceroid-lipofuscinoses (NCL1); Alpers disease; Barth syndrome; Beta-oxidation defects; carnitine-acyl-carnitine deficiency; carnitine deficiency; creatine deficiency syndromes; co-enzyme Q10 deficiency; complex I deficiency; complex II deficiency; complex III deficiency; complex IV deficiency; complex V deficiency; COX deficiency; chronic progressive external ophthalmoplegia syndrome (CPEO); CPT I deficiency; CPT II deficiency; glutaric aciduria type II; Kearns-Sayre syndrome; lactic acidosis; long-chain acyl-CoA dehydrongenase deficiency (LCHAD); Leigh disease or syndrome; lethal infantile cardiomyopathy (LIC); Luft disease; glutaric aciduria type II; medium-chain acyl-CoA dehydrongenase deficiency (MCAD); myoclonic epilepsy and ragged-red fiber (MERRF) syndrome; mitochondrial recessive ataxia syndrome; mitochondrial cytopathy; mitochondrial DNA depletion syndrome; myoneurogastointestinal disorder and encephalopathy; Pearson syndrome; pyruvate carboxylase deficiency; pyruvate dehydrogenase deficiency; POLG mutations; medium/short-chain 3-hydroxyacyl-CoA dehydrogenase (M/SCHAD) deficiency; and very long-chain acyl-CoA dehydrongenase (VLCAD) deficiency.

A “pathogenic mutation” in Parkin or PINK1 refers to a mutation or mutations in the respective protein or gene that results in reduced activity in a cell, and may involve loss of function and/or gain of function (such as dominant negative mutations, for example, Parkin Q311stop). Such reduced activity in a cell may include, but is not limited to, reduced enzymatic activity (such as reduced ubiquitination or kinase activity), reduced activity due to the presence of a dominant negative mutant protein, reduced binding to another cellular factor, reduced activity due to subcellular localization changes, and/or reduced activity due to reduced levels of protein in the cell or in a cellular compartment. In some embodiments, a pathogenic mutation in Parkin and/or PINK1 results in reduced ubiquitination of mitochondria, which may result in reduced mitophagy. Pathogenic mutations may also occur outside of the coding region of the protein, e.g., in an intron (affecting, for example, splicing), the promoter, the 5′ untranslated region, the 3′ untranslated region, etc. Further, Parkin mutations may involve substitutions, deletions, insertions, duplications, etc., or any combination of those. Nonlimiting exemplary pathogenic mutations in Parkin are shown in Table 1. Nonlimiting exemplary pathogenic mutations in PINK1 protein are shown in Table 2. Databases of Parkinson's disease mutations are publicly available, such as Parkinson Disease Mutation Database, http://www.molgen.ua.ac.be/PDmutDB/.

TABLE 1
Exemplary pathogenic mutations in Parkin (PARK2)

| | | | |
|---|---|---|---|
| Ala291fs | ex10del | ex4-7del | Gln311Stop |
| Ala31Asp | ex10dup | ex4del | Gln34fs |
| Ala398Thr | ex11del | ex4dup | Gln34fs |
| Arg234Gln | ex11dup | ex5-12del | Gln40Stop |
| Arg334Cys | ex12dup | ex5-6del | Glu395Stop |
| Arg33Gln | ex1-4del | ex5-7del | Glu409Stop |
| Arg33Stop | ex1del | ex5-8dup | Glu444Gln |
| Arg348fs | ex1dup | ex5-9dup | Glu79Stop |
| Arg366Trp | ex2-3del | ex5del | Gly179fs |
| Arg392fs | ex2-3dup | ex5dup | Gly328Glu |
| Arg42His | ex2-4del | ex6-7del | Gly359Asp |
| Arg42Pro | ex2-4dup | ex6-8dup | Gly429Glu |
| Asn428fs | ex2-4trip | ex6del | Gly430Asp |
| Asn52fs | ex2-5del | ex6dup | IVS1 + 1G > A |
| Asp280Asn | ex2del | ex7-8del | IVS11 − 3C > G |
| Asp460fs | ex2dup | ex7-9del | Leu283Pro |
| Asp53Stop | ex2trip | ex7del | Lys161Asn |
| c. − 39G > T | ex3-4del | ex7dup | Lys211Asn |
| Cys212Gly | ex3-4dup | ex8-10del | Lys349fs |
| Cys212Tyr | ex3-5del | ex8-11del | Met192Leu |
| Cys238fs | ex3-6del | ex8-9del | Met192Val |
| Cys268Stop | ex3-7del | ex8del | Met1Leu |
| Cys289Gly | ex3-9del | ex8dup | partial ex4del |
| Cys323fs | ex3del | ex9del | Pro113fs/ex3 Δ40 bp |
| Cys431Phe | ex3dup | ex9dup | Pro133del |
| ex10-12del | ex4-5del | Gln171Stop | Thr240Arg |
| ex10-12dup | ex4-6del | Gln311His | Thr240Met |
| Val56Glu | Val258Met | Trp453Stop | Thr351Pro |
| prom + ex1del | Val324fs | Trp74fs | Thr415Asn |

del = deletion; dup = duplication; fs = frameshift; ex = exon; IVS = intervening sequence; prom = promoter

TABLE 2
Exemplary pathogenic mutations in PINK1

| | | | |
|---|---|---|---|
| Tyr258Stop | IVS7 + 1G > A | ex6-8del | Asp297fs |
| Trp437Stop | Gly440Glu | ex4-8del | Arg492Stop |
| Thr313Met | Glu239Stop | ex3-8del | Arg464His |
| Stop582Leu | Gln456Stop | delPINK1 | Arg246Stop |
| Pro196fs | Gln129Stop | Cys92Phe | Ala168Pro |
| Lys520fs | Gln129fs | Cys549fs | 23 bp del ex7 |
| Lys24fs | ex7del | Asp525fs | |

del = deletion; fs = frameshift; ex = exon

The term “oxidative stress” refers to an increase in reactive oxygen species (ROS) and/or reactive nitrogen species (RNS) in a cell. In some embodiments, oxidative stress leads to protein aggregation and/or mitochondrial dysfunction. In some embodiments, mitochondrial dysfunction leads to oxidative stress.

The term “USP30,” as used herein, refers to any native USP30 (“ubiquitin specific peptidase 30” or “ubiquitin specific protease 30”) from any vertebrate source, including mammals such as primates (e.g. humans) and rodents (e.g., mice and rats), unless otherwise indicated. The term encompasses “full-length,” unprocessed USP30 as well as any form of USP30 that results from processing in the cell. The term also encompasses naturally occurring variants of USP30, e.g., splice variants or allelic variants. The amino acid sequence of an exemplary human USP30 is shown in SEQ ID NO: 26 (Table 4).

The term “Parkin” as used herein, refers to any native Parkin from any vertebrate source, including mammals such as primates (e.g. humans) and rodents (e.g., mice and rats), unless otherwise indicated. The term encompasses “full-length,” unprocessed Parkin as well as any form of Parkin that results from processing in the cell. The term also encompasses naturally occurring variants of Parkin, e.g., splice variants or allelic variants. The amino acid sequence of an exemplary human Parkin is shown in SEQ ID NO: 29 (Table 4).

The term “PINK1” as used herein, refers to any native PINK1 (PTEN-induced putative kinase protein 1) from any vertebrate source, including mammals such as primates (e.g. humans) and rodents (e.g., mice and rats), unless otherwise indicated. The term encompasses “full-length,” unprocessed PINK1 as well as any form of PINK1 that results from processing in the cell. The term also encompasses naturally occurring variants of PINK1, e.g., splice variants or allelic variants. The amino acid sequence of an exemplary human PINK1 is shown in SEQ ID NO: 30 (Table 4).

The term “Tom20” as used herein, refers to any native Tom20 from any vertebrate source, including mammals such as primates (e.g. humans) and rodents (e.g., mice and rats), unless otherwise indicated. The term encompasses “full-length,” unprocessed Tom20 as well as any form of Tom20 that results from processing in the cell. The term also encompasses naturally occurring variants of Tom20, e.g., splice variants or allelic variants. The amino acid sequence of an exemplary human Tom20 is shown in SEQ ID NO: 27 (Table 4).

The terms “MIRO1” and “MIRO” as used herein, refer to any native MIRO1 (mitochondrial Rho GTPase 1) from any vertebrate source, including mammals such as primates (e.g. humans) and rodents (e.g., mice and rats), unless otherwise indicated. The term encompasses “full-length,” unprocessed MIRO1 as well as any form of MIRO1 that results from processing in the cell. The term also encompasses naturally occurring variants of MIRO1, e.g., splice variants or allelic variants. The amino acid sequence of an exemplary human MIRO1 is shown in SEQ ID NO: 28 (Table 4).

The term “MUL1” as used herein, refers to any native MUL1 (mitochondrial ubiquitin ligase activator of NFκB) from any vertebrate source, including mammals such as primates (e.g. humans) and rodents (e.g., mice and rats), unless otherwise indicated. The term encompasses “full-length,” unprocessed MUL1 as well as any form of MUL1 that results from processing in the cell. The term also encompasses naturally occurring variants of MUL1, e.g., splice variants or allelic variants. The amino acid sequence of an exemplary human MUL1 is shown in SEQ ID NO: 32 (Table 4).

The term “ASNS” as used herein, refers to any native ASNS (asparagine synthetase [glutamine hydrolyzing]) from any vertebrate source, including mammals such as primates (e.g. humans) and rodents (e.g., mice and rats), unless otherwise indicated. The term encompasses “full-length,” unprocessed ASNS as well as any form of ASNS that results from processing in the cell. The term also encompasses naturally occurring variants of ASNS, e.g., splice variants or allelic variants. The amino acid sequence of an exemplary human ASNS is shown in SEQ ID NO: 33 (Table 4).

The term “FKBP8” as used herein, refers to any native FKBP8 (FK506 binding protein 8) from any vertebrate source, including mammals such as primates (e.g. humans) and rodents (e.g., mice and rats), unless otherwise indicated. The term encompasses “full-length,” unprocessed ASNS as well as any form of FKBP8 that results from processing in the cell. The term also encompasses naturally occurring variants of FKBP8, e.g., splice variants or allelic variants. The amino acid sequence of an exemplary human FKBP8 is shown in SEQ ID NO: 34 (Table 4).

The term “TOM70” as used herein, refers to any native TOM70 (translocase of outer membrane 70 kDa subunit) from any vertebrate source, including mammals such as primates (e.g. humans) and rodents (e.g., mice and rats), unless otherwise indicated. The term encompasses “full-length,” unprocessed TOM70 as well as any form of TOM70 that results from processing in the cell. The term also encompasses naturally occurring variants of TOM70, e.g., splice variants or allelic variants. The amino acid sequence of an exemplary human TOM70 is shown in SEQ ID NO: 35 (Table 4).

The term “MAT2B” as used herein, refers to any native MAT2B (methionine adenosyltransferase 2 subunit beta) from any vertebrate source, including mammals such as primates (e.g. humans) and rodents (e.g., mice and rats), unless otherwise indicated. The term encompasses “full-length,” unprocessed MAT2B as well as any form of MAT2B that results from processing in the cell. The term also encompasses naturally occurring variants of MAT2B, e.g., splice variants or allelic variants. The amino acid sequence of an exemplary human MAT2B is shown in SEQ ID NO: 36 (Table 4).

The term “PRDX3” as used herein, refers to any native PRDX3 (peroxiredoxin III) from any vertebrate source, including mammals such as primates (e.g. humans) and rodents (e.g., mice and rats), unless otherwise indicated. The term encompasses “full-length,” unprocessed PRDX3 as well as any form of PRDX3 that results from processing in the cell. The term also encompasses naturally occurring variants of PRDX3, e.g., splice variants or allelic variants. The amino acid sequence of an exemplary human PRDX3 is shown in SEQ ID NO: 37 (Table 4).

The term “IDE” as used herein, refers to any native IDE (insulin degrading enzyme) from any vertebrate source, including mammals such as primates (e.g. humans) and rodents (e.g., mice and rats), unless otherwise indicated. The term encompasses “full-length,” unprocessed IDE as well as any form of IDE that results from processing in the cell. The term also encompasses naturally occurring variants of IDE, e.g., splice variants or allelic variants. The amino acid sequence of an exemplary human IDE is shown in SEQ ID NO: 38 (Table 4).

The term “VDAC1” as used herein, refers to any native VDAC1 (voltage-dependent anion selective channel protein 1) from any vertebrate source, including mammals such as primates (e.g. humans) and rodents (e.g., mice and rats), unless otherwise indicated. The term encompasses “full-length,” unprocessed VDAC1 as well as any form of VDAC1 that results from processing in the cell. The term also encompasses naturally occurring variants of VDAC1, e.g., splice variants or allelic variants. The amino acid sequence of an exemplary human VDAC1 is shown in SEQ ID NO: 39 (Table 4).

The term “VDAC2” as used herein, refers to any native VDAC2 (voltage-dependent anion selective channel protein 2) from any vertebrate source, including mammals such as primates (e.g. humans) and rodents (e.g., mice and rats), unless otherwise indicated. The term encompasses “full-length,” unprocessed VDAC2 as well as any form of VDAC2 that results from processing in the cell. The term also encompasses naturally occurring variants of VDAC2, e.g., splice variants or allelic variants. The amino acid sequence of an exemplary human VDAC2 is shown in SEQ ID NO: 44 (Table 4).

The term “VDAC3” as used herein, refers to any native VDAC3 (voltage-dependent anion selective channel protein 3) from any vertebrate source, including mammals such as primates (e.g. humans) and rodents (e.g., mice and rats), unless otherwise indicated. The term encompasses “full-length,” unprocessed VDAC3 as well as any form of VDAC3 that results from processing in the cell. The term also encompasses naturally occurring variants of VDAC3, e.g., splice variants or allelic variants. The amino acid sequence of an exemplary human VDAC3 is shown in SEQ ID NO: 45 (Table 4).

The term “IP05” as used herein, refers to any native IP05 (importin 5) from any vertebrate source, including mammals such as primates (e.g. humans) and rodents (e.g., mice and rats), unless otherwise indicated. The term encompasses “full-length,” unprocessed IP05 as well as any form of IP05 that results from processing in the cell. The term also encompasses naturally occurring variants of IP05, e.g., splice variants or allelic variants. The amino acid sequence of an exemplary human IP05 is shown in SEQ ID NO: 40 (Table 4).

The term “PTH2” as used herein, refers to any native PTH2 (peptidyl-tRNA hydrolase 2, mitochondrial) from any vertebrate source, including mammals such as primates (e.g. humans) and rodents (e.g., mice and rats), unless otherwise indicated. The term encompasses “full-length,” unprocessed PTH2 as well as any form of PTH2 that results from processing in the cell. The term also encompasses naturally occurring variants of PTH2, e.g., splice variants or allelic variants. The amino acid sequence of an exemplary human PTH2 is shown in SEQ ID NO: 41 (Table 4).

The term “PSD13” as used herein, refers to any native PSD13 (26S proteasome non-ATPase regulatory subunit 13) from any vertebrate source, including mammals such as primates (e.g. humans) and rodents (e.g., mice and rats), unless otherwise indicated. The term encompasses “full-length,” unprocessed PSD13 as well as any form of PSD13 that results from processing in the cell. The term also encompasses naturally occurring variants of PSD13, e.g., splice variants or allelic variants. The amino acid sequence of an exemplary human PSD13 is shown in SEQ ID NO: 42 (Table 4).

The term “UBP13” as used herein, refers to any native UBP13 (ubiquitin carboxyl-terminal hydrolase 13) from any vertebrate source, including mammals such as primates (e.g. humans) and rodents (e.g., mice and rats), unless otherwise indicated. The term encompasses “full-length,” unprocessed UBP13 as well as any form of UBP13 that results from processing in the cell. The term also encompasses naturally occurring variants of UBP13, e.g., splice variants or allelic variants. The amino acid sequence of an exemplary human UBP13 is shown in SEQ ID NO: 43 (Table 4).

The term “package insert” is used to refer to instructions customarily included in commercial packages of therapeutic products, that contain information about the indications, usage, dosage, administration, combination therapy, contraindications and/or warnings concerning the use of such therapeutic products.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

Administration “in combination with” one or more further therapeutic agents includes simultaneous (concurrent) and consecutive administration, in any order.

An “effective amount” of an agent, e.g., a pharmaceutical formulation, refers to an amount effective, at dosages and for periods of time necessary, to achieve the desired therapeutic or prophylactic result.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### II. COMPOSITIONS AND METHODS

In various aspects, the invention is based, in part, on inhibitors of USP30 and methods of treating diseases and disorders comprising inhibiting USP30.

A. Exemplary Inhibitors of USP30

The present invention is based in part on the discovery that inhibitors of USP30 activity and/or expression are effective for increasing and/or restoring mitochondrial ubiquitination and mitophagy. In some embodiments, inhibitors of USP30 are effective for treating neurodegenerative diseases, such as Parkinson's disease, as well as conditions that involve mitochondrial defects, such as those involving mitophagy defects, mutations in mitochondrial DNA, mitochondrial oxidative stress, and/or lysosomal storage defects.

Inhibitors of USP30 include inhibitors of USP30 activity and inhibitors of USP30 expression. Nonlimiting exemplary such inhibitors include antisense oligonucleotides, short interfering RNAs (siRNAs), antibodies, peptides, peptibodies, aptamers, and small molecules. In some embodiments, antisense oligonucleotides or short interfering RNAs (siRNAs) may be used to inhibit USP30 expression. In some embodiments, antibodies, peptides, peptibodies, aptamers, and small molecules may be used to inhibit USP30 activity. Some nonlimiting examples of inhibitors of USP30 are described herein. Further inhibitors can be identified using standard methods in the art, including those discussed herein.

Antisense Oligonucleotides

In some embodiments, antisense oligonucleotides that hybridize to USP30 mRNA and/or USP30 pre-mRNA are provided. A nonlimiting exemplary human mRNA sequence encoding USP30 is shown in SEQ ID NO: 30 (Table 4). In some embodiments, an antisense oligonucleotide hybridizes to a region of USP30 mRNA and/or USP30 pre-mRNA and directs its degradation through RNase H, which cleaves double-stranded RNA/DNA hybrids. By mediating cleavage of USP30 mRNA and/or USP30 pre-mRNA, an antisense oligonucleotide may reduce the amount of USP30 protein in a cell (i.e., may inhibit expression of USP30). In some embodiments, an antisense oligonucleotide does not mediate degradation through RNase H, but rather “blocks” translation of the mRNA, e.g., through interference with translational machinery binding or processivity, or “blocks” proper splicing of the pre-mRNA, e.g., through interference with the splicing machinery and/or accessibility of a splice site. In some embodiments, an antisense oligonucleotide may mediate degradation of an mRNA and/or pre-mRNA through a mechanism other than RNase H Any inhibitory mechanism of an antisense oligonucleotide is contemplated herein.

In some embodiments, an antisense oligonucleotide is 10 to 500 nucleotides long, or 10 to 400 nucleotides long, or 10 to 300 nucleotides long, or 10 to 200 nucleotides long, or 10 to 100 nucleotides long, or 15 to 100 nucleotides long, or 10 to 50 nucleotides long, or 15 to 50 nucleotides long. In various embodiments, an antisense oligonucleotide hybridizes to a region of the USP30 mRNA and/or pre-mRNA comprising at least 10, at least 15, at least 20, at least 25, at least 30, at least 40, at least 50, at least 60, at least 70, at least 80, at least 90, or at least 100 nucleotides. Further, in various embodiments, an antisense oligonucleotide need not be 100% complementary to a region USP30 mRNA and/or a region of USP30 pre-mRNA, but may have 1 or more mismatches. Thus, in some embodiments, an antisense oligonucleotide is at least 80% complementary, at least 85% complementary, at least 90% complementary, at least 95% complementary, or 100% complementary to a region of USP30 mRNA and/or a region of USP30 pre-mRNA. In some embodiments, the region of USP30 mRNA or the region of USP pre-mRNA is at least at least 10, at least 15, at least 20, at least 25, at least 30, at least 40, at least 50, at least 60, at least 70, at least 80, at least 90, or at least 100 nucleotides long.

Antisense oligonucleotides may comprise modifications to one or more of the internucleoside linkages, sugar moieties, and/or nucleobases. Further, the sequence of nucleotides may be interrupted by non-nucleotide components, and/or non-nucleotide components may be attached at one or both ends of the oligonucleotide.

Nonlimiting exemplary nucleotide modifications include sugar modifications, in which any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Oligonucleotides can also contain analogous forms of ribose or deoxyribose sugars that are generally known in the art, including, for example, 2′-O-methyl-2′-0-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by modified internucleoside linkages. These modified internucleoside linkages include, but are not limited to, embodiments wherein phosphate is replaced by P(O)S (“thioate”), P(S)S (“dithioate”), (O)NR₂ (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all oligonucleotides referred to herein, including antisense oligonucleotides and siRNA.

In some embodiments, one or more internucleoside linkages in an antisense oligonucleotide are phosphorothioates. In some embodiments, one or more sugar moieties in an antisense oligonucleotide comprise 2′ modifications, such as 2′-O-alkyl (such as 2′-OMe) and 2′-fluoro; or are bicyclic sugar moieties (such as LNA). Nonlimiting exemplary nucleobase modifications include 5-methylcytosine. An antisense oligonucleotide may comprise more than one type of modification within a single oligonucleotide. That is, as a nonlimiting example, an antisense oligonucleotide may comprise 2′-O alkyl modifications, bicyclic nucleotides, and phosphorothioate linkages in the same oligonucleotide. In some embodiments, an antisense oligonucleotide is a “gapmer.” Gapmers comprise a central region of deoxyribonucleotides for mediating RNase H cleavage, and 5′ and 3′ “wings” comprising modified sugar moieties that increase the stability of the duplex.

Antisense oligonucleotide design and mechanisms are described, e.g., in van Roon-Mom et al., Methods Mol. Biol., 867: 79-96 (20120); Prakash, Chem. Biodivers., 8: 1616-1641 (2011); Yamamoto et al., Future Med. Chem., 3: 339-365 (2011); Chan et al., Clin. Exper. Pharmacol. Physiol., 33: 533-540 (2006); Kurreck et al., Nucl. Acids Res., 30: 1911-1918 (2002); Kurreck, Eur. J. Biochem., 270: 1628-1644 (2003); Geary, Expert Opin. Drug Metab. Toxicol., 5: 381-391 (2009); “Designing Antisense Oligonucleotides,” available online from Integrated DNA Technologies (2011).

Short Interfering RNAs (siRNAs)

In some embodiments, the expression of USP30 is inhibited with a short interfering RNA (siRNA). As used herein, siRNAs are synonymous with double-stranded RNA (dsRNA) and include double-stranded RNA oligomers with or without hairpin structures at each end (also referred to as small hairpin RNA, or shRNA). Short interfering RNAs are also known as small interfering RNAs, silencing RNAs, short inhibitory RNA, and/or small inhibitory RNAs, and these terms are considered to be equivalent herein.

The term “short-interfering RNA (siRNA)” refers to small double-stranded RNAs that interfere with gene expression. siRNAs are mediators of RNA interference, the process by which double-stranded RNA silences homologous genes. In some embodiments, siRNAs are comprised of two single-stranded RNAs of about 15-25 nucleotides in length that form a duplex, which may include single-stranded overhang(s). In some embodiments, siRNAs are comprised of a single RNA that forms a hairpin structure that includes a double-stranded portion that may be 15-25 nucleotides in length and may include a single-stranded overhang. Such hairpin siRNAs may be referred to as a short hairpin RNA (shRNA). Processing of the double-stranded RNA by an enzymatic complex, for example, polymerases, may result in cleavage of the double-stranded RNA to produce siRNAs. The antisense strand of the siRNA is used by an RNA interference (RNAi) silencing complex to guide mRNA cleavage, thereby promoting mRNA degradation. To silence a specific gene using siRNAs, for example, in a mammalian cell, a base pairing region is selected to avoid chance complementarity to an unrelated mRNA. RNAi silencing complexes have been identified in the art, such as, for example, by Fire et al., Nature 391:806-811, 1998, and McManus et al., Nat. Rev. Genet. 3(10):737-747, 2002.

In some embodiments, small interfering RNAs comprise at least about 10 to about 200 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides, at least about 130 nucleotides, at least about 140 nucleotides, at least about 150 nucleotides, or greater than 150 nucleotides. In some embodiments, an siRNA is 10 to 200 nucleotides long, or 10 to 100 nucleotides long, or 15 to 100 nucleotides long, or 10 to 60 nucleotides long, or 15 to 60 nucleotides long, or 10 to 50 nucleotides long, or 15 to 50 nucleotides long, or 10 to 30 nucleotides long, or 15 to 30 nucleotides long. In certain embodiments, the siRNA comprises an oligonucleotide from about 21 to about 25 nucleotides in length. In some embodiments, the siRNA molecule is a heteroduplex of RNA and DNA.

As with antisense oligonucleotides, siRNAs can include modifications to the sugar, internucleoside linkages, and/or nucleobases. Nonlimiting exemplary modifications suitable for use in siRNAs are described herein and also, e.g., in Peacock et al., J. Org. Chem., 76: 7295-7300 (2011); Bramsen et al., Methods Mol. Biol., 721: 77-103 (2011); Pasternak et al., Org. Biomol. Chem., 9: 3591-3597 (2011); Gaglione et al., Mini Rev. Med. Chem., 10: 578-595 (2010); Chernolovskaya et al., Curr. Opin. Mol. Ther., 12: 158-167 (2010).

A process for inhibiting expression of USP30 in a cell comprises introduction of an siRNA with partial or fully double-stranded character into the cell. In some embodiments, an siRNA comprises a nucleotide sequence that is at least 75%, at least 80%, at least 85%, at least 90%, at least 95%, or 100% identical to a nucleotide sequence found in the USP30 gene coding region or pre-mRNA.

In some embodiments, an siRNA specific to the USP30 gene is synthesized and introduced directly into a subject. In other embodiments, the siRNA can be formulated as part of a targeted delivery system, such as a target specific liposome, which specifically recognizes and delivers the siRNA to an appropriate tissue or cell type. Upon administration of the targeted siRNA to a subject, the siRNA is delivered to the appropriate cell type, thereby increasing the concentration siRNA within the cell type. Depending on the dose of siRNA delivered, this process can provide partial or complete loss of USP30 protein expression.

In other embodiments, an appropriate cell or tissue is provided with an expression construct that comprises a nucleic acid encoding one or both strands of an siRNA that is specific to the USP30 gene. In these embodiments, the nucleic acid that encodes one or both strands of the siRNA can be placed under the control of either a constitutive or a regulatable promoter. In some embodiments, the nucleic acid encodes an siRNA that forms a hairpin structure, e.g., a shRNA.

Various carriers and drug-delivery systems for siRNAs are described, e.g., in Seth et al., Ther. Deliv., 3: 245-261 (2012); Kanasty et al., Mol. Ther., 20: 513-524 (2012); Methods Enzymol., 502: 91-122 (2012); Vader et al., Curr. Top. Med. Chem., 12: 108-119 (2012); Naeye et al., Curr. Top. Med. Chem., 12: 89-96 (2012); Foged, Curr. Top. Med. Chem., 12: 97-107 (2012); Chaturvedi et al., Expert Opin. Drug Deliv., 8: 1455-1468 (2011); Gao et al., Int. J. Nanomed., 6: 1017-1025 (2011); Shegokar et al., Pharmazie., 66: 313-318 (2011); Kumari et al., Expert Opin. Drug Deliv., 11: 1327-1339 (2011).

Antibodies

In some embodiments, an inhibitor of USP30 is an antibody. The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term “antibody” as used herein refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, and antibodies of various species such as mouse, human, cynomolgus monkey, etc.

In some embodiments, an antibody comprises a heavy chain variable region and a light chain variable region, one or both of which may or may not comprise a respective constant region. A heavy chain variable region comprises heavy chain CDR1, framework (FR) 2, CDR2, FR3, and CDR3. In some embodiments, a heavy chain variable region also comprises at least a portion of an FR1, which is N-terminal to CDR1, and/or at least a portion of an FR4, which is C-terminal to CDR3. Similarly, a light chain variable region comprises light chain CDR1, framework (FR) 2, CDR2, FR3, and CDR3. In some embodiments, a light chain variable region also comprises an FR1 and/or an FR4.

Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies. Nonlimiting exemplary light chain constant regions include λ and κ.

In some embodiments, an antibody is a chimeric antibody, which comprises at least one variable region from a first species (such as mouse, rat, cynomolgus monkey, etc.) and at least one constant region from a second species (such as human, cynomolgus monkey, chicken, etc.). The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

In some embodiments, an antibody is a humanized antibody, in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc. Exemplary humanized antibodies include CDR-grafted antibodies, in which the complementarity determining regions (CDRs) of a first (non-human) species have been grafted onto the framework regions (FRs) of a second (human) species. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic. An antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

In some embodiments, an antibody is a human antibody, such as an antibody produced in a non-human animal that comprises human immunoglobulin genes, such as XenoMouse®, and antibodies selected using in vitro methods, such as phage display, wherein the antibody repertoire is based on a human immunoglobulin sequences. Transgenic mice that comprise human immunoglobulin loci and their use in making human antibodies are described, e.g., in Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806. Methods of making human antibodies using phage display libraries are described, e.g., in Hoogenboom et al., J. Mol. Biol. 227: 381-8 (1992); Marks et al., J. Mol. Biol. 222: 581-97 (1991); and PCT Publication No. WO 99/10494.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function. In some embodiments, effector function is not desirable. In some such embodiments, a human IgG4 or IgG2 heavy chain constant region may be selected or engineered.

Peptides

In some embodiments, an inhibitor of USP30 is a peptide. A peptide is a sequence of amino acids of made up of a single chain of D- or L-amino acids or a mixture of D- and L-amino acids joined by peptide bonds. The amino acid subunits of the peptide may be naturally-occurring amino acids or may be non-naturally occurring amino acids. Many non-naturally occurring amino acids are known in the art and are available commercially. Further, the peptide bonds joining the amino acid subunits may be modified. See, e.g., Sigma-Aldrich; Gentilucci et al., Curr. Pharm. Des. 16: 3185-3203 (2010); US 2008/0318838. Generally, peptides contain at least two amino acid residues and are less than about 50 amino acids in length. In various embodiments, peptide inhibitors may comprise or consist of between 3 and 50, between 5 and 50, between 10 and 50, between 10 and 40, between 10 and 35, between 10 and 30, or between 10 and 25 amino acids. In various embodiments, peptide inhibitors may comprise 10, 11, 12, 13, 14, 15, 16, 17, 18, 19, 20, 21, 22, 23, 24, or 25 amino acids. In various embodiments, peptide inhibitors may consist of 10, 11, 12, 13, 14, 15, 16, 17, 18, 19, 20, 21, 22, 23, 24, or 25 amino acids.

Methods of developing peptides that specifically bind a target molecule are known in the art, including phage display methods. See, e.g., U.S. Pat. No. 5,010,175; WO 1996/023899; WO 1998/015833; Bratkovic, Cell. Mol. Life Sci., 67: 749-767 (2010); Pande et al., Biotech. Adv. 28: 849-858 (2010). In some embodiments, following selection of a peptide, the peptide may be modified, e.g., by incorporating non-natural amino acids and/or peptide bonds. A nonlimiting exemplary method of selecting a peptide inhibitor of USP30 is described herein.

Amino acids that are important for peptide inhibition may be determined, in some embodiments, by alanine scanning mutagenesis. Each residue is replaced in turn with a single amino acid, typically alanine, and the effect on USP30 inhibition is assessed. See, e.g., U.S. Pat. Nos. 5,580,723 and 5,834,250. Truncation analyses may also be used to determine not only the importance of the amino acids at the ends of a peptide, but also the importance of the length of the peptide, on inhibitory activity. In some instances, truncation analysis may reveal a shorter peptide that binds more tightly than the parent peptide. The results of various mutational analyses, such as alanine scanning mutagenesis and truncation analyses, may be used to inform further modifications of an inhibitor peptide.

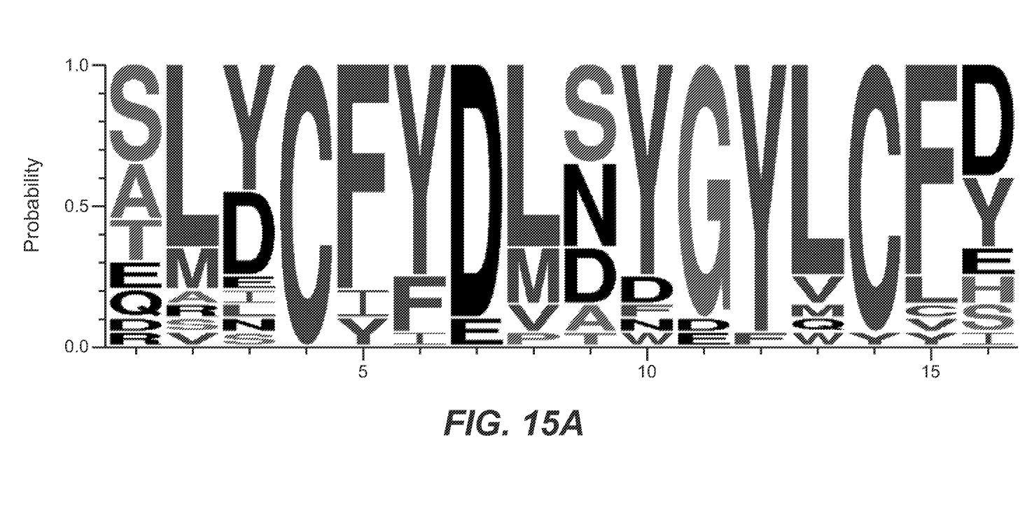

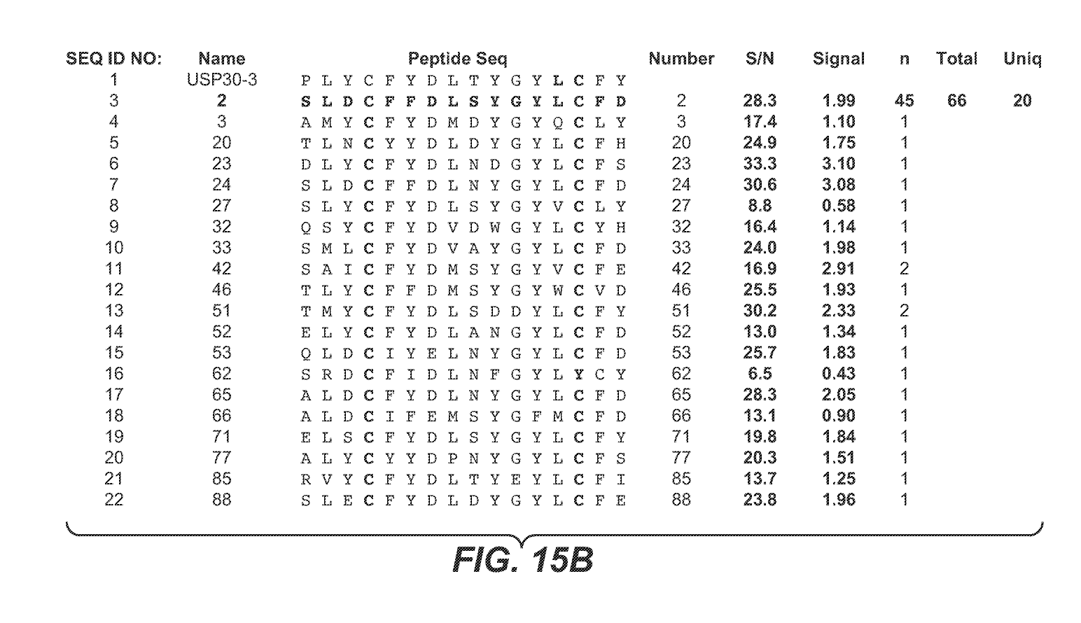

Nonlimiting exemplary peptide inhibitors are described herein, e.g., in Example 10 and FIG. 15. One skilled in the art will appreciate that, in some embodiments, the peptide sequences described herein may be modified in order to generate further peptide inhibitors with desirable properties, such as improved specificity for USP30, stronger binding to USP30, improved solubility, and/or improved cell membrane permeability. In some embodiments, a peptide inhibitor of USP30 comprises an amino acid sequence that is at least 80%, at least 85%, at least 90%, at least 95%, or 100% identical to a sequence selected from SEQ ID NOs: 1 to 22.

In some embodiments, a peptide inhibitor comprises the amino acid sequence:

| | |
|---|---|
| X₁X₂CX₃X₄X₅X₆X₇X₈X₉X₁₀X₁₁CX₁₂ | (SEQ ID NO: 48) |

wherein:

X₁ is selected from L, M, A, S, and V;

X₂ is selected from Y, D, E, I, L, N, and S;

X₃ is selected from F, I, and Y;

X₄ is selected from F, I, and Y;

X₅ is selected from D and E;

X₆ is selected from L, M, V, and P;

X₇ is selected from S, N, D, A, and T;

X₈ is selected from Y, D, F, N, and W;

X₉ is selected from G, D, and E;

X₁₀ is selected from Y and F;

X₁₁ is selected from L, V, M, Q, and W; and

X₁₂ is selected from F, L, C, V, and Y;

In some embodiments, the peptide inhibits USP30 with an IC50 of less than 10 μM. In some embodiments, X₁ is selected from L and M. In some embodiments, X₃ is selected from Y and D. In some embodiments, X₃ is F. In some embodiments, X₄ is selected from Y and F. In some embodiments, X₄ is Y. In some embodiments, X₅ is D. In some embodiments, X₆ is selected from L and M. In some embodiments, X₇ is selected from S, N, and D. In some embodiments, X₈ is Y. In some embodiments, X₉ is G. In some embodiments, X₁₀ is Y. In some embodiments, X₁₁ is L. In some embodiments, X₁₂ is selected from F and L. In some embodiments, X₁₂ is F.

In some embodiments, a peptide inhibitor comprises the amino acid sequence:

(SEQ ID NO: 49)
X_{A}X₁X₂CX₃X₄X₅X₆X₇X₈X₉X₁₀X₁₁CX₁₂X_{B}

wherein X₁ to X₁₂ are as defined above, and X_{A} and X_{B} are each independently any amino acid. In some embodiments, X_{A} is selected from S, A, T, E Q, D, and R. In some embodiments, X_{B} is selected from D, Y, E, H, S, and I.

Peptibodies

In some embodiments, an inhibitor of USP30 is a peptibody. A peptibody is peptide sequence linked to vehicle. In some embodiments, the vehicle portion of the peptibody reduces degradation and/or increases half-life, reduces toxicity, reduces immunogenicity, and/or increases biological activity of the peptide. In some embodiments, the vehicle portion of the peptibody is an antibody Fc domain. Other vehicles include linear polymers (e.g., polyethylene glycol (PEG), polylysine, dextran, etc.); branched-chain polymers (see, e.g., U.S. Pat. No. 4,289,872 and U.S. Pat. No. 5,229,490; WO 1993/0021259); a lipid; a cholesterol group (such as a steroid); a carbohydrate or oligosaccharide; or any natural or synthetic protein, polypeptide, or peptide vehicle. The peptide portion of the peptibody typically binds to the target, e.g., USP30. In some embodiments, the peptide portion of the peptibody is a peptide described herein.

In some embodiments, peptibodies retain certain desirable characteristics of antibodies, such as a long lifetime in plasma and increased affinity for binding partners (for example, due to the dimerization of Fc domains). The production of peptibodies is generally described, e.g., in WO 2000/0024782 and U.S. Pat. No. 6,660,843.

Aptamers

In some embodiments, an inhibitor of USP30 is an aptamer. The term “aptamer” as used herein refers to a nucleic acid molecule that specifically binds to a target molecule, such as USP30. Aptamers can be selected to be highly specific, relatively small in size, and/or non-immunogenic. See, e.g., Ni, et al., Curr. Med. Chem. 18: 4206 (2011). In some embodiments, a aptamer is a small RNA, DNA, or mixed RNA/DNA molecule that forms a secondary and/or tertiary structure capable of specifically binding and inhibiting USP30.

In some embodiments, an aptamer includes one or more modified nucleosides (e.g., nucleosides with modified sugars, modified nucleobases, and/or modified internucleoside linkages), for example, that increase stability in vivo, increase target affinity, increase solubility, increase serum half-life, increase resistance to degradation, and/or increase membrane permeability, etc. In some embodiments, aptamers comprise one or more modified or inverted nucleotides at their termini to prevent terminal degradation, e.g., by an exonuclease.

The generation and therapeutic use of aptamers are well established in the art. See, e.g., U.S. Pat. No. 5,475,096. In some embodiments, aptamers are produced by systematic evolution of ligands by exponential enrichment (SELEX), e.g., as described in Ellington et al., Nature 346: 818 (1990); and Tuerk et al., Science 249: 505 (1990). In some embodiments, aptamers are produced by an AptaBid method, e.g., as described in Berezovski et al., J. Am. Chem. Soc. 130: 913 (2008). Slow off-rate aptamers and methods of selecting such aptamers are described, e.g., in Brody et al., Expert Rev. Mol. Diagn., 10: 1013-22 (2010); and U.S. Pat. No. 7,964,356.

Small Molecules

In some embodiments, small molecule inhibitors of USP30 are provided. In some embodiments, a small molecule inhibitor of USP30 binds to USP30 and inhibits USP30 enzymatic activity (e.g., peptidase activity) and/or interferes with USP30 target binding and/or alters USP30 conformation such that the efficiency of enzymatic activity or target binding is reduced.

A “small molecule” is defined herein to have a molecular weight below about 1000 Daltons, for example, below about 900 Daltons, below about 800 Daltons, below about 700 Daltons, below about 600 Daltons, or below about 500 Daltons. Small molecules may be organic or inorganic, and may be isolated from, for example, compound libraries or natural sources, or may be obtained by derivatization of known compounds.

In some embodiments, a small molecule inhibitor of USP30 is identified by screening a library of small molecules. The generation and screening of small molecule libraries is well known in the art. See, e.g., Thompson et al., Chem. Rev. 96: 555-600 (1996); and the National Institutes of Health Molecular Libraries Program. A combinatorial chemical library, for example, may be formed by mixing a set of chemical building blocks in various combinations, and may result in millions of chemical compounds. For example, the systematic, combinatorial mixing of 100 interchangeable chemical building blocks theoretically results in the synthesis of 100 million tetrameric compounds or 10 billion pentameric compounds. See, e.g., Gallop et al. 1994, J. Med. Chem. 37: 1233-1250). Various other types of small molecule libraries may also be designed and used, such as, for example, natural product libraries. Small molecule libraries can be obtained from various commercial vendors. See, e.g., ChemBridge, Enzo Life Sciences, Sigma-Aldrich, AMRI Global, etc.

To identify a small molecule inhibitor of USP30, in some embodiments, a small molecule library may be screened using an assay described herein. In some embodiments, the characteristics of each small molecule that inhibits USP30 are considered in order to identify features common to the small molecule inhibitors, which may be used to inform further modifications of the small molecules.

In some embodiments, one or more small molecule inhibitors of USP30 identified, for example, in an initial library screen, may be used to generate a subsequent library comprising modifications of the initial small molecule inhibitors. Using this method, subsequent iterations of candidate compounds may be developed that possess greater specificity for USP30 (versus other DUBs), and/or greater binding affinity for USP30, and/or other desirable properties, such as low toxicity, greater solubility, greater cell permeability, etc.

Various small molecule inhibitors of deubiquitylating enzymes are known in the art, some of which are shown in Table 3.

TABLE 3
Inhibitors of ubiquitin specific proteases

| Name | Structure | Target | Reference |
|---|---|---|---|
| HBX 41, 108 | 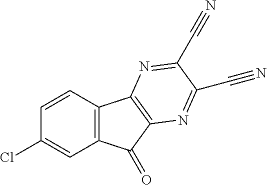 [SMILES: N#Cc1nc2c(nc1C#N)-c1ccc(Cl)cc1C2=O] | USP7 | Colland et al., Mol. Cancer Therap., 8:2286 (2009) |
| HBX 90, 397 | 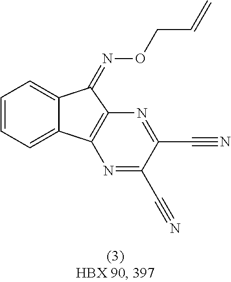 [SMILES: C=CCO/N=C1/c2ccccc2-c2nc(C#N)c(C#N)nc21] | USP8 | WO 2007/017758; |
| IU1 | 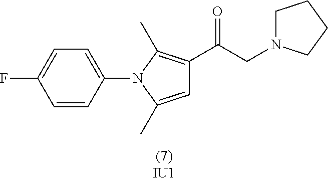 [SMILES: Cc1cc(C(=O)CN2CCCC2)c(C)n1-c1ccc(F)cc1] | USP14 | Lee et al., Nature, 467: 179-184 (2010) |
| PR619 | 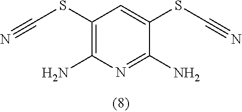 [SMILES: N#CSc1cc(SC#N)c(N)nc1N] | Broad specificity DUB inhibitor | Tian et al., Assay Drug Develop. Technol., 9: 165-173 (2011) |
| Isatin O-acyl oxime | 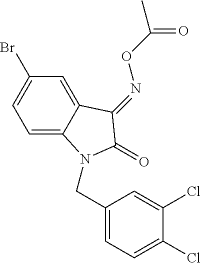 [SMILES: CC(=O)O/N=C1/C(=O)N(Cc2ccc(Cl)c(Cl)c2)c2ccc(Br)cc21] | UCH-L1 | Liu et al., Chemistry & Biology, 10: 837-846 (2003) |
| Isatin derivative | 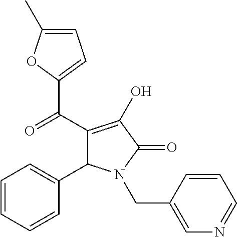 [SMILES: Cc1ccc(C(=O)C2=C(O)C(=O)N(Cc3cccnc3)C2c2ccccc2)o1] | UCH-L3 | Liu et al., Neurobiol. Disease, 41: 318-328 (2010); Koharudin et al., PNAS, 107: 6835- 6840 (2010) |
| PGA₁ | 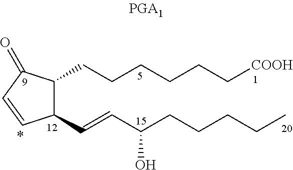 [SMILES: CCCCC[C@H](O)/C=C/[C@H]1C=CC(=O)[C@@H]1CCCCCCC(=O)O] | Ubiquitin isopeptidase | Mullally et al., J. Biol. Chem., 276: 30366-73 (2001) |
| PGA₂ | 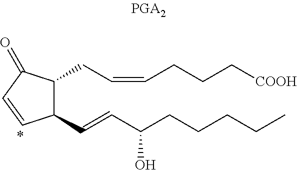 [SMILES: CCCCC[C@H](O)/C=C/[C@H]1C=CC(=O)[C@@H]1C/C=C\CCCC(=O)O] | Ubiquitin isopeptidase | Mullally et al., J. Biol. Chem., 276: 30366-73 (2001) |
| Δ12-PGJ₂ | 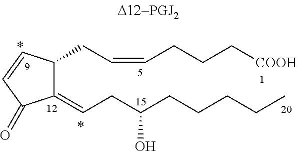 [SMILES: CCCCC[C@H](O)C/C=C1/C(=O)C=C[C@@H]1C/C=C\CCCC(=O)O] | Ubiquitin isopeptidase | Mullally et al., J. Biol. Chem., 276: 30366-73 (2001) |
| Dibenzylidene- acetone (DBA) | 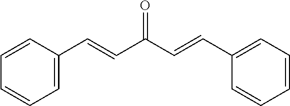 [SMILES: O=C(/C=C/c1ccccc1)/C=C/c1ccccc1] | Ubiquitin isopeptidase | WO 2004/009023 |
| Curcumin | 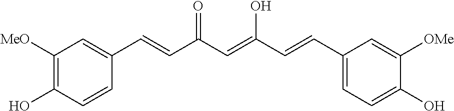 [SMILES: COc1cc(/C=C/C(=O)/C=C(O)/C=C/c2ccc(O)c(OC)c2)ccc1O] | Ubiquitin isopeptidase | WO 2004/009023 |
| Shikoccin (NSC-302979) | 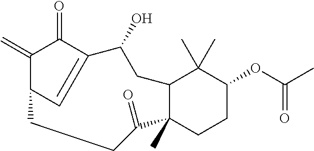 [SMILES: C=C1C(=O)C2=C[C@@H]1CCC(=O)[C@@]1(C)CC[C@@H](OC(C)=O)C(C)(C)C1C[C@H]2O] | Ubiquitin isopeptidase | WO 2004/009023 |

The inhibitors shown in Table 3 and the references cited therein, as well as additional inhibitors known in the art, can form the basis for developing additional deubiquitylation enzyme inhibitors, including specific inhibitors of USP30. See also WO 2007/009715. One skilled in the art can, for example, make modifications to any of the above structures to form a library of putative deubiquitylation enzyme inhibitors and screen for modified compounds with specificity for USP30 using the assays described herein.

B. Assays

Various assays may be used to identify and test inhibitors of USP30. For inhibitors that reduce expression of USP30 protein, any assay that detects protein levels may be suitable for measuring inhibition. As an example, protein levels can be detected by various immunoassays using antibodies that bind USP30, such as ELISA, Western blotting, immunohistochemistry, etc. If an inhibitor affects the subcellular localization of USP30, changes in subcellular localization may be detected, e.g., by immunohistochemistry, or by fractionating cellular components and detecting levels of USP30 in the various fractions using one or more antibodies. For inhibitors that reduce levels of USP30 mRNA, amplification-based assays, such as reverse transcriptase PCR (RT-PCR) may be used to detect changes in mRNA levels.

For inhibitors that affect USP30 enzymatic activity, a nonlimiting exemplary assay is as follows: USP30 is contacted with the inhibitor or candidate inhibitor in the presence of a USP30 substrate. Nonlimiting exemplary USP30 substrates include a Ub-β-galactosidase fusion protein (see, e.g., Quesada et al., Biochem. Biophys. Res. Commun 314:54-62 (2004)), Ub4 chains (e.g., Lys-48- and Lys-63-linked Ub), the linear product of UBIQ gene translation; the post-translationally formed branched peptide bonds in mono- or multi-ubiquitylated conjugates; ubiquitylated remnants resulting from proteasome-mediated degradation, and other small amide or ester adducts. USP30 activity (e.g., processing of Ub substrates) is measured in the presence of USP30 and the inhibitor or candidate inhibitor. This activity is compared with the processing of Ub substrates in the presence USP30 without the inhibitor or candidate inhibitor. If the inhibitor or candidate inhibitor inhibits the activity of USP30, the amount of UB substrate processing will decrease compared to the amount of UB substrate processing in the presence of USP30 without the inhibitor or candidate inhibitor.

A further nonlimiting exemplary assay to determine inhibition and/or specificity is described in Example 10. Briefly, a range of concentrations of inhibitor are mixed with ubiquitin-AMC and USP30 (the inhibitor may be mixed with the substrate, and then USP30 added to start the reaction). If specificity is to be determined, similar reactions may be set up with one or more additional DUBs or ubiquitin C-terminal hydrolases (UCHs) in place of USP30. Immediately after addition of the enzyme, fluorescence is monitored (with excitation at 340 nm and emission at 465 nm). The initial rate of enzymatic activity may be calculated as described in Example 10.

C. Pharmaceutical Formulations

Pharmaceutical formulations of an inhibitor of USP30 as described herein are prepared by mixing such inhibitor having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the inhibitor, which matrices are in the form of shaped articles, e.g. films, or microcapsules.

The formulations to be used for in vivo administration are generally sterile. Sterility may be readily accomplished, e.g., by filtration through sterile filtration membranes.

D. Therapeutic Methods and Compositions

Any of the inhibitors of USP30 provided herein may be used in methods, e.g., therapeutic methods. In some embodiments, a method of increasing mitophagy in a cell is provided, the method comprising contacting the cell with an inhibitor of USP30 under conditions allowing inhibition of USP30 in the cell. In some embodiments, a method of increasing mitochondrial ubiquitination in a cell is provided, the method comprising contacting the cell with an inhibitor of USP30 under conditions allowing inhibition of USP30 in the cell. Increased mitophagy may be determined, e.g., using immunofluorescence as described in Example 6. Increased ubiquitination may be determined, e.g., by immunoaffinity enrichment of ubiquitinated peptides after trypsin digestion, followed by mass spectrometry as described in Example 5. In some embodiments, an increase in mitochondrial ubiquitination may be determined by comparing the ubiquitination of a mitochondrial proteins a cell or population of cells contacted with an inhibitor of USP30 with the ubiquitination of mitochondrial proteins in a matched cell or population of cells not contacted with the inhibitor.

In some embodiments, increased mitophagy means a reduction in the average number of mitochondria per cell of at least 10%, at least 15%, at least 20%, at least 25%, at least 30%, at least 35%, at least 40%, at least 45%, at least 50%, at least 55%, at least 60%, at least 65%, at least 70%, or at least 75%, in a population of cells contacted with an inhibitor of USP30, as compared to matched population of cells not contacted with the inhibitor. In some embodiments, increased mitochondrial ubiquitination means an increase in overall ubiquitination of mitochondrial proteins in a cell or population of cells contacted with an inhibitor of USP30 of at least 10%, at least 20%, at least 30%, at least 40%, at least 50%, at least 60%, at least 70%, at least 80%, at least 90%, at least 100% (i.e., 2-fold), at least 150%, or at least 200% (i.e., 3-fold) as compared to a matched cell or population of cells not contacted with the inhibitor.

In some embodiments, a method of increasing ubiquitination of at least one protein selected from Tom20, MIRO, MUL1, ASNS, FKBP8, TOM70, MAT2B, PRDX3, IDE, VDAC, IP05, PSD13, UBP13, and PTH2 in a cell is provided, the method comprising contacting the cell with an inhibitor of USP30 under conditions allowing inhibition of USP30 in the cell. In some such embodiments, ubiquitination increases at at least one, at least two, or three amino acids selected from K56, K61, and K68 of Tom 20; and/or ubiquitination increases at at least one, at least two, at least three, at least four, at least five, at least six, at least seven, or eight amino acids selected from K153, K187, K330, K427, K512, K535, K567, and K572 of MIRO. In some such embodiments, ubiquitination increases at at least one, at least two, or three amino acids selected from K56, K61, and K68 of Tom 20; and/or ubiquitination increases at at least one, at least two, at least three, at least four, at least five, at least six, at least seven, or eight amino acids selected from K153, K187, K330, K427, K512, K535, K567, and K572 of MIRO; and/or ubiquitination increases at at least one, at least two, or three amino acids selected from K273, K299, and K52 of MUL1; and/or ubiquitination increases at at least one, at least two, at least three, at least four, at least five, at least six, at least seven, or eight amino acids selected from K249, K271, K273, K284, K307, K317, K334, and K340 of FKBP8; and/or ubiquitination increases at at least one, at least two, at least three, at least four, at least five, at least six, at least seven, at least eight, or nine amino acids selected from K147, K168, K176, K221, K244, K275, K478, K504, and K556 of ASNS; and/or ubiquitination increases at at least one, at least two, at least three, at least four, at least five, at least six, at least seven, at least eight, at least nine, or at least ten amino acids selected from K78, K120, K123, K126, K129, K148, K168, K170, K178, K185, K204, K230, K233, K245, K275, K278, K312, K326, K349, K359, K441, K463, K470, K471, K494, K501, K524, K536, K563, K570, K599, K600, and K604 of TOM70; and/or ubiquitination increases at at least one, at least two, at least three, or four amino acids selected from K209, K245, K316, and K326 of MAT2B; and/or ubiquitination increases at at least one, at least two, at least three, at least four, or five amino acids selected from K83, K91, K166, K241, and K253 of PRDX3; and/or ubiquitination increases at at least one, at least two, at least three, at least four, at least five, or six amino acids selected from K558, K657, K854, K884, K929, and K933 of IDE; and/or ubiquitination increases at at least one, at least two, at least three, at least four, at least five, at least six, or seven amino acids selected from K20, K53, K61, K109, K110, K266, and K274 of VDAC1; and/or ubiquitination increases at at least one, at least two, at least three, at least four, at least five, or six amino acids selected from K31, K64, K120, K121, K277, and K285 of VDAC2; and/or ubiquitination increases at at least one, at least two, at least three, at least four, at least five, at least six, at least seven, or eight amino acids selected from K20, K53, K61, K109, K110, K163, K266, and K274 of VDAC3; and/or ubiquitination increases at at least one, at least two, at least three, at least four, at least five, at least six, at least seven, at least eight, at least nine, or at least ten amino acids selected from K238, K353, K436, K437, K548, K556, K613, K678, K690, K705, K775, and K806 of IP05; and/or ubiquitination increases at at least one, at least two, at least three, at least four, at least five, at least six, at least seven, at least eight, at least nine, or at least ten amino acids selected from K2, K32, K99, K115, K122, K132, K161, K186, K313, K321, K347, K350, and K361 of PSD13; and/or ubiquitination increases at at least one, at least two, at least three, at least four, at least five, at least six, at least seven, at least eight, at least nine, or at least ten amino acids selected from K18, K190, K259, K326, K328, K401, K405, K414, K418, K435, K586, K587, and K640 of UBP13; and/or ubiquitination increases at at least one, at least two, at least three, at least four, at least five, at least six, at least seven, at least eight, or nine amino acids selected from K47, K76, K81, K95, K106, K119, K134, K171, K177 of PTH2. In some embodiments, ubiquitination of one or more additional proteins increases upon contacting a cell with an inhibitor of USP30. Nonlimiting exemplary proteins whose ubiquitination may be increased in the presence of an inhibitor of USP30 are listed in Appendix A, which is incorporated herein by reference. Increased ubiquitination of a target protein can be determined, e.g., by immunoaffinity enrichment of ubiquitinated peptides after trypsin digestion, followed by mass spectrometry, as described in Example 5. In some embodiments, an increase in ubiquitination may be determined by comparing the ubiquitination of a target protein a cell or population of cells contacted with an inhibitor of USP30 with the ubiquitination of the same target protein in a matched cell or population of cells not contacted with the inhibitor.

In some embodiments, increased ubiquitination of a protein means an increase in ubiquitination of the protein in a cell or population of cells contacted with an inhibitor of USP30 of at least 10%, at least 20%, at least 30%, at least 40%, at least 50%, at least 60%, at least 70%, at least 80%, at least 90%, at least 100% (i.e., 2-fold), at least 150%, or at least 200% (i.e., 3-fold) as compared to a matched cell or population of cells not contacted with the inhibitor.

In some embodiments, the cell is under oxidative stress. Further, in some embodiments, a method of reducing oxidative stress in a cell is provided, the method comprising contacting the cell with an inhibitor of USP30 under conditions allowing inhibition of USP30 in the cell.

In any of the foregoing methods, the cell may comprise a pathogenic mutation in Parkin, a pathogenic mutation in PINK1, or a pathogenic mutation in Parkin and a pathogenic mutation in PINK1. Nonlimiting exemplary pathogenic mutations in Parkin and PINK1 are shown, e.g., in Tables 1 and 2 herein.

In some embodiments, the cell is a neuron. In some embodiments, the cell is a substantia nigra neuron. In some embodiments, the cell is a cardiac cell. In some embodiments, the cell is a cardiomyocyte cell. In some embodiments, the cell is a muscle cell.

In some embodiments of any of the foregoing methods, the cell is comprised in a subject. In some embodiments of any of the foregoing methods, the cell may be in vitro or ex vivo.

In another aspect, an inhibitor of USP30 for use as a medicament is provided. In further aspects, an inhibitor of USP30 for use in a method of treatment is provided. In some embodiments, a method of treating a condition involving a mitochondrial defect in a subject is provided, the method comprising administering to the subject an effective amount of an inhibitor of USP30. A condition involving a mitochondrial defect may involve a mitophagy defect, one or more mutations in mitochondrial DNA, mitochondrial oxidative stress, defects in mitochondrial shape/morphology, mitochondrial membrane potential defects, and/or a lysosomal storage defect. Nonlimiting exemplary conditions involving mitochondrial defects include neurodegenerative diseases; mitochondrial myopathy, encephalopathy, lactic acidosis, and stroke-like episodes (MELAS) syndrome; Leber's hereditary optic neuropathy (LHON); neuropathy, ataxia, retinitis pigmentosa-maternally inherited Leigh syndrome (NARP-MILS); Danon disease; ischemic heart disease leading to myocardial infarction; multiple sulfatase deficiency (MSD); mucolipidosis II (ML II); mucolipidosis III (ML III); mucolipidosis IV (ML IV); GM1-gangliosidosis (GM1); neuronal ceroid-lipofuscinoses (NCL1); Alpers disease; Barth syndrome; Beta-oxidation defects; carnitine-acyl-carnitine deficiency; carnitine deficiency; creatine deficiency syndromes; co-enzyme Q10 deficiency; complex I deficiency; complex II deficiency; complex III deficiency; complex IV deficiency; complex V deficiency; COX deficiency; chronic progressive external ophthalmoplegia syndrome (CPEO); CPT I deficiency; CPT II deficiency; glutaric aciduria type II; Kearns-Sayre syndrome; lactic acidosis; long-chain acyl-CoA dehydrongenase deficiency (LCHAD); Leigh disease or syndrome; lethal infantile cardiomyopathy (LIC); Luft disease; glutaric aciduria type II; medium-chain acyl-CoA dehydrongenase deficiency (MCAD); myoclonic epilepsy and ragged-red fiber (MERRF) syndrome; mitochondrial recessive ataxia syndrome; mitochondrial cytopathy; mitochondrial DNA depletion syndrome; myoneurogastointestinal disorder and encephalopathy; Pearson syndrome; pyruvate carboxylase deficiency; pyruvate dehydrogenase deficiency; POLG mutations; medium/short-chain 3-hydroxyacyl-CoA dehydrogenase (M/SCHAD) deficiency; and very long-chain acyl-CoA dehydrongenase (VLCAD) deficiency. Nonlimiting exemplary neurodegenerative diseases that involve mitochondrial defects include Parkinson's disease, Alzheimer's disease, Huntington's disease, amyotrophic lateral sclerosis (ALS), ischemia, stroke, dementia with Lewy bodies, and frontotemporal dementia. Additional exemplary neurodegenerative diseases that may involve mitochondrial defects include, but are not limited to, intracranial hemorrhage, cerebral hemorrhage, trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, multiple system atrophy, progressive supranuclear palsy, corticobasal degeneration, demyelinating diseases, Guillain-Barre syndrome, multiple sclerosis, Charcot-Marie-Tooth disease, prion disease, Creutzfeldt-Jakob disease, Gerstmann-Straussler-Scheinker syndrome (GSS), and fatal familial insomnia. In some such embodiments, the method further comprises administering to the individual an effective amount of at least one additional therapeutic agent, e.g., as described below.

In some embodiments, an inhibitor of USP30 is provided for use in the manufacture or preparation of a medicament. In some such embodiments, the medicament is for treatment of conditions involving a mitochondrial defect, such as, for example, conditions involving a mitophagy defect, conditions involving mutations in mitochondrial DNA, conditions involving mitochondrial oxidative stress, conditions involving defects in mitochondrial shape/morphology, conditions involving defects in mitochondrial membrane potential, and conditions involving lysosomal storage defects. In further embodiments, the medicament is for use in a method of treating a condition involving a mitochondrial defect, the method comprising administering to an individual having the condition involving a mitochondrial defect an effective amount of the medicament. In one such embodiment, the method further comprises administering to the individual an effective amount of at least one additional therapeutic agent, e.g., as described below.

An “individual” according to any of the embodiments herein may be a human.

In a further aspect, pharmaceutical formulations comprising any of the inhibitors of USP30 provided herein, e.g., for use in any of the above therapeutic methods are provided. In one embodiment, a pharmaceutical formulation comprises any of the inhibitors of USP30 provided herein and a pharmaceutically acceptable carrier. In another embodiment, a pharmaceutical formulation comprises any of the inhibitors of USP30 provided herein and at least one additional therapeutic agent, e.g., as described below.

Inhibitors of USP30 can be used either alone or in combination with other agents in a therapy. For instance, an inhibitor of USP30 may be co-administered with at least one additional therapeutic agent.

Exemplary therapeutic agents that may be combined with an inhibitor of USP30, e.g., for the treatment of Parkinson's disease, include levodopa, dopamine agonists (such as pramipexole, ropinirole, and apomorphine), monoamine oxygenase (MAO) B inhibitors (such as selegiline and rasagiline), catechol O-methyltransferase (COMT) inhibitors (such as entacapone and tolcapone), anticholinergics (such as benzotropine and trihexylphenidyl), and amantadine. A further exemplary therapeutic agent that may be combined with an inhibitor of USP30, e.g., for the treatment of amyotrophic lateral sclerosis, is riluzole. Exemplary therapeutic agents that may be combined with an inhibitor of USP30, e.g., for the treatment of Alzheimer's disease, include cholinesterase inhibitors (such as donepezil, rivastigmine, galantamine, and tacrine), and memantine. Exemplary therapeutic agents that may be combined with an inhibitor of USP30, e.g., for the treatment of Huntington's disease, include tetrabenazine, antipsychotic drugs (such as haloperidol and clozapine), clonazepam, diazepam, antidepressants (such as escitalopram, fluoxetine, and sertraline), and mood-stabilizing drugs (such as lithium), and anti-convulsants (such as valproic acid, divalproex, and lamotrigine).

Administration “in combination” encompasses combined administration (where two or more therapeutic agents are included in the same or separate formulations), and separate administration, in which case, administration of the inhibitor of the invention can occur prior to, simultaneously, and/or following, administration of the additional therapeutic agent and/or adjuvant. In some embodiments, administration of the inhibitor of USP30 and administration of an additional therapeutic agent occur within about one month, or within about one, two, or three weeks, or within about one, two, three, four, five, or six days of one another Inhibitors of the invention can also be used in combination with other types of therapies.

An inhibitor of the invention (and any additional therapeutic agent) can be administered by any suitable means, including oral, parenteral, intrapulmonary, intranasal, and intralesional administration. Parenteral administration includes, but is not limited to, intramuscular, intravenous, intraarterial, intracerebral, intracerebroventricular, intrathecal, intraocular, intraperitoneal, and subcutaneous administration. An inhibitor of the invention (and any additional therapeutic agent) may also be administered using an implanted delivery device, such as, for example, an intracerebral implant. Nonlimiting exemplary central nervous system delivery methods are reviewed, e.g., in Pathan et al., Recent Patents on Drug Delivery & Formulation, 2009, 3: 71-89. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Inhibitors of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The inhibitor need not be, but is optionally formulated with one or more agents currently used to prevent or treat the disorder in question. The effective amount of such other agents depends on the amount of inhibitor present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an inhibitor of USP30 (when used alone or in combination with one or more other additional therapeutic agents) will depend on the type of disease to be treated, the type of inhibitor, the severity and course of the disease, whether the inhibitor is administered for preventive or therapeutic purposes, previous therapy, the patient's clinical history and response to the inhibitor, and the discretion of the attending physician. The inhibitor is suitably administered to the patient at one time or over a series of treatments. Depending on the type and severity of the disease, about 1 μg/kg to 15 mg/kg (e.g. 0.1 mg/kg-10 mg/kg) of inhibitor can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the inhibitor would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the inhibitor). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using more than one inhibitor of USP30.

E. Articles of Manufacture

In another aspect of the invention, an article of manufacture containing materials useful for the treatment, prevention and/or diagnosis of the disorders described herein is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is an inhibitor of the invention. The label or package insert indicates that the composition is used for treating the condition of choice. Moreover, the article of manufacture may comprise (a) a first container with a composition contained therein, wherein the composition comprises an inhibitor of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

### III. EXAMPLES

The following are examples of methods and compositions of the invention. It is understood that various other embodiments may be practiced, given the general description provided above.

### Example 1

### Materials and Methods

DUB cDNA Overexpression Screen:

To identify regulators of mitophagy, individual cDNAs from a FLAG-tagged DUB library were cotransfected into HeLa cells with GFP-Parkin using Lipofectamine 2000 (Invitrogen) (1:3 DUB-FLAG: GFP-Parkin cDNA ratio). After 24 hours of expression, cells were treated with 10 μM CCCP for 24 hours, and fixed and stained using anti-Tom20 (Santa Cruz Biotechnology), anti-GFP (Ayes Labs) and anti-FLAG (Sigma) primary antibodies. Following staining with secondary antibodies, images of random fields were acquired with a Leica SP5 Laser Scanning Confocal Microscope using a 40×/1.25 oil-objective (0.34 μm/pixel resolution, 1 μm confocal z-step size). Percent of GFP-Parkin and FLAG-DUB cotransfected cells containing Tom20 staining was scored blindly.

### Hippocampal Culture, Transfection and Mt-Keima Imaging:

Dissociated hippocampal neuron cultures were prepared as described (Seeburg et al., Neuron 58: 571-583 (2008)) and transfected using Lipofectamine LTX PLUS at DIV 8-10. Constructs were expressed for 1-3 days for overexpression experiments and 3-4 days for knockdown experiments. mt-Keima-transfected neurons were imaged with a Leica TCS SP5 Laser Scanning Confocal microscope with a 40×/1.25 oil objective (0.07 μm/pixel resolution, 1 μm confocal z-step size). Cells were kept in a humidified chamber maintained at 37° C./5% CO₂ during imaging. Two images were acquired using a hybrid detector in sequential mode with 458 nm (neutral pH signal) and 543 nm (acidic pH signal) laser excitation, and emission fluorescence collected between 630-710 nm. All image quantification was performed by custom-written macros in ImageJ. For mt-Keima quantification, cell bodies were manually outlined and total area of high ratio (543 nm/458 nm) lysosomal signal was divided by the total area of somatic mitochondrial signal (mitophagy index).

### Mass Spectrometry

To determine Parkin substrates, HEK-293 GFP-Parkin inducible cells were treated with doxycycline for 24 hours, and then treated with 5 μM CCCP or DMSO vehicle control for 2 hours. To determine USP30 substrates, HEK-293T cells were transfected with human USP30 shRNA using Lipofectamine 2000 (Invitrogen) for 6 days, then treated as before. In both experiments, cells were lysed (20 mM HEPES pH 8.0, 8M urea, 1 mM sodium orthovanadate, 2.5 mM sodium pyrophosphate, 1 mM β-glycerophosphate), sonicated, and cleared by centrifugation prior to proteolytic digestion and immunoaffinity enrichment of peptides bearing the ubiquitin remnant, and mass spectrometry analysis.

### Preparation of Cellular Lysates and Immunoprecipitation

For total lysate experiments, transfected HEK-293 cells were lysed at 24 hours post-transfection in SDS sample buffer (Invitrogen) containing sample reducing agent (Invitrogen). For immunoprecipitation experiments, cells were lysed at 24 hours (overexpression experiments) or 6 days (knockdown experiments) post-transfection in TBS buffer containing 0.5% SDS, and lysates were diluted with a buffer containing 1% Triton-X-100 and protease and phosphatase inhibitors. Ubiquitinated proteins were immunoprecipated from lysates of HA-ubiquitin transfected cells with anti-HA affinity matrix beads (Roche Applied Science). Inputs and precipitates were resolved by SDS-PAGE and analyzed by immunoblotting.

### Statistical Analysis

Error bars indicate standard error of the mean (S.E.M.), To compute p values, non-paired Student's t-test, One-way ANOVA with Dunnett's Multiple Comparison test (for comparisons to a single condition) or Bonferroni's Multiple Comparison test (for comparisons between multiple conditions), and Two-way ANOVA were used, as indicated in figure legends. All statistical analysis was performed in GraphPad Prism v.5 software.

### DNA Construction

For the DUB overexpression screen, a FLAG-tagged DUB library consisting of 100 cDNAs was used. For transfection, the following constructs were subcloned into β-actin promoter-based pCAGGS plasmid: USP30-FLAG (rat), USP30-FLAG (human), GFP-Parkin (human), FLAG-Parkin (human), PINK1-GFP (human), myc-Parkin (human), RHOT1 (MIRO)-myc-FLAG (human), TOM20-myc (human), HA-ubiquitin, PSD-95-FLAG, and mt-mKeima (Katayama et al., Chemistry & Biology 18: 1042-1094 (2011)). Point mutations were generated using QuikChange II XL (Agilent Technologies) for the following constructs: USP30-C77S-FLAG (rat), USP30-C77A-FLAG (rat), USP30-C77S-FLAG (human), GFP-Parkin K161N (human), and GFP-Parkin G430D (human). Mito-tagGFP2 (Evrogen), Tom20-3KR-myc, and HA-ubiquitin mutants (Blue Heron) were purchased. β-Gal (Seeburg and Sheng, J. Neurosci. 28: 6583-6591 (2008)) and mito-ro-GFP (Dooley et al., J. Biol. Chem. 279: 22284-22293 (2004)) expression plasmids were previously described. Short-hairpin sequences targeting the following regions were cloned into pSuper or pSuper-GFP-neo plasmids: rat PINK1 #1 (TCAGGAGATCCAGGCAATT), rat PINK1 #2 (CCAGTACCTTGAAGAGCAA), rat Parkin #1 (GGAAGTGGTTGCTAAGCGA), rat Parkin #2 (GAGGAAAAGTCACGAAACA), rat USP30 (CCAGAGCCCTGTTCGGTTT), human USP30 (CCAGAGTCCTGTTCGATTT), and firefly luciferase (CGTACGCGGAATACTTCGA).

### Antibodies and Reagents:

The following antibodies were used for immunocytochemistry: rabbit anti-TOM20, mouse anti-TOM20, goat anti-HSP60 (Santa Cruz Biotechnology); mouse anti-FLAG, rabbit anti-FLAG, mouse anti-myc (Sigma-Aldrich); and chicken anti-GFP (Ayes Labs).

The following antibodies were used for immunoblotting: rabbit anti-TOM20, goat anti-HSP60 (Santa Cruz Biotechnology); mouse anti-MFN1, HRP-conjugated anti-FLAG, mouse anti-myc, rabbit anti-USP30, rabbit anti-RHOT1 (MIRO), rabbit anti-TIMM8A (Sigma-Aldrich); rabbit anti-GFP, chicken anti-GFP (Invitrogen); HRP-conjugated anti-GAPDH, HRP-conjugated anti-α-actin, HRP-conjugated anti-β-tubulin, rabbit anti-VDAC (Cell Signaling Technology); rabbit anti-TOM70 (Proteintech Group); anti-ubiquitin (FK2) (Enzo Life Sciences); mouse anti-LAMP1 (StressGen); HRP-conjugated anti-HA (Roche); and anti-USP30 rabbit (generated by immunizing rabbits with purified human USP30 amino acids 65-517).

For immunoprecipitation experiments anti-FLAG M2 affinity gel beads (Sigma) and anti-HA affinity matrix beads (Roche Applied Science) were used.

Adeno-associated virus type2 (AAV2) particles expressing Parkin, PINK1 and USP30 shRNAs were produced by Vector Biolabs, Inc. from pAAV-BASIC-CAGeGFP-WPRE vector containing the H1 promoter and shRNA expression cassette of the pSuper vectors.

The following reagents were purchased as indicated: blasticidin S, zeocin, Lipofectamine 2000, Lipofectamine LTX PLUS, LysoTracker Green DND-626 (Invitrogen); PhosSTOP phosphatase inhibitor tablets, cOmplete EDTA-free protease inhibitor tablets, DNase I (Roche Applied Science); carbonyl cyanide 3-chlorophenylhydrazone (CCCP), doxycycline, dimethyl sulfoxide, ammonium chloride, rotenone, DTT, aldrithiol, paraquat dichloride (Sigma-Aldrich); N-Ethylmaleimide (Thermo Scientific); and hygromycin (Clontech Laboratories).

### Transfection and Immunocytochemistry:

All heterologous cells were transfected with Lipofectamine 2000 for cDNA expression and Lipofectamine RNAiMAX for siRNA knockdown experiments, according to manufacturer's instructions (Invitrogen). siRNAs were purchased from Dharmacon as siGenome pools (non-Silencing pool #2 was used control siRNA transfection). Hippocampal cultures were prepared as described previously (Seeburg et al., Neuron 58: 571-583 (2008)) and transfected with Lipofectamine LTX PLUS (Invitrogen) with 1.8 μg DNA, 1.8 μl PLUS reagent and 6.3 μl LTX reagent. Following drug treatments, cells were fixed with 4% paraformaldehyde/4% sucrose in phosphate-buffered saline (PBS, pH 7.4) (Electron Microscopy Sciences). Following permeabilization (0.1% Triton-X in PBS), blocking (2% BSA in PBS) and primary antibody incubation, antibodies were visualized using Alexa dye-conjugated secondary antibodies (Invitrogen). All immunocytochemistry images were acquired with a Leica SP5 laser scanning microscope with a 40×/1.25 oil objective (0.34 μm/pixel resolution, 1 μm confocal z-step size).

### HEK293 and SH-SY5Y Stable Cell Line Generation

Stably transfected HEK cell lines expressing GFP-Parkin (human) wild-type, K161N, and G430D were generated by co-transfecting FLP-In 293 cells with a pOG44 Flp-recombinase expression vector (Invitrogen) and a pcDNA5-FRT vector (Invitrogen) expressing the corresponding constructs under a CMV promoter. Cell lines were selected and maintained using 50 μg/mL hygromycin selection. Inducible HEK stable cell line expressing GFP-Parkin (human) was generated by co-transfecting FLP-In T-Rex 293 cells with pOG44 and a pcDNA5-FRT-TO vector (Invitrogen) expressing GFP-Parkin (human). The line was selected and maintained using 50 μg/mL hygromycin and 15 μg/mL blasticidin. SH-SY5Y stable cells were generated similarly with a Flp-In inducible parental cell line using pcDNA5-FRT-TO and maintained under 75 μg/ml hygromycin and 3 μg/ml blasticidin.

### Isolation and Identification of Ubiquitin Modifications by Mass Spectrometry

To identify Parkin substrates, HEK 293T cells stably expressing inducible GFP-Parkin (human) were induced using doxycycline (1 μg/mL) for 24 hours, then treated with 5 μM CCCP or DMSO vehicle control for 2 hours. To determine USP30 substrates, HEK 293T cells were transfected with human USP30 shRNA using Lipofectamine 2000 (Invitrogen) for 6 days, then treated as above.

Immunoaffinity isolation and mass spectrometry methods were used to enrich and identify K-GG peptides from digested protein lysates as previously described (Xu et al., Nat. Biotech., 28: 868-873 (2010); Kim et al., Mol. Cell, 44: 325-340 (2011)). Cell lysates were prepared in lysis buffer (8M urea 20 mM HEPES pH 8.0 with 1 mM sodium orthovanadate, 2.5 mM sodium pyrophosphate, 1 mM B-glycerophosphate) by brief sonication on ice. Protein samples (60 mg) were reduced at 60° C. for 20 min in 4.1 mM DTT, cooled 10 min on ice, and alkylated with 9.1 mM iodoacetamide for 15 min at room temperature in the dark. Samples were diluted 4X using 20 mM HEPES pH 8.0 and digested in 10 μg/ml trypsin overnight at room temperature. Following digestion, TFA was added to a final concentration of 1% to acidify the peptides prior to desalting on a Sep-Pak C18 cartridge (Waters). Peptides were eluted from the cartridge in 40% ACN/0.1% TFA, flash frozen and lyophilized for 48 hr. Dry peptides were gently resuspended in 1.4 ml 1×IAP buffer (Cell Signaling Technology) and cleared by centrifugation for 5 min at 1800×g. Precoupled anti-KGG beads (Cell Signaling Technology) were washed in 1×IAP buffer prior to contacting the digested peptides.

Immunoaffinity enrichment was performed for 2 hours at 4° C. Beads were washed 2X with IAP buffer and 4X with water prior to 2X elution of peptides in 0.15% TFA for 10 min each at room temperature. Immunoaffinity enriched peptides were desalted using STAGE-Tips as previously described (Rappsilber et al., Anal. Chem., 75: 663-670 (2003)).

Liquid chromatography-mass spectrometry (LC-MS) analysis was performed on an LTQ-Orbitrap Velos mass spectrometer operating in data dependent top 15 mode. Peptides were injected onto a 0.1×100-mm Waters 1.7-um BEH-130 C18 column using a NanoAcquity UPLC and separated at 1 ul/min using a two stage linear gradient where solvent B ramped first from 2% to 25% over 85 min and then 25% to 40% over 5 min. Peptides eluting from the column were ionized and introduced to the mass spectrometer using an ADVANCE source (Michrom-Bruker). In each duty cycle, one full MS scan collected was at 60,000 resolution in the Orbitrap followed by up to 15 MS/MS scans in the ion trap on monoisotopic, charge state defined precursors (z>1). Ions selected for MS/MS (+20 ppm) were subjected to dynamic exclusion for 30 sec duration.

Mass spectral data were converted to mzxml for loading into a relational database. MS/MS spectra were searched using Mascot against a concatenated target-decoy database of tryptic peptides from human proteins (Uniprot) and common contaminants. Precursor ion mass tolerance was set to +50 ppm. Fixed modification of carbamidomethyl cysteine (+57.0214) and variable modifications of oxidized methionine (+15.9949) and K-GG (+114.0429) considered. Linear discriminant analysis (LDA) was used to filter peptide spectral matches (PSMs) from each run to 5% false discovery rate (FDR) at the peptide level, and subsequently to a 2% protein level FDR as an aggregate of all runs (<0.5% peptide level FDR). Localization scores were generated for each K-GG PSM using a modified version of the AScore algorithm and positions of the modifications localized accordingly as the AScore sequence. (Beausoleil et al., Nat. Biotech. 24(10): 1285-1292 (2006)). Given work showing that trypsin cannot cut adjacent to ubiquitin modified lysines PSMs where the AScore sequence reports a -GG modification on the C-terminal lysine are dubious (Bustos et al., Mol. Cell. Proteomics, published online Jun. 23, 2012, doi: 10.1074/mcp.R112.019117; Seyfried et al., Anal. Chem., 80: 4161-4169 (2008)). Possible exceptions to this would be lysines at the C-termini of proteins (or in vivo truncation products), PSMs stemming from in source fragmentation of a bona fide K-GG peptide. To establish the most reliable dataset for downstream analysis, PSMs where the AScore sequence reports a C-terminal lysine were split into two groups: those with an available internal lysine residue to which the -GG could be alternatively localized, and those which lacked an available lysine. PSMs bearing a C-terminal K-GG but lacking an available lysine were removed from consideration in downstream analyses. For the remaining PSMs, the -GG modification was relocalized to the available lysine closest to the C-terminus.

Confidently identified peptides with ambiguous localization (AScore<13) bearing only a single internal lysine residue were reported with the modification localized to that internal lysine. Peptides where the modification has been assigned to the C-terminal lysine with an AScore>13 were discarded based on evidence suggesting that trypsin cannot cleave at a ubiquitin modified lysine residue.

A modified version of the VistaGrande algorithm, termed XQuant, was employed to interrogate the unlabeled peak areas for individual K-GG peptides, guided by direct PSMs or accurate precursor ion and retention time matching (cross quantitation). For direct PSMs, quantification of the unlabeled peak area was performed as previously described using fixed mass and retention tolerances (Bakalarski et al., J. Proteome Res., 7: 4756-4765 (2008)). To enable cross quantitation within XQuant, retention time correlation across pairwise instrument analyses was determined based on high-scoring peptide sequences identified by between one and four PSMs across all analyses within an experiment. Matched retention time pairings were modeled using a linear least squares regression model to yield the retention time correlation equation. In instrument analyses where a peptide was not identified by a discrete MS/MS, cross quantification was carried out by seeding the XQuant algorithm with the calculated mass of the precursor ion and its predicted retention time derived from the regression model. While the m/z tolerance was fixed, the retention time tolerance was dynamically adjusted for each pairwise instrument run. In cases where peptides were not confidently identified within a given instrument run but were identified in multiple other runs, multiple cross quantification events were performed to ensure data quality. XQuant results were filtered to a heuristic confidence score of 83 or greater, as previously described (Bakalarski et al., J. Proteome Res., 7: 4756-4765 (2008)). Full scan peak area measurements arising from multiple quantification events of the same m/z within a single run were grouped together if their peak boundaries in retention time overlapped. From such a group, the peak with the largest total peak area was chosen as its single representative.

To identify candidate substrates of Parkin and USP30, graphical analysis and mixed-effect modeling were applied to the XQuant data. A mixed-effect model was fit to the AUC data for each protein. “Treatment” (e.g. Control, Parkin overexpression/USP30 knockdown, CCCP, Combo) was a categorical fixed effect and “Peptide” was fit as random effect. False discovery rates (FDR) are calculated based on the P-values of each treatment vs. Control. Fold-changes and P-values of mean AUC from Combo vs. Control and Combo vs. CCCP were utilized in preparing plots. Mixed-effect model was fit in R by ‘nlme’ (Pinheiro et al., nlme: Linear and Nonlinear Mixed Effects Models. R package version 3, 1-101 (2011)).

### Preparation of Cell Lysates, and Immunoprecipitation

For total lysate experiments, cells were lysed after 24 hours in SDS sample buffer (Invitrogen) containing sample reducing agent (Invitrogen) and boiled at 95° C. for 10 minutes. Total lysates were resolved by SDS-PAGE and analyzed by immunoblotting. For immunoprecipitation experiments, cells were treated with 5 μM MG132 and the indicated concentrations and durations of CCCP at 24 hours (overexpression experiments) or 6 days (knockdown experiments) in 0.5% SDS in Tris-Buffered Saline (10 mM TRIS, 150 mM NaCl, pH 8.0) and boiled at 70° C. for 10 minutes. Lysates were diluted in immunoprecipitation buffer (50 mM HEPES, 150 mM NaCl, 10% glycerol, 1% Triton-X, protease inhibitors (Roche Applied Science), phosphatase inhibitors (Roche Applied Science), DNAse I (Roche Applied Science), 2 mM N-Ethylmaleimide (Thermo Scientific), pH 7.4), cleared by centrifugation at 31,000 g for 10 minutes, and incubated overnight with anti-HA affinity matrix beads (Roche Applied Science). Inputs and anti-HA immunoprecipitates were resolved by SDS-PAGE and analyzed by immunoblotting.

### Mitochondria Fractionation

Subcellular fractionation was performed using the FOCUS SubCell Kit (G Biosciences) from ˜P60 adult male rat forebrain.

### Drosophila Stocks

The following Drosophila lines were obtained for analysis: y, w; Actin5C-GAL4/CyO, y+(Bloomington Drosophila Stock Center, 4414), UAS-CG3016^{RNAi} (referred to here as UAS-dUSP30^{RNAi}; NIG-Fly Stock Center, 3016R-2). For USP30 knockdown experiments, Actin5C-GAL4 and UAS-dUSP30^{RNAi} were recombined onto the same chromosome using standard genetic techniques.

Flies were raised on Nutri-Fly “German Food” Formulation (Genesee, 66-115), prepared per manufacturer's instructions. All flies were raised at 25° C. and crossed using standard genetic techniques. All experiments were performed using age-matched male flies.

### Quantitative RT-PCR

RNA and subsequent cDNA was obtained from single flies following manufacturer's instructions (Qiagen RNeasy Plus kit, Applied Biosystems High Capacity cDNA Reverse Transcription kit). Quantitative RT-PCR was performed using an Applied Biosystems ViiA7 Real-Time PCR system using TaqMan Assays Dm01796115_g1 and Dm01796116_g1 (Drosophila CG3016 (USP30)), Dm01795269_g1 (Drosophila CG5486 (USP47)), and Dm01840115_s1 (Drosophila CG4603 (YOD1)). Dm02134593_g1 (RpII140) was used as a control.

### Determination of Ingested Paraquat Concentration

1-day old adult males were fed a solution containing 5% sucrose only (in water) or 5% sucrose+10 mM paraquat (in water) on saturated Whatman paper. After 48 hours of treatment, 15 flies were collected per condition and homogenized in 100 μL water. Standard curve samples were generated by spiking appropriate amounts of paraquat to homogenates from untreated flies. Then the samples were vortex mixed, 200 μl of acetonitrile containing internal standard (Propranolol) was added. The samples were vortexed again and centrifuged at 10,000×g for 10 min. Supernatants were transferred to a new plate that contained 200 μl of water and analyzed by LC-MS/MS to quantify for concentrations of paraquat. The LC-MS/MS consisted of an Agilent 1100 series HPLC system (Santa Clara, Calif.) and an HTS PAL autosampler from CTC Analytics (Carrboro, N.C.) coupled with a 4000 Q TRAP® MS and TurbolonSpray® ion source from Applied Biosystems (Foster City, Calif.). HPLC separation was performed on a Waters Atlantis dC18 column (3 μm 100×2.1 mm) with a Krud Katcher guard column from Phenomenex. Quantitation was carried out using the multiple reaction monitoring (MRM) with transition 185.1→165.1 for paraquat and 260.2→183.1 for propranolol. The lower and upper limit of the assay is 10 μM and 1000 μM, respectively. The quatitation of the assay employed a calibration curve which was constructed through plotting the analyte/IS peak area ration versus the nominal concentration of paraquat with a weighed 1/x² linear regression.

### Transmission Electron Microscopy of Drosophila Indirect Flight Muscles

Adult male thoraxes were isolated from the remainder of the body, then longitudinally hemi-sectioned and immediately fixed and processed as previously described (Greene et al., Proc. Natl. Acad. Sci. USA, 100: 4078-4083 (2003)).

### Climbing Assays

Climbing assays were performed using the following Drosophila lines: y, w; Actin5C-GAL4/CyO, y+(Actin only); y, w; UAS-CG3016-RNAi/CyO, y+(RNAi only); y, w; UAS-CG3016^{RNAi} Actin5C-GAL4/CyO, y+(USP30 knockdown).

1-day old adult males were fed a solution containing 5% sucrose only (in water) or 5% sucrose+10 mM paraquat (in water) on saturated Whatman paper. After 48 hours of treatment, flies were anesthetized using carbon dioxide and transferred in groups of ten to vials containing only 1% agarose for a 1-hour recovery period from the effects of carbon dioxide. The flies were then transferred into a new glass tube, gently tapped to the bottom and scored for their ability to climb. The number of flies climbing vertically >15 cm in 30 seconds was recorded.

### Survival Assays

Ten adult 1-day old males per vial were fed a solution containing 5% sucrose only (in water) or 5% sucrose+10 mM paraquat (in water) on saturated Whatman paper. The number of live flies was counted at described intervals.

### MultiTox Cell Death Assay

SH-SY5Y cells transfected with control or USP30 siRNAs are treated with rotenone in normal growth medium (DMEM/F12 and 1× GlutaMax) containing 1% Fetal Bovine Serum. Following 24 hours of incubation, Multi-Tox Fluor assay (Promega) is used to measure cell viability according to manufacturer's instructions. GF-AFC fluorescence is normalized to bis-AAF-R110 fluorescence for each condition and presented as a fraction of control (control RNAi+DMSO).

### Example 2

### USP30 Antagonizes Parkin-Mediated Clearance of Damaged Mitochondria

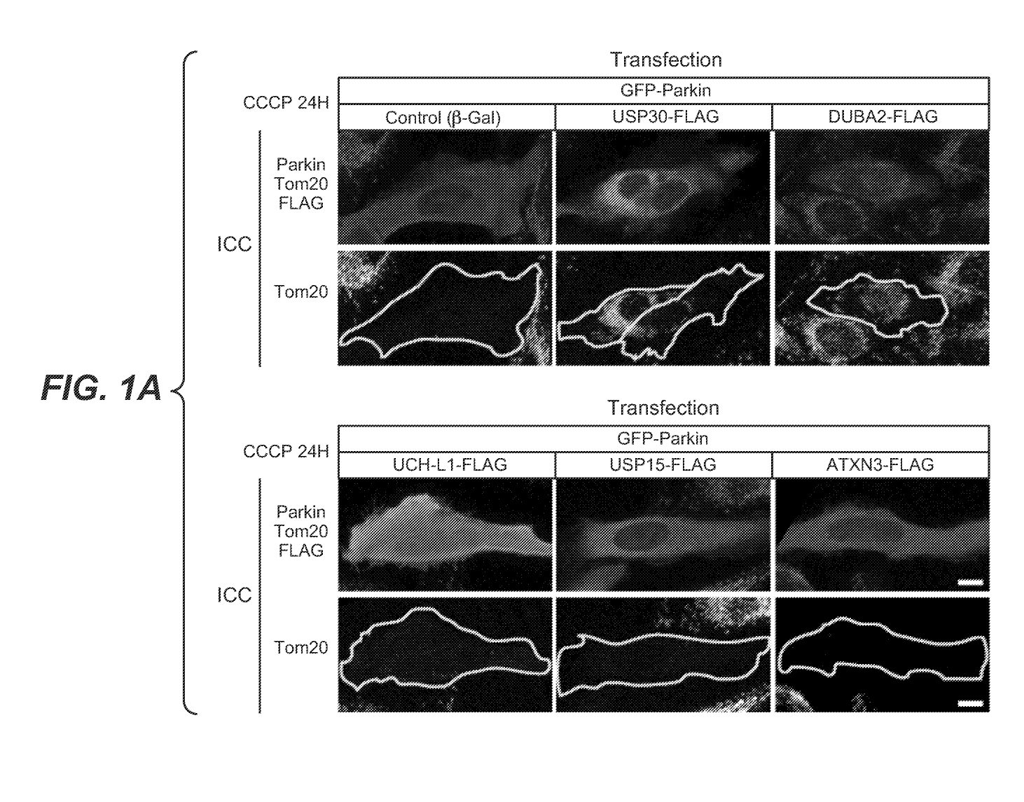

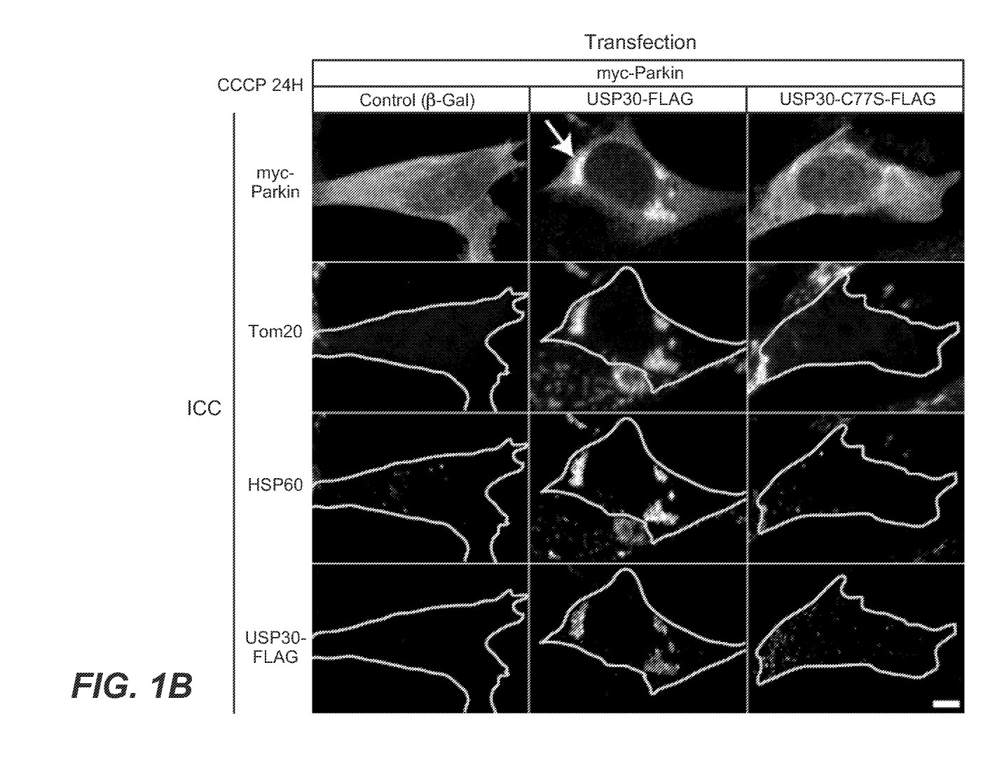

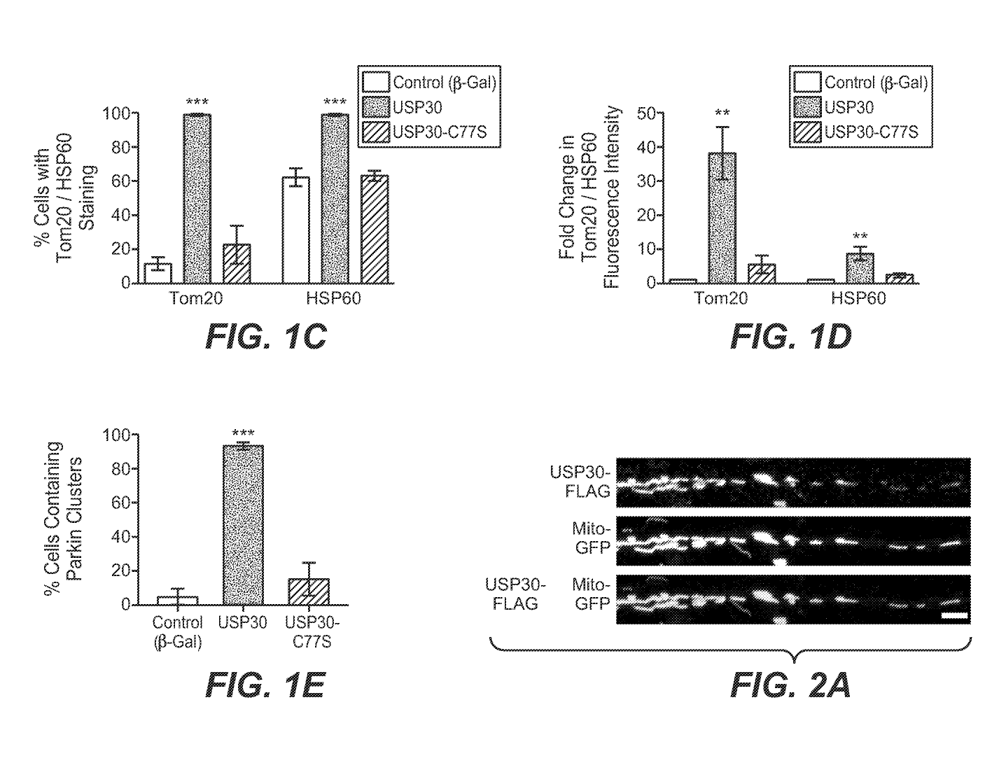

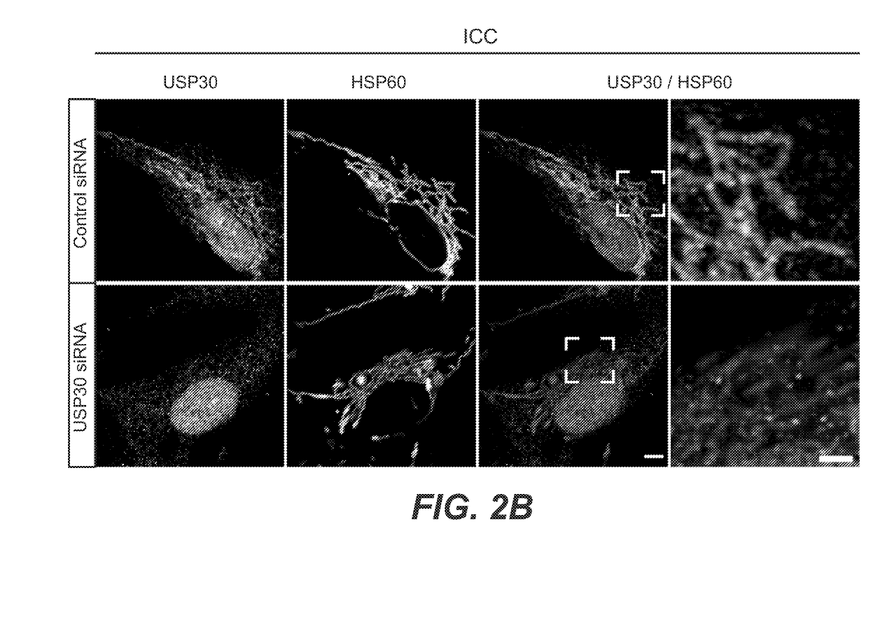

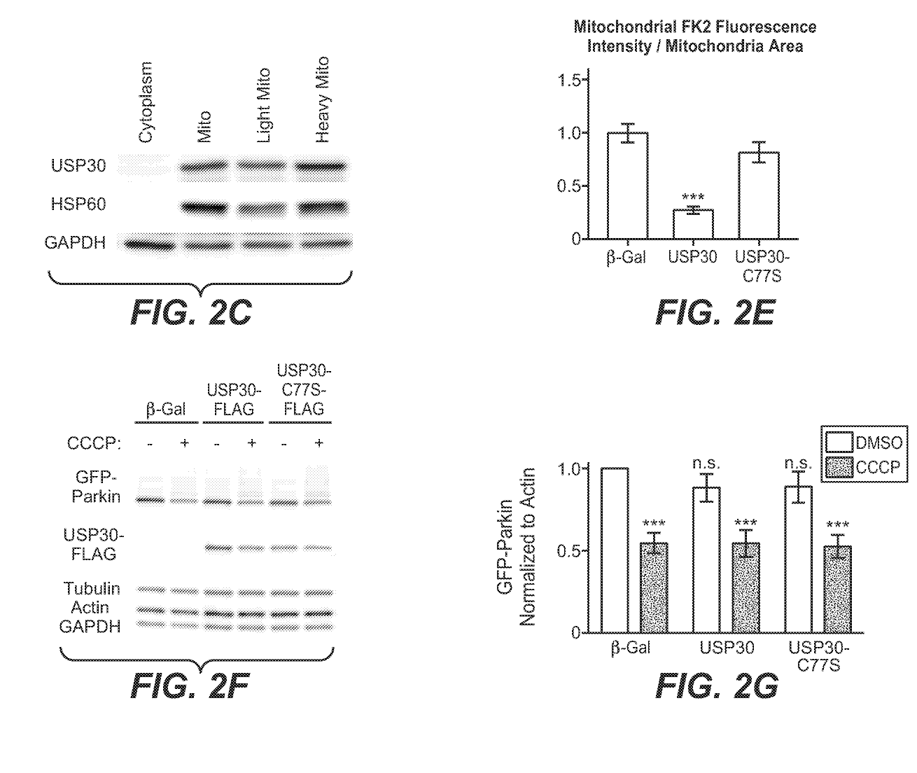

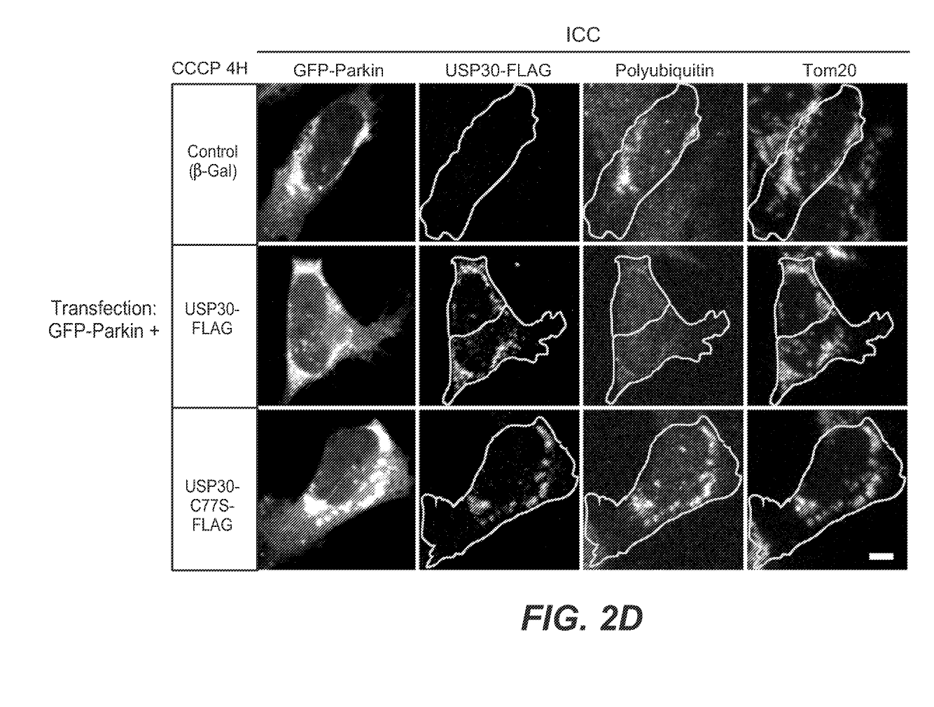

To identify DUBs that regulate mitochondria clearance, a FLAG-tagged human DUB cDNA library (97 DUBs) was screened in an established mitochondrial degradation assay (Narendra et al., J. Cell Biol., 183: 795-803 (2008)). In this assay, mitochondria depolarization induced by protonophore carbonyl cyanide 3-chlorophenylhydrazone (CCCP, 20 μM, 24 hours) results in marked loss of mitochondria in cultured cells overexpressing Parkin (as measured by staining for mitochondria outer membrane protein marker Tom20). CCCP treatment led to a robust disappearance of Tom20 staining in the great majority of cells transfected with GFP-Parkin (>80% of Parkin-transfected cells lacked Tom20 staining after CCCP—FIG. 1A). Individual FLAG-tagged DUB cDNAs were cotransfected with GFP-Parkin, and their effects on CCCP-induced mitochondrial (Tom20) clearance were measured. Out of the library of ˜100 different DUBs, 2 DUBs, USP30 and DUBA2, robustly blocked the loss of Tom20 staining in CCCP-treated GFP-Parkin-transfected cells, whereas others had little effect (FIG. 1A—% of cells with Tom20 staining: control (β-Gal): 15.3%, USP30: 97.4%, DUBA2: 94.7%, UCH-L1: 36%, USP15: 23.3%, ATXN3: 8.3%; other negative DUBs not shown). USP30 rather than DUBA2 was selected for further study since USP30 has been reported to be localized in the mitochondrial outer membrane with its enzymatic domain putatively facing the cytoplasm (Nakamura and Hirose, Mol. Biol. Cell, 19: 1903-1914 (2008)); thus it would be in the right subcellular compartment to counteract the action of Parkin on mitochondria. The specific mitochondrial association of USP30 was confirmed by immuno-colocalization of transfected USP30-FLAG and of endogenous USP30 with mitochondrial markers in neurons (FIG. 2A, B), as well as by cofractionation of USP30 with purified mitochondria from rat brain (FIG. 2C).

The ability of USP30 overexpression to prevent CCCP-induced mitophagy was also shown in a different cell line (dopaminergic SH-SY5Y cells) transfected with myc-Parkin (FIG. 1B). To confirm that the effects of USP30 were not specific to Tom20, whether USP30 overexpression also prevented the CCCP-induced loss of the mitochondrial matrix protein HSP60 was tested. Indeed, USP30 overexpression also prevented the CCCP-induced loss of HSP60, implying USP30 blocks en masse degradation of the organelle (FIG. 1B-D). In contrast, expression of an catalytically-inactive USP30 C77S mutant (Nakamura and Hirose, Mol. Biol. Cell, 19: 1903-1914 (2008)) was ineffective at preventing Parkin-mediated mitochondria degradation, supporting the idea that USP30 counteracts mitophagy through deubiquitination of mitochondrial substrates (FIG. 1B-D).

Since USP30 enzymatic activity was necessary for blocking mitophagy, whether USP30 and Parkin have opposing effects on mitochondria ubiquitination was examined. As reported previously, short-term CCCP treatment (20 μM, 4 hours) caused Parkin redistribution to mitochondria (marked by Tom20) and led to accumulation of ubiquitination signal on mitochondria (measured by staining with polyubiquitin antibody FK2, FIG. 2D; (Lee et al., J. Cell Biol., 189: 671-680 (2010)). When USP30 was co-expressed with Parkin, the amount of ubiquitin signal accumulated on mitochondria was reduced by ˜75% —an effect that also required USP30 enzymatic activity (FIGS. 2D, E). These data support the idea that USP30 functions as a DUB that opposes the ubiquitin ligase action of Parkin on mitochondrial proteins, thereby inhibiting mitophagy.

Previous studies indicated that Parkin pathogenic mutants defective in ligase activity cannot support mitochondrial degradation in response to CCCP, leading to clustering of uncleared mitochondria in the perinuclear region in association with translocated Parkin (Geisler et al., Nat. Cell Biol., 12: 119-131 (2010); Lee et al., J. Cell Biol., 189: 671-680 (2010)). Remarkably, in cells co-transfected with USP30 plus Parkin and treated with CCCP, wild-type myc-Parkin behaved similarly to mutant Parkin in that it remained associated with the perinuclear clusters of non-degraded mitochondria (FIG. 1B (white arrow), E). Co-expression of USP30 did not alter Parkin expression level (FIGS. 2F, G). These data indicate that USP30 blocks mitophagy by enzymatic removal of ubiquitin signal on damaged mitochondria, rather than by inhibiting the translocation of Parkin to mitochondria.

### Example 3

### Pink1, Parkin Required for Mitophagy in Neurons

To measure mitophagy in neurons, mt-Keima, a ratiometric pH-sensitive fluorescent protein that is targeted into the mitochondrial matrix, was monitored. A low ratio mt-Keima-derived fluorescence (543 nm/458 nm) reports neutral environment whereas a high ratio fluorescence reports acidic pH (Katayama et al., Chemistry & Biology 18: 1042-1094 (2011)). Thus mt-Keima enables differential imaging of mitochondria in the cytoplasm and mitochondria in acidic lysosomes. Because mt-Keima is resistant to lysosomal proteases, it allows for measurement of cumulative lysosomal delivery of mitochondria over time.

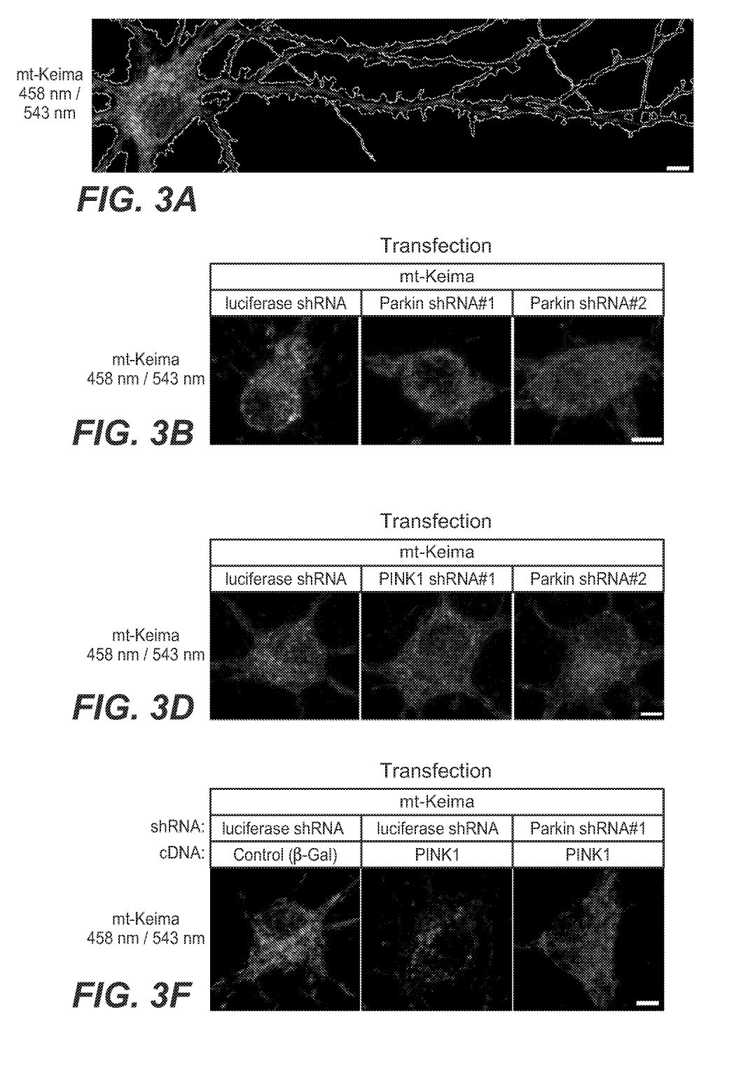

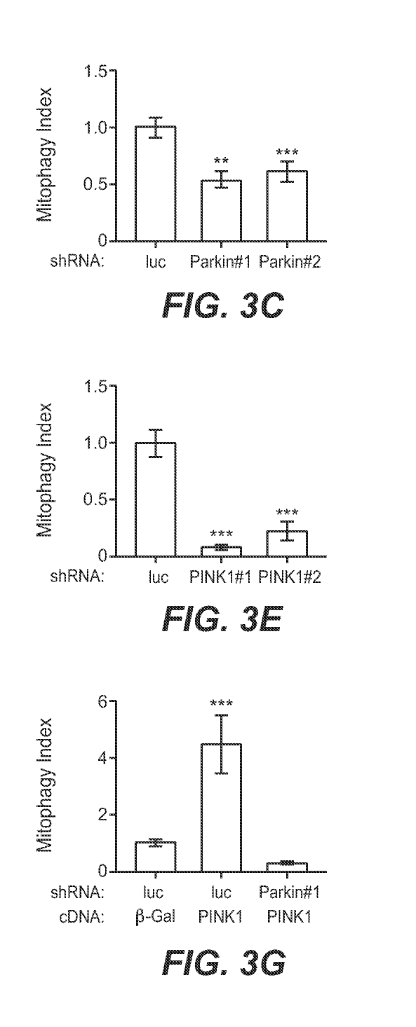

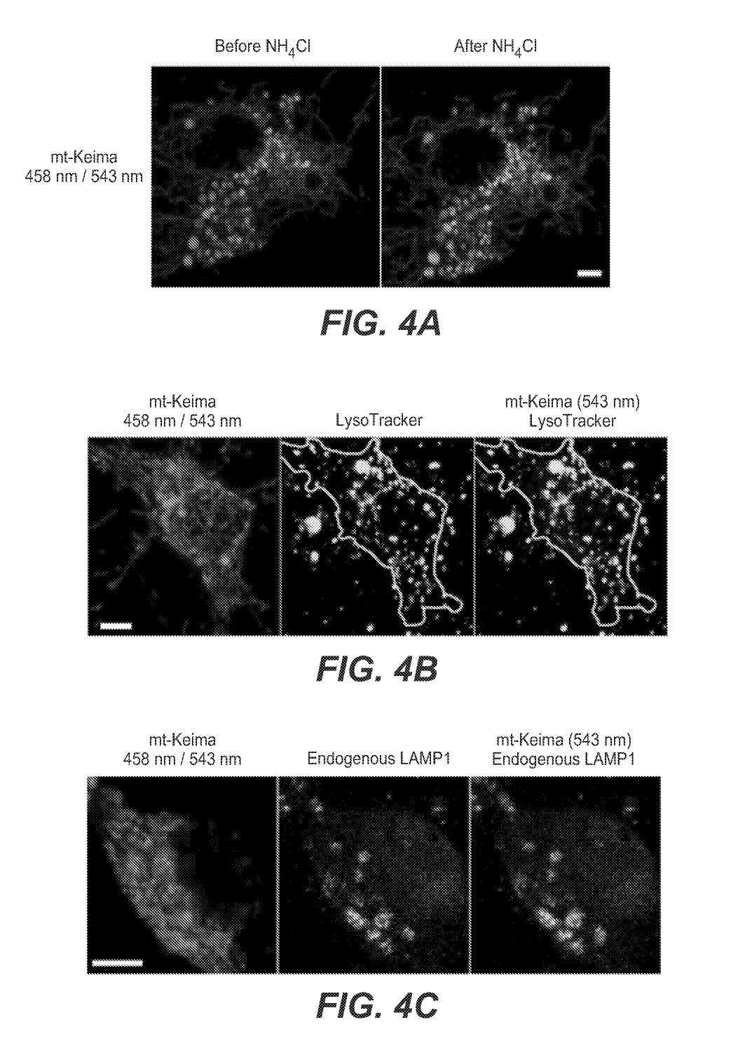

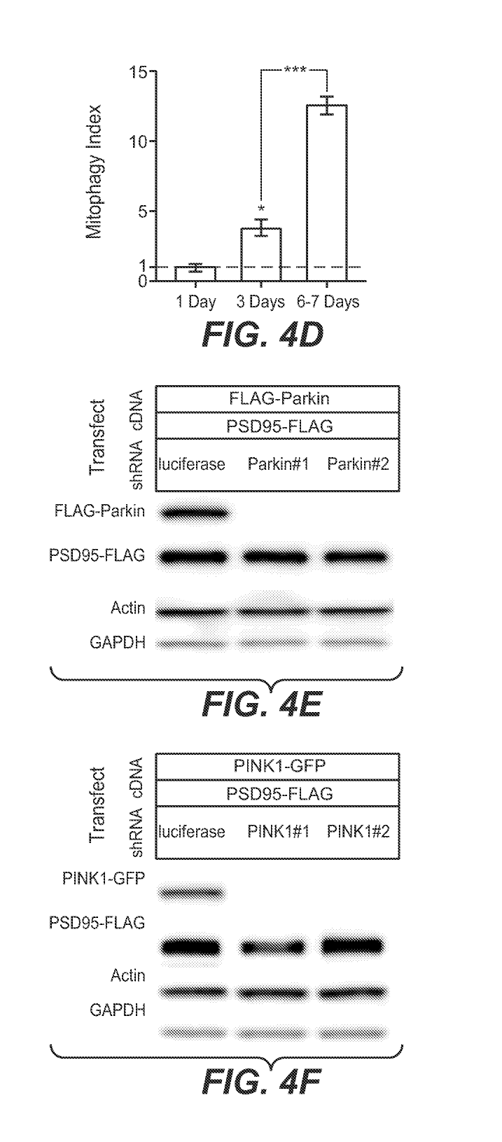

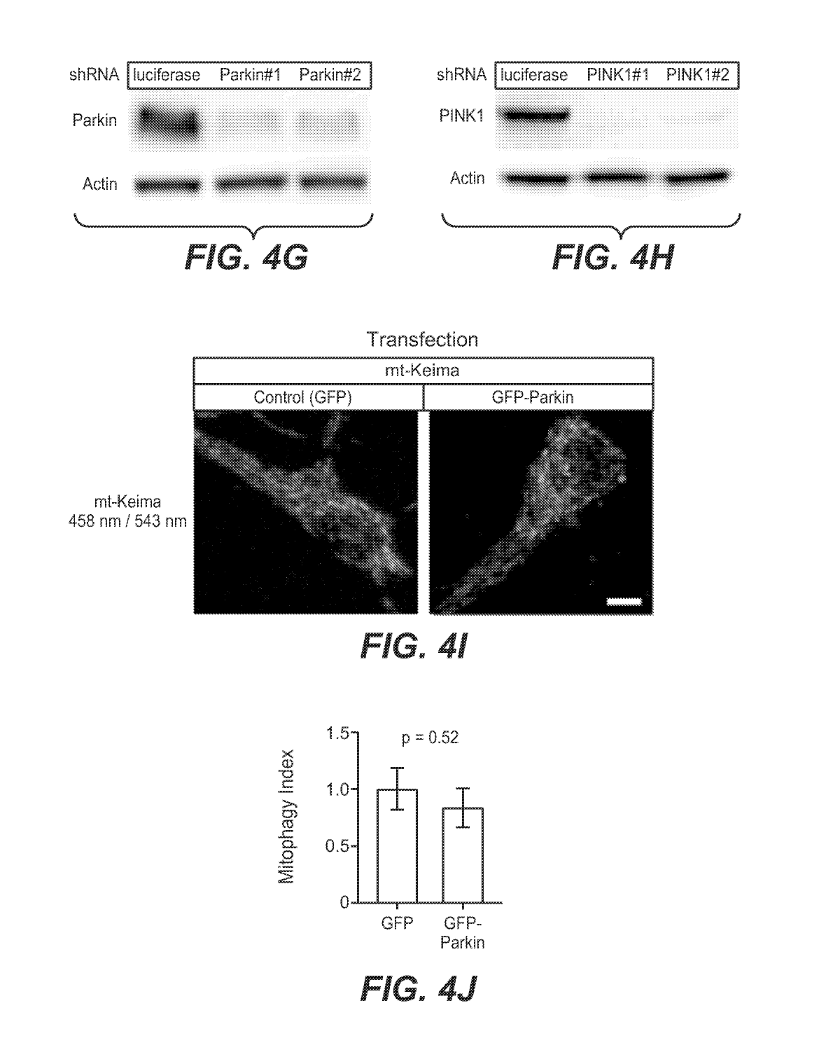

Following transfection in rat dissociated hippocampal cultures, mt-Keima signal accumulated initially in elongated structures characteristic of mitochondria and with low 543/458 ratio values (shown in green—FIG. 3A). After 2-3 days of expression, multiple round mt-Keima structures with high ratio (acidic) signal also appeared throughout the cell body (shown in red—FIG. 3A). These round mt-Keima-positive structures most likely represent lysosomes since (1) neutralizing cells with NH4Cl completely reversed the high ratio (543/458) pixels to low ratio signal specifically in these round structures without affecting the tubular-reticular mitochondrial signal (FIG. 4A); (2) an independent lysosomal marker dye (lysotracker green DND-26) stained high ratio mt-Keima structures, though there were also many Lysotracker-positive structures that were not associated with mt-Keima (FIG. 4B); (3) in post-hoc immunostaining experiments, high ratio pixels colocalized with endogenous lysosomal protein LAMP-1 (FIG. 4C). Since almost all of the “acidic” mt-Keima signal was found in neuronal cell bodies (cell body contained 95.6±2.2% of the total high ratio (543/458) signal), the ratio of the area of lysosomal (red) signal/mitochondrial (green) signal within the cell body was used as a measure of lysosomal delivery of mitochondria in neurons (“mitophagy index”) (Katayama et al., Chemistry & Biology 18: 1042-1094 (2011)). As quantified by this mitophagy index, the abundance of mt-Keima in lysosomes increased over a time course of days (FIG. 4D), implying active mitophagy in cultured neurons under basal conditions.

In heterologous cells, Parkin overexpression can drive mitochondrial degradation upon mitochondria depolarization; however, it is not yet established whether endogenous Parkin and PINK1 are required for mitophagy in non-neural or neural cells (Youle and Narendra, Nat. Rev. Mol. Cell Biol. 12: 9-14 (2011)). To examine the role of the PINK1/Parkin pathway in neuronal mitophagy, Parkin or PINK1 was knocked down using small hairpin RNAs (shRNAs) expressed from pSuper-based vectors. These Parkin and PINK1 shRNAs efficiently knocked down the cDNA-driven expression of their respective targets in heterologous cells (FIGS. 4E, F), and suppressed the protein levels of endogenous Parkin or PINK1 in neuronal cultures by ˜80% and ˜90%, respectively (FIGS. 4G, H). Compared to control luciferase shRNA, neurons transfected with Parkin shRNAs (two independent sequences) showed ˜50% reduction in the mitophagy index, indicative of decreased mitochondria delivery to lysosomes (FIGS. 3B, C). PINK1 shRNAs were even more effective in reducing the acidic mt-Keima signal (˜80-90% reduction in mitophagy index (FIGS. 3D, E)). Previous genetic studies placed PINK1 upstream of Parkin in maintaining healthy mitochondria (Clark et al., Nature, 441: 1162-1166 (2006); Park et al. Nature, 441: 1157-1161 (2006)). Consistent with the genetic epistasis, our mt-Keima experiments showed that PINK1 overexpression strongly enhanced mitophagy in neurons, an effect that was completely eliminated by Parkin knockdown (FIGS. 3F, G). On the other hand, Parkin overexpression by itself had no apparent effect on basal mitophagy, as measured by the mt-Keima assay (FIGS. 4I, J). Thus, neuronal mitophagy requires both PINK1 and Parkin, with PINK1—apparently limiting—acting upstream of Parkin.

### Example 4

### USP30 Antagonizes Mitophagy in Neurons

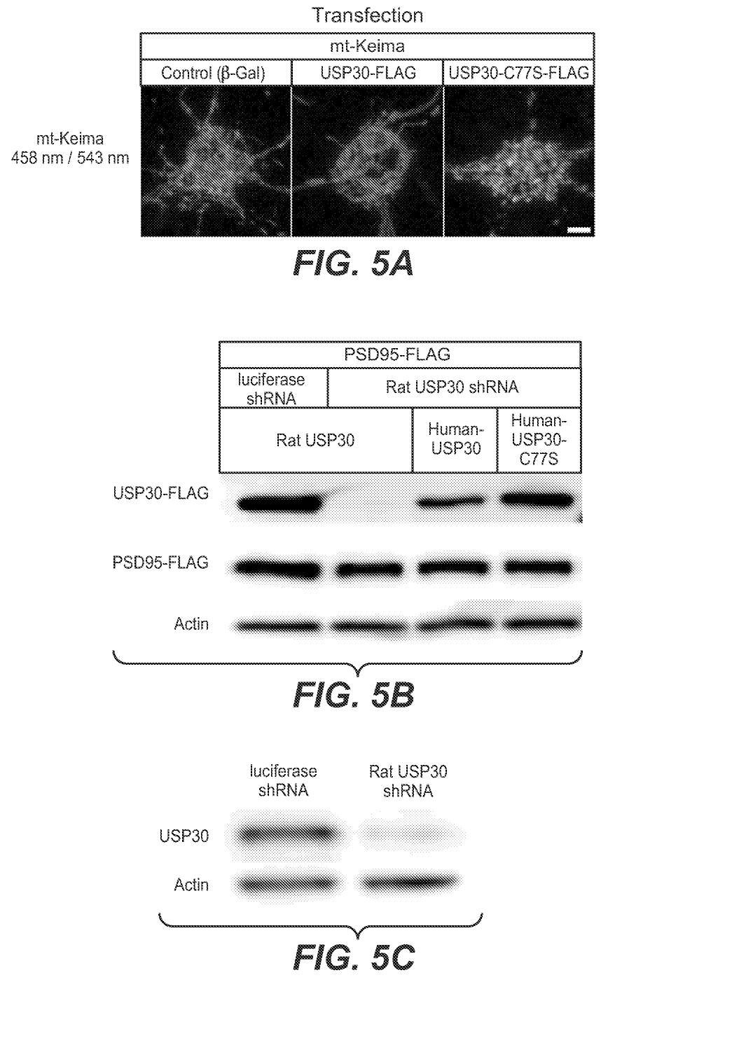

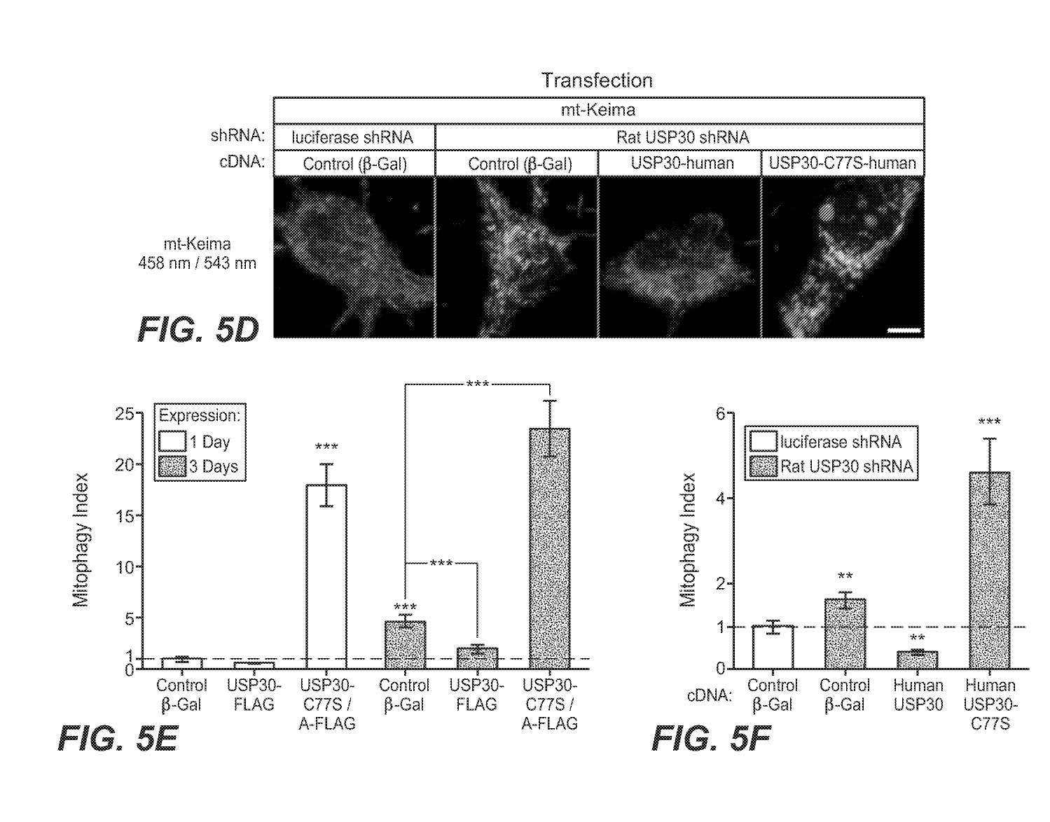

Next, whether USP30 suppresses mitophagy in neurons as in heterologous cells was investigated. Compared with control neurons transfected with β-Gal and mt-Keima, co-expression of wild-type USP30 caused a ˜60% reduction in mitophagy index at 3 days, indicating that USP30 inhibits lysosomal delivery of mitochondria in neurons (FIGS. 5A, E). In contrast, overexpression of enzymatically-inactive USP30 (C77S or C77A) induced a robust increase in mitophagy signal (FIGS. 5A, E). The enhanced delivery of mitochondria to lysosomes likely reflects a dominant-negative action of catalytically-inactive USP30, presumably by interacting with substrates or pro-mitophagy ubiquitin chains, and sequestering them from endogenous USP30 (Berlin et al., J. Biol. Chem., 285: 34909-34921 (2010); Bomberger et al., J. Biol. Chem., 284: 18778-18789 (2009); Ogawa et al., J. Biol. Chem., 286: 41455-41465 (2011)).

To test the function of endogenous USP30, USP30 was knocked down using shRNAs. In heterologous cells, rat USP30 shRNA plasmid specifically eliminated the expression of transfected rat USP30 cDNA (FIG. 5B). The same rat USP30 shRNA led to a ˜85% reduction in endogenous USP30 in neuronal cultures (FIG. 5C). In neurons, USP30 knockdown increased the lysosomal delivery of mt-Keima (˜60% increase in mitophagy index), relative to negative control luciferase shRNA (FIGS. 5D, F). Co-transfection of shRNA-resistant human USP30 cDNA “rescued” this effect, i.e. it restored the brake on mitochondrial degradation, indicating that USP30 shRNA was not exerting a non-specific effect (FIGS. 5B, D, F). In fact, neurons co-transfected with human USP30 cDNA plus rat USP30 shRNA showed lower levels of lysosomal accumulation of mt-Keima than controls, similar to neurons overexpressing wild-type USP30 by itself (FIGS. 5D, F). Moreover, enzymatically-inactive human USP30 (C77S) failed to reverse the enhanced mitophagy induced by USP30 shRNA, and actually enhanced mitophagic activity even more than USP30 shRNA (FIGS. 5D, F), the latter result suggesting that USP30 knockdown is incomplete. These results provide strong evidence that endogenous USP30 restrains mitophagy in neurons through its DUB activity.

### Example 5

### USP30 Deubiquitinates Multiple Mitochondrial Proteins

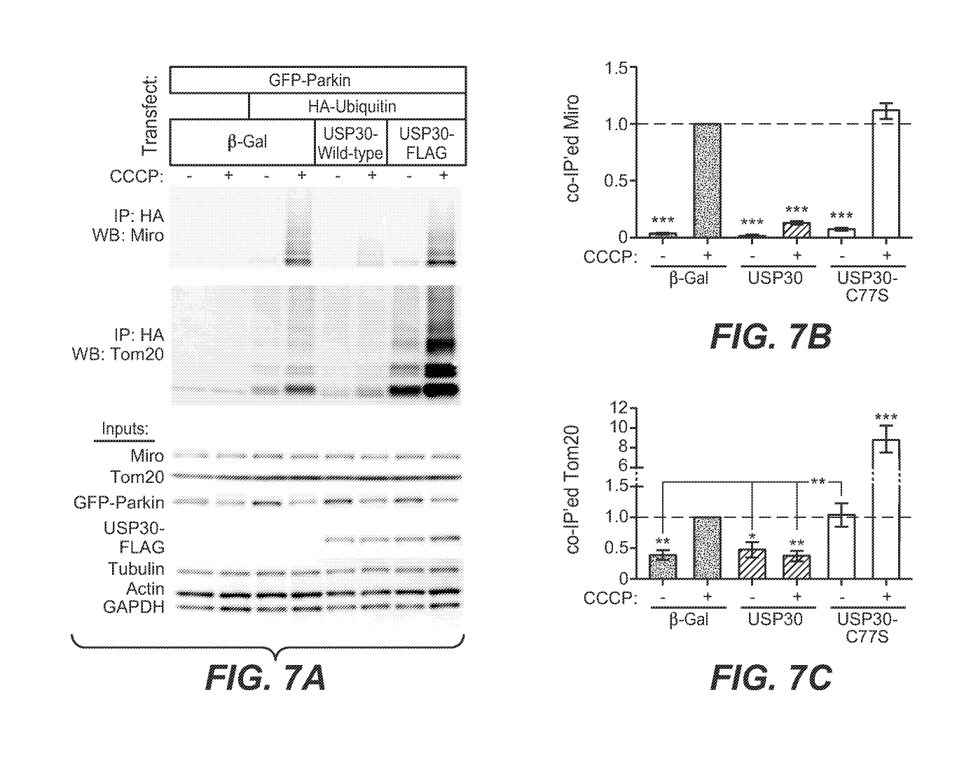

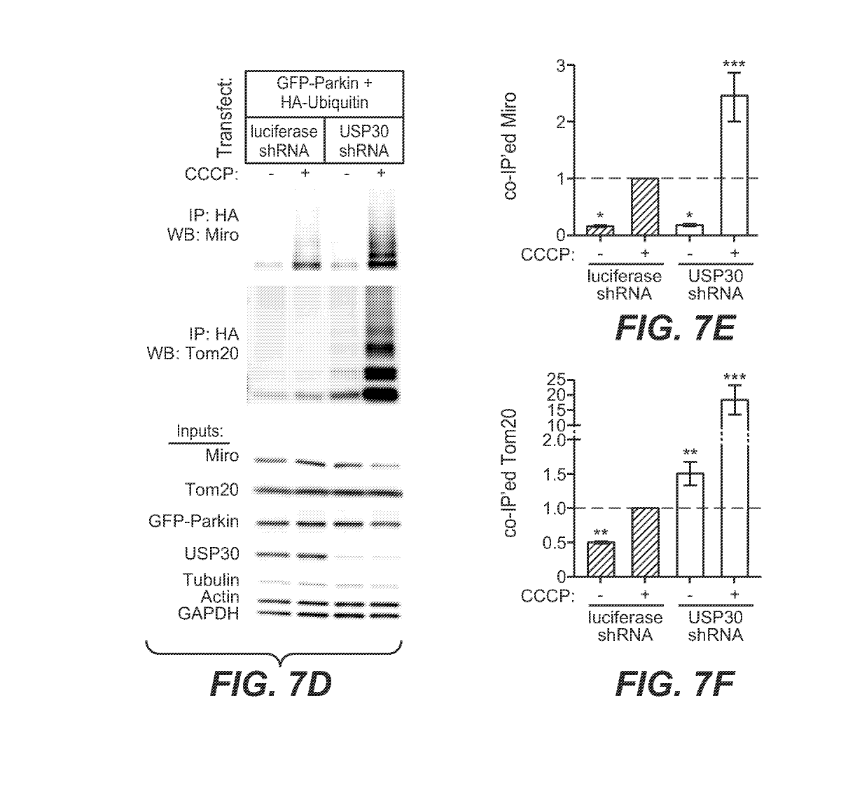

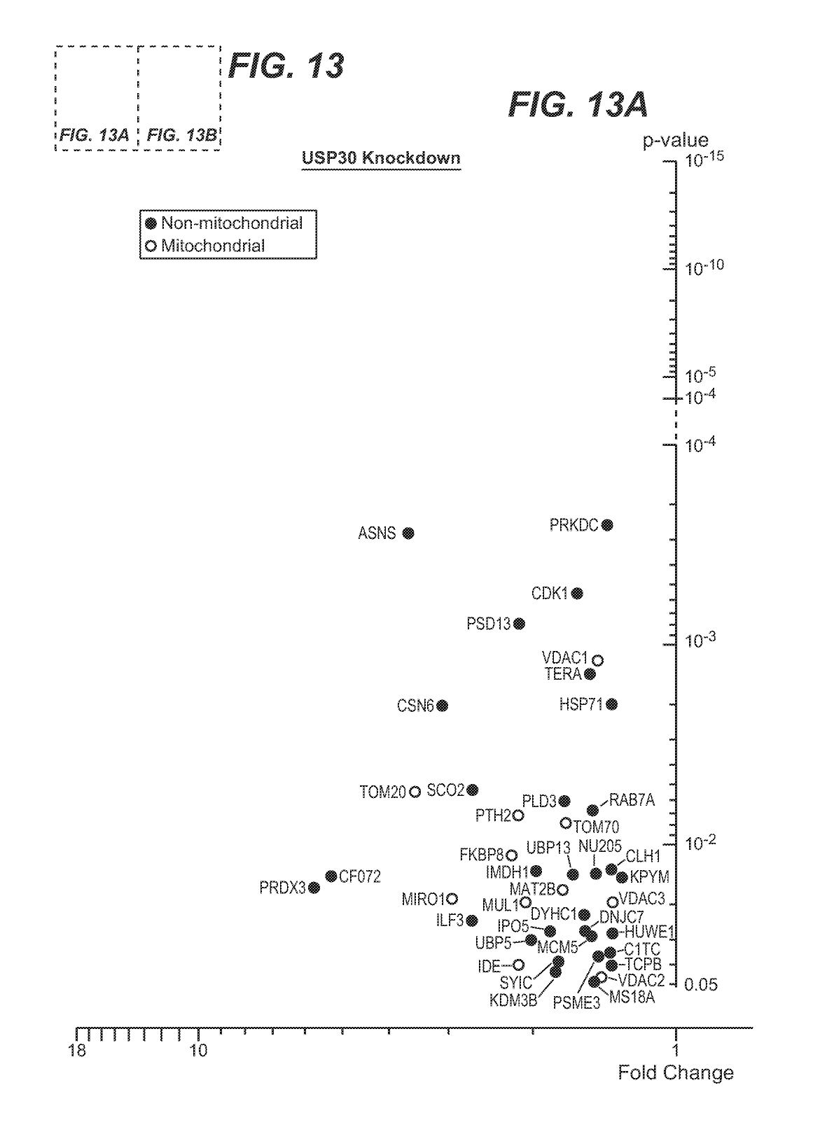

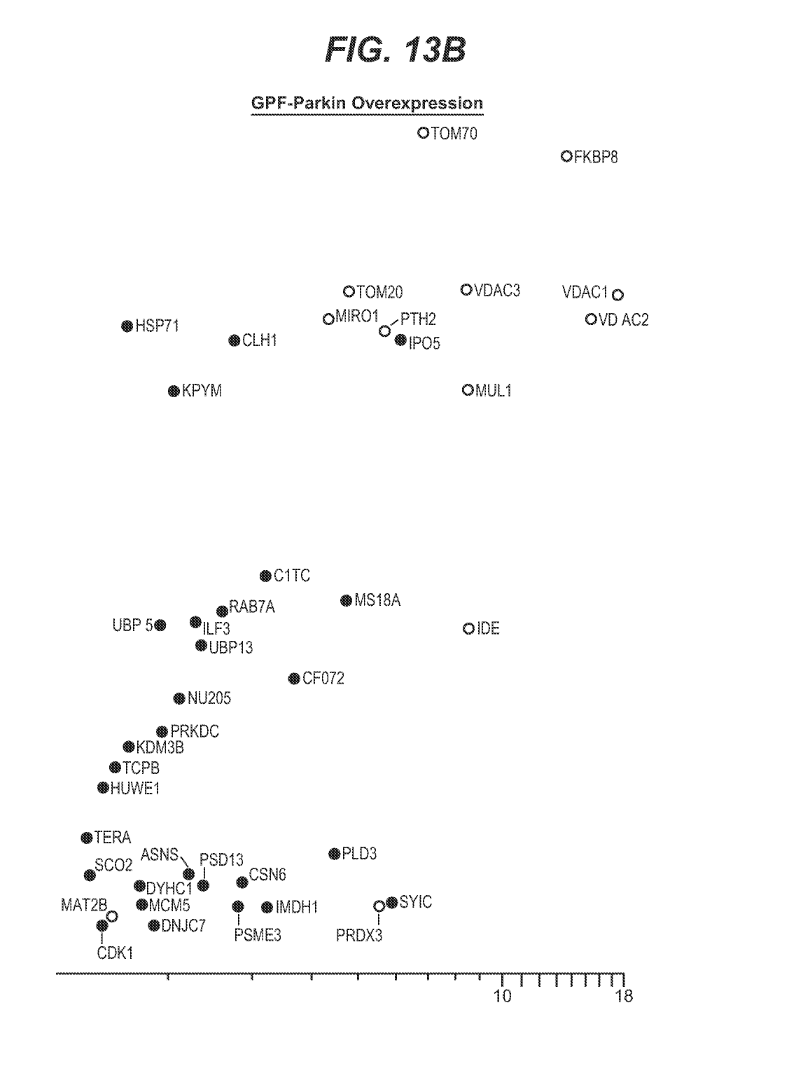

Since Parkin and USP30 antagonistically regulate mitochondrial degradation, it was hypothesized that this E3 ligase and DUB act on some common substrates. To identify Parkin and USP30 substrates, global ubiquitination in cells was analyzed by mass spectrometry (MS) following immunoaffinity enrichment of ubiquitinated peptides from trypsin-digested extracts using the ubiquitin branch-specific (K-GG) antibodies. Global ubiquitination was analyzed and quantified by MS in HEK-293 cells in two different sets of conditions: 1) inducible Parkin overexpression, or 2) USP30 knockdown (USP30 knockdown efficiency was 85±5% (see FIG. 7C)). In each set, cells were treated with CCCP (5 μM, 2 hours) or vehicle control (DMSO). In aggregate, MS analysis revealed >15,000 unique ubiquitination sites on ˜3200 proteins of which a subset responded to either CCCP alone (endogenous Parkin and USP30 levels) or Parkin overexpression/USP30 knockdown (see Appendix A for a list of the ˜3200 proteins). MS identified 233 and 335 proteins whose ubiquitination increased by parkin overexpression or USP30 knockdown, respectively (i.e. exhibited significantly more ubiquitination in ‘parkin overexpression+CCCP’ or ‘USP30 knockdown+CCCP’ vs. CCCP-alone. 41 of these proteins were regulated by both Parkin overexpression and USP30 knockdown (FIG. 13). Twelve of these 41 proteins are mitochondrial or associated with mitochondria (Tom20, MIRO1, FKBP8, PTH2, MUL1, MAT2B, TOM70, PRDX3, IDE, and all three VDAC isoforms—based on Human MitoCarta database). Others included nuclear import proteins (e.g. IP05), demethylases (e.g. KDM3B), and components of the ubiquitin/proteasome system (e.g., PSD13, UBP13) (FIG. 13).

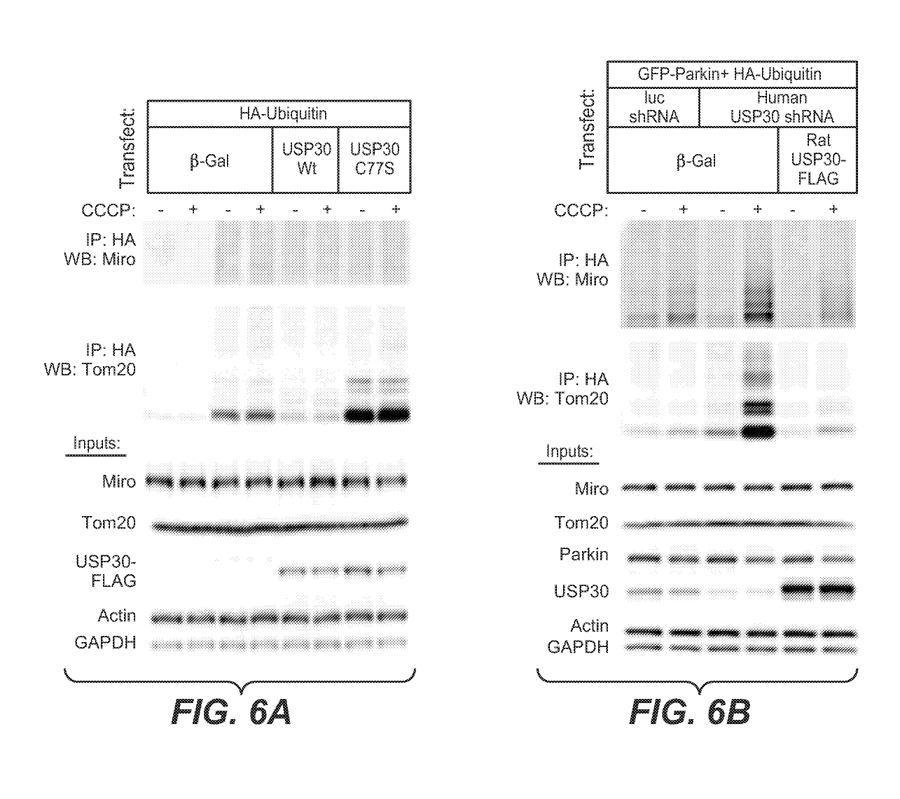

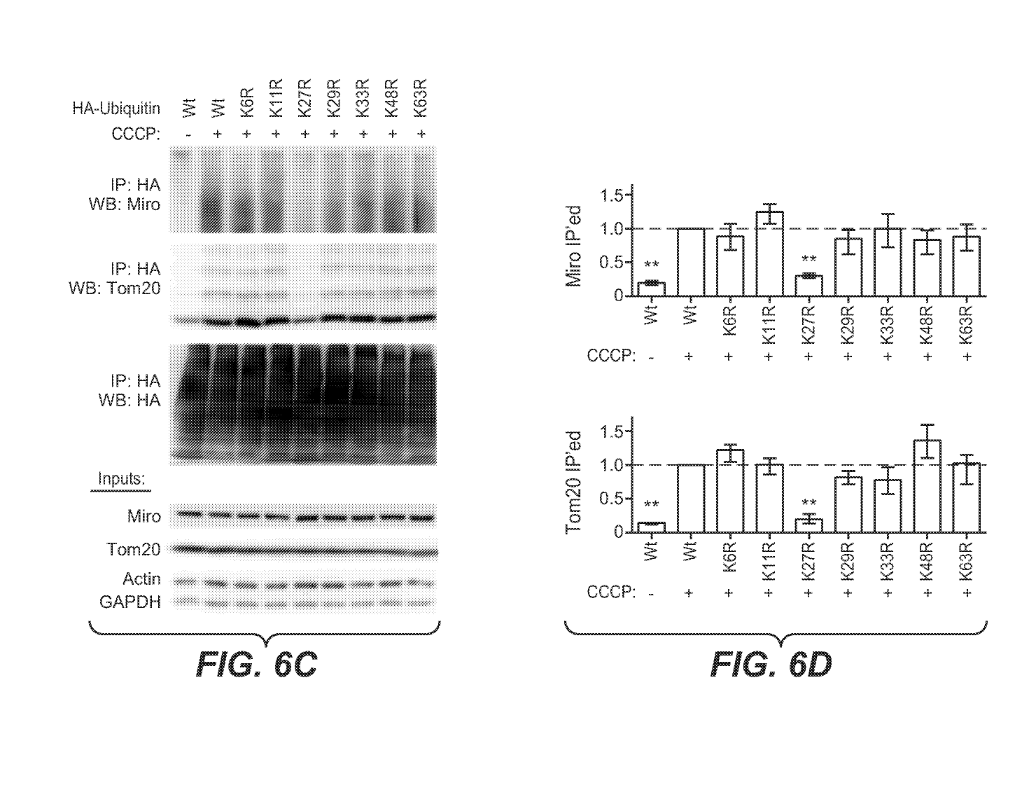

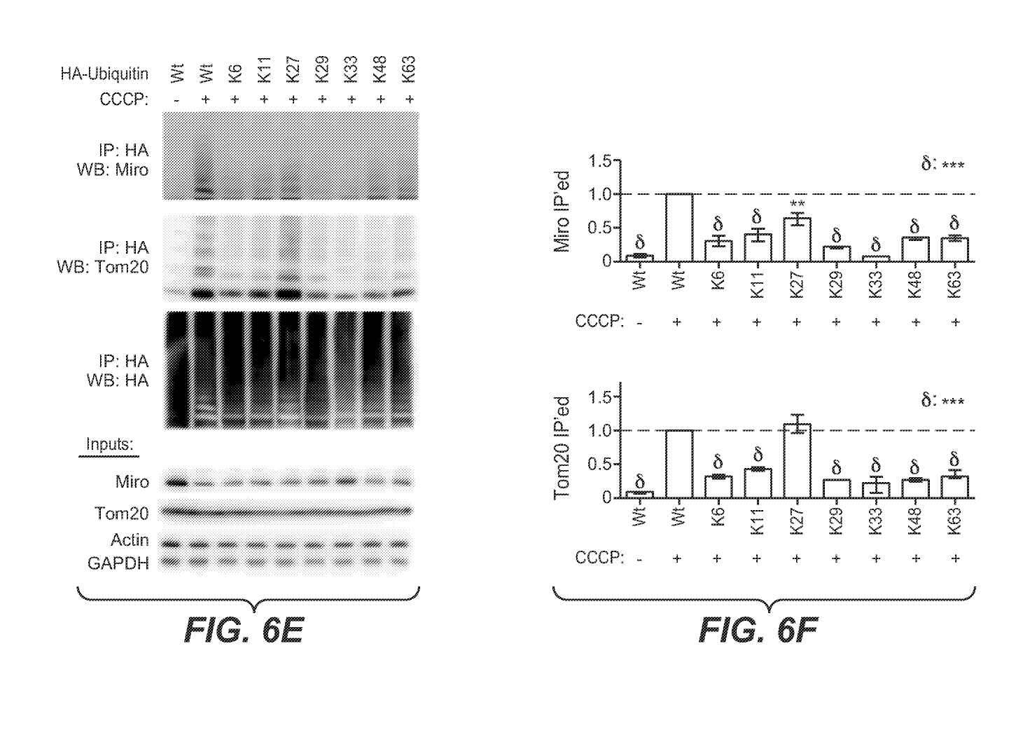

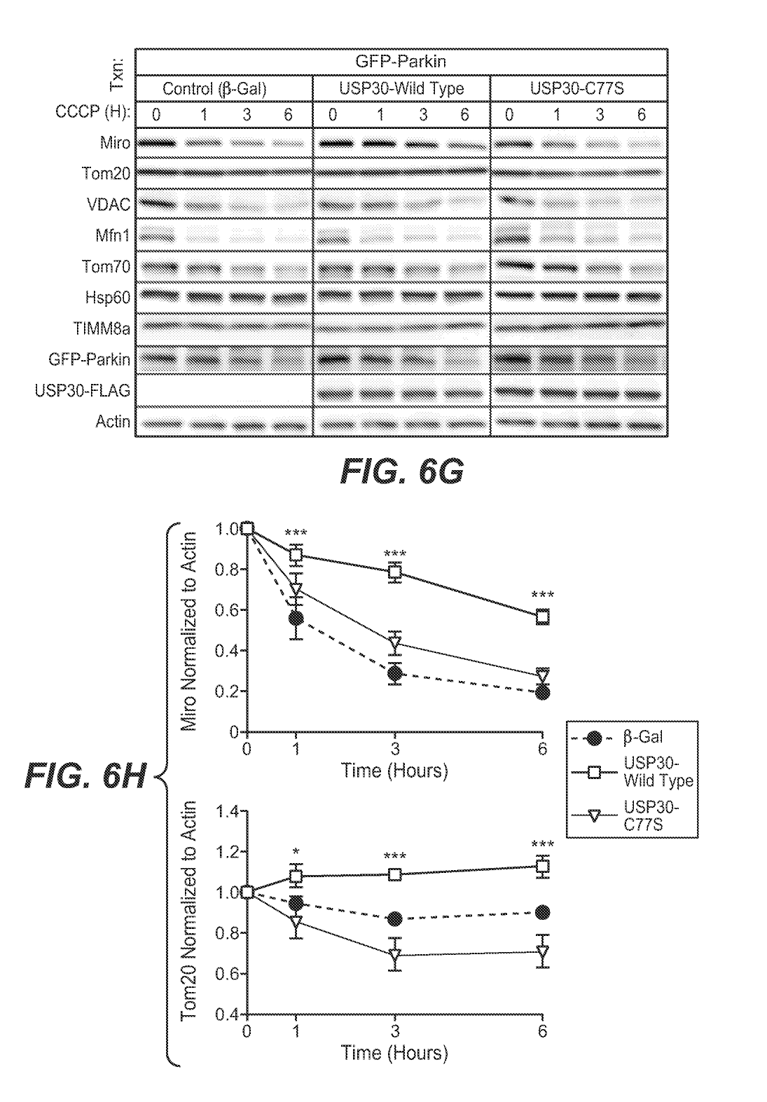

We focused additional studies on two mitochondrial proteins—Tom20 and MIRO—that showed large increases in ubiquitination with USP30 knockdown (USP30 shRNA+CCCP/CCCP ubiquitination ratio for Tom20=3.52, p=0.005; for MIRO=2.95, p=0.019; see FIG. 13, left). Tom20 and MIRO also showed large magnitude and highly significant increases in ubiquitination with Parkin overexpression (FIG. 13, right). To confirm that USP30 can deubiquitinate these proteins, cell lines stably overexpressing GFP-Parkin were transfected with HA-ubiquitin and immunoprecipitated (IP) ubiquitinated proteins using anti-HA antibodies. Following mitochondrial depolarization (CCCP, 5 μM, 2 hours), GFP-Parkin stable cells showed robust enhanced ubiquitination of endogenous MIRO, as measured by immunoblotting for MIRO in the anti-HA-immunoprecipitates (FIG. 7A). In control transfections without HA-ubiquitin, anti-HA-beads did not immunoprecipitate MIRO, indicating the specificity of MIRO ubiquitination signal (FIG. 7A, left lanes). Compared to β-Gal control, cotransfection of wildtype USP30, but not DUB-dead USP30-C77S, decreased the amount of ubiquitininated MIRO by ˜85% (FIGS. 7A, B). Similarly, wildtype USP30 overexpression reduced the ubiquitination of Tom20 (FIG. 7A); whereas USP30-C77S actually increased basal Tom20 ubiquitination ˜2-fold (without CCCP), and CCCP-induced ubiquitination ˜8-fold, consistent with a dominant-negative mechanism (FIGS. 7A, C). CCCP did not induce detectable Tom20 or MIRO ubiquitination in the parental HEK-293 cell line (lacking GFP-Parkin) (FIG. 6A). In this cell line, however, overexpression of USP30-C77S was still able to enhance basal Tom20 ubiquitination and USP30 to suppress it (FIG. 6A). Taken together, our data indicate that MIRO and Tom20 are substrates of USP30 and that USP30 can counteract Parkin-mediated ubiquitination of both MIRO and Tom20 following mitochondria damage.

It was found that a subset of the shared substrates were regulated by USP30 even under basal conditions (exemplified by Tom20, discussed above). MUL1, ASNS and FKBP8—but not MIRO—were substrates that behaved similarly to Tom20; i.e. they also exhibited a basal increase in ubiquitination with USP30 knockdown in the absence of CCCP. Thus, USP30 basally deubiquitinates this set of proteins, possibly counterbalancing against a mitochondrial E3 ligase that is active in the absence of CCCP and that acts on Tom20 but not MIRO. On the other hand, proteins such as TOM70, MAT2B and PTH2 behaved similarly to MIRO in that they exhibited enhanced ubiquitination with USP30 knockdown only following CCCP, suggesting that USP30 engages in deubiquitination of these proteins only after Parkin is recruited to mitochondria. Parkin, following recruitment to damaged mitochondria, may target both Tom20 and MIRO types of USP30 substrates, shifting the balance towards their polyubiquitination.

Using the same experimental system (cells overexpressing GFP-Parkin and HA-ubiquitin), the function of endogenous USP30 was tested by shRNA suppression. USP30 knockdown did not affect basal ubiquitination of MIRO (in the absence of CCCP-induced mitochondria damage). After mitochondrial depolarization (CCCP 5 μM, 2 hours), however, and consistent with the MS experiments, USP30 knockdown increased the level of ubiquitinated MIRO ˜2.5-fold, as measured in HA-ubiquitin immunoprecipitates (FIGS. 7D, E). Notably, USP30 knockdown increased both basal and CCCP-induced Tom20 ubiquitination, similar to enzymatically-inactive USP30 (FIGS. 7D, F). The increase in MIRO and Tom20 ubiquitination caused by USP30 shRNA was prevented by expression of the rat USP30 cDNA that is insensitive to human USP30 shRNA, indicating the specificity of the RNAi effect (FIG. 6B). Thus, these biochemical data corroborate the MS findings that endogenous USP30 acts as a brake on ubiquitination of both Tom20 and MIRO.

Parkin has previously been shown to assemble K27-, K48- and K63-type polyubiquitin chains on various mitochondrial substrates (Geisler et al., Nat. Cell Biol., 12: 119-131 (2010)). To examine the polyubiquitin chain topology on Tom20 and Miro, we repeated the ubiquitination assays with HA-ubiquitin mutants where all seven lysine residues were individually replaced with arginine (single K-to-R mutants), or with mutants where a single lysine was left intact and all other six lysines were replaced with arginine. We compared the amount of CCCP-induced Tom20 and Miro ubiquitination afforded by these ubiquitin mutants to wild-type ubiquitin. Among all “single K-to-R mutants”, only the K27R mutation blocked the CCCP-induced ubiquitination of Tom20, whereas the other K-to-R mutants (K6R, K11R, K29R, K33R, K48R, K63R) supported normal Tom20 ubiquitination (FIGS. 6C, D). Conversely, normal Tom20 ubiquitination was only supported by ubiquitin with K27 intact (all other lysines mutated), whereas all other single lysine mutants (K6, K11, K29, K33, K48, K63) had impaired Tom20 ubiquitination (FIGS. 6E, F). Thus, K27 on ubiquitin is both necessary and sufficient for building polyubiquitin chains on Tom20, suggesting the primary polyubiquitin topology on Tom20 is K27-type chains. Similar to Tom20, Miro also required K27 (and not the other lysines on ubiquitin) for its normal ubiquitination (FIGS. 6C, D). Although the ubiquitin mutant that contains only K27 supported Miro ubiquitination the best, significantly less ubiquitin was attached on Miro as compared to wild-type ubiquitin (˜65% of wild-type ubiquitin), suggesting that Miro accumulates other chain-types in addition to K27 (FIGS. 6E, F). Our data are consistent with Parkin's ability to assembly K27-linked chains on other substrates (Geisler et al., Nat. Cell Biol., 12: 119-131 (2010)).

Beyond ubiquitination, does USP30 regulate protein turnover in addition to ubiquitination? Published evidence suggests that Parkin mediates the degradation of multiple mitochondrial outer membrane proteins (Chan et al., Human Mol. Genet., 20: 1726-1737 (2011)). Consistent with this, all of the several outer membrane proteins examined (MIRO, MFN-1, TOM70, VDAC, Tom20) showed significant drop in protein level during the 6 hours of CCCP treatment (5 μM) of GFP-Parkin stable cell lines (FIGS. 6G, H). Tom20 levels were also reduced but to a lesser extent than MIRO and other outer membrane proteins (10+/−1% decrease with CCCP at t=6 h, p<0.01) (FIGS. 6G, H). In contrast to the outer membrane proteins, mitochondrial matrix protein HSP60 and inner membrane protein TIMM8A were unchanged by CCCP within this time frame. Overexpression of USP30 in GFP-Parkin stable cells greatly attenuated or abolished the CCCP-induced depletion of MIRO and Tom20 (FIGS. 6G, H). Stabilization by USP30 overexpression appeared to be relatively specific for MIRO and Tom20, since CCCP-induced degradation of other mitochondria membrane proteins (MFN-1, TOM70, VDAC) was unaffected (FIGS. 6G, H). Unlike wildtype USP30, the inactive C77A- or C77S-USP30 mutants did not inhibit degradation of MIRO or Tom20 induced by CCCP, implying requirement for DUB activity (FIGS. 6G, H). These data indicate USP30 can specifically counteract degradation of MIRO and Tom20 without affecting the turnover of other mitochondrial proteins.

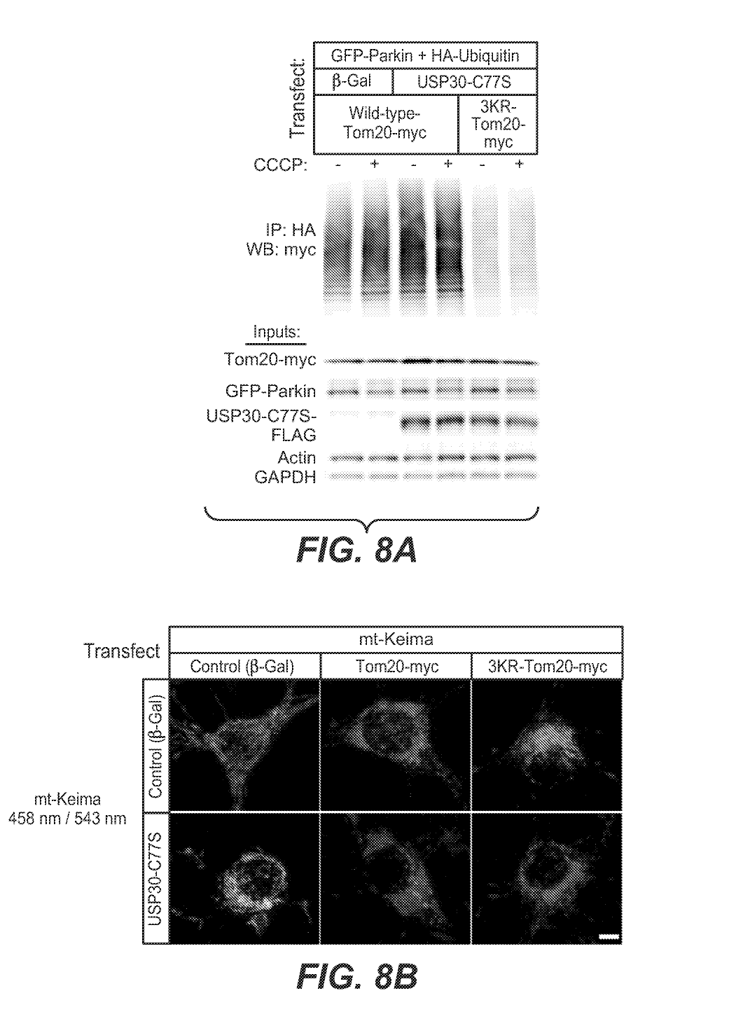

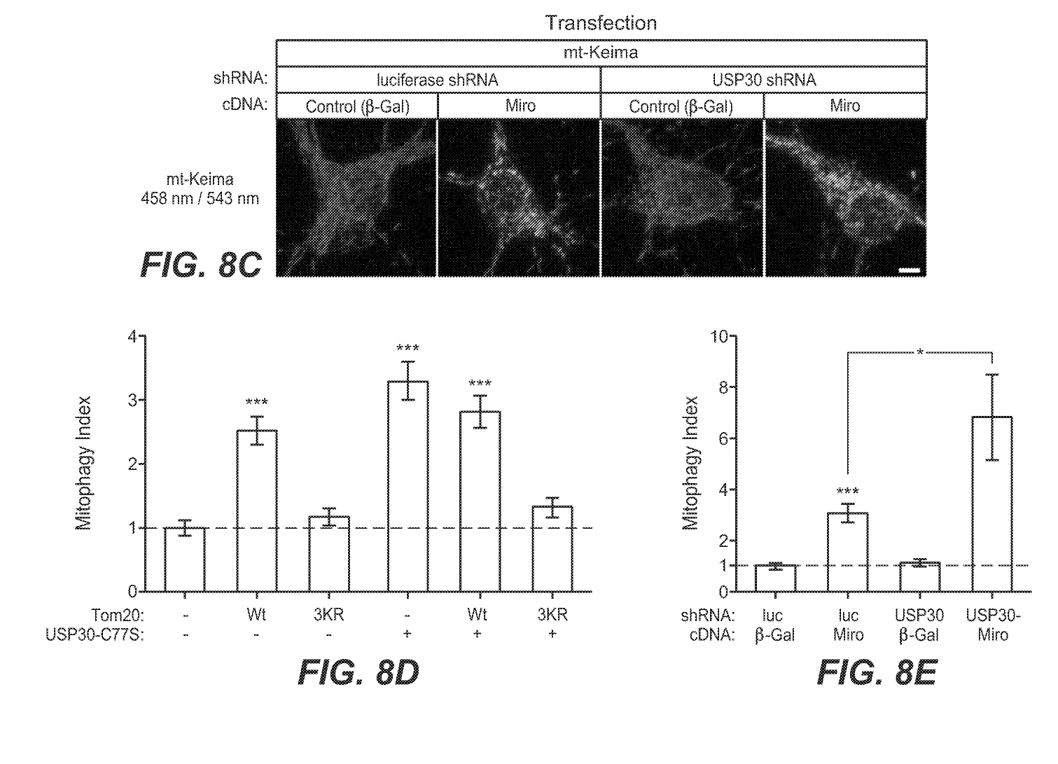

Since MIRO and Tom20 degradation accompanies mitophagy (FIGS. 6G, H and (Chan et al., Human Mol. Genet., 20: 1726-1737 (2011)) and USP30 knockdown enhances mitophagy (FIGS. 5C, E) and ubiquitination of MIRO and Tom20 (FIG. 7), it was speculated that the depletion of these proteins might trigger mitophagy. In this model, overexpression of these proteins would block mitophagy induced by USP30-knockdown. Instead, it was found that overexpression of MIRO or Tom20 in neurons—even by themselves—led to a robust increase in mitophagy in the mt-Keima assay (FIGS. 8B, C, D, E), an effect similar to USP30 knockdown. It was therefore hypothesized that it is the ubiquitination of MIRO and Tom20 that serves as the signal for mitophagy (rather than their degradation, which occurs secondary to ubiquitination), and that overexpression of MIRO and Tom20 promotes mitophagy by increasing the pool of these substrates available for ubiquitination.

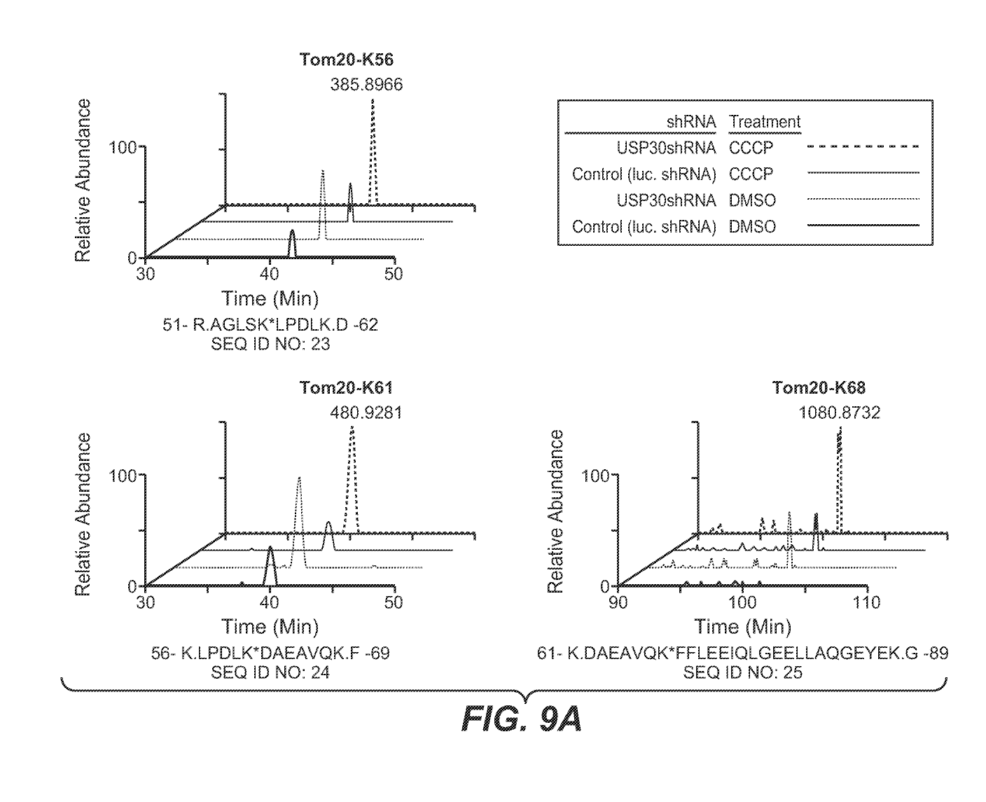

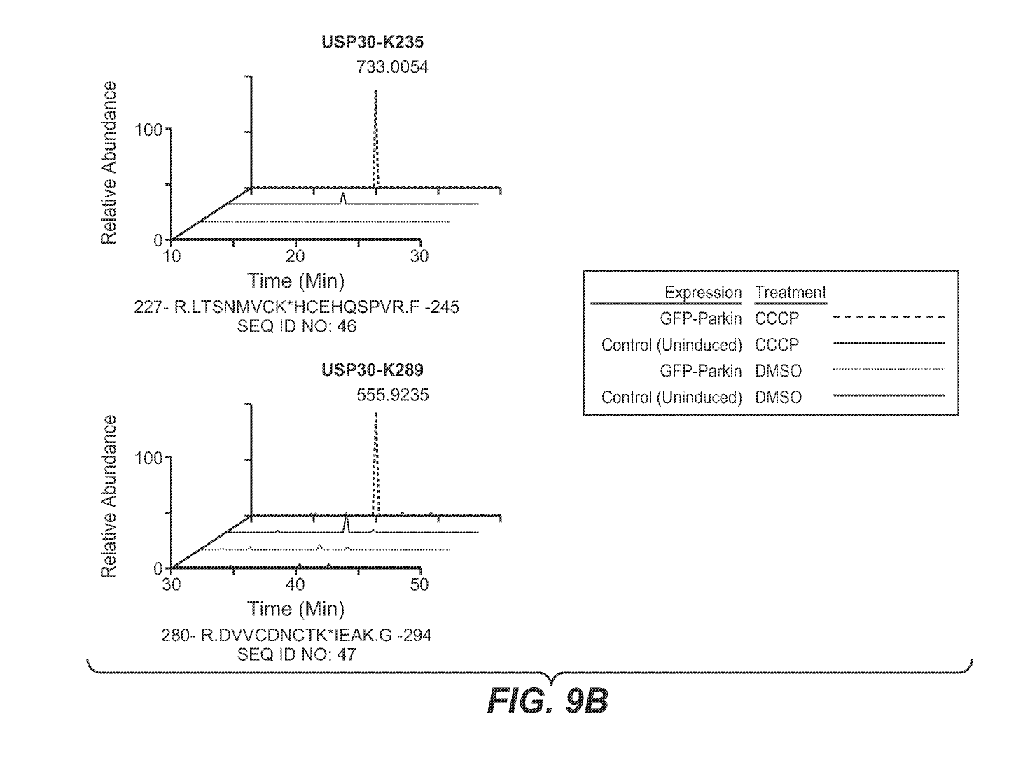

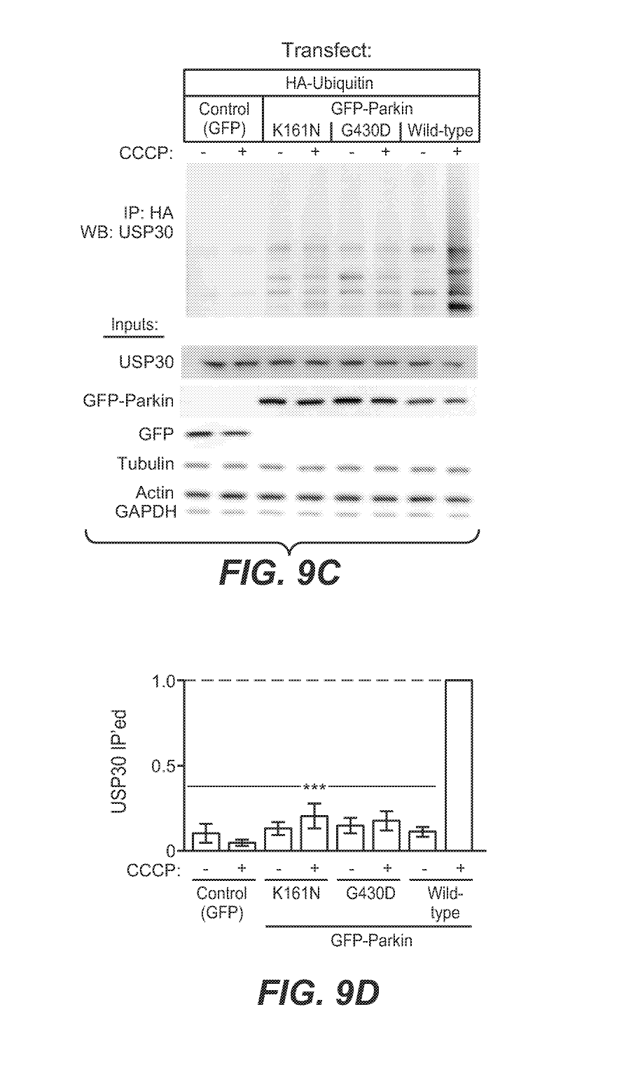

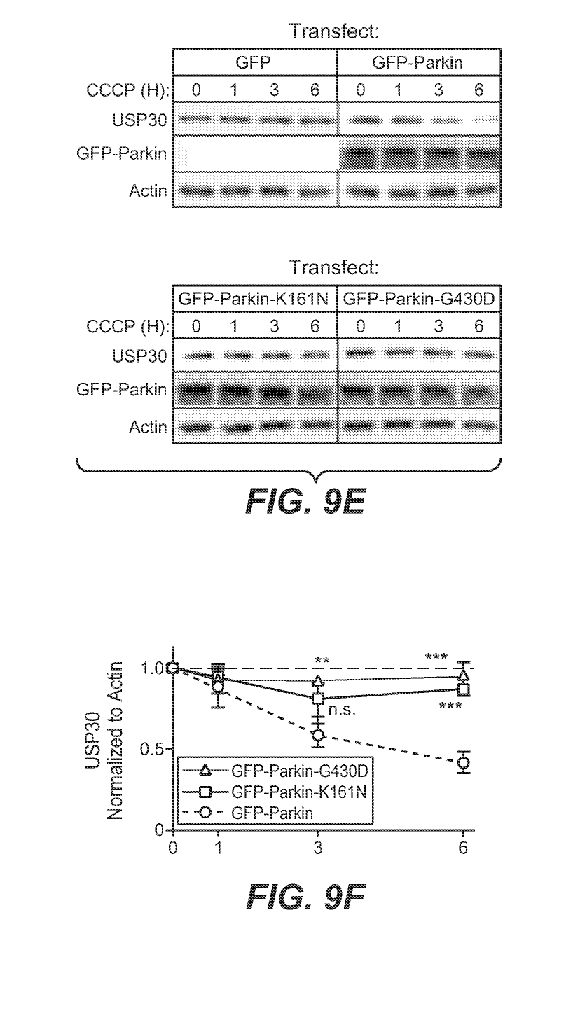

MS analysis of ubiquitinated peptides derived from Tom20 identified 3 lysine residues (K56, K61 and K68) whose ubiquitination increased upon CCCP or USP30 knockdown, and that increased even further in response to the combination of CCCP treatment+USP30 knockdown (FIG. 9). To confirm ubiquitination on these particular sites, the three lysine residues in Tom20 were mutated to arginine (“3KR-Tom20” (K56R, K61R, K68R mutant)). In GFP-Parkin overexpressing cells, myc-tagged wildtype Tom20 exhibited an increase in ubiquitination with coexpression of enzymatically-inactive USP30-C77S (FIG. 8A), similar to endogenous Tom20 (FIGS. 7A, C). In contrast, 3KR-Tom20 showed less basal ubiquitination than wild-type Tom20 and additionally it was unaffected by the dominant negative USP30-C77S (FIG. 8A), indicating that these three lysine residues are the major USP30 target residues on Tom20.

In the mt-Keima assay in neurons, overexpression of wild-type Tom20 enhanced mitophagy, whereas the 3KR-Tom20 mutant failed to do so (FIGS. 8B, D); thus Tom20 is sufficient to drive mitophagy, but this ability depends on its ubiquitination. Moreover, 3KR-Tom20 blocked the increase in mitophagy induced by USP30-C77S (FIGS. 8B, D), implying that the increased mitophagic flux caused by dominant-negative USP30 requires Tom20 ubiquitination. Alternatively, overexpressed 3KR-Tom20 may be able to oppose USP30-C77S-induced mitophagy by physically associating with USP30 in a non-catalytic manner.

Mass spectrometry identified nine lysine-ubiquitination sites on MIRO regulated by USP30 and Parkin, some of which are known to be required for normal MIRO function (e.g. K427 required for GTPase activity (Fransson et al., Bioch. Biophys. Res. Comm., 344: 500-510 (2006)). Thus, instead of a pursuing a combinatorial mutagenesis, the effect of USP30 on MIRO's ability to induce mitophagy was studied since USP30 knockdown increases MIRO ubiquitination (FIGS. 7D, E). Consistent with the idea that MIRO ubiquitination drives mitophagy, USP30 knockdown further enhanced mitophagy beyond what was observed following MIRO overexpression alone (FIGS. 8C, E). Taken together, these data indicate that ubiquitination of MIRO or Tom20 can drive mitophagy in neurons, and that inhibition of mitophagy by USP30 can be explained at least in part by deubiquitination of these proteins.

### Example 6

### USP30 Knockdown Rescues Mitophagy Defect Associated with PD Mutations

If mitochondrial degradation defects associated with PD-linked mutations of Parkin are due to impaired ubiquitination of damaged mitochondria, and USP30 indeed functions as a biochemical and functional antagonist of Parkin, then inhibiting USP30 should restore mitochondria ubiquitination and degradation. To test this hypothesis, we focused on PD-linked Parkin pathogenic mutants, such as G430D and K161N, that display attenuated ligase activity (Sriram et al., Human Mol. Genet., 14: 2571-2586 (2005)) with accompanying defects in mitophagy (Geisler et al., Nat. Cell Biol., 12: 119-131 (2010); Lee et al., J. Cell Biol., 189: 671-680 (2010)) were studied (e.g. G430D and K161N).

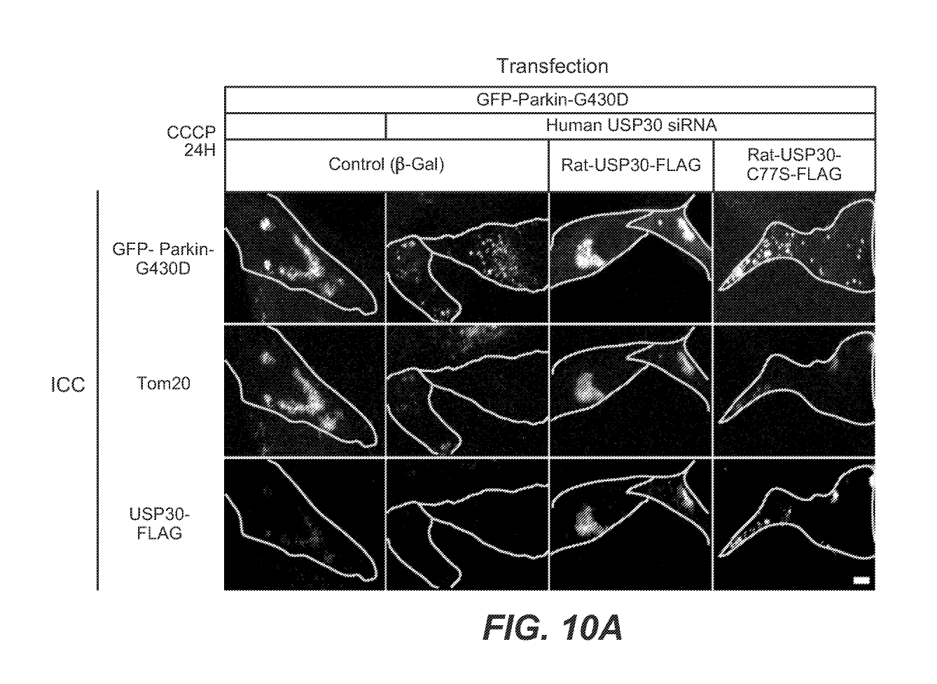

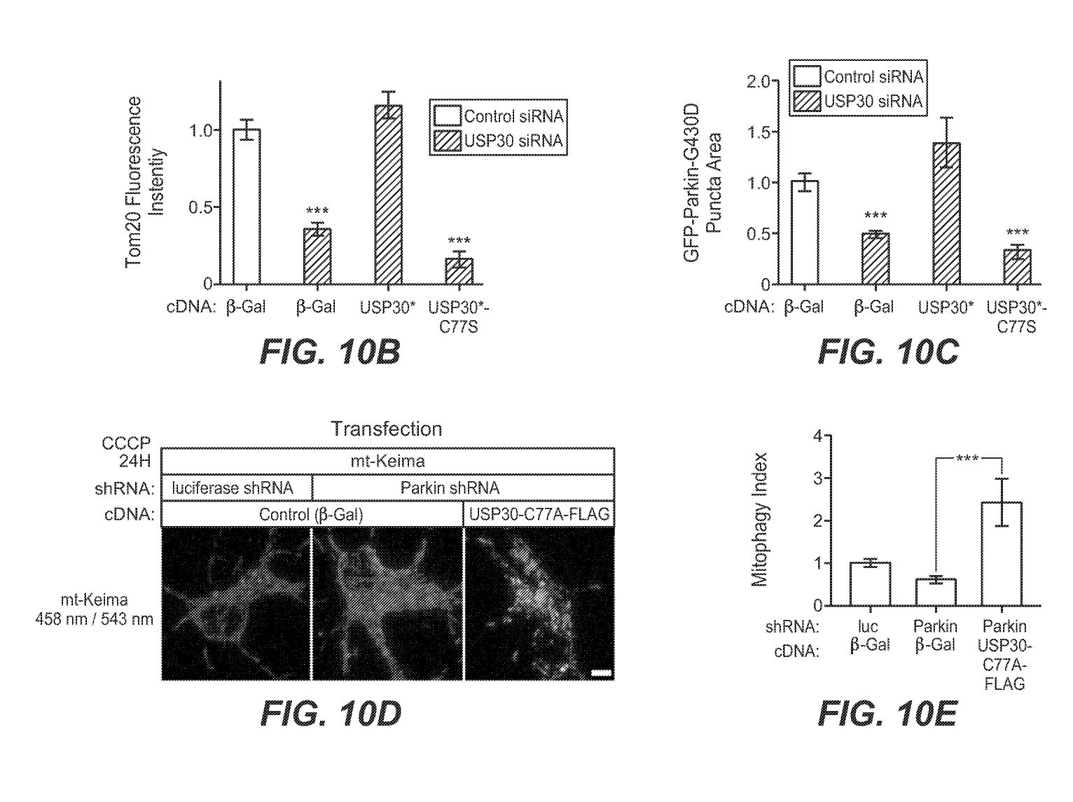

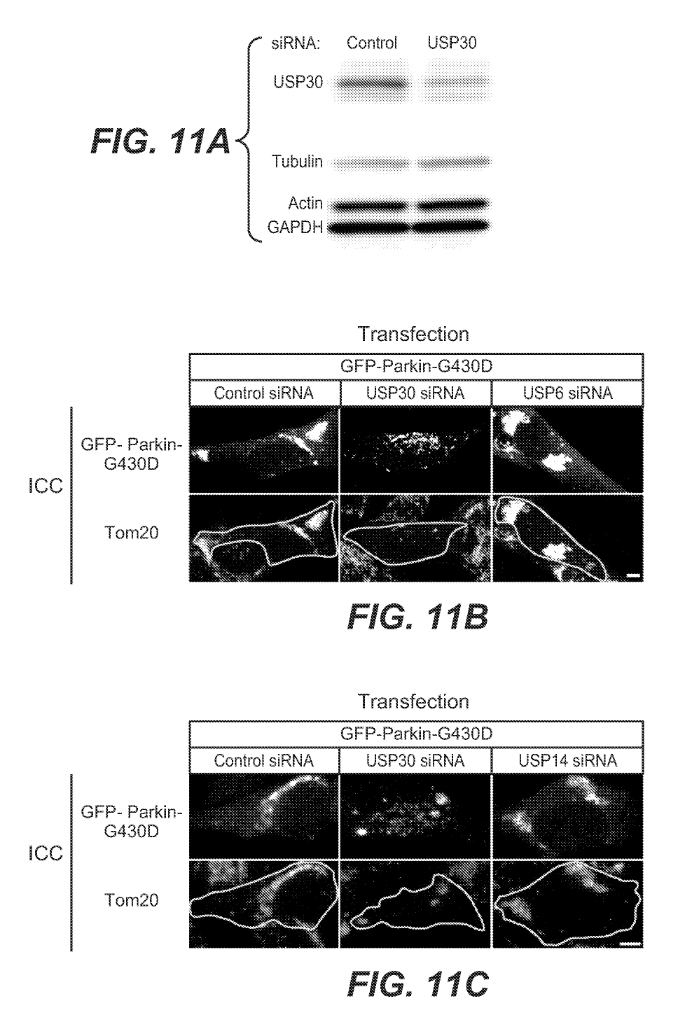

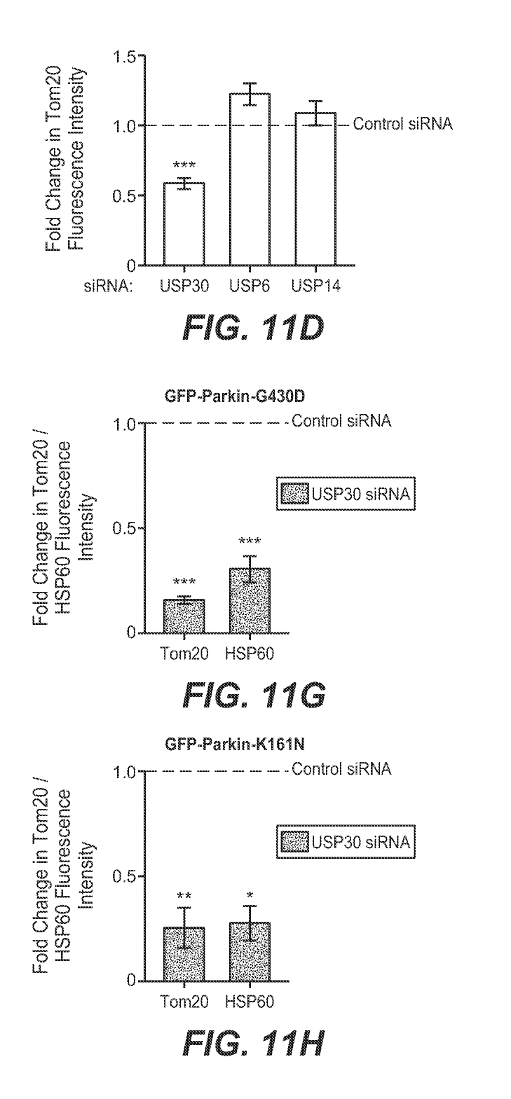

In SH-SY5Y cells transfected with pathogenic mutant GFP-Parkin-G430D and treated with CCCP, mitochondria fail to be cleared and form perinuclear clusters in association with the defective Parkin protein (FIG. 10A, first column). The same cells doubly transfected with Parkin-G430D and USP30 siRNA, which led to knockdown of USP30 protein by ˜60% (FIG. 11A), showed a 60% decrease in mitochondria (as measured by total Tom20 fluorescence) compared to cells transfected with Parkin-G430D and control siRNA (FIG. 10A, quantified in B). This result shows that siRNA knockdown of USP30 protein level can largely rescue mitophagy in the face of defective Parkin. Mitochondria degradation was not rescued by knockdown of other DUBs (USP6, USP14) (FIG. 11B-D). Re-introduction of an RNAi-resistant wildtype USP30 (rat USP30 cDNA), but not the inactive rat USP30-C77S mutant, prevented the rescue of mitochondrial degradation by USP30 siRNA (FIGS. 10A, B). Rescue of mitochondrial degradation was correlated with loss of perinuclear clusters of mutant G430D Parkin (usually associated with mitochondria) and appearance of smaller dispersed Parkin-containing puncta throughout the cytoplasm (FIGS. 10A, C; see also FIGS. 11B, C). In CCCP-treated GFP-Parkin-G430D expressing cells, USP30 knockdown not only led to loss of Tom20 immunoreactivity but also decreased staining for matrix protein HSP60 suggesting that USP30 suppression restored degradation of the whole mitochondrion (FIGS. 11E, G). The mitochondrial degradation defect associated with another PD-associated Parkin mutant (K161N) was similarly rescued with USP30 siRNA knockdown (FIGS. 11F, H). In neurons, reduced mitophagy associated with Parkin knockdown (as measured in the mt-Keima assay) was also rescued with dominant-negative USP30-C77A (FIGS. 10D, E). Thus, suppressing the expression or activity of USP30 allows cells to overcome defective Parkin or Parkin knockdown and restore the clearance of damaged mitochondria.

While not intending to be bound by any particular theory, since Parkin ligase activity marks mitochondria through ubiquitination, some residual ligase activity present in Parkin mutants may be needed in order for USP30 knockdown to rescue mitophagy. It is currently unknown whether USP30 knockdown would be effective with complete loss of Parkin activity. It is possible, however that there are other E3s that have overlapping substrates or that can compensate for lack of Parkin.

### Example 7

### USP30 is a Parkin Substrate

Since Parkin has broad activity towards outer mitochondrial membrane proteins, we wondered whether Parkin ubiquitinates USP30, which also resides at this mitochondrial compartment. Supporting this possibility, we identified USP30-derived ubiquitinated peptides in proteomics experiments in GFP-Parkin expressing cells treated with CCCP (fold change in ubiquitination of USP30 in ‘GFP-Parkin+CCCP’ over ‘DMSO’=27.23, p<0.001). To confirm USP30 ubiquitination by Parkin, we repeated the ubiquitination assay in cells overexpressing GFP-Parkin and HA-ubiquitin, and found GFP-Parkin induced ubiquitination of endogenous USP30 following CCCP treatment (20 μM, 2 hours, FIGS. 9C, D). The ubiquitination sites of USP30 (K235 and K289) identified by mass spectrometry were not required for its ubiquitination suggesting other lysine residues in USP30 can accept ubiquitin (data not shown). CCCP treatment (20 μM) also induced a significant drop in USP30 levels in GFP-Parkin expressing cells (FIGS. 9E, F). Importantly, Parkin with pathogenic mutations G430D or K161N were not able to ubiquitinate (FIGS. 9C, D) or degrade USP30 (FIG. 9E-F). These data indicate that Parkin ubiquitinates and degrades USP30, thus removing the brake on mitophagy.

### Example 8

### USP30 Knockdown Decreases Oxidative Stress and Provides Protection In Vivo

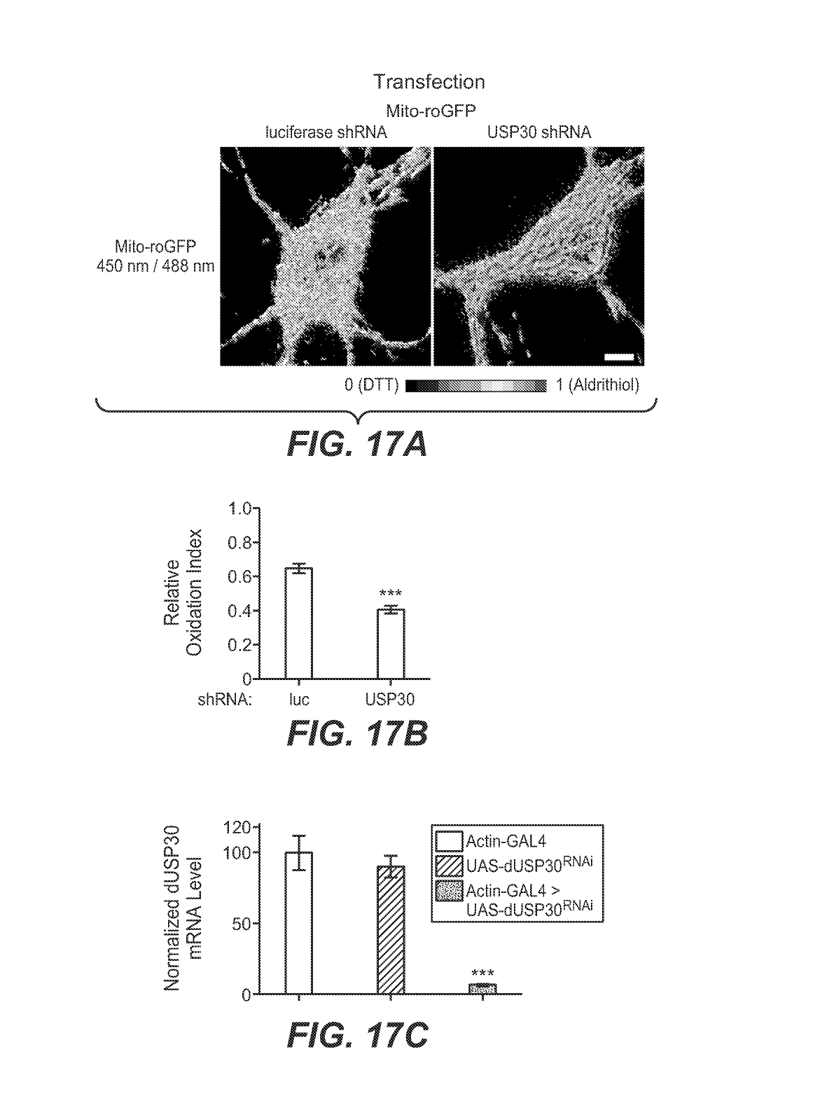

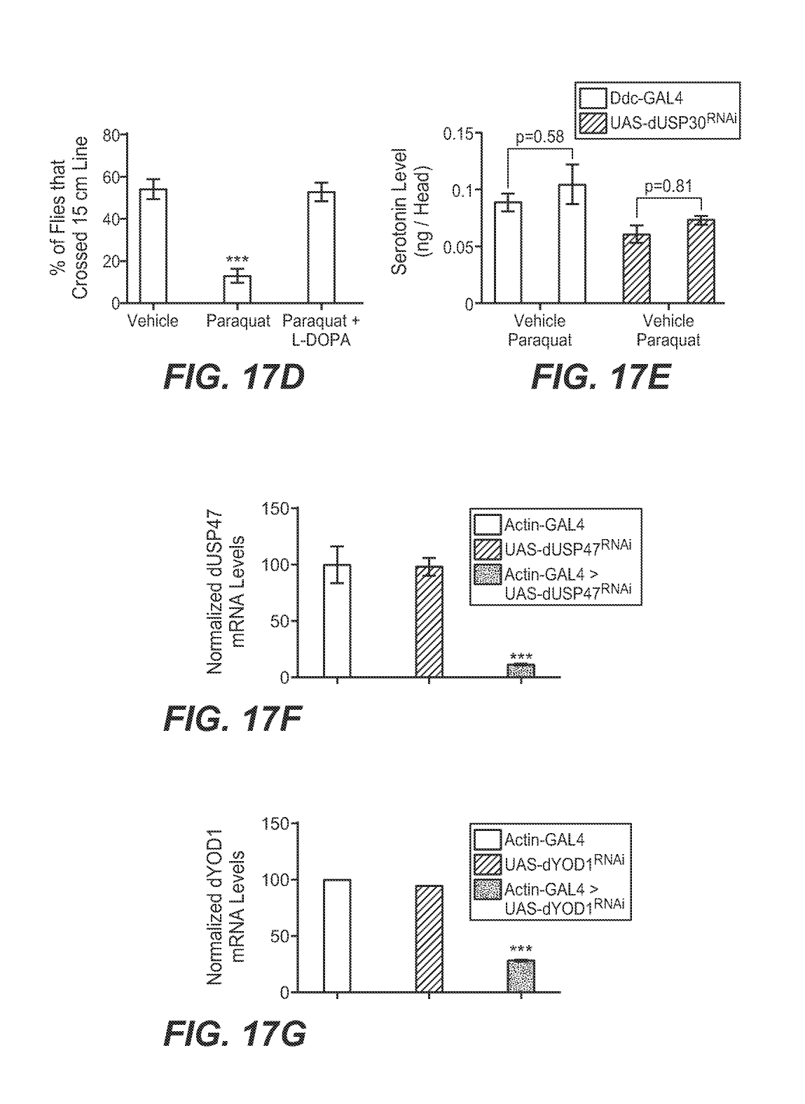

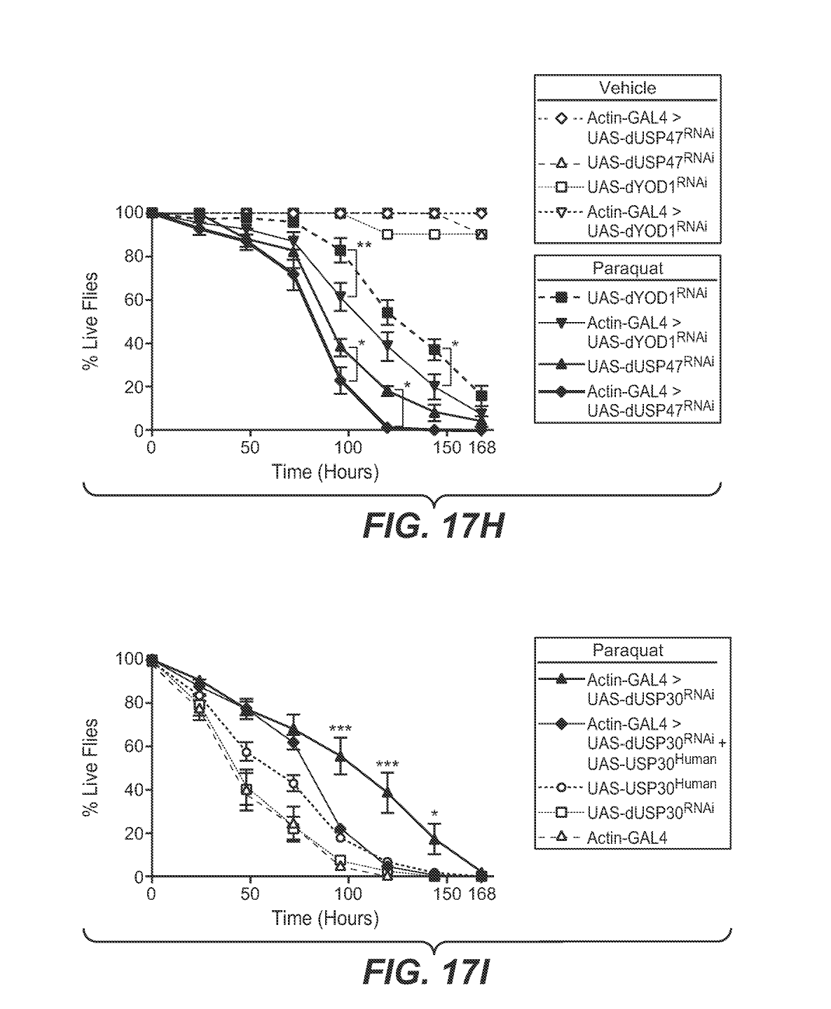

Whether USP30 knockdown provides functional benefit to mitochondria and cells was examined next. ROS—which largely derive from mitochondria—is associated with neurodegenerative disorders and mitochondria dysfunction may contribute to increased oxidative stress in PD (Lee et al., Biochem. J., 441: 523-540 (2012)). To measure oxidative stress in mitochondria, mitochondria matrix-targeted ro-GFP (mito-roGFP), a redox-sensitive fluorescent protein that allows quantitative ratiometric imaging of mitochondrial redox potential was used (Dooley et al., J. Biol. Chem. 279: 22284-22293 (2004)). Following measurement of ratiometric mito-roGFP signal in individual cells, the dynamic range of the probe was calibrated by treating cultures sequentially with DTT (1 mM) to fully reduce the probe and aldrithiol (100 μM) to fully oxidize the probe (Guzman et al., Nature, 468: 696-700 (2010). Ratios of mito-roGFP measured after DTT and aldrithiol were set to 0 and 1, respectively, to calibrate the relative oxidation index. In control cells transfected with control luciferase shRNA, neurons had a mean relative oxidation index of ˜0.6 (FIGS. 17A, B). USP30 knockdown dropped the relative oxidation index to ˜0.4, suggesting that suppression of USP30 protein led to a reduction in mitochondrial oxidative stress.

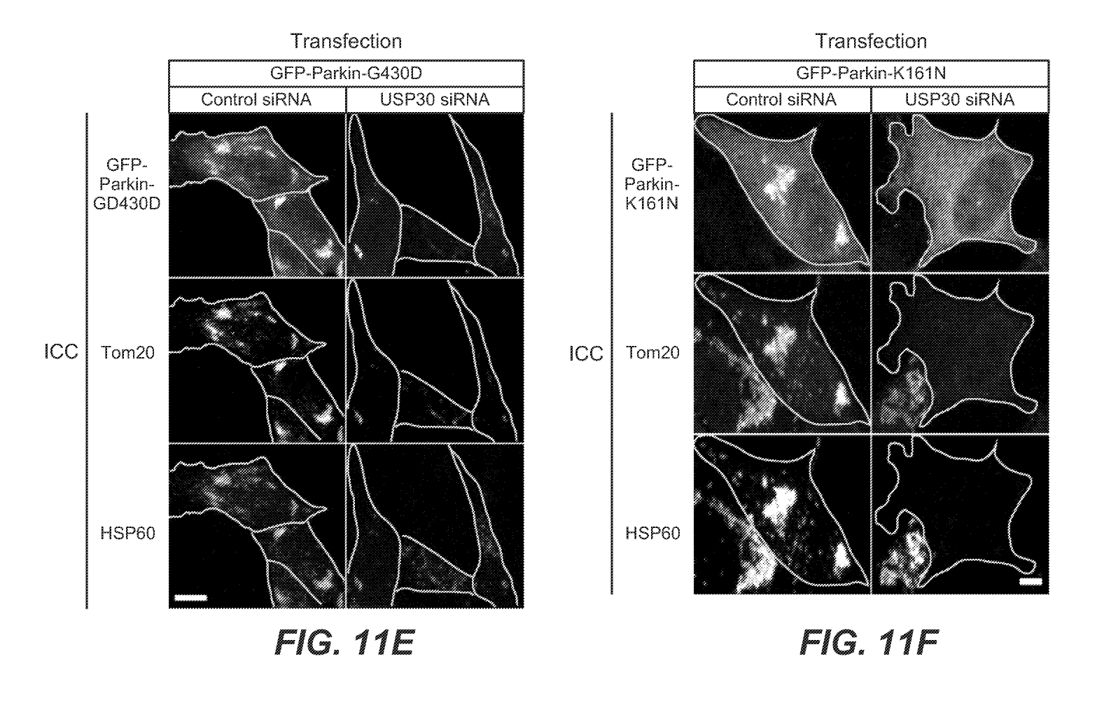

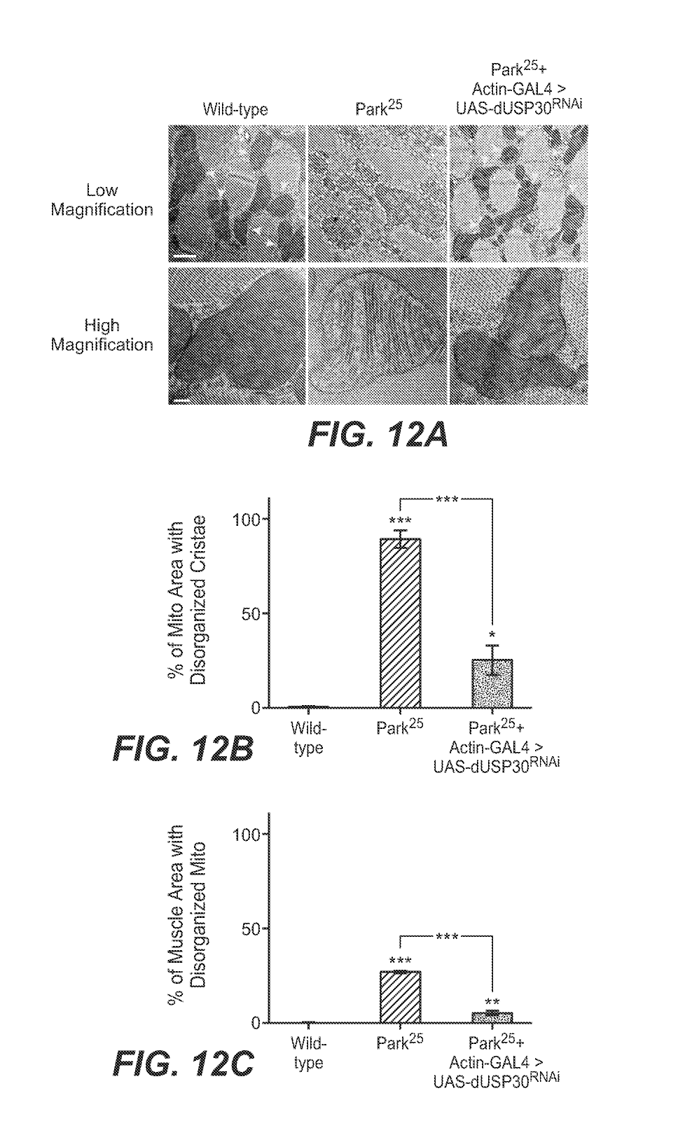

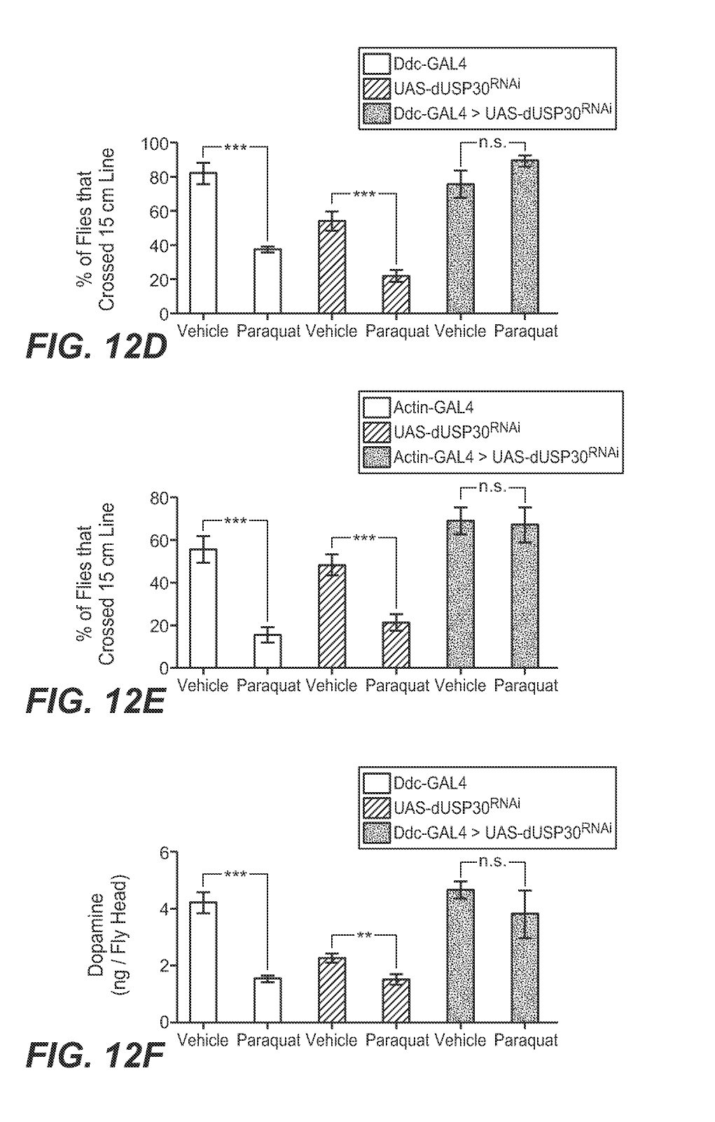

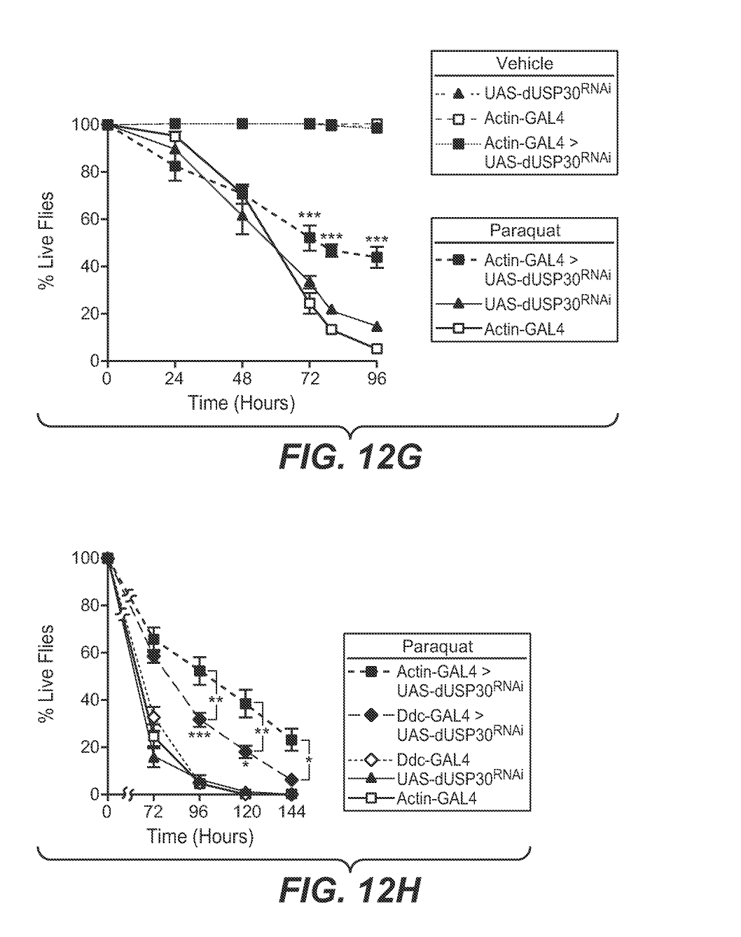

To test whether knocking down USP30 would provide protection under stress conditions in vivo, we used Drosophila, which has emerged as an effective model system for studying PD molecular pathogenesis (Guo, Cold Spring Harb. Perspect. Med. 2(11) pii: a009944 (2012)). To knock down fly USP30 (CG3016, hereafter called dUSP30), we employed the GAL4/UAS system (Brand et al., Development, 118: 401-415 (1993)). We crossed an Actin-GAL4 driver line with a UAS-dUSP30^{RNAi} transgenic line, which allows expression of dUSP30 RNAi under the control of the Actin promoter (this Actin-GAL4>dUSP30^{RNAi} line is referred to as ‘dUSP30 knockdown’ line). Activation of UAS-dUSP30^{RNAi} by Actin-GAL4 led to a ˜90% reduction of dUSP30 mRNA by quantitative RT-PCR, compared to control parental lines containing only Actin-GAL4 or only UAS-dUSP30^{RNAi} (FIG. 17C). To test the protective effects of dUSP30 knockdown, we crossed the ‘dUSP30 knockdown’ line with parkin mutant flies (park²⁵) (Greene et al., Proc. Natl. Acad. Sci. USA, 100: 4078-4083 (2003)). Flies lacking parkin show severe defects in mitochondrial morphology in their indirect flight muscles (IFMs), with mitochondria that are malformed with sparse, disorganized cristae, giving rise to a “pale” appearance of mitochondria under EM (FIG. 12A, and Greene et al., Proc. Natl. Acad. Sci. USA, 100: 4078-4083 (2003)). In contrast, wild-type flies have many dark-staining mitochondria evenly packed with cristae (FIG. 12A). To determine the effect of USP30 inhibition on Parkin-deficient mitochondria, we crossed parkin mutants to dUSP30 knockdown flies. In the “parkin mutant; dUSP30 knockdown” flies, most of the IFM mitochondria were electron-dense and contained numerous cristae, although “pale” mitochondria with fragmented cristae were also occasionally found (FIG. 12A). Quantification of the percent area of mitochondria containing disorganized cristae over total mitochondria area revealed a strong improvement in mitochondrial integrity with dUSP30 knockdown (FIG. 12B—˜90% in parkin mutants versus ˜25% in “parkin mutant; dUSP30 knockdown”). “Parkin mutant; dUSP30 knockdown” flies also had less damaged mitochondria per muscle area (FIG. 12C). Thus, suppressing dUSP30 expression is able to largely restore morphological mitochondrial integrity in vivo in parkin-deficient Drosophila.

To examine the effect of suppressing USP30 in neurons that are relevant to PD, we used dopamine decarboxylase (Ddc)-GAL439 to drive dUSP30^{RNAi} specifically in aminergic neurons of the fly nervous system. As a model of mitochondrial damage and PD, we treated flies with paraquat, a mitochondrial toxin linked to PD (Castello et al., J. Biol. Chem., 282: 14186-14193 (2007); Cocheme et al., J. Biol. Chem., 283: 1786-1798 (2008); Tanner et al., Environmental Health Perspect., 119: 866-872 (2011)). Following treatment with paraquat (10 mM, 48 hours), both the Ddc-GAL4 and UAS-dUSP30^{RNAi} control fly lines showed reduced ability to climb up beyond 15 cm (FIG. 12D). This climbing defect was fully rescued by additional treatment with L-DOPA (FIG. 17D), showing that this behavioral deficit is likely due to depletion of dopamine. Consistently, in control fly lines (Ddc-GAL4 or UAS-dUSP30^{RNAi} transgenics alone), paraquat treatment (10 mM, 48 hours) caused a 30-60% reduction in dopamine levels in fly heads without altering serotonin neurotransmitter levels (indicating specific toxicity of paraquat on the dopaminergic system in this model—FIG. 12F and FIG. 17E). Similar to L-DOPA, dUSP30 knockdown in Ddc-GAL4>UAS-dUSP30^{RNAi} flies also completely rescued the paraquat-induced climbing impairment (FIG. 12D), indicating that complete protection against paraquat toxicity in this behavioral test can be afforded by suppression of USP30 specifically in aminergic neurons. A similar complete protection was also observed with whole body knockdown of USP30 in Actin-GAL4>UAS-dUSP30^{RNAi} flies (FIG. 12E). Strikingly, USP30 knockdown in Ddc neurons (Ddc-GAL4>UAS-dUSP30^{RNAi} flies) also prevented the paraquat-induced dopamine depletion (FIG. 12F). Since USP30 knockdown rescued both depletion of dopamine and motor impairment, these results show that suppression of USP30 can benefit dopaminergic neurons and the organism in both neurochemical and functional terms.

To test whether USP30 knockdown has an effect on organism survival, we monitored the percentage of live flies over prolonged treatment with paraquat (10 mM, 96 hours). Flies expressing dUSP30 RNAi survived significantly longer than controls (FIG. 12G). Only <15% of flies treated with paraquat were alive in Actin-GAL4 and UAS-dUSP30^{RNAi} control groups at 96 hours whereas ˜45% of flies were alive in the Actin-GAL4>UAS-dUSP30^{RNAi} (‘whole body dUSP30 knockdown’) group (FIG. 12G). We confirmed that the benefit of USP30 knockdown was not due to differences in exposure to paraquat since all three fly lines ingested roughly equal amounts of paraquat as measured by LC-MS/MS (average mass of paraquat per fly: UAS-dUSP30^{RNAi}: 3.2 μg, Actin-GAL4: 2.7 μg, Actin-GAL4>UAS-dUSP30^{RNAi}: 2.7 μg). Knockdown of other DUBs in flies (dUSP47 (CG5486) or dYOD1 (CG4603)) did not provide benefit in the survival assay; if anything, they exacerbated the rate of death in response to paraquat (FIG. 17F-H) Furthermore, introduction of a human USP30 cDNA into flies expressing dUSP30^{RNAi} reversed the survival benefit provided by dUSP30^{RNAi} (FIG. 171), demonstrating the specificity of the RNAi effect. Remarkably, USP30 knockdown specifically in Ddc neurons was sufficient to provide significant survival benefit, albeit less than the whole body USP30 knockdown (FIG. 12H). This result implies that a significant portion of the organismal benefit of USP30 suppression is mediated in dopaminergic neurons, and it further reinforces the idea that USP30 plays a critical role in dopaminergic neuron dysfunction.

### Example 9

### Discussion

Better understanding of the pathogenic mechanisms in PD would be helpful for rational design of disease-modifying therapies for this neurodegenerative disease. Impaired activity of oxidative phosphorylation enzymes (Schapira et al., Lancet, 1: 1269 (1989)), elevated levels of oxidative stress markers (Lee et al., Biochem. J., 441: 523-540 (2012)) and mtDNA mutations (Bender et al., Nature Genet., 38: 515-517 (2006); Kraytsberg et al., Nature Genet., 38: 518-520 (2006)) in PD suggest accumulation of defective mitochondria (Zheng et al., Science Transl. Med., 2: 52ra73 (2010)). PINK1/Parkin genetics further implicate aberrant mitochondrial biology and point to impaired mitochondrial quality control as a causative factor in the etiology of PD (Youle et al., J. Biol. Chem., 12: 9-23 (2011)). Uncleared damaged mitochondria can be a source of toxicity and “pollute” the mitochondrial network through fusion with healthy mitochondria (Tanaka et al., J. Cell. Biol., 191: 1367-1380 (2010)).

We have identified USP30, a DUB localized to mitochondria, as a negative regulator of mitophagy. USP30, through its deubiquitinase activity, opposes Parkin-mediated ubiquitination and degradation of mitochondrial proteins and reverses the marking of damaged mitochondria for mitophagy. Knockdown inhibition of USP30 accelerated mitophagy, and it restored CCCP-induced mitochondrial degradation in cells expressing PD-associated mutants of Parkin. USP30 knockdown improves mitochondrial integrity in parkin mutant flies, confirming that Parkin and USP30 have opposing actions on mitochondrial quality in vivo. USP30 knockdown also conferred motor behavior and survival benefits in wildtype flies treated with paraquat, further supporting the idea that USP30 inhibition might ameliorate the effects of mitochondrial damage.

### Parkin, USP30 and Mitochondrial Quality Control

Although Parkin and PINK1 are identified as key players in mitophagy, a detailed mechanistic understanding of the mitophagy pathway, especially in mammalian cells, is lacking. The fact that basal mitophagy in neurons depends on Parkin and PINK1 (FIG. 3) suggests that these proteins actively monitor normally occurring mitochondrial damage. Mitochondria fission—which appears to be required for Parkin-mediated mitophagy (Tanaka et al., J. Cell. Biol., 191: 1367-1380 (2010))—may contribute to basal mitochondrial turnover by eliciting a transient drop in membrane potential in one of the two daughter mitochondria (Twig et al., EMBO J., 27: 433-446 (2008)). This transient drop in membrane potential creates an opportunity for PINK1 accumulation and Parkin recruitment, leading to eventual mitophagy if membrane potential is not quickly re-established. Thus under basal conditions, USP30 knockdown may accelerate mitophagy by favoring Parkin-mediated ubiquitination during the fission-associated drops in mitochondrial membrane potential. As damaged mitochondria are more likely to accumulate Parkin, it is expected that suppression of USP30 function will preferentially clear unhealthy mitochondria.

Since Parkin ligase activity marks mitochondria through ubiquitination, some residual ligase activity present in Parkin mutants is presumably required in order for USP30 knockdown to rescue mitophagy. It would be expected that with complete loss of Parkin activity, USP30 knockdown would be ineffective at rescuing clearance unless other E3 ligases have overlapping substrates and can compensate for lack of Parkin. The rescue of mitochondrial integrity with USP30 knockdown in parkin mutant flies, even though a large portion of parkin gene is missing, supports the latter possibility.

As part of normal turnover, the cleared mitochondria presumably need to be replaced through mitochondrial biogenesis. In culture cell lines, mitochondrial damage increases overall mitochondrial mass (Narendra et al., PLoS Biology, 8: e1000298 (2010)). In this context it is interesting to note that Parkin can also boost mitochondrial biogenesis by degrading negative transcriptional regulators (Shin et al., Cell 144: 689-702 (2011)). Further studies are required to determine whether USP30 also regulates the biogenesis pathway and whether mitophagy induced by USP30 inhibition is accompanied by new mitochondria production.

### USP30 Versus Parkin on Common Substrates

Global ubiquitination site mapping experiments identified multiple substrates whose ubiquitination is affected by both Parkin overexpression and USP30 knockdown. Amongst these shared presumptive substrates, we confirmed that Miro and Tom20 have ubiquitination levels that are antagonistically regulated by Parkin and USP30, i.e. GFP-Parkin overexpression or USP30 knockdown increased ubiquitination induced by CCCP treatment. Interestingly, a subset of these shared substrates, exemplified by Tom20, was regulated by USP30 even under basal conditions. Mull, Asns and Fkbp8—but not Miro—were mitochondrial substrates that behaved similarly to Tom20, exhibiting a basal increase in ubiquitination with USP30 knockdown in the absence of CCCP. Thus, USP30 basally deubiquitinates this set of proteins, presumably by counterbalancing against a mitochondrial E3 ligase that is active in the absence of CCCP and that acts on Tom20 but not Miro. Following CCCP, USP30 also counteracts Parkin dependent ubiquitination of this set of substrates. On the other hand, proteins such as Tom70, Mat2b and Pth2, behaved similarly to Miro in that they exhibited enhanced ubiquitination with USP30 knockdown only following CCCP. This observation suggests that these set of proteins undergo low levels of basal ubiquitination in the absence of recruited Parkin (i.e. Parkin is their major E3 ligase), or that USP30 is inactive toward those proteins under basal conditions. Mitochondrial depolarization regulates Parkin's E3 ligase activity (Matsuda et al., J. Cell Biol., 189: 211-221 (2010)) possibly via PINK1-mediated phosphorylation (Kondapalli et al., Open Biology, 2: 120080 (2012); but see Vives-Bauza et al., Proc. Natl. Acad. Sci. USA, 107: 378-383 (2010)); it remains to be studied whether the activity of USP30—which is constitutively localized on mitochondria—is also regulated by mitochondrial damage.

Global ubiquitination analysis also revealed a series of non-mitochondrial proteins whose ubiquitination was inversely regulated by Parkin and USP30 (including nuclear proteins, metabolic enzymes and components of the ubiquitin-proteasome system (UPS)). This suggests that the antagonistic functional relationship between Parkin and USP30 may extend beyond mitochondria. These non-mitochondrial “substrates” may be indirectly regulated by Parkin and USP30, or may have subpopulations on mitochondria, but have not formally been assigned as having mitochondrial localization due to the strict filtering criteria employed by computational tools (Pagliarini et al., Cell, 134: 112-123 (2008)). MS also identified some proteins that showed enhanced ubiquitination with CCCP (under endogenous Parkin and USP30 levels) with no further increase in ubiquitination upon Parkin overexpression or USP30 knockdown (e.g. Ssbp, ZO1, Rab10, Vamp1), suggesting engagement of alternative UPS pathways following mitochondrial depolarization. It is appropriate to note that in some cases, elevated levels of ubiquitinated species can derive from increases in the level of the total protein substrate itself.

Interestingly, Parkin also ubiquitinates and degrades USP30, and pathogenic Parkin mutations blocked this ability to downregulate USP30. Failure to remove a negative regulator of mitophagy may exacerbate the inefficient ubiquitination associated with these Parkin mutants, and could also partly explain the rescue of mitophagy by USP30 knockdown.

### Parkin, USP30 and Neurodegeneration

PD-linked mutations in Parkin may lead to decreased catalytic activity, enhanced aggregation and/or reduced expression (Hampe et al., Human Mol. Genet., 15: 2059-2075 (2006); Matsuda et al., J. Biol. Chem., 281: 3204-3209 (2006); Wang et al., J. Neurochem., 93: 422-431 (2005); Winklhofer et al., J. Biol. Chem. 278: 47199-47208 (2003)). In sporadic PD, Parkin activity can be also inhibited due to cellular stress (Corti et al., Physiol. Rev., 91: 1161-1218 (2011)). PD-linked PINK1 mutations impair translocation of Parkin to damaged mitochondria (Matsuda et al., J. Cell Biol., 189: 211-221 (2010); Narendra et al., PLoS Biology, 8: e1000298 (2010); Vives-Bauza et al., Proc. Natl. Acad. Sci. USA, 107: 378-383 (2010)). Thus, reduced function of Parkin in mitochondrial quality control is likely more prevalent in PD than as represented by rare Parkin mutations, providing further support for a possible utility of USP30 inhibition in idiopathic PD. Consistent with a benefit of USP30 inhibition, knockdown of USP30 restores mitophagy in cells expressing PD-associated mutant Parkin and reduces oxidative stress in neurons. In Drosophila parkin mutants, knockdown of USP30 improves mitochondrial integrity. Furthermore, USP30 knockdown provides a benefit in behavior and survival assays against paraquat, an oxidative stressor linked to mitochondria (Castello et al., J. Biol. Chem., 282: 14186-14193 (2007); Cocheme et al., J. Biol. Chem., 283: 1786-1798 (2008)). Since paraquat is a mitochondrial poison epidemiologically linked to PD (Tanner et al., Environmental Health Perspect., 119: 866-872 (2011)), our findings provide in vivo evidence that inhibition of USP30 might be helpful in diseases caused by mitochondrial damage and dysfunction.

In PD, mitochondrial dysfunction is not specific to substantia nigra neurons and is present systemically (Schapira et al, Parkinson's Dis., 2011: 159160 (2011)). Since USP30 expression seems to be widespread (Nakamura and Hirose, Mol. Biol. Cell, 19: 1903-1914 (2008)), USP30 inhibition has the potential to provide wide benefit by promoting clearance of damaged mitochondria. In addition to neurons, long-lived metabolically active cells such as cardiomyocytes also rely on an efficient mitochondrial quality control system (Gottlieb et al., Am. J. Physiol. Cell Physiol., 299: C203-210 (2010)). In this context, Parkin has been shown to protect cardiac myocytes against ischemia/reperfusion injury through activating mitophagy and clearing damaged mitochondria in response to ischemic stress (Huang et al., PLoS One, 6: e20975 (2011)). In inherited mitochondrial diseases, mtDNA mutations co-exist with wildtype mtDNA within the same cells, and mitochondrial dysfunction and disease ensue only when the proportion of mutated mtDNAs is high (Bayona-Bafaluy et al., Proc. Natl. Acad. Sci. USA, 102: 14392-14397 (2005)). Interestingly, Parkin overexpression eliminates mitochondria with deleterious mtDNA mutations and restores mitochondrial function, presumably by degrading mitochondria containing mutant mtDNA (Suen et al., Proc. Natl. Acad. Sci. USA, 107: 11835-11840 (2010)). Thus, USP30 inhibition has the potential to benefit diseases beyond PD by enhancing mitochondrial quality.

### Example 10

### Peptide Inhibitors of USP30

Two types of phage-displayed naïve peptide libraries, Linear-lib and Cyclic-lib, were cycled through rounds of binding selections with biotinylated USP30_cd (USP30 catalytic domain with C77A mutation) in solution as described previously (Stanger, et al., 2012, Nat. Chem. Bio., 7: 655-660). The selection identified peptide USP30_{—}3 and USP30_{—}8, which had moderate spot ELISA signals (signal/noise ratios of ˜5). USP_{—}30 and USP_{—}8 have the sequences:

| | | |
|---|---|---|
| USP30_3: | PLYCFYDLTYGYLCFY; | (SEQ ID NO: 1) |
| USP30_8: | VSRCYIFWNEMFCDVE. | (SEQ ID NO: 2) |

USP30_{—}3 and USP30_{—}8 were then assayed for inhibition of USP30 and also inhibition of USP7, USP5, UCHL3, and USP2, to determine each peptide's specificity for USP30, as follows. USP30 peptide ligands at a concentration range of 0-100 μM (for USP30_{—}3) and 0-500 μM (for USP30_{—}8) were mixed with 250 nM ubiquitin-AMC (Boston Biochem., Boston, Mass.; Cat. No. U-550). A panel of DUBs at 5.6, 2, 2, 5 and 0.05 nM for USP30, USP7, USP2, USP5, and UCHL3, respectively, in PBS buffer containing 0.05% Tween20, 0.1% BSA and 1 mM DTT for 30 minutes were added to the ubiquitin-AMC/USP30 peptide ligands mixture and the initial velocity was immediately measured by monitoring fluorescence (excitation at 340 nm and emission at 465 nm) using SpectraMax®M5e (Molecular Device, Sunnyvale, Calif.). The initial rates were calculated based on slopes of increasing fluorescence signal. The velocity was normalized to the percentage of the rate when the peptide ligand concentration was zero, and the data was processed using KaleidaGraph by fitting to the following equation:

$$v = {v}_{0} + \frac{{v}_{max} - {v}_{0}}{1 + {( \frac{I}{{IC}_{50}} )}^{n}}$$

in which v is the percentage of maximum rate; I is the concentration of inhibitor (USP30 peptide ligands); v₀ and vₘₐₓ are minimum and maximum percentage of the rate, respectively.

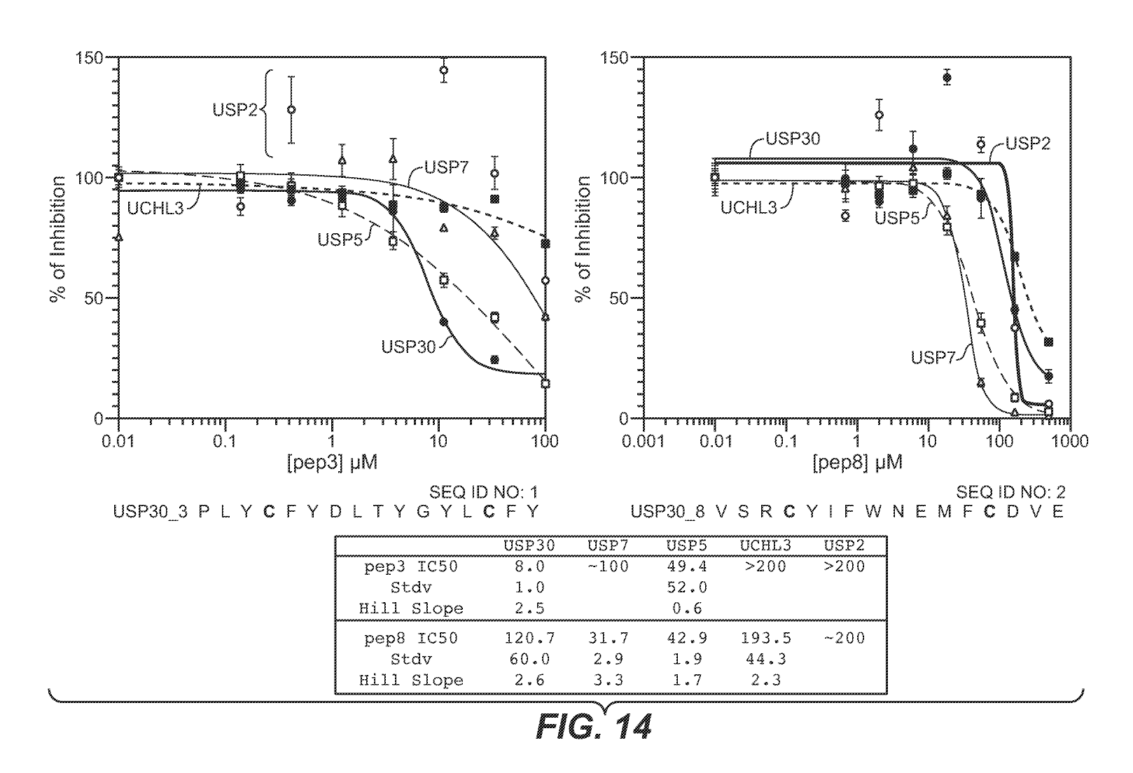

The results of that experiment are shown in FIG. 14. Peptide USP30_{—}3 showed good specificity for USP30, with an IC50 of 8.0 μM. The IC50 of peptide USP30_{—}3 for USP5 was more than 6-fold higher, at 49.4 μM, and for USP2 was more than 10-fold higher, at about 100 μM. The IC50 of peptide USP30_{—}3 for UCHL3 and USP2 was >200 μM.

Peptide USP30_{—}3 was selected for affinity maturation. To improve the affinity, a soft randomized library was constructed using the USP30_{—}3 sequence as the targeted parent, and panned against USP30_cd in solution as described previously (Stanger, et al., 2012, Nat. Chem. Bio., 7: 655-660). After four rounds of solution panning, 20 peptides were identified that bound to USP30 catalytic domain with stronger spot phage ELISA signal than the parent USP30_{—}3, manifested by an improvement in the signal/noise ratio of about 3-6 fold.

FIG. 15 shows a graph of residue probability by position in USP30_{—}3 and the affinity matured peptides. The sequences for certain affinity matured peptides are shown below the graph, along with the signal to noise ratio (“S/N”), which is the ratio of the spot phage ELISA signal (“signal”) detected against biotinylated USP30 cd captured on NeutrAvidin-coated 384 well Maxisorb plates versus the ELISA signal against the NeutrAvidin-coated plate alone. FIG. 15 also shows the number of occurrences of each sequence in the selection (“n”), the total number of clones (“Total”; 66), and the number of unique sequences (“Uniq”; 20). All of the affinity matured peptides shown had signal to noise ratios of greater than 10 except for USP30_{—}3.27 and USP30_3.62.

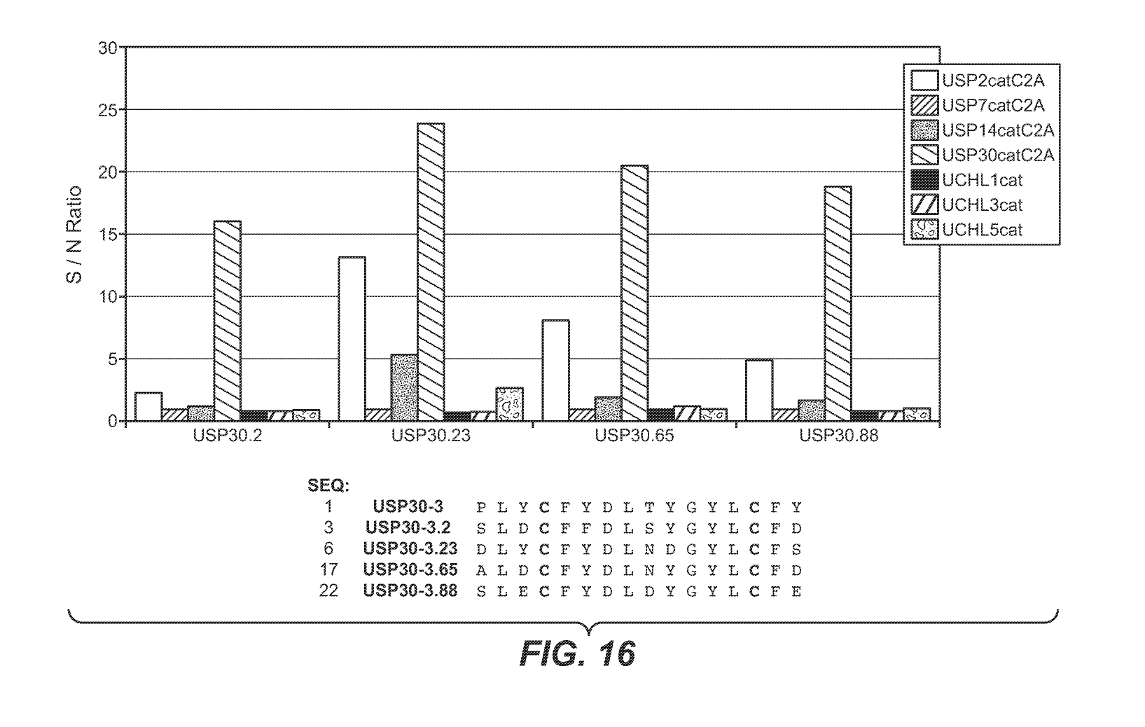

Certain peptides were then tested for specificity for USP30 versus other deubiquitinating enzymes. Binding was tested by ELISA. FIG. 16 shows the signal to noise ratio (“s/n ratio”) for binding of parent USP30_{—}3 peptide and affinity matured peptides USP30_{—}3.2, USP30_{—}3.23, USP30_{—}3.65, and USP30_{—}3.88 to the catalytic domains of USP2, USP7, USP14, and USP30 (each with the active site cysteine mutated to alanine), and to the catalytic domains of UCHL1, UCHL3, and UCHL5. For the set of bar graphs for each peptide, the targets tested, from left to right, were USP2, USP7, USP14, USP30, UCHL1, UCHL3, and UCHL5.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

TABLE 4
Table of Sequences

| SEQ | | |
|---|---|---|
| ID | | |
| NO | Description | Sequence |
| 26 | Human | MLSSRAEAAM TAADRAIQRF LRTGAAVRYK VMKNWGVIGG IAAALAAGIY |
| | ubiquitin- | VIWGPITERK KRRKGLVPGL VNLGNTCFMN SLLQGLSACP AFIRWLEEFT |
| | specific | SQYSRDQKEP PSHQYLSLTL LHLLKALSCQ EVTDDEVLDA SCLLDVLRMY |
| | peptidase 30 | RWQISSFEEQ DAHELFHVIT SSLEDERDRQ PRVTHLFDVH SLEQQSEITP |
| | (USP30); | KQITCRTRGS PHPTSNHWKS QHPFHGRLTS NMVCKHCEHQ SPVRFDTFDS |
| | SwissProt | LSLSIPAATW GHPLTLDHCL HHFISSESVR DVVCDNCTKI EAKGTLNGEK |
| | Q70CQ3.1 | VEHQRTTFVK QLKLGKLPQC LCIHLQRLSW SSHGTPLKRH EHVQFNEFLM |
| | | MDIYKYHLLG HKPSQHNPKL NKNPGPTLEL QDGPGAPTPV LNQPGAPKTQ |
| | | IFMNGACSPS LLPTLSAPMP FPLPVVPDYS SSTYLFRLMA VVVHHGDMHS |
| | | GHFVTYRRSP PSARNPLSTS NQWLWVSDDT VRKASLQEVL SSSAYLLFYE |
| | | RVLSRMQHQS QECKSEE |
| 27 | Human | MVGRNSAIAA GVCGALFIGY CIYFDRKRRS DPNFKNRLRE RRKKQKLAKE |
| | mitochondrial | RAGLSKLPDL KDAEAVQKFF LEEIQLGEEL LAQGEYEKGV DHLTNAIAVC |
| | import receptor | GQPQQLLQVL QQTLPPPVFQ MLLTKLPTIS QRIVSAQSLA EDDVE |
| | subunit 20 | |
| | homolog | |
| | (Tom20); | |
| | GenBank | |
| | NP_055580.1 | |
| 28 | Human | MKKDVRILLV GEPRVGKTSL IMSLVSEEFP EEVPPRAEEI TIPADVTPER |
| | MIRO1; | VPTHIVDYSE AEQSDEQLHQ EISQANVICI VYAVNNKHSI DKVTSRWIPL |
| | SwissProt | INERTDKDSR LPLILVGNKS DLVEYSSMET ILPIMNQYTE IETCVECSAK |
| | Q8IXI2.2 | NLKNISELFY YAQKAVLHPT GPLYCPEEKE MKPACIKALT RIFKISDQDN |
| | | DGTLNDAELN FFQRICFNTP LAPQALEDVK NVVRKHISDG VADSGLTLKG |
| | | FLFLHTLFIQ RGRHETTWTV LRRFGYDDDL DLTPEYLFPL LKIPPDCTTE |
| | | LNHHAYLFLQ STFDKHDLDR DCALSPDELK DLFKVFPYIP WGPDVNNTVC |
| | | TNERGWITYQ GFLSQWTLTT YLDVQRCLEY LGYLGYSILT EQESQASAVT |
| | | VTRDKKIDLQ KKQTQRNVFR CNVIGVKNCG KSGVLQALLG RNLMRQKKIR |
| | | EDHKSYYAIN TVYVYGQEKY LLLHDISESE FLTEAEIICD VVCLVYDVSN |
| | | PKSFEYCARI FKQHFMDSRI PCLIVAAKSD LHEVKQEYSI SPTDFCRKHK |
| | | MPPPQAFTCN TADAPSKDIF VKLTTMAMYP HVTQADLKSS TFWLRASFGA |
| | | TVFAVLGFAM YKALLKQR |
| 29 | Human Parkin; | MIVFVRFNSS HGFPVEVDSD TSIFQLKEVV AKRQGVPADQ LRVIFAGKEL |
| | GenBank | RNDWTVQNCD LDQQSIVHIV QRPWRKGQEM NATGGDDPRN AAGGCEREPQ |
| | NP_004553.2 | SLTRVDLSSS VLPGDSVGLA VILHTDSRKD SPPAGSPAGR SIYNSFYVYC |
| | | KGPCQRVQPG KLRVQCSTCR QATLTLTQGP SCWDDVLIPN RMSGECQSPH |
| | | CPGTSAEFFF KCGAHPTSDK ETSVALHLIA TNSRNITCIT CTDVRSPVLV |
| | | FQCNSRHVIC LDCFHLYCVT RLNDRQFVHD PQLGYSLPCV AGCPNSLIKE |
| | | LHHFRILGEE QYNRYQQYGA EECVLQMGGV LCPRPGCGAG LLPEPDQRKV |
| | | TCEGGNGLGC GFAFCRECKE AYHEGECSAV FEASGTTTQA YRVDERAAEQ |
| | | ARWEAASKET IKKTTKPCPR CHVPVEKNGG CMHMKCPQPQ CRLEWCWNCG |
| | | CEWNRVCMGD HWFDV |
| 30 | Human | MAVRQALGRG LQLGRALLLR FTGKPGRAYG LGRPGPAAGC VRGERPGWAA |
| | PINK1; | GPGAEPRRVG LGLPNRLRFF RQSVAGLAAR LQRQFVVRAW GCAGPCGRAV |
| | SwissProt | FLAFGLGLGL IEEKQAESRR AVSACQEIQA IFTQKSKPGP DPLDTRRLQG |
| | Q9BXM7.1 | FRLEEYLIGQ SIGKGCSAAV YEATMPTLPQ NLEVTKSTGL LPGRGPGTSA |
| | | PGEGQERAPG APAFPLAIKM MWNISAGSSS EAILNTMSQE LVPASRVALA |
| | | GEYGAVTYRK SKRGPKQLAP HPNIIRVLRA FTSSVPLLPG ALVDYPDVLP |
| | | SRLHPEGLGH GRTLFLVMKN YPCTLRQYLC VNTPSPRLAA MMLLQLLEGV |
| | | DHLVQQGIAH RDLKSDNILV ELDPDGCPWL VIADFGCCLA DESIGLQLPF |
| | | SSWYVDRGGN GCLMAPEVST ARPGPRAVID YSKADAWAVG AIAYEIFGLV |
| | | NPFYGQGKAH LESRSYQEAQ LPALPESVPP DVRQLVRALL QREASKRPSA |
| | | RVAANVLHLS LWGEHILALK NLKLDKMVGW LLQQSAATLL ANRLTEKCCV |
| | | ETKMKMLFLA NLECETLCQA ALLLCSWRAA L |
| 31 | USP30 | TGCGGCCGCA GGTTCCGCTG TCTCGGGAAC CGTCGTATCC CTCGGTCCGG |
| | mRNA; | CGGCGGCGGC GGCGGTAGCG GAGGAGACGG TTTCAGGCCT CCGGTGCGGC |
| | GenBank | TGCAATGCTG AGCTCCCGGG CCGAGGCGGC GATGACCGCG GCCGACAGGG |
| | NM_032663.3 | CCATCCAGCG CTTCCTGCGG ACCGGGGCGG CCGTCAGATA TAAAGTCATG |
| | | AAGAACTGGG GAGTTATAGG TGGAATTGCT GCTGCTCTTG CAGCAGGAAT |
| | | ATATGTTATT TGGGGTCCCA TTACAGAAAG AAAGAAGCGT AGAAAAGGGC |
| | | TTGTGCCTGG CCTTGTTAAT TTAGGGAACA CCTGCTTCAT GAACTCCCTG |
| | | CTACAAGGCC TGTCTGCCTG TCCTGCTTTC ATCAGGTGGC TGGAAGAGTT |
| | | CACCTCCCAG TACTCCAGGG ATCAGAAGGA GCCCCCCTCA CACCAGTATT |
| | | TATCCTTAAC ACTCTTGCAC CTTCTGAAAG CCTTGTCCTG CCAAGAAGTT |
| | | ACTGATGATG AGGTCTTAGA TGCAAGCTGC TTGTTGGATG TCTTAAGAAT |
| | | GTACAGATGG CAGATCTCAT CATTTGAAGA ACAGGATGCT CACGAATTAT |
| | | TCCATGTCAT TACCTCGTCA TTGGAAGATG AGCGAGACCG CCAGCCTCGG |
| | | GTCACACATT TGTTTGATGT GCATTCCCTG GAGCAGCAGT CAGAAATAAC |
| | | TCCCAAACAA ATTACCTGCC GCACAAGAGG GTCACCTCAC CCTACATCCA |
| | | ATCACTGGAA GTCTCAACAT CCTTTTCATG GAAGACTCAC TAGTAATATG |
| | | GTCTGCAAAC ACTGTGAACA CCAGAGTCCT GTTCGATTTG ATACCTTTGA |
| | | TAGCCTTTCA CTAAGTATTC CAGCCGCCAC ATGGGGTCAC CCATTGACCC |
| | | TGGACCACTG CCTTCACCAC TTCATCTCAT CAGAATCAGT GCGGGATGTT |
| | | GTGTGTGACA ACTGTACAAA GATTGAAGCC AAGGGAACGT TGAACGGGGA |
| | | AAAGGTGGAA CACCAGAGGA CCACTTTTGT TAAACAGTTA AAACTAGGGA |
| | | AGCTCCCTCA GTGTCTCTGC ATCCACCTAC AGCGGCTGAG CTGGTCCAGC |
| | | CACGGCACGC CTCTGAAGCG GCATGAGCAC GTGCAGTTCA ATGAGTTCCT |
| | | GATGATGGAC ATTTACAAGT ACCACCTCCT TGGACATAAA CCTAGTCAAC |
| | | ACAACCCTAA ACTGAACAAG AACCCAGGGC CTACACTGGA GCTGCAGGAT |
| | | GGGCCGGGAG CCCCCACACC AGTTCTGAAT CAGCCAGGGG CCCCCAAAAC |
| | | ACAGATTTTT ATGAATGGCG CCTGCTCCCC ATCTTTATTG CCAACGCTGT |
| | | CAGCGCCGAT GCCCTTCCCT CTCCCAGTTG TTCCCGACTA CAGCTCCTCC |
| | | ACATACCTCT TCCGGCTGAT GGCAGTTGTC GTCCACCATG GAGACATGCA |
| | | CTCTGGACAC TTTGTCACTT ACCGACGGTC CCCACCTTCT GCCAGGAACC |
| | | CTCTCTCAAC TAGCAATCAG TGGCTGTGGG TCTCCGATGA CACTGTCCGC |
| | | AAGGCCAGCC TGCAGGAGGT CCTGTCCTCC AGCGCCTACC TGCTGTTCTA |
| | | CGAGCGCGTC CTTTCCAGGA TGCAGCACCA GAGCCAGGAG TGCAAGTCTG |
| | | AAGAATGACT GTGCCCTCCT GCAAGGCTAG AGCTGATGGC ACTGTCTGCA |
| | | CTGTCCAGGA AAAAAGTAAA ACTGTACTGT TGCGTGTGCA AGCGGCCCCA |
| | | CTAGAGCCTT CCAGCCTTCT GGTGTGTTCT AAGAGCAGGC TCCACCTGGG |
| | | AGCCAGCCCC AGTTCACACC AAACCAGGCT CCCTGAACAG TCCTGTTCAT |
| | | GTGTGTAGGT GGTTCTGTTG TGTTAAGAAA GCATTCATTA TGTCCGGAGT |
| | | GTCTTTTTAC TCATCTGATA CAGGTAATTA AAAGAACTCA GATTCTTGAA |
| | | GCCACCGTTT TCATATTGTA ATGTTAGGTG TTCTCAGAGG GGAGGTACCT |
| | | TTGTCTAATC AACGTTTCCA CTTAGATCTT TTATTTTTAA TAAGCAGGCC |
| | | CATAAAAATT GTTGACAAGA ATTAATGAAA TTATTAAAGG CAACAATTTA |
| | | GAAGAAAAAG TGCCTTTCAC TTTCGATTGC TTTTGTAGCA CGTCCATTGT |
| | | GAAATATTCC TTCCAGGCTA CTCAAAGGAT AGCAAGAGAA CAGGTAAATG |
| | | ATGCCTAAAG AACACCTTCC TTTTTCTATG CCTTTTCTAA TCTTTCAATT |
| | | CTTTCTATGG AGTAAAGGCT CATCTGCCAA ATCTGCCCCC TGGGGAAACT |
| | | CTTTCACTAC TTTGTCAGTT ATAAGTGAAG AGCTTACTTG TTGCTTTTAT |
| | | CTTTTGTATA TTGGACTGAG ATGTAATTAC ACTGTATTAT AAAACTCTGT |
| | | GAATAGCCAG AACTGAGCTG GATCTTTGCA ACACCTGATT CCTCTGCTCT |
| | | GTGGAAAACT TTTTCTTACA CAAGGATCCA CTGTGGACGG TTACTTTCAT |
| | | CTGTTTATTT ATTGCCCATG CAGAGCTCTT AAGGTTTACA GGTGGGAGCT |
| | | TGGGGCTGTA TAAAAAAATA ATCCCTGCCC TGAGTTGACA CCTGGCTTAG |
| | | GAAGGAAGGG CTGACTATGG GGCTGCAGTC TCTCTGAACC TCAGTTTCCT |
| | | CATTTGTGAA GTGAAGGGTT AGATTTGATG ACCACCAAAG TTCAGCCCTT |
| | | TTCACGAAAA GGAGAAAGCA GCTTTTGACT TTTTAAAAAA CATATAACTA |
| | | CAGCTGGCAT CTAGTATTGT CATGTTGCTC TAGGTCCATA TTCTGAATTT |
| | | ATTCATTTCC AATAGCCTAA TACAAAAAGT ATATATTGAG CACTTTCTTC |
| | | CCTTTTCAGG TAAGTCTCTG AATGCAGCCC AGGGCCAAAG GAATTTTGAT |
| | | GACACAGTAG TACCTATGTT TTAAGCTATA TTTTTAATTT AGAAAAATGG |
| | | ATACCAAATT CAAACCGACT CATCAGAGGT AAGATTTGGA ATCAGACCTT |
| | | TCCAAAAGGT CATCTGAGGT AAGGCTAAGA CCGCACTTCC TCTGCTGGGG |
| | | GTGAGCTGGC AGACACACCA AACAGTGCCT TGGCAGCAGC TCACAGTGCA |
| | | GGAAGCCCAG GTGATCACTC TTCTGCTGGG CCCAGGCTGC ACCCTGAGGA |
| | | CTCAGTAACT CACTCTCAAC AGAATATTCT GTGCAGGCTC TCCAGGCTCT |
| | | GGGCGTCAGG GTGCAAGGGG CAGCTTGAAC TGTACGGTCC GTCCTGCACT |
| | | CACCCGATGC AGACCTTGAC TTTGATGTTG AAATGAACAC ACTTGTTTTA |
| | | CCCAAGTCTG GTGGAACAAA TGCCCAATCA TGTGACCTTA AAGTGTACTG |
| | | CAAAGCTGTA GCTTTAAGTA ATTGCTGTTC TGCCACTGCT TACTCTGAAA |
| | | TCTACCATCA AAGAAAGATA GAGAAAAGGG GCTGAGCCTT GGAATATATG |
| | | GTTATAAGCA GATCTTTCTT TGGTCAGAGA CCAGGGTTTG AGCCAAGGCT |
| | | GTAAATGTGA ACAATAGCTG TGCAAAGCCT TTTAACCTGA CTTCTTCATT |
| | | TTGTAAATTA TTATGCATTA AGTAGCAGCC CAATAATCTG ATTTCTAGTT |
| | | TTATTTTCAA AGTAAGTAGC TTCTTTTGGG AAAAACCTAA GTTAAACTAG |
| | | TAGTTTTGCC ATAATAACTG CTGATTTATG TATTTGCTAA AGGTACTTTT |
| | | GTATCTGCTG TGTATTATAG CAATAAAATA ATCATTTTGT TAGAAAAAAA |
| | | TCAAAAAAAA AAAAAA |
| 32 | Human MUL1; | MESGGRPSLC QFILLGTTSV VTAALYSVYR QKARVSQELK GAKKVHLGED |
| | SwissProt | LKSILSEAPG KCVPYAVIEG AVRSVKETLN SQFVENCKGV IQRLTLQEHK |
| | Q969V5.1 | MVWNRTTHLW NDCSKIIHQR TNTVPFDLVP HEDGVDVAVR VLKPLDSVDL |
| | | GLETVYEKFH PSIQSFTDVI GHYISGERPK GIQETEEMLK VGATLTGVGE |
| | | LVLDNNSVRL QPPKQGMQYY LSSQDFDSLL QRQESSVRLW KVLALVFGFA |
| | | TCATLFFILR KQYLQRQERL RLKQMQEEFQ EHEAQLLSRA KPEDRESLKS |
| | | ACVVCLSSFK SCVFLECGHV CSCTECYRAL PEPKKCPICR QAITRVIPLY |
| | | NS |
| 33 | Human ASNS; | MCGIWALFGS DDCLSVQCLS AMKIAHRGPD AFRFENVNGY TNCCFGFHRL |
| | GenBank | AVVDPLFGMQ PIRVKKYPYL WLCYNGEIYN HKKMQQHFEF EYQTKVDGEI |
| | NP_899199.2 | ILHLYDKGGI EQTICMLDGV FAFVLLDTAN KKVFLGRDTY GVRPLFKAMT |
| | | EDGFLAVCSE AKGLVTLKHS ATPFLKVEPF LPGHYEVLDL KPNGKVASVE |
| | | MVKYHHCRDV PLHALYDNVE KLFPGFEIET VKNNLRILFN NAVKKRLMTD |
| | | RRIGCLLSGG LDSSLVAATL LKQLKEAQVQ YPLQTFAIGM EDSPDLLAAR |
| | | KVADHIGSEH YEVLFNSEEG IQALDEVIFS LETYDITTVR ASVGMYLISK |
| | | YIRKNTDSVV IFSGEGSDEL TQGYIYFHKA PSPEKAEEES ERLLRELYLF |
| | | DVLRADRTTA AHGLELRVPF LDHRFSSYYL SLPPEMRIPK NGIEKHLLRE |
| | | TFEDSNLIPK EILWRPKEAF SDGITSVKNS WFKILQEYVE HQVDDAMMAN |
| | | AAQKFPFNTP KTKEGYYYRQ VFERHYPGRA DWLSHYWMPK WINATDPSAR |
| | | TLTHYKSAVK A |
| 34 | Human | MASCAEPSEP SAPLPAGVPP LEDFEVLDGV EDAEGEEEEE EEEEEEDDLS |
| | FKBP8; | ELPPLEDMGQ PPAEEAEQPG ALAREFLAAM EPEPAPAPAP EEWLDILGNG |
| | SwissProt | LLRKKTLVPG PPGSSRPVKG QVVTVHLQTS LENGTRVQEE PELVFTLGDC |
| | Q14318.2 | DVIQALDLSV PLMDVGETAM VTADSKYCYG PQGRSPYIPP HAALCLEVTL |
| | | KTAVDGPDLE MLTGQERVAL ANRKRECGNA HYQRADFVLA ANSYDLAIKA |
| | | ITSSAKVDMT FEEEAQLLQL KVKCLNNLAA SQLKLDHYRA ALRSCSLVLE |
| | | HQPDNIKALF RKGKVLAQQG EYSEAIPILR AALKLEPSNK TIHAELSKLV |
| | | KKHAAQRSTE TALYRKMLGN PSRLPAKCPG KGAWSIPWKW LFGATAVALG |
| | | GVALSVVIAA RN |
| 35 | Human | MAASKPVEAA VVAAAVPSSG SGVGGGGTAG PGTGGLPRWQ LALAVGAPLL |
| | TOM70; | LGAGAIYLWS RQQRRREARG RGDASGLKRN SERKTPEGRA SPAPGSGHPE |
| | SwissProt | GPGAHLDMNS LDRAQAAKNK GNKYFKAGKY EQAIQCYTEA ISLCPTEKNV |
| | O94826.1 | DLSTFYQNRA AAFEQLQKWK EVAQDCTKAV ELNPKYVKAL FRRAKAHEKL |
| | | DNKKECLEDV TAVCILEGFQ NQQSMLLADK VLKLLGKEKA KEKYKNREPL |
| | | MPSPQFIKSY FSSFTDDIIS QPMLKGEKSD EDKDKEGEAL EVKENSGYLK |
| | | AKQYMEEENY DKIISECSKE IDAEGKYMAE ALLLRATFYL LIGNANAAKP |
| | | DLDKVISLKE ANVKLRANAL IKRGSMYMQQ QQPLLSTQDF NMAADIDPQN |
| | | ADVYHHRGQL KILLDQVEEA VADFDECIRL RPESALAQAQ KCFALYRQAY |
| | | TGNNSSQIQA AMKGFEEVIK KFPRCAEGYA LYAQALTDQQ QFGKADEMYD |
| | | KCIDLEPDNA TTYVHKGLLQ LQWKQDLDRG LELISKAIEI DNKCDFAYET |
| | | MGTIEVQRGN MEKAIDMFNK AINLAKSEME MAHLYSLCDA AHAQTEVAKK |
| | | YGLKPPTL |
| 36 | Human | MVGREKELSI HFVPGSCRLV EEEVNIPNRR VLVTGATGLL GRAVHKEFQQ |
| | MAT2B; | NNWHAVGCGF RRARPKFEQV NLLDSNAVHH IIHDFQPHVI VHCAAERRPD |
| | SwissProt | VVENQPDAAS QLNVDASGNL AKEAAAVGAF LIYISSDYVF DGTNPPYREE |
| | Q9NZL9.1 | DIPAPLNLYG KTKLDGEKAV LENNLGAAVL RIPILYGEVE KLEESAVTVM |
| | | FDKVQFSNKS ANMDHWQQRF PTHVKDVATV CRQLAEKRML DPSIKGTFHW |
| | | SGNEQMTKYE MACAIADAFN LPSSHLRPIT DSPVLGAQRP RNAQLDCSKL |
| | | ETLGIGQRTP FRIGIKESLW PFLIDKRWRQ TVFH |
| 37 | Human | MAAAVGRLLR ASVARHVSAI PWGISATAAL RPAACGRTSL TNLLCSGSSQ |
| | PRDX3; | AKLFSTSSSC HAPAVTQHAP YFKGTAVVNG EFKDLSLDDF KGKYLVLFFY |
| | SwissProt | PLDFTFVCPT EIVAFSDKAN EFHDVNCEVV AVSVDSHFSH LAWINTPRKN |
| | P30048.3 | GGLGHMNIAL LSDLTKQISR DYGVLLEGSG LALRGLFIID PNGVIKHLSV |
| | | NDLPVGRSVE ETLRLVKAFQ YVETHGEVCP ANWTPDSPTI KPSPAASKEY |
| | | FQKVNQ |
| 38 | Human IDE; | MRYRLAWLLH PALPSTFRSV LGARLPPPER LCGFQKKTYS KMNNPAIKRI |
| | SwissProt | GNHITKSPED KREYRGLELA NGIKVLLISD PTTDKSSAAL DVHIGSLSDP |
| | P14735.4 | PNIAGLSHFC EHMLFLGTKK YPKENEYSQF LSEHAGSSNA FTSGEHTNYY |
| | | FDVSHEHLEG ALDRFAQFFL CPLFDESCKD REVNAVDSEH EKNVMNDAWR |
| | | LFQLEKATGN PKHPFSKFGT GNKYTLETRP NQEGIDVRQE LLKFHSAYYS |
| | | SNLMAVCVLG RESLDDLTNL VVKLFSEVEN KNVPLPEFPE HPFQEEHLKQ |
| | | LYKIVPIKDI RNLYVTFPIP DLQKYYKSNP GHYLGHLIGH EGPGSLLSEL |
| | | KSKGWVNTLV GGQKEGARGF MFFIINVDLT EEGLLHVEDI ILHMFQYIQK |
| | | LRAEGPQEWV FQECKDLNAV AFRFKDKERP RGYTSKIAGI LHYYPLEEVL |
| | | TAEYLLEEFR PDLIEMVLDK LRPENVRVAI VSKSFEGKTD RTEEWYGTQY |
| | | KQEAIPDEVI KKWQNADLNG KFKLPTKNEF IPTNFEILPL EKEATPYPAL |
| | | IKDTAMSKLW FKQDDKFFLP KACLNFEFFS PFAYVDPLHC NMAYLYLELL |
| | | KDSLNEYAYA AELAGLSYDL QNTIYGMYLS VKGYNDKQPI LLKKIIEKMA |
| | | TFEIDEKRFE IIKEAYMRSL NNFRAEQPHQ HAMYYLRLLM TEVAWTKDEL |
| | | KEALDDVTLP RLKAFIPQLL SRLHIEALLH GNITKQAALG IMQMVEDTLI |
| | | EHAHTKPLLP SQLVRYREVQ LPDRGWFVYQ QRNEVHNNCG IEIYYQTDMQ |
| | | STSENMFLEL FCQIISEPCF NTLRTKEQLG YIVFSGPRRA NGIQGLRFII |
| | | QSEKPPHYLE SRVEAFLITM EKSIEDMTEE AFQKHIQALA IRRLDKPKKL |
| | | SAECAKYWGE IISQQYNFDR DNTEVAYLKT LTKEDIIKFY KEMLAVDAPR |
| | | RHKVSVHVLA REMDSCPVVG EFPCQNDINL SQAPALPQPE VIQNMTEFKR |
| | | GLPLFPLVKP HINFMAAKL |
| 39 | Human | MAVPPTYADL GKSARDVFTK GYGFGLIKLD LKTKSENGLE FTSSGSANTE |
| | VDAC1; | TTKVTGSLET KYRWTEYGLT FTEKWNTDNT LGTEITVEDQ LARGLKLTFD |
| | SwissProt | SSFSPNTGKK NAKIKTGYKR EHINLGCDMD FDIAGPSIRG ALVLGYEGWL |
| | P21796.2 | AGYQMNFETA KSRVTQSNFA VGYKTDEFQL HTNVNDGTEF GGSIYQKVNK |
| | | KLETAVNLAW TAGNSNTRFG IAAKYQIDPD ACFSAKVNNS SLIGLGYTQT |
| | | LKPGIKLTLS ALLDGKNVNA GGHKLGLGLE FQA |
| 44 | Human | MATHGQTCAR PMCIPPSYAD LGKAARDIFN KGFGFGLVKL DVKTKSCSGV |
| | VDAC2; | EFSTSGSSNT DTGKVTGTLE TKYKWCEYGL TFTEKWNTDN TLGTEIAIED |
| | SwissProt | QICQGLKLTF DTTFSPNTGK KSGKIKSSYK RECINLGCDV DFDFAGPAIH |
| | P45880.2 | GSAVFGYEGW LAGYQMTFDS AKSKLTRNNF AVGYRTGDFQ LHTNVNDGTE |
| | | FGGSIYQKVC EDLDTSVNLA WTSGTNCTRF GIAAKYQLDP TASISAKVNN |
| | | SSLIGVGYTQ TLRPGVKLTL SALVDGKSIN AGGHKVGLAL ELEA |
| 45 | Human | MCNTPTYCDL GKAAKDVFNK GYGFGMVKID LKTKSCSGVE FSTSGHAYTD |
| | VDAC3; | TGKASGNLET KYKVCNYGLT FTQKWNTDNT LGTEISWENK LAEGLKLTLD |
| | SwissProt | TIFVPNTGKK SGKLKASYKR DCFSVGSNVD IDFSGPTIYG WAVLAFEGWL |
| | Q9Y277.1 | AGYQMSFDTA KSKLSQNNFA LGYKAADFQL HTHVNDGTEF GGSIYQKVNE |
| | | KIETSINLAW TAGSNNTRFG IAAKYMLDCR TSLSAKVNNA SLIGLGYTQT |
| | | LRPGVKLTLS ALIDGKNFSA GGHKVGLGFE LEA |
| 40 | Human IPO5; | MAAAAAEQQQ FYLLLGNLLS PDNVVRKQAE ETYENIPGQS KITFLLQAIR |
| | SwissProt | NTTAAEEARQ MAAVLLRRLL SSAFDEVYPA LPSDVQTAIK SELLMIIQME |
| | O00410.4 | TQSSMRKKVC DIAAELARNL IDEDGNNQWP EGLKFLFDSV SSQNVGLREA |
| | | ALHIFWNFPG IFGNQQQHYL DVIKRMLVQC MQDQEHPSIR TLSARATAAF |
| | | ILANEHNVAL FKHFADLLPG FLQAVNDSCY QNDDSVLKSL VEIADTVPKY |
| | | LRPHLEATLQ LSLKLCGDTS LNNMQRQLAL EVIVTLSETA AAMLRKHTNI |
| | | VAQTIPQMLA MMVDLEEDED WANADELEDD DFDSNAVAGE SALDRMACGL |
| | | GGKLVLPMIK EHIMQMLQNP DWKYRHAGLM ALSAIGEGCH QQMEGILNEI |
| | | VNFVLLFLQD PHPRVRYAAC NAVGQMATDF APGFQKKFHE KVIAALLQTM |
| | | EDQGNQRVQA HAAAALINFT EDCPKSLLIP YLDNLVKHLH SIMVLKLQEL |
| | | IQKGTKLVLE QVVTSIASVA DTAEEKFVPY YDLFMPSLKH IVENAVQKEL |
| | | RLLRGKTIEC ISLIGLAVGK EKFMQDASDV MQLLLKTQTD FNDMEDDDPQ |
| | | ISYMISAWAR MCKILGKEFQ QYLPVVMGPL MKTASIKPEV ALLDTQDMEN |
| | | MSDDDGWEFV NLGDQQSFGI KTAGLEEKST ACQMLVCYAK ELKEGFVEYT |
| | | EQVVKLMVPL LKFYFHDGVR VAAAESMPLL LECARVRGPE YLTQMWHFMC |
| | | DALIKAIGTE PDSDVLSEIM HSFAKCIEVM GDGCLNNEHF EELGGILKAK |
| | | LEEHFKNQEL RQVKRQDEDY DEQVEESLQD EDDNDVYILT KVSDILHSIF |
| | | SSYKEKVLPW FEQLLPLIVN LICPHRPWPD RQWGLCIFDD VIEHCSPASF |
| | | KYAEYFLRPM LQYVCDNSPE VRQAAAYGLG VMAQYGGDNY RPFCTEALPL |
| | | LVRVIQSADS KTKENVNATE NCISAVGKIM KFKPDCVNVE EVLPHWLSWL |
| | | PLHEDKEEAV QTFNYLCDLI ESNHPIVLGP NNTNLPKIFS IIAEGEMHEA |
| | | IKHEDPCAKR LANVVRQVQT SGGLWTECIA QLSPEQQAAI QELLNSA |
| 41 | Human PTH2; | MPSKSLVMEY LAHPSTLGLA VGVACGMCLG WSLRVCFGML PKSKTSKTHT |
| | SwissProt | DTESEASILG DSGEYKMILV VRNDLKMGKG KVAAQCSHAA VSAYKQIQRR |
| | Q9Y3E5.1 | NPEMLKQWEY CGQPKVVVKA PDEETLIALL AHAKMLGLTV SLIQDAGRTQ |
| | | IAPGSQTVLG IGPGPADLID KVTGHLKLY |
| 42 | Human | MKDVPGFLQQ SQNSGPGQPA VWHRLEELYT KKLWHQLTLQ VLDFVQDPCF |
| | PSD13; | AQGDGLIKLY ENFISEFEHR VNPLSLVEII LHVVRQMTDP NVALTFLEKT |
| | SwissProt | REKVKSSDEA VILCKTAIGA LKLNIGDLQV TKETIEDVEE MLNNLPGVTS |
| | Q9UNM6.2 | VHSRFYDLSS KYYQTIGNHA SYYKDALRFL GCVDIKDLPV SEQQERAFTL |
| | | GLAGLLGEGV FNFGELLMHP VLESLRNTDR QWLIDTLYAF NSGNVERFQT |
| | | LKTAWGQQPD LAANEAQLLR KIQLLCLMEM TFTRPANHRQ LTFEEIAKSA |
| | | KITVNEVELL VMKALSVGLV KGSIDEVDKR VHMTWVQPRV LDLQQIKGMK |
| | | DRLEFWCTDV KSMEMLVEHQ AHDILT |
| 43 | Human | MQRRGALFGM PGGSGGRKMA AGDIGELLVP HMPTIRVPRS GDRVYKNECA |
| | UBP13; | FSYDSPNSEG GLYVCMNTFL AFGREHVERH FRKTGQSVYM HLKRHVREKV |
| | SwissProt | RGASGGALPK RRNSKIFLDL DTDDDLNSDD YEYEDEAKLV IFPDHYEIAL |
| | Q92995.2 | PNIEELPALV TIACDAVLSS KSPYRKQDPD TWENELPVSK YANNLTQLDN |
| | | GVRIPPSGWK CARCDLRENL WLNLTDGSVL CGKWFFDSSG GNGHALEHYR |
| | | DMGYPLAVKL GTITPDGADV YSFQEEEPVL DPHLAKHLAH FGIDMLHMHG |
| | | TENGLQDNDI KLRVSEWEVI QESGTKLKPM YGPGYTGLKN LGNSCYLSSV |
| | | MQAIFSIPEF QRAYVGNLPR IFDYSPLDPT QDFNTQMTKL GHGLLSGQYS |
| | | KPPVKSELIE QVMKEEHKPQ QNGISPRMFK AFVSKSHPEF SSNRQQDAQE |
| | | FFLHLVNLVE RNRIGSENPS DVFRFLVEER IQCCQTRKVR YTERVDYLMQ |
| | | LPVAMEAATN KDELIAYELT RREAEANRRP LPELVRAKIP FSACLQAFSE |
| | | PENVDDFWSS ALQAKSAGVK TSRFASFPEY LVVQIKKFTF GLDWVPKKFD |
| | | VSIDMPDLLD INHLRARGLQ PGEEELPDIS PPIVIPDDSK DRLMNQLIDP |
| | | SDIDESSVMQ LAEMGFPLEA CRKAVYFTGN MGAEVAFNWI IVHMEEPDFA |
| | | EPLTMPGYGG AASAGASVFG ASGLDNQPPE EIVAIITSMG FQRNQAIQAL |
| | | RATNNNLERA LDWIFSHPEF EEDSDFVIEM ENNANANIIS EAKPEGPRVK |
| | | DGSGTYELFA FISHMGTSTM SGHYICHIKK EGRWVIYNDH KVCASERPPK |
| | | DLGYMYFYRRIPS |

### APPENDIX A

| | | | |
|---|---|---|---|
| 1433B_HUMAN | ABCE1_HUMAN | AGM1_HUMAN | AN32B_HUMAN |
| 1433E_HUMAN | ABCF1_HUMAN | AGO1_HUMAN | AN32E_HUMAN |
| 1433F_HUMAN | ABCF2_HUMAN | AGO2_HUMAN | ANFY1_HUMAN |
| 1433G_HUMAN | ABCF3_HUMAN | AHNK_HUMAN | ANLN_HUMAN |
| 1433T_HUMAN | ABHD2_HUMAN | AHSA1_HUMAN | ANM1_HUMAN |
| 1433Z_HUMAN | ABT1_HUMAN | AIBP_HUMAN | ANM5_HUMAN |
| 1A01_HUMAN | ACACA_HUMAN | AIF1L_HUMAN | ANR26_HUMAN |
| 1A02_HUMAN | ACBD6_HUMAN | AIFM1_HUMAN | ANR46_HUMAN |
| 1B07_HUMAN | ACBP_HUMAN | AIMP1_HUMAN | ANX11_HUMAN |
| 1C07_HUMAN | ACHA5_HUMAN | AIMP2_HUMAN | ANXA1_HUMAN |
| 2A5D_HUMAN | ACINU_HUMAN | AINX_HUMAN | ANXA2_HUMAN |
| 2AAA_HUMAN | ACLY_HUMAN | AIP_HUMAN | ANXA5_HUMAN |
| 2ABA_HUMAN | ACO13_HUMAN | AKA11_HUMAN | ANXA6_HUMAN |
| 2ABD_HUMAN | ACOD_HUMAN | AKA12_HUMAN | AOFA_HUMAN |
| 3BP5_HUMAN | ACSL1_HUMAN | AKAP1_HUMAN | AP1G1_HUMAN |
| 41_HUMAN | ACSL3_HUMAN | AKAP9_HUMAN | AP1M1_HUMAN |
| 4F2_HUMAN | ACSL4_HUMAN | AKIB1_HUMAN | AP2A1_HUMAN |
| 5NT3_HUMAN | ACTA_HUMAN | AKP13_HUMAN | AP2A2_HUMAN |
| 6PGD_HUMAN | ACTB_HUMAN | AKP8L_HUMAN | AP2A_HUMAN |
| 6PGL_HUMAN | ACTN1_HUMAN | AKT2_HUMAN | AP2B1_HUMAN |
| A0PJ76_HUMAN | ACTN4_HUMAN | AL3A2_HUMAN | AP2M1_HUMAN |
| A2I9Y7_HUMAN | ACTZ_HUMAN | AL7A1_HUMAN | AP2S1_HUMAN |
| A4UCU2_HUMAN | ACV1B_HUMAN | AL9A1_HUMAN | AP3B1_HUMAN |
| A4_HUMAN | ACYP1_HUMAN | ALBU_HUMAN | AP3D1_HUMAN |
| A7UJ17_HUMAN | ADAM9_HUMAN | ALDOA_HUMAN | AP3M1_HUMAN |
| A8K781_HUMAN | ADCY3_HUMAN | ALDR_HUMAN | AP3S1_HUMAN |
| A8K7N0_HUMAN | ADCY9_HUMAN | ALG5_HUMAN | AP3S2_HUMAN |
| A8KAM7_HUMAN | ADDA_HUMAN | ALG6_HUMAN | APBA2_HUMAN |
| AAAS_HUMAN | ADHX_HUMAN | ALKB5_HUMAN | APC1_HUMAN |
| AAAT_HUMAN | ADNP_HUMAN | ALO17_HUMAN | APC4_HUMAN |
| AACS_HUMAN | ADPPT_HUMAN | AMD_HUMAN | APC5_HUMAN |
| AAKG1_HUMAN | ADRM1_HUMAN | AMOL1_HUMAN | APC7_HUMAN |
| AAMP_HUMAN | ADT1_HUMAN | AMOT_HUMAN | APC_HUMAN |
| AAPK2_HUMAN | ADT2_HUMAN | AMPL_HUMAN | APEX1_HUMAN |
| AATF_HUMAN | ADT3_HUMAN | AMPM2_HUMAN | API5_HUMAN |
| ABC3C_HUMAN | AES_HUMAN | AMRA1_HUMAN | APLP2_HUMAN |
| ABCA3_HUMAN | AF1Q_HUMAN | AN13A_HUMAN | APOL2_HUMAN |
| ABCB6_HUMAN | AFF4_HUMAN | AN13B_HUMAN | APOO_HUMAN |
| ABCBA_HUMAN | AGAL_HUMAN | AN13C_HUMAN | APR_HUMAN |
| ABCD3_HUMAN | AGK_HUMAN | AN32A_HUMAN | APT_HUMAN |
| AR6P1_HUMAN | AT12A_HUMAN | B3KPC1_HUMAN | BCCIP_HUMAN |
| AR6P4_HUMAN | AT131_HUMAN | B3KRI9_HUMAN | BCD1_HUMAN |
| ARF1_HUMAN | AT132_HUMAN | B3KTN8_HUMAN | BCLF1_HUMAN |
| ARF4_HUMAN | AT1A1_HUMAN | B4DE27_HUMAN | BCOR_HUMAN |
| ARF5_HUMAN | AT2A2_HUMAN | B4DIH6_HUMAN | BDH2_HUMAN |
| ARF6_HUMAN | AT2B1_HUMAN | B4DIM0_HUMAN | BET1L_HUMAN |
| ARFG1_HUMAN | AT2B4_HUMAN | B4DKA3_HUMAN | BET1_HUMAN |
| ARFG2_HUMAN | AT2C1_HUMAN | B4DKB3_HUMAN | BEX4_HUMAN |
| ARH40_HUMAN | AT5F1_HUMAN | B4DL94_HUMAN | BHLH9_HUMAN |
| ARHG7_HUMAN | ATAD1_HUMAN | B4DLR3_HUMAN | BI1_HUMAN |
| ARI1A_HUMAN | ATBD4_HUMAN | B4DMT9_HUMAN | BIEA_HUMAN |
| ARI1_HUMAN | ATF1_HUMAN | B4DNE0_HUMAN | BIRC2_HUMAN |
| ARI2_HUMAN | ATF2_HUMAN | B4DSP0_HUMAN | BIRC5_HUMAN |
| ARID2_HUMAN | ATF7_HUMAN | B4DW33_HUMAN | BIRC6_HUMAN |
| ARIP4_HUMAN | ATG3_HUMAN | B4E184_HUMAN | BL1S1_HUMAN |
| ARL1_HUMAN | ATLA2_HUMAN | B4E2Y0_HUMAN | BMP2K_HUMAN |
| ARL2_HUMAN | ATLA3_HUMAN | B7H6_HUMAN | BNI3L_HUMAN |
| ARL3_HUMAN | ATM_HUMAN | B7Z2A7_HUMAN | BNIP2_HUMAN |
| ARL6_HUMAN | ATOX1_HUMAN | B7Z4W9_HUMAN | BOD1L_HUMAN |
| ARL8B_HUMAN | ATP7B_HUMAN | B7Z613_HUMAN | BOD1_HUMAN |
| ARM10_HUMAN | ATPA_HUMAN | B7Z6F8_HUMAN | BOP1_HUMAN |
| ARMC1_HUMAN | ATPB_HUMAN | B7Z780_HUMAN | BOREA_HUMAN |
| ARMC6_HUMAN | ATPG_HUMAN | B7Z8Y4_HUMAN | BORG5_HUMAN |
| ARMC8_HUMAN | ATPO_HUMAN | B9D1_HUMAN | BPNT1_HUMAN |
| ARMX3_HUMAN | ATR_HUMAN | BABA1_HUMAN | BRAP_HUMAN |
| ARP19_HUMAN | ATX10_HUMAN | BACD3_HUMAN | BRAT1_HUMAN |
| ARP2_HUMAN | ATX2_HUMAN | BACH_HUMAN | BRCA1_HUMAN |
| ARP3_HUMAN | ATX3_HUMAN | BAF_HUMAN | BRCC3_HUMAN |
| ARP5L_HUMAN | AUP1_HUMAN | BAG2_HUMAN | BRAP_HUMAN |
| ARP8_HUMAN | AURKA_HUMAN | BAG5_HUMAN | BRD4_HUMAN |
| ARPC2_HUMAN | AURKB_HUMAN | BAG6_HUMAN | BRE1A_HUMAN |
| ARPC3_HUMAN | AVEN_HUMAN | BAP18_HUMAN | BRE1B_HUMAN |
| ARPC4_HUMAN | AZI1_HUMAN | BAP29_HUMAN | BRE_HUMAN |
| ARPC5_HUMAN | AZI2_HUMAN | BAP31_HUMAN | BRK1_HUMAN |
| ARV1_HUMAN | AZIN1_HUMAN | BARD1_HUMAN | BROX_HUMAN |
| ASB13_HUMAN | B1AK87_HUMAN | BASI_HUMAN | BRWD3_HUMAN |
| ASCC2_HUMAN | B1ALK7_HUMAN | BASP1_HUMAN | BSDC1_HUMAN |
| ASNA_HUMAN | B2CI53_HUMAN | BAX_HUMAN | BT2A1_HUMAN |
| ASNS_HUMAN | B2L12_HUMAN | BAZ1A_HUMAN | BT3L4_HUMAN |
| ASPP1_HUMAN | B2L13_HUMAN | BAZ1B_HUMAN | BTBD1_HUMAN |
| ASPP2_HUMAN | B2RDE1_HUMAN | BAZ2A_HUMAN | BTBD2_HUMAN |
| ASXL2_HUMAN | B3A2_HUMAN | BBS1_HUMAN | BTBDA_HUMAN |
| AT11C_HUMAN | B3KNS4_HUMAN | BBS2_HUMAN | BTF3_HUMAN |
| BUB1B_HUMAN | CBR3_HUMAN | CDC16_HUMAN | CFDP1_HUMAN |
| BUB1_HUMAN | CBS_HUMAN | CDC20_HUMAN | CG044_HUMAN |
| BUB3_HUMAN | CBWD1_HUMAN | CDC23_HUMAN | CG074_HUMAN |
| BYST_HUMAN | CBX1_HUMAN | CDC27_HUMAN | CGL_HUMAN |
| BZW1_HUMAN | CBX2_HUMAN | CDC37_HUMAN | CH055_HUMAN |
| BZW2_HUMAN | CBX3_HUMAN | CDC42_HUMAN | CH059_HUMAN |
| C19L1_HUMAN | CBX5_HUMAN | CDC45_HUMAN | CH10_HUMAN |
| C1QBP_HUMAN | CBX6_HUMAN | CDC5L_HUMAN | CH60_HUMAN |
| C1TC_HUMAN | CC037_HUMAN | CDC73_HUMAN | CHC10_HUMAN |
| C8AP2_HUMAN | CC038_HUMAN | CDC7_HUMAN | CHCH2_HUMAN |
| C99L2_HUMAN | CC075_HUMAN | CDIPT_HUMAN | CHCH3_HUMAN |
| CA031_HUMAN | CC104_HUMAN | CDK1_HUMAN | CHD1_HUMAN |
| CA043_HUMAN | CC138_HUMAN | CDK2_HUMAN | CHD4_HUMAN |
| CA052_HUMAN | CC167_HUMAN | CDK4_HUMAN | CHD8_HUMAN |
| CA055_HUMAN | CC85B_HUMAN | CDK5_HUMAN | CHIC1_HUMAN |
| CA124_HUMAN | CCD14_HUMAN | CDKAL_HUMAN | CHIC2_HUMAN |
| CAB39_HUMAN | CCD22_HUMAN | CDV3_HUMAN | CHK1_HUMAN |
| CAB45_HUMAN | CCD47_HUMAN | CDYL1_HUMAN | CHM1A_HUMAN |
| CACO2_HUMAN | CCD50_HUMAN | CE025_HUMAN | CHM1B_HUMAN |
| CADH2_HUMAN | CCD58_HUMAN | CE170_HUMAN | CHM2A_HUMAN |
| CADM1_HUMAN | CCD72_HUMAN | CE192_HUMAN | CHM2B_HUMAN |
| CAF1A_HUMAN | CCD86_HUMAN | CE290_HUMAN | CHM4B_HUMAN |
| CAF1B_HUMAN | CCD94_HUMAN | CEBPZ_HUMAN | CHMP5_HUMAN |
| CAH2_HUMAN | CCD97_HUMAN | CEGT_HUMAN | CHRD1_HUMAN |
| CAH8_HUMAN | CCDB1_HUMAN | CELF1_HUMAN | CHSP1_HUMAN |
| CALD1_HUMAN | CCDC6_HUMAN | CENPB_HUMAN | CHTOP_HUMAN |
| CALM_HUMAN | CCDC8_HUMAN | CENPF_HUMAN | CI040_HUMAN |
| CALU_HUMAN | CCNA2_HUMAN | CENPH_HUMAN | CI041_HUMAN |
| CALX_HUMAN | CCNB1_HUMAN | CENPL_HUMAN | CI064_HUMAN |
| CAN1_HUMAN | CCNB2_HUMAN | CENPN_HUMAN | CI078_HUMAN |
| CAN7_HUMAN | CCND1_HUMAN | CENPQ_HUMAN | CIB1_HUMAN |
| CANB1_HUMAN | CCNK_HUMAN | CEP44_HUMAN | CING_HUMAN |
| CAND1_HUMAN | CCZ1L_HUMAN | CEP55_HUMAN | CIP2A_HUMAN |
| CAP1_HUMAN | CD032_HUMAN | CEP78_HUMAN | CIR1A_HUMAN |
| CAPR1_HUMAN | CD11A_HUMAN | CERS2_HUMAN | CISD1_HUMAN |
| CAPZB_HUMAN | CD123_HUMAN | CETN1_HUMAN | CISD2_HUMAN |
| CARM1_HUMAN | CD151_HUMAN | CETN2_HUMAN | CISY_HUMAN |
| CASC3_HUMAN | CD276_HUMAN | CF072_HUMAN | CJ032_HUMAN |
| CASC5_HUMAN | CD2AP_HUMAN | CF106_HUMAN | CK046_HUMAN |
| CAV1_HUMAN | CD320_HUMAN | CF115_HUMAN | CK067_HUMAN |
| CAZA1_HUMAN | CD81_HUMAN | CF130_HUMAN | CK5P2_HUMAN |
| CBPD_HUMAN | CD97_HUMAN | CF192_HUMAN | CK5P3_HUMAN |
| CBR1_HUMAN | CD99_HUMAN | CF211_HUMAN | CKAP2_HUMAN |
| CKAP5_HUMAN | COPG_HUMAN | CSN4_HUMAN | CYBP_HUMAN |
| CKS1_HUMAN | COPZ1_HUMAN | CSN5_HUMAN | CYBP_HUMAN |
| CL023_HUMAN | COQ2_HUMAN | CSN6_HUMAN | CYC_HUMAN |
| CL16A_HUMAN | COR1B_HUMAN | CSN7A_HUMAN | CYFP1_HUMAN |
| CLCA_HUMAN | COR1C_HUMAN | CSN7B_HUMAN | CYFP2_HUMAN |
| CLCB_HUMAN | COX17_HUMAN | CSPG5_HUMAN | CYLD_HUMAN |
| CLCC1_HUMAN | COX41_HUMAN | CSTF2_HUMAN | CYTB_HUMAN |
| CLH1_HUMAN | CP013_HUMAN | CSTF3_HUMAN | CYTSA_HUMAN |
| CLIC1_HUMAN | CP072_HUMAN | CSTFT_HUMAN | CYTSB_HUMAN |
| CLIC4_HUMAN | CP080_HUMAN | CT004_HUMAN | D3DQ69_HUMAN |
| CMIP_HUMAN | CP110_HUMAN | CT011_HUMAN | D3VVH3_HUMAN |
| CN166_HUMAN | CP135_HUMAN | CT030_HUMAN | D6RDG3_HUMAN |
| CN37_HUMAN | CP250_HUMAN | CTBP1_HUMAN | DACH1_HUMAN |
| CNBP_HUMAN | CP51A_HUMAN | CTBP2_HUMAN | DAD1_HUMAN |
| CND1_HUMAN | CPIN1_HUMAN | CTCF_HUMAN | DAG1_HUMAN |
| CND2_HUMAN | CPNE1_HUMAN | CTNA1_HUMAN | DAXX_HUMAN |
| CND3_HUMAN | CPNE3_HUMAN | CTNB1_HUMAN | DAZP1_HUMAN |
| CNDG2_HUMAN | CPNE5_HUMAN | CTND1_HUMAN | DBLOH_HUMAN |
| CNN3_HUMAN | CPNE8_HUMAN | CTR1_HUMAN | DBNL_HUMAN |
| CNNM3_HUMAN | CPNS1_HUMAN | CTR2_HUMAN | DBPA_HUMAN |
| CNNM4_HUMAN | CPSF1_HUMAN | CU059_HUMAN | DC1L1_HUMAN |
| CNOT1_HUMAN | CPSF2_HUMAN | CUED2_HUMAN | DC1L2_HUMAN |
| CNOT8_HUMAN | CPSF3_HUMAN | CUL1_HUMAN | DCA13_HUMAN |
| CNOTA_HUMAN | CPSF5_HUMAN | CUL2_HUMAN | DCAF5_HUMAN |
| CNO_HUMAN | CPSF6_HUMAN | CUL3_HUMAN | DCAF6_HUMAN |
| CO038_HUMAN | CPSF7_HUMAN | CUL4A_HUMAN | DCAF7_HUMAN |
| CO044_HUMAN | CPT1A_HUMAN | CUL4B_HUMAN | DCAF8_HUMAN |
| CO057_HUMAN | CR021_HUMAN | CUL5_HUMAN | DCAKD_HUMAN |
| COBL1_HUMAN | CREB5_HUMAN | CUL7_HUMAN | DCAM_HUMAN |
| COF1_HUMAN | CRIPT_HUMAN | CUL9_HUMAN | DCK_HUMAN |
| COF2_HUMAN | CRKL_HUMAN | CUTA_HUMAN | DCNL1_HUMAN |
| COG2_HUMAN | CRNL1_HUMAN | CUTC_HUMAN | DCNL5_HUMAN |
| COG4_HUMAN | CS010_HUMAN | CWC15_HUMAN | DCPS_HUMAN |
| COMD1_HUMAN | CS043_HUMAN | CWC22_HUMAN | DCTN1_HUMAN |
| COMD4_HUMAN | CSDE1_HUMAN | CWC27_HUMAN | DCTN2_HUMAN |
| COMD9_HUMAN | CSK21_HUMAN | CX026_HUMAN | DCTN4_HUMAN |
| COMT_HUMAN | CSK22_HUMAN | CX056_HUMAN | DCTP1_HUMAN |
| COPA_HUMAN | CSK2B_HUMAN | CX057_HUMAN | DCUP_HUMAN |
| COPB2_HUMAN | CSKP_HUMAN | CX6B1_HUMAN | DCXR_HUMAN |
| COPB_HUMAN | CSK_HUMAN | CX7A2_HUMAN | DD19A_HUMAN |
| COPD_HUMAN | CSN1_HUMAN | CXA1_HUMAN | DDB1_HUMAN |
| COPE_HUMAN | CSN2_HUMAN | CY561_HUMAN | DDB2_HUMAN |
| COPG2_HUMAN | CSN3_HUMAN | CYB5B_HUMAN | DDHD2_HUMAN |
| DDI1_HUMAN | DHX40_HUMAN | DRG1_HUMAN | EDRF1_HUMAN |
| DDI2_HUMAN | DHX57_HUMAN | DRG2_HUMAN | EEA1_HUMAN |
| DDIT4_HUMAN | DHX9_HUMAN | DRS7B_HUMAN | EF1A1_HUMAN |
| DDTL_HUMAN | DHYS_HUMAN | DSC3_HUMAN | EF1A2_HUMAN |
| DDX17_HUMAN | DIAP1_HUMAN | DSCR3_HUMAN | EF1B_HUMAN |
| DDX18_HUMAN | DICER_HUMAN | DSG2_HUMAN | EF1D_HUMAN |
| DDX1_HUMAN | DIDO1_HUMAN | DSRAD_HUMAN | EF1G_HUMAN |
| DDX20_HUMAN | DIM1_HUMAN | DTL_HUMAN | EF2K_HUMAN |
| DDX21_HUMAN | DIP2B_HUMAN | DUS3L_HUMAN | EF2_HUMAN |
| DDX23_HUMAN | DJC11_HUMAN | DUS3_HUMAN | EFHD1_HUMAN |
| DDX24_HUMAN | DJC21_HUMAN | DUT_HUMAN | EFNB1_HUMAN |
| DDX27_HUMAN | DKC1_HUMAN | DVL1L_HUMAN | EFTU_HUMAN |
| DDX3X_HUMAN | DLL1_HUMAN | DVL2_HUMAN | EHD4_HUMAN |
| DDX41_HUMAN | DLRB1_HUMAN | DX39A_HUMAN | EHMT1_HUMAN |
| DDX46_HUMAN | DMD_HUMAN | DX39B_HUMAN | EHMT2_HUMAN |
| DDX47_HUMAN | DMKN_HUMAN | DYH7_HUMAN | EI2BA_HUMAN |
| DDX59_HUMAN | DNA2L_HUMAN | DYHC1_HUMAN | EI2BB_HUMAN |
| DDX5_HUMAN | DNJA1_HUMAN | DYHC2_HUMAN | EI2BD_HUMAN |
| DDX6_HUMAN | DNJA2_HUMAN | DYL1_HUMAN | EID1_HUMAN |
| DEK_HUMAN | DNJB1_HUMAN | DYL2_HUMAN | EIF1A_HUMAN |
| DEN4C_HUMAN | DNJB2_HUMAN | DYLT1_HUMAN | EIF1_HUMAN |
| DENR_HUMAN | DNJB3_HUMAN | DYM_HUMAN | EIF3A_HUMAN |
| DEP1A_HUMAN | DNJB4_HUMAN | DYN1_HUMAN | EIF3B_HUMAN |
| DESM_HUMAN | DNJB6_HUMAN | DYN2_HUMAN | EIF3C_HUMAN |
| DESP_HUMAN | DNJC7_HUMAN | DYR_HUMAN | EIF3D_HUMAN |
| DEST_HUMAN | DNJC8_HUMAN | DZIP3_HUMAN | EIF3E_HUMAN |
| DFFA_HUMAN | DNJC9_HUMAN | E2AK2_HUMAN | EIF3F_HUMAN |
| DHAK_HUMAN | DNLI1_HUMAN | E41L2_HUMAN | EIF3G_HUMAN |
| DHB11_HUMAN | DNLI3_HUMAN | E41L5_HUMAN | EIF3H_HUMAN |
| DHB12_HUMAN | DNM1L_HUMAN | E7EW20_HUMAN | EIF3I_HUMAN |
| DHB4_HUMAN | DNMT1_HUMAN | E9PDP1_HUMAN | EIF3K_HUMAN |
| DHB7_HUMAN | DOCK7_HUMAN | E9PHA7_HUMAN | EIF3L_HUMAN |
| DHC24_HUMAN | DP13A_HUMAN | E9PIE5_HUMAN | EIF3M_HUMAN |
| DHCR7_HUMAN | DPM1_HUMAN | EAA1_HUMAN | ELAV1_HUMAN |
| DHRS1_HUMAN | DPOA2_HUMAN | EAPP_HUMAN | ELAV2_HUMAN |
| DHRS3_HUMAN | DPOD1_HUMAN | EBP2_HUMAN | ELMD2_HUMAN |
| DHRS4_HUMAN | DPOE1_HUMAN | ECH1_HUMAN | ELOB_HUMAN |
| DHRS7_HUMAN | DPOE2_HUMAN | ECHA_HUMAN | ELOC_HUMAN |
| DHSO_HUMAN | DPOE3_HUMAN | ECHM_HUMAN | ELP1_HUMAN |
| DHX15_HUMAN | DPOLA_HUMAN | ECM29_HUMAN | ELP2_HUMAN |
| DHX30_HUMAN | DPY30_HUMAN | EDC3_HUMAN | ELP3_HUMAN |
| DHX32_HUMAN | DPYL2_HUMAN | EDC4_HUMAN | EM55_HUMAN |
| DHX36_HUMAN | DREB_HUMAN | EDF1_HUMAN | EMAL3_HUMAN |
| EMAL4_HUMAN | EXOS9_HUMAN | FBX42_HUMAN | FWCH2_HUMAN |
| EMD_HUMAN | EZRI_HUMAN | FBXL3_HUMAN | FXR1_HUMAN |
| ENAH_HUMAN | F10A1_HUMAN | FBXL4_HUMAN | FYV1_HUMAN |
| ENOA_HUMAN | F115A_HUMAN | FCF1_HUMAN | FZD1_HUMAN |
| ENOPH_HUMAN | F120A_HUMAN | FCHO2_HUMAN | FZR_HUMAN |
| ENPLL_HUMAN | F125A_HUMAN | FCL_HUMAN | G2E3_HUMAN |
| ENPL_HUMAN | F127A_HUMAN | FDFT_HUMAN | G3BP1_HUMAN |
| ENSA_HUMAN | F127B_HUMAN | FEM1A_HUMAN | G3BP2_HUMAN |
| EP15R_HUMAN | F136A_HUMAN | FEM1B_HUMAN | G3P_HUMAN |
| EP400_HUMAN | F175B_HUMAN | FEN1_HUMAN | G6PI_HUMAN |
| EPCAM_HUMAN | F188A_HUMAN | FETUA_HUMAN | GA45A_HUMAN |
| EPHA2_HUMAN | F195B_HUMAN | FHL1_HUMAN | GAK_HUMAN |
| EPHA7_HUMAN | F208A_HUMAN | FHL3_HUMAN | GANAB_HUMAN |
| EPIPL_HUMAN | F263_HUMAN | FIBP_HUMAN | GAPD1_HUMAN |
| EPN1_HUMAN | F6XY72_HUMAN | FIP1_HUMAN | GAR1_HUMAN |
| EPN2_HUMAN | F8VZ13_HUMAN | FIS1_HUMAN | GASP2_HUMAN |
| EPN4_HUMAN | F92A1_HUMAN | FKB1A_HUMAN | GATL1_HUMAN |
| EPS15_HUMAN | FA40A_HUMAN | FKBP3_HUMAN | GBB1_HUMAN |
| ERBB4_HUMAN | FA49B_HUMAN | FKBP4_HUMAN | GBB2_HUMAN |
| ERC6L_HUMAN | FA50A_HUMAN | FKBP5_HUMAN | GBB4_HUMAN |
| ERCC2_HUMAN | FA54A_HUMAN | FKBP8_HUMAN | GBF1_HUMAN |
| ERCC3_HUMAN | FA54B_HUMAN | FL2D_HUMAN | GBG12_HUMAN |
| ERCC5_HUMAN | FA63A_HUMAN | FLII_HUMAN | GBG5_HUMAN |
| ERCC6_HUMAN | FA98A_HUMAN | FLNA_HUMAN | GBLP_HUMAN |
| ERF1_HUMAN | FABP5_HUMAN | FLNB_HUMAN | GBRAP_HUMAN |
| ERF3A_HUMAN | FACD2_HUMAN | FLOT1_HUMAN | GBRL2_HUMAN |
| ERG1_HUMAN | FACE1_HUMAN | FLOT2_HUMAN | GCC2_HUMAN |
| ERG7_HUMAN | FACR1_HUMAN | FLVC1_HUMAN | GCF_HUMAN |
| ERH_HUMAN | FADS2_HUMAN | FMR1_HUMAN | GCN1L_HUMAN |
| ERI3_HUMAN | FAF1_HUMAN | FNBP1_HUMAN | GCP2_HUMAN |
| ESPL1_HUMAN | FAF2_HUMAN | FOPNL_HUMAN | GCP4_HUMAN |
| ESTD_HUMAN | FAIM1_HUMAN | FOXC1_HUMAN | GCP60_HUMAN |
| ESYT1_HUMAN | FAKD1_HUMAN | FPPS_HUMAN | GDAP1_HUMAN |
| ETFA_HUMAN | FANCA_HUMAN | FRYL_HUMAN | GDAP2_HUMAN |
| ETUD1_HUMAN | FANCI_HUMAN | FTM_HUMAN | GDE_HUMAN |
| EWS_HUMAN | FANCJ_HUMAN | FTO_HUMAN | GDIA_HUMAN |
| EXD2_HUMAN | FAS_HUMAN | FUBP1_HUMAN | GDIB_HUMAN |
| EXOC1_HUMAN | FBRL_HUMAN | FUBP2_HUMAN | GDIR1_HUMAN |
| EXOC2_HUMAN | FBX21_HUMAN | FUBP3_HUMAN | GDPD1_HUMAN |
| EXOC4_HUMAN | FBX28_HUMAN | FUMH_HUMAN | GDS1_HUMAN |
| EXOS5_HUMAN | FBX32_HUMAN | FUND1_HUMAN | GEMI4_HUMAN |
| EXOS6_HUMAN | FBX38_HUMAN | FUND2_HUMAN | GEMI5_HUMAN |
| EXOS8_HUMAN | FBX3_HUMAN | FUS_HUMAN | GEMI6_HUMAN |
| GEMI_HUMAN | GORS2_HUMAN | H2B1A_HUMAN | HES1_HUMAN |
| GFPT1_HUMAN | GOSR1_HUMAN | H2B1B_HUMAN | HEXI1_HUMAN |
| GFRP_HUMAN | GOT1B_HUMAN | H2B1C_HUMAN | HGB1A_HUMAN |
| GGA1_HUMAN | GPAA1_HUMAN | H2B1D_HUMAN | HGS_HUMAN |
| GGA3_HUMAN | GPAT1_HUMAN | H2B1H_HUMAN | HIF1N_HUMAN |
| GGCT_HUMAN | GPHRA_HUMAN | H2B1J_HUMAN | HINT1_HUMAN |
| GGPPS_HUMAN | GPI8_HUMAN | H31T_HUMAN | HINT3_HUMAN |
| GIPC1_HUMAN | GPKOW_HUMAN | H33_HUMAN | HIP1_HUMAN |
| GKAP1_HUMAN | GPM6B_HUMAN | H4_HUMAN | HLTF_HUMAN |
| GLCNE_HUMAN | GPTC4_HUMAN | H90B2_HUMAN | HM13_HUMAN |
| GLMN_HUMAN | GPTC8_HUMAN | H90B3_HUMAN | HMCS1_HUMAN |
| GLNA_HUMAN | GRB2_HUMAN | HACD2_HUMAN | HMDH_HUMAN |
| GLO2_HUMAN | GRHL2_HUMAN | HACD3_HUMAN | HMG3M_HUMAN |
| GLOD4_HUMAN | GRHPR_HUMAN | HAP28_HUMAN | HMGB1_HUMAN |
| GLP3L_HUMAN | GRK6_HUMAN | HAT1_HUMAN | HMGB2_HUMAN |
| GLPK3_HUMAN | GRP75_HUMAN | HAUS1_HUMAN | HMGB3_HUMAN |
| GLPK5_HUMAN | GRP78_HUMAN | HAUS3_HUMAN | HMGN1_HUMAN |
| GLPK_HUMAN | GRSF1_HUMAN | HAUS5_HUMAN | HMGN2_HUMAN |
| GLRX3_HUMAN | GSHR_HUMAN | HAUS6_HUMAN | HMGN3_HUMAN |
| GLTP_HUMAN | GSK3A_HUMAN | HAUS7_HUMAN | HMGN4_HUMAN |
| GLYC_HUMAN | GSTA4_HUMAN | HAUS8_HUMAN | HMGN5_HUMAN |
| GLYR1_HUMAN | GSTM3_HUMAN | HAX1_HUMAN | HMOX2_HUMAN |
| GMFB_HUMAN | GSTO1_HUMAN | HBS1L_HUMAN | HN1_HUMAN |
| GMPPB_HUMAN | GSTP1_HUMAN | HCD2_HUMAN | HNRCL_HUMAN |
| GNA11_HUMAN | GTF2I_HUMAN | HCFC1_HUMAN | HNRDL_HUMAN |
| GNA13_HUMAN | GTPB1_HUMAN | HDAC1_HUMAN | HNRH1_HUMAN |
| GNA1_HUMAN | GTR1_HUMAN | HDAC2_HUMAN | HNRH2_HUMAN |
| GNAI1_HUMAN | GUAA_HUMAN | HDDC2_HUMAN | HNRH3_HUMAN |
| GNAI3_HUMAN | GWL_HUMAN | HDGF_HUMAN | HNRL1_HUMAN |
| GNAL_HUMAN | GYS1_HUMAN | HDGR2_HUMAN | HNRL2_HUMAN |
| GNAQ_HUMAN | H11_HUMAN | HD_HUMAN | HNRLL_HUMAN |
| GNAS1_HUMAN | H12_HUMAN | HEAT1_HUMAN | HNRPC_HUMAN |
| GNAS2_HUMAN | H1X_HUMAN | HEAT2_HUMAN | HNRPD_HUMAN |
| GNAZ_HUMAN | H2A1A_HUMAN | HEAT3_HUMAN | HNRPF_HUMAN |
| GNL3_HUMAN | H2A1B_HUMAN | HECD1_HUMAN | HNRPG_HUMAN |
| GNPAT_HUMAN | H2A1D_HUMAN | HECD3_HUMAN | HNRPK_HUMAN |
| GNPI1_HUMAN | H2A2B_HUMAN | HELC1_HUMAN | HNRPL_HUMAN |
| GOGA5_HUMAN | H2A2C_HUMAN | HELLS_HUMAN | HNRPM_HUMAN |
| GOGA7_HUMAN | H2AV_HUMAN | HEM3_HUMAN | HNRPQ_HUMAN |
| GOGB1_HUMAN | H2AW_HUMAN | HERC1_HUMAN | HNRPR_HUMAN |
| GOLI_HUMAN | H2AX_HUMAN | HERC2_HUMAN | HNRPU_HUMAN |
| GOLP3_HUMAN | H2AY_HUMAN | HERC3_HUMAN | HOIL1_HUMAN |
| GOPC_HUMAN | H2AZ_HUMAN | HERC5_HUMAN | HOOK1_HUMAN |
| HPBP1_HUMAN | IF2P_HUMAN | IQGA2_HUMAN | KAP0_HUMAN |
| HPRT_HUMAN | IF4A1_HUMAN | IQGA3_HUMAN | KAP2_HUMAN |
| HPS3_HUMAN | IF4A2_HUMAN | IR3IP_HUMAN | KAPCA_HUMAN |
| HS105_HUMAN | IF4A3_HUMAN | IRAK1_HUMAN | KAT5_HUMAN |
| HS71L_HUMAN | IF4B_HUMAN | IREB2_HUMAN | KBRS2_HUMAN |
| HS74L_HUMAN | IF4E2_HUMAN | IRF3_HUMAN | KC1A_HUMAN |
| HS902_HUMAN | IF4E_HUMAN | IRS4_HUMAN | KC1D_HUMAN |
| HS904_HUMAN | IF4G1_HUMAN | ISOC2_HUMAN | KC1G1_HUMAN |
| HS905_HUMAN | IF4G2_HUMAN | IST1_HUMAN | KC1G3_HUMAN |
| HS90A_HUMAN | IF4H_HUMAN | ITB1_HUMAN | KCC2B_HUMAN |
| HS90B_HUMAN | IF5A1_HUMAN | ITCH_HUMAN | KCC2D_HUMAN |
| HSBP1_HUMAN | IF5_HUMAN | ITFG3_HUMAN | KCMF1_HUMAN |
| HSDL1_HUMAN | IFT27_HUMAN | ITM2B_HUMAN | KCRB_HUMAN |
| HSF2_HUMAN | IFT43_HUMAN | ITM2C_HUMAN | KCT2_HUMAN |
| HSP71_HUMAN | IGBP1_HUMAN | ITPA_HUMAN | KCTD3_HUMAN |
| HSP72_HUMAN | IKKB_HUMAN | ITPR2_HUMAN | KCTD5_HUMAN |
| HSP74_HUMAN | ILF2_HUMAN | ITPR3_HUMAN | KCTD9_HUMAN |
| HSP7C_HUMAN | ILF3_HUMAN | ITSN1_HUMAN | KDIS_HUMAN |
| HSPB1_HUMAN | ILKAP_HUMAN | ITSN2_HUMAN | KDM1A_HUMAN |
| HTAI2_HUMAN | ILK_HUMAN | IWS1_HUMAN | KDM3A_HUMAN |
| HTR5A_HUMAN | ILVBL_HUMAN | JAK1_HUMAN | KDM3B_HUMAN |
| HTSF1_HUMAN | IMA2_HUMAN | JAM1_HUMAN | KDM4A_HUMAN |
| HUWE1_HUMAN | IMA3_HUMAN | JIP4_HUMAN | KDM4B_HUMAN |
| HXB9_HUMAN | IMB1_HUMAN | JMJD6_HUMAN | KDM5C_HUMAN |
| HXK1_HUMAN | IMDH1_HUMAN | JOS1_HUMAN | KDM6A_HUMAN |
| HXK2_HUMAN | IMDH2_HUMAN | JUN_HUMAN | KEAP1_HUMAN |
| HYOU1_HUMAN | IMMT_HUMAN | K0090_HUMAN | KHDR1_HUMAN |
| I2BP1_HUMAN | IMPCT_HUMAN | K0195_HUMAN | KHNYN_HUMAN |
| I2BP2_HUMAN | INAR1_HUMAN | K0664_HUMAN | KI20A_HUMAN |
| ICAL_HUMAN | INGR1_HUMAN | K0889_HUMAN | KI67_HUMAN |
| ICLN_HUMAN | INO1_HUMAN | K1328_HUMAN | KIF11_HUMAN |
| ID4_HUMAN | INT3_HUMAN | K1797_HUMAN | KIF14_HUMAN |
| IDE_HUMAN | INT7_HUMAN | K1967_HUMAN | KIF1A_HUMAN |
| IDHC_HUMAN | IPO11_HUMAN | K1C18_HUMAN | KIF1B_HUMAN |
| IDI1_HUMAN | IPO4_HUMAN | K1C19_HUMAN | KIF22_HUMAN |
| IF1AX_HUMAN | IPO5_HUMAN | K2C8_HUMAN | KIF23_HUMAN |
| IF2A_HUMAN | IPO7_HUMAN | K6PF_HUMAN | KIF2A_HUMAN |
| IF2B1_HUMAN | IPO8_HUMAN | K6PL_HUMAN | KIF2C_HUMAN |
| IF2B2_HUMAN | IPO9_HUMAN | K6PP_HUMAN | KIF4A_HUMAN |
| IF2B3_HUMAN | IPYR2_HUMAN | KAD1_HUMAN | KIF5A_HUMAN |
| IF2B_HUMAN | IPYR_HUMAN | KAD2_HUMAN | KIF7_HUMAN |
| IF2GL_HUMAN | IQCB1_HUMAN | KAD6_HUMAN | KIFC1_HUMAN |
| IF2G_HUMAN | IQGA1_HUMAN | KAISO_HUMAN | KIN17_HUMAN |
| KINH_HUMAN | LIMS1_HUMAN | LZTL1_HUMAN | MD1L1_HUMAN |
| KIRR1_HUMAN | LIN7C_HUMAN | LZTR1_HUMAN | MD2L1_HUMAN |
| KLC1_HUMAN | LIPA1_HUMAN | M1IP1_HUMAN | MD2L2_HUMAN |
| KLH11_HUMAN | LIS1_HUMAN | M89BB_HUMAN | MDC1_HUMAN |
| KLH13_HUMAN | LITFL_HUMAN | MA7D1_HUMAN | MDHC_HUMAN |
| KLH15_HUMAN | LKHA4_HUMAN | MA7D3_HUMAN | MDHM_HUMAN |
| KLHL7_HUMAN | LLPH_HUMAN | MACOI_HUMAN | MDM2_HUMAN |
| KLHL9_HUMAN | LLR1_HUMAN | MAGD1_HUMAN | MDN1_HUMAN |
| KNTC1_HUMAN | LMAN1_HUMAN | MAGD2_HUMAN | MED10_HUMAN |
| KPCD_HUMAN | LMBD1_HUMAN | MAGD4_HUMAN | MED1_HUMAN |
| KPCI_HUMAN | LMBD2_HUMAN | MAGE1_HUMAN | MED22_HUMAN |
| KPRA_HUMAN | LMBL3_HUMAN | MALD2_HUMAN | MED25_HUMAN |
| KPRB_HUMAN | LMCD1_HUMAN | MAP1B_HUMAN | MED29_HUMAN |
| KPYM_HUMAN | LMNA_HUMAN | MAP4_HUMAN | MED4_HUMAN |
| KT3K_HUMAN | LMNB1_HUMAN | MARCS_HUMAN | MEIS1_HUMAN |
| KTN1_HUMAN | LMNB2_HUMAN | MARE1_HUMAN | MEIS2_HUMAN |
| KTNA1_HUMAN | LN28B_HUMAN | MARH5_HUMAN | MELK_HUMAN |
| L2GL1_HUMAN | LNP_HUMAN | MARH6_HUMAN | MERL_HUMAN |
| L2GL2_HUMAN | LPP3_HUMAN | MARK3_HUMAN | MERTK_HUMAN |
| LAMC1_HUMAN | LPPRC_HUMAN | MAT1_HUMAN | MET7A_HUMAN |
| LANC1_HUMAN | LRBA_HUMAN | MAT2B_HUMAN | METH_HUMAN |
| LANC2_HUMAN | LRC20_HUMAN | MATR3_HUMAN | METK2_HUMAN |
| LAP2A_HUMAN | LRC40_HUMAN | MAZ_HUMAN | MET_HUMAN |
| LAP2B_HUMAN | LRC41_HUMAN | MBB1A_HUMAN | MFA3L_HUMAN |
| LAP4A_HUMAN | LRC47_HUMAN | MBD3_HUMAN | MFAP1_HUMAN |
| LAR4B_HUMAN | LRC57_HUMAN | MBIP1_HUMAN | MFF_HUMAN |
| LARP1_HUMAN | LRC58_HUMAN | MBLC2_HUMAN | MFN1_HUMAN |
| LARP4_HUMAN | LRC59_HUMAN | MBNL1_HUMAN | MFN2_HUMAN |
| LAS1L_HUMAN | LRRC3_HUMAN | MBRL_HUMAN | MFSD1_HUMAN |
| LAT1_HUMAN | LRSM1_HUMAN | MCA3_HUMAN | MGAP_HUMAN |
| LAT3_HUMAN | LS14B_HUMAN | MCAF1_HUMAN | MGN2_HUMAN |
| LAT4_HUMAN | LSM12_HUMAN | MCES_HUMAN | MGRN1_HUMAN |
| LA_HUMAN | LSM4_HUMAN | MCL1_HUMAN | MIA3_HUMAN |
| LBR_HUMAN | LSM7_HUMAN | MCM10_HUMAN | MIA40_HUMAN |
| LC7L2_HUMAN | LSR_HUMAN | MCM2_HUMAN | MIB1_HUMAN |
| LC7L3_HUMAN | LST8_HUMAN | MCM3_HUMAN | MIB2_HUMAN |
| LCHN_HUMAN | LTOR1_HUMAN | MCM4_HUMAN | MICA3_HUMAN |
| LDHA_HUMAN | LTV1_HUMAN | MCM5_HUMAN | MID49_HUMAN |
| LDHB_HUMAN | LYN_HUMAN | MCM6_HUMAN | MIF_HUMAN |
| LEG8_HUMAN | LYPA1_HUMAN | MCM7_HUMAN | MIMIT_HUMAN |
| LEO1_HUMAN | LYPA2_HUMAN | MCM8_HUMAN | MINA_HUMAN |
| LGUL_HUMAN | LYPL1_HUMAN | MCMBP_HUMAN | MINT_HUMAN |
| LIFR_HUMAN | LYRIC_HUMAN | MCRS1_HUMAN | MIO_HUMAN |
| MIRO1_HUMAN | MRP_HUMAN | NAA15_HUMAN | NELFA_HUMAN |
| MIRO2_HUMAN | MRT4_HUMAN | NAA16_HUMAN | NEMF_HUMAN |
| MK01_HUMAN | MS18A_HUMAN | NAA25_HUMAN | NEMO_HUMAN |
| MK03_HUMAN | MSH2_HUMAN | NAA40_HUMAN | NEP1_HUMAN |
| MK14_HUMAN | MSH6_HUMAN | NAA50_HUMAN | NEUA_HUMAN |
| MK67I_HUMAN | MTA1_HUMAN | NACAD_HUMAN | NEUL4_HUMAN |
| MKLN1_HUMAN | MTA2_HUMAN | NACA_HUMAN | NEUL_HUMAN |
| MKRN1_HUMAN | MTAP_HUMAN | NACC1_HUMAN | NFIP1_HUMAN |
| MKRN2_HUMAN | MTBP_HUMAN | NADAP_HUMAN | NFIP2_HUMAN |
| MLL1_HUMAN | MTCH2_HUMAN | NAMPT_HUMAN | NFL_HUMAN |
| MLL2_HUMAN | MTFR1_HUMAN | NASP_HUMAN | NFX1_HUMAN |
| MMGT1_HUMAN | MTL13_HUMAN | NAT10_HUMAN | NFXL1_HUMAN |
| MMS19_HUMAN | MTL14_HUMAN | NB5R1_HUMAN | NFYC_HUMAN |
| MMS22_HUMAN | MTMR3_HUMAN | NB5R3_HUMAN | NGLY1_HUMAN |
| MMTA2_HUMAN | MTMR6_HUMAN | NBN_HUMAN | NH2L1_HUMAN |
| MO4L1_HUMAN | MTMR8_HUMAN | NBR1_HUMAN | NHP2_HUMAN |
| MO4L2_HUMAN | MTMR9_HUMAN | NC2A_HUMAN | NIBL1_HUMAN |
| MOB1A_HUMAN | MTOR_HUMAN | NCBP1_HUMAN | NIP7_HUMAN |
| MOC2A_HUMAN | MTPN_HUMAN | NCDN_HUMAN | NIPA_HUMAN |
| MOC2B_HUMAN | MTR1_HUMAN | NCKP1_HUMAN | NIPBL_HUMAN |
| MOES_HUMAN | MTRR_HUMAN | NCOAT_HUMAN | NISCH_HUMAN |
| MOFA1_HUMAN | MTX1_HUMAN | NDC1_HUMAN | NIT2_HUMAN |
| MON2_HUMAN | MTX2_HUMAN | NDK3_HUMAN | NKAPL_HUMAN |
| MORC3_HUMAN | MTX3_HUMAN | NDK8_HUMAN | NKAP_HUMAN |
| MORC4_HUMAN | MUL1_HUMAN | NDKA_HUMAN | NKRF_HUMAN |
| MOSC1_HUMAN | MXRA7_HUMAN | NDKB_HUMAN | NLTP_HUMAN |
| MOSC2_HUMAN | MYCBP_HUMAN | NDRG1_HUMAN | NMD3_HUMAN |
| MOT10_HUMAN | MYCBP_HUMAN | NDUA1_HUMAN | NMNA1_HUMAN |
| MOT1_HUMAN | MYC_HUMAN | NDUA4_HUMAN | NMT1_HUMAN |
| MOV10_HUMAN | MYH10_HUMAN | NDUA5_HUMAN | NOB1_HUMAN |
| MP2K1_HUMAN | MYH11_HUMAN | NDUA6_HUMAN | NOC2L_HUMAN |
| MP2K3_HUMAN | MYH9_HUMAN | NDUAD_HUMAN | NOL11_HUMAN |
| MP2K6_HUMAN | MYL6B_HUMAN | NDUA9_HUMAN | NOL9_HUMAN |
| MPCP_HUMAN | MYL6_HUMAN | NDUAD_HUMAN | NOLC1_HUMAN |
| MPI_HUMAN | MYO19_HUMAN | NDUB6_HUMAN | NOMO1_HUMAN |
| MPP6_HUMAN | MYO1B_HUMAN | NDUB8_HUMAN | NOMO2_HUMAN |
| MPRIP_HUMAN | MYO1C_HUMAN | NDUBA_HUMAN | NONO_HUMAN |
| MPRI_HUMAN | MYO1D_HUMAN | NDUC2_HUMAN | NOP56_HUMAN |
| MPZL1_HUMAN | MYO6_HUMAN | NDUS5_HUMAN | NOP58_HUMAN |
| MR1L1_HUMAN | MYPT1_HUMAN | NECP1_HUMAN | NOSIP_HUMAN |
| MRE11_HUMAN | MYSM1_HUMAN | NEDD8_HUMAN | NOTC3_HUMAN |
| MRP1_HUMAN | MZT1_HUMAN | NEK2_HUMAN | NP1L1_HUMAN |
| MRP4_HUMAN | NAA10_HUMAN | NEK9_HUMAN | NP1L4_HUMAN |
| NPA1P_HUMAN | NUP62_HUMAN | P66B_HUMAN | PDCD5_HUMAN |
| NPDC1_HUMAN | NUP85_HUMAN | P73_HUMAN | PDCL3_HUMAN |
| NPL4_HUMAN | NUP93_HUMAN | PA1B2_HUMAN | PDE12_HUMAN |
| NPM_HUMAN | NUP98_HUMAN | PA2G4_HUMAN | PDIA1_HUMAN |
| NPRL3_HUMAN | NVL_HUMAN | PAAF1_HUMAN | PDIA3_HUMAN |
| NRDC_HUMAN | NXT1_HUMAN | PABP1_HUMAN | PDIP3_HUMAN |
| NRP1_HUMAN | NYNRI_HUMAN | PABP2_HUMAN | PDK1L_HUMAN |
| NSD1_HUMAN | OBSL1_HUMAN | PABP4_HUMAN | PDLI1_HUMAN |
| NSD2_HUMAN | OCAD1_HUMAN | PACE1_HUMAN | PDLI5_HUMAN |
| NSDHL_HUMAN | OCLN_HUMAN | PACN3_HUMAN | PDPK1_HUMAN |
| NSE4A_HUMAN | OCRL_HUMAN | PAF1_HUMAN | PDRG1_HUMAN |
| NSF1C_HUMAN | ODFP2_HUMAN | PAF_HUMAN | PDS5A_HUMAN |
| NSF_HUMAN | ODPB_HUMAN | PAG16_HUMAN | PDXD1_HUMAN |
| NSMA3_HUMAN | OFD1_HUMAN | PAIP2_HUMAN | PDZ11_HUMAN |
| NSUN2_HUMAN | OGFD1_HUMAN | PAIRB_HUMAN | PEA15_HUMAN |
| NSUN5_HUMAN | OGFR_HUMAN | PALM_HUMAN | PEBP1_HUMAN |
| NT5D1_HUMAN | OGT1_HUMAN | PAMM_HUMAN | PEG10_HUMAN |
| NTCP4_HUMAN | OLA1_HUMAN | PANK3_HUMAN | PELO_HUMAN |
| NTF2_HUMAN | OPTN_HUMAN | PANX1_HUMAN | PEPD_HUMAN |
| NTM1A_HUMAN | ORC2_HUMAN | PAPOA_HUMAN | PERI_HUMAN |
| NTPCR_HUMAN | ORC5_HUMAN | PAPS1_HUMAN | PERQ2_HUMAN |
| NU107_HUMAN | ORN_HUMAN | PAPS2_HUMAN | PESC_HUMAN |
| NU133_HUMAN | OSB10_HUMAN | PAR12_HUMAN | PEX13_HUMAN |
| NU153_HUMAN | OSBL3_HUMAN | PAR1_HUMAN | PEX19_HUMAN |
| NU155_HUMAN | OSBL9_HUMAN | PARG_HUMAN | PEX3_HUMAN |
| NU160_HUMAN | OSGEP_HUMAN | PARK7_HUMAN | PEX5_HUMAN |
| NU188_HUMAN | OST48_HUMAN | PARP1_HUMAN | PFD2_HUMAN |
| NU205_HUMAN | OSTC_HUMAN | PAWR_HUMAN | PFD3_HUMAN |
| NUB1_HUMAN | OSTM1_HUMAN | PB1_HUMAN | PFDS_HUMAN |
| NUCKS_HUMAN | OTU1_HUMAN | PBX2_HUMAN | PFD6_HUMAN |
| NUCL_HUMAN | OTU6B_HUMAN | PCBP1_HUMAN | PGAM1_HUMAN |
| NUD19_HUMAN | OTUB1_HUMAN | PCBP2_HUMAN | PGAM5_HUMAN |
| NUDC1_HUMAN | OTUD5_HUMAN | PCGF6_HUMAN | PGES2_HUMAN |
| NUDC2_HUMAN | OXA1L_HUMAN | PCH2_HUMAN | PGK1_HUMAN |
| NUDC_HUMAN | OXR1_HUMAN | PCID2_HUMAN | PGM1_HUMAN |
| NUDT5_HUMAN | P121A_HUMAN | PCM1_HUMAN | PGM2_HUMAN |
| NUF2_HUMAN | P20D2_HUMAN | PCNA_HUMAN | PGP_HUMAN |
| NUFP2_HUMAN | P3C2A_HUMAN | PCNP_HUMAN | PGRC1_HUMAN |
| NUMA1_HUMAN | P3C2B_HUMAN | PCNT_HUMAN | PGRC2_HUMAN |
| NUP37_HUMAN | P4K2A_HUMAN | PCX3_HUMAN | PGTB2_HUMAN |
| NUP50_HUMAN | P4K2B_HUMAN | PDC10_HUMAN | PHB2_HUMAN |
| NUP53_HUMAN | P4R3A_HUMAN | PDC6I_HUMAN | PHB_HUMAN |
| NUP54_HUMAN | P53_HUMAN | PDCD4_HUMAN | PHC2_HUMAN |
| PHF10_HUMAN | PLST_HUMAN | PPME1_HUMAN | PRS8_HUMAN |
| PHF14_HUMAN | PLXA1_HUMAN | PPP5_HUMAN | PSA1_HUMAN |
| PHF5A_HUMAN | PLXA2_HUMAN | PPT1_HUMAN | PSA2_HUMAN |
| PHF6_HUMAN | PLXB2_HUMAN | PPWD1_HUMAN | PSA3_HUMAN |
| PHIP_HUMAN | PM14_HUMAN | PR38A_HUMAN | PSA4_HUMAN |
| PHLP_HUMAN | PMF1_HUMAN | PR38B_HUMAN | PSA5_HUMAN |
| PHP14_HUMAN | PMGE_HUMAN | PR40A_HUMAN | PSA6_HUMAN |
| PI42A_HUMAN | PML_HUMAN | PRAF1_HUMAN | PSA7L_HUMAN |
| PI42C_HUMAN | PMVK_HUMAN | PRAF3_HUMAN | PSA7_HUMAN |
| PI4KA_HUMAN | PNKP_HUMAN | PRAME_HUMAN | PSA_HUMAN |
| PI51A_HUMAN | PNMA1_HUMAN | PRC1_HUMAN | PSB1_HUMAN |
| PI51C_HUMAN | PNMA2_HUMAN | PRC2A_HUMAN | PSB2_HUMAN |
| PIAS1_HUMAN | PNML1_HUMAN | PRC2C_HUMAN | PSB3_HUMAN |
| PIBF1_HUMAN | PNO1_HUMAN | PRCC_HUMAN | PSB4_HUMAN |
| PICAL_HUMAN | PNPH_HUMAN | PRDX1_HUMAN | PSB5_HUMAN |
| PIGU_HUMAN | PO2F1_HUMAN | PRDX2_HUMAN | PSB7_HUMAN |
| PIMT_HUMAN | POGK_HUMAN | PRDX3_HUMAN | PSD10_HUMAN |
| PIN1_HUMAN | POLH_HUMAN | PRDX4_HUMAN | PSD11_HUMAN |
| PIN4_HUMAN | POLI_HUMAN | PRDX5_HUMAN | PSD12_HUMAN |
| PININ_HUMAN | POLK_HUMAN | PRDX6_HUMAN | PSD13_HUMAN |
| PIPNA_HUMAN | POMP_HUMAN | PREB_HUMAN | PSD7_HUMAN |
| PIPNB_HUMAN | POP1_HUMAN | PRI1_HUMAN | PSDE_HUMAN |
| PIPSL_HUMAN | POP7_HUMAN | PRI2_HUMAN | PSF1_HUMAN |
| PJA1_HUMAN | PP1A_HUMAN | PRKDC_HUMAN | PSIP1_HUMAN |
| PJA2_HUMAN | PP1G_HUMAN | PRKN2_HUMAN | PSMD1_HUMAN |
| PK3CA_HUMAN | PP1RA_HUMAN | PROF1_HUMAN | PSMD2_HUMAN |
| PKHA1_HUMAN | PP2AA_HUMAN | PROF2_HUMAN | PSMD3_HUMAN |
| PKHA7_HUMAN | PP2AB_HUMAN | PROSC_HUMAN | PSMD4_HUMAN |
| PKHH3_HUMAN | PP4C_HUMAN | PRP16_HUMAN | PSMD6_HUMAN |
| PKN1_HUMAN | PP4R2_HUMAN | PRP19_HUMAN | PSMD8_HUMAN |
| PKN2_HUMAN | PP6R3_HUMAN | PRP31_HUMAN | PSMD9_HUMAN |
| PKNX1_HUMAN | PPAC_HUMAN | PRP4_HUMAN | PSME1_HUMAN |
| PKP4_HUMAN | PPCEL_HUMAN | PRP6_HUMAN | PSME2_HUMAN |
| PLAK_HUMAN | PPCE_HUMAN | PRP8_HUMAN | PSME3_HUMAN |
| PLAP_HUMAN | PPDPF_HUMAN | PRPF3_HUMAN | PSMG1_HUMAN |
| PLCE_HUMAN | PPIA_HUMAN | PRPS1_HUMAN | PSMG2_HUMAN |
| PLCG1_HUMAN | PPIB_HUMAN | PRPS2_HUMAN | PSMG3_HUMAN |
| PLD3_HUMAN | PPID_HUMAN | PRR11_HUMAN | PTBP1_HUMAN |
| PLEC_HUMAN | PPIG_HUMAN | PRS10_HUMAN | PTBP2_HUMAN |
| PLIN3_HUMAN | PPIH_HUMAN | PRS4_HUMAN | PTH2_HUMAN |
| PLK1_HUMAN | PPIL4_HUMAN | PRS6A_HUMAN | PTK7_HUMAN |
| PLRG1_HUMAN | PPM1B_HUMAN | PRS6B_HUMAN | PTMA_HUMAN |
| PLSL_HUMAN | PPM1G_HUMAN | PRS7_HUMAN | PTMS_HUMAN |
| PTN11_HUMAN | QRIC1_HUMAN | RB39A_HUMAN | RER1_HUMAN |
| PTN23_HUMAN | QTRD1_HUMAN | RB3GP_HUMAN | RERE_HUMAN |
| PTN2_HUMAN | R13AX_HUMAN | RB6I2_HUMAN | RFA1_HUMAN |
| PTOV1_HUMAN | RA1L2_HUMAN | RBBP4_HUMAN | RFA2_HUMAN |
| PTPRA_HUMAN | RA51C_HUMAN | RBBP5_HUMAN | RFA3_HUMAN |
| PTPRF_HUMAN | RAB10_HUMAN | RBBP6_HUMAN | RFC2_HUMAN |
| PTPRG_HUMAN | RAB13_HUMAN | RBBP7_HUMAN | RFC3_HUMAN |
| PTPS_HUMAN | RAB14_HUMAN | RBGPR_HUMAN | RFC4_HUMAN |
| PTRF_HUMAN | RAB1A_HUMAN | RBM12_HUMAN | RFC5_HUMAN |
| PTSS1_HUMAN | RAB21_HUMAN | RBM14_HUMAN | RFIP1_HUMAN |
| PTTG1_HUMAN | RAB24_HUMAN | RBM15_HUMAN | RFWD3_HUMAN |
| PTTG_HUMAN | RAB2A_HUMAN | RBM22_HUMAN | RGAP1_HUMAN |
| PUF60_HUMAN | RAB34_HUMAN | RBM23_HUMAN | RHBD2_HUMAN |
| PUM1_HUMAN | RAB35_HUMAN | RBM26_HUMAN | RHBT3_HUMAN |
| PUR2_HUMAN | RAB3B_HUMAN | RBM27_HUMAN | RHEB_HUMAN |
| PUR6_HUMAN | RAB5A_HUMAN | RBM28_HUMAN | RHG05_HUMAN |
| PUR8_HUMAN | RAB5B_HUMAN | RBM39_HUMAN | RHG22_HUMAN |
| PUR9_HUMAN | RAB5C_HUMAN | RBM42_HUMAN | RHOA_HUMAN |
| PURA2_HUMAN | RAB7A_HUMAN | RBM4B_HUMAN | RHOU_HUMAN |
| PUS7_HUMAN | RAB8A_HUMAN | RBM4_HUMAN | RIF1_HUMAN |
| PVRL2_HUMAN | RABE1_HUMAN | RBMS1_HUMAN | RIFK_HUMAN |
| PVRL3_HUMAN | RABE2_HUMAN | RBP2_HUMAN | RING2_HUMAN |
| PWP1_HUMAN | RABP2_HUMAN | RBP56_HUMAN | RINI_HUMAN |
| PWP2_HUMAN | RABX5_HUMAN | RBX1_HUMAN | RIOK1_HUMAN |
| PYGB_HUMAN | RAC1_HUMAN | RB_HUMAN | RIOK2_HUMAN |
| PYGL_HUMAN | RAD18_HUMAN | RCC1_HUMAN | RIOK3_HUMAN |
| PYR1_HUMAN | RAD1_HUMAN | RCC2_HUMAN | RIR1_HUMAN |
| PYRG1_HUMAN | RAD21_HUMAN | RCCD1_HUMAN | RIR2B_HUMAN |
| Q13384_HUMAN | RAD50_HUMAN | RCD1_HUMAN | RIR2_HUMAN |
| Q59GX9_HUMAN | RADI_HUMAN | RCL1_HUMAN | RL10A_HUMAN |
| Q5FWY2_HUMAN | RAE1L_HUMAN | RCN1_HUMAN | RL10L_HUMAN |
| Q5JWE8_HUMAN | RAGP1_HUMAN | RCN2_HUMAN | RL10_HUMAN |
| Q5LJA5_HUMAN | RAI14_HUMAN | RD23A_HUMAN | RL11_HUMAN |
| Q6FG99_HUMAN | RALYL_HUMAN | RD23B_HUMAN | RL12_HUMAN |
| Q6IPH7_HUMAN | RALY_HUMAN | RDH11_HUMAN | RL13A_HUMAN |
| Q6IQ27_HUMAN | RANG_HUMAN | RDH14_HUMAN | RL13_HUMAN |
| Q7Z5V0_HUMAN | RAN_HUMAN | RECQ1_HUMAN | RL14_HUMAN |
| Q8NDP0_HUMAN | RAP1A_HUMAN | RED_HUMAN | RL15_HUMAN |
| Q9HBI2_HUMAN | RAP2B_HUMAN | REEP4_HUMAN | RL17_HUMAN |
| Q9ULW9_HUMAN | RASK_HUMAN | REEP5_HUMAN | RL18A_HUMAN |
| QCR2_HUMAN | RASN_HUMAN | REN3B_HUMAN | RL18_HUMAN |
| QCR9_HUMAN | RB11A_HUMAN | RENT1_HUMAN | RL19_HUMAN |
| QKI_HUMAN | RB11B_HUMAN | REPI1_HUMAN | RL1D1_HUMAN |
| RL21_HUMAN | RN122_HUMAN | RPC2_HUMAN | RS2_HUMAN |
| RL22_HUMAN | RN123_HUMAN | RPC4_HUMAN | RS30_HUMAN |
| RL23A_HUMAN | RN138_HUMAN | RPF1_HUMAN | RS3A_HUMAN |
| RL23_HUMAN | RN141_HUMAN | RPIA_HUMAN | RS3_HUMAN |
| RL24_HUMAN | RN146_HUMAN | RPN1_HUMAN | RS4X_HUMAN |
| RL26L_HUMAN | RN166_HUMAN | RPN2_HUMAN | RS5_HUMAN |
| RL27A_HUMAN | RN167_HUMAN | RPP29_HUMAN | RS6_HUMAN |
| RL27_HUMAN | RN168_HUMAN | RPP30_HUMAN | RS7_HUMAN |
| RL28_HUMAN | RN185_HUMAN | RPR1B_HUMAN | RS8_HUMAN |
| RL29_HUMAN | RN187_HUMAN | RPRD2_HUMAN | RS9_HUMAN |
| RL30_HUMAN | RN213_HUMAN | RRAGA_HUMAN | RSBNL_HUMAN |
| RL31_HUMAN | RN216_HUMAN | RRAGC_HUMAN | RSCA1_HUMAN |
| RL32_HUMAN | RN219_HUMAN | RRBP1_HUMAN | RSF1_HUMAN |
| RL34_HUMAN | RN220_HUMAN | RRMJ1_HUMAN | RSMB_HUMAN |
| RL35A_HUMAN | RNBP6_HUMAN | RRMJ3_HUMAN | RSPRY_HUMAN |
| RL35_HUMAN | RNF10_HUMAN | RRP12_HUMAN | RSRC2_HUMAN |
| RL36A_HUMAN | RNF12_HUMAN | RRP1B_HUMAN | RSSA_HUMAN |
| RL36_HUMAN | RNF13_HUMAN | RRP1_HUMAN | RSU1_HUMAN |
| RL37A_HUMAN | RNF25_HUMAN | RRP44_HUMAN | RT06_HUMAN |
| RL37_HUMAN | RNF31_HUMAN | RRP5_HUMAN | RT21_HUMAN |
| RL38_HUMAN | RNF4_HUMAN | RRS1_HUMAN | RT27_HUMAN |
| RL3L_HUMAN | RNF5_HUMAN | RS10L_HUMAN | RTC1_HUMAN |
| RL3_HUMAN | RNH2A_HUMAN | RS10_HUMAN | RTCB_HUMAN |
| RL40_HUMAN | RNPS1_HUMAN | RS11_HUMAN | RTF1_HUMAN |
| RL4_HUMAN | RNZ2_HUMAN | RS12_HUMAN | RTN3_HUMAN |
| RL5_HUMAN | RO60_HUMAN | RS13_HUMAN | RTN4_HUMAN |
| RL6_HUMAN | ROA0_HUMAN | RS14_HUMAN | RU17_HUMAN |
| RL7A_HUMAN | ROA1_HUMAN | RS15A_HUMAN | RU1C_HUMAN |
| RL7L_HUMAN | ROA2_HUMAN | RS15_HUMAN | RU2A_HUMAN |
| RL7_HUMAN | ROA3_HUMAN | RS16_HUMAN | RU2B_HUMAN |
| RL8_HUMAN | ROAA_HUMAN | RS17L_HUMAN | RUFY1_HUMAN |
| RL9_HUMAN | ROBO1_HUMAN | RS18_HUMAN | RUVB1_HUMAN |
| RLA0L_HUMAN | RPA1_HUMAN | RS19_HUMAN | RUVB2_HUMAN |
| RLA0_HUMAN | RPA49_HUMAN | RS20_HUMAN | RUXE_HUMAN |
| RLA1_HUMAN | RPAB1_HUMAN | RS21_HUMAN | RUXF_HUMAN |
| RLA2_HUMAN | RPAB5_HUMAN | RS23_HUMAN | RUXG_HUMAN |
| RM12_HUMAN | RPAP2_HUMAN | RS24_HUMAN | RWDD1_HUMAN |
| RM20_HUMAN | RPB11_HUMAN | RS25_HUMAN | RXRB_HUMAN |
| RM43_HUMAN | RPB1_HUMAN | RS26L_HUMAN | RYBP_HUMAN |
| RM53_HUMAN | RPB2_HUMAN | RS26_HUMAN | S10AA_HUMAN |
| RMD2_HUMAN | RPB7_HUMAN | RS27A_HUMAN | S10AB_HUMAN |
| RMD3_HUMAN | RPC10_HUMAN | RS28_HUMAN | S12A2_HUMAN |
| RN114_HUMAN | RPC1_HUMAN | RS29_HUMAN | S12A4_HUMAN |
| S12A6_HUMAN | SAS10_HUMAN | SETB1_HUMAN | SLK_HUMAN |
| S12A7_HUMAN | SAT1_HUMAN | SETD7_HUMAN | SLN11_HUMAN |
| S14L1_HUMAN | SATT_HUMAN | SET_HUMAN | SLU7_HUMAN |
| S15A4_HUMAN | SBDS_HUMAN | SF01_HUMAN | SMAD3_HUMAN |
| S18L2_HUMAN | SC11A_HUMAN | SF3A1_HUMAN | SMAD4_HUMAN |
| S19A1_HUMAN | SC11C_HUMAN | SF3A3_HUMAN | SMAP_HUMAN |
| S20A1_HUMAN | SC22B_HUMAN | SF3B1_HUMAN | SMC1A_HUMAN |
| S20A2_HUMAN | SC23A_HUMAN | SF3B2_HUMAN | SMC2_HUMAN |
| S22A5_HUMAN | SC23B_HUMAN | SF3B3_HUMAN | SMC3_HUMAN |
| S23A2_HUMAN | SC24C_HUMAN | SF3B5_HUMAN | SMC4_HUMAN |
| S23IP_HUMAN | SC31A_HUMAN | SFPQ_HUMAN | SMC6_HUMAN |
| S2546_HUMAN | SC5A3_HUMAN | SFR15_HUMAN | SMCA1_HUMAN |
| S2611_HUMAN | SC5D_HUMAN | SFR19_HUMAN | SMCA2_HUMAN |
| S26A6_HUMAN | SC6A8_HUMAN | SFSWA_HUMAN | SMCA4_HUMAN |
| S27A2_HUMAN | SCAFB_HUMAN | SFT2C_HUMAN | SMCA5_HUMAN |
| S29A1_HUMAN | SCAM1_HUMAN | SFXN1_HUMAN | SMCE1_HUMAN |
| S29A2_HUMAN | SCAM3_HUMAN | SGPL1_HUMAN | SMD1_HUMAN |
| S30BP_HUMAN | SCFD1_HUMAN | SGT1_HUMAN | SMD2_HUMAN |
| S35B2_HUMAN | SCLY_HUMAN | SGTA_HUMAN | SMD3_HUMAN |
| S35E1_HUMAN | SCML2_HUMAN | SGTB_HUMAN | SMG1_HUMAN |
| S38A1_HUMAN | SCO2_HUMAN | SH3G1_HUMAN | SMG8_HUMAN |
| S38A2_HUMAN | SCOC_HUMAN | SH3L1_HUMAN | SMHD1_HUMAN |
| S38A9_HUMAN | SCPDL_HUMAN | SH3L2_HUMAN | SMN_HUMAN |
| S39A6_HUMAN | SCRIB_HUMAN | SHIP1_HUMAN | SMOX_HUMAN |
| S39AA_HUMAN | SDC2_HUMAN | SHIP2_HUMAN | SMRC1_HUMAN |
| S39AE_HUMAN | SDCB1_HUMAN | SHKB1_HUMAN | SMRC2_HUMAN |
| S4A7_HUMAN | SDCG3_HUMAN | SHLB2_HUMAN | SMRCD_HUMAN |
| S61A1_HUMAN | SDSL_HUMAN | SHOT1_HUMAN | SMRD1_HUMAN |
| S6A15_HUMAN | SEC20_HUMAN | SHPK_HUMAN | SMU1_HUMAN |
| SAAL1_HUMAN | SEC62_HUMAN | SHPRH_HUMAN | SNAA_HUMAN |
| SAE1_HUMAN | SEC63_HUMAN | SHQ1_HUMAN | SNAG_HUMAN |
| SAE2_HUMAN | SEH1_HUMAN | SHRPN_HUMAN | SND1_HUMAN |
| SAFB1_HUMAN | SELR1_HUMAN | SIAS_HUMAN | SNF5_HUMAN |
| SAHH2_HUMAN | SENP3_HUMAN | SIN3A_HUMAN | SNF8_HUMAN |
| SAHH_HUMAN | SEP11_HUMAN | SIRT1_HUMAN | SNP23_HUMAN |
| SALL2_HUMAN | SEPT2_HUMAN | SIRT2_HUMAN | SNP29_HUMAN |
| SAM50_HUMAN | SEPT6_HUMAN | SIVA_HUMAN | SNP47_HUMAN |
| SAMH1_HUMAN | SEPT7_HUMAN | SK2L2_HUMAN | SNR40_HUMAN |
| SAP18_HUMAN | SEPT9_HUMAN | SKA2L_HUMAN | SNR48_HUMAN |
| SAR1A_HUMAN | SERA_HUMAN | SKIV2_HUMAN | SNRPA_HUMAN |
| SARM1_HUMAN | SERC1_HUMAN | SKI_HUMAN | SNTB2_HUMAN |
| SARNP_HUMAN | SERC_HUMAN | SKP1_HUMAN | SNUT1_HUMAN |
| SART3_HUMAN | SESN1_HUMAN | SKP2_HUMAN | SNW1_HUMAN |
| SNX12_HUMAN | SRPK2_HUMAN | STRUM_HUMAN | SYRC_HUMAN |
| SNX1_HUMAN | SRPRB_HUMAN | STT3A_HUMAN | SYSC_HUMAN |
| SNX27_HUMAN | SRRM1_HUMAN | STX10_HUMAN | SYTC_HUMAN |
| SNX2_HUMAN | SRRM2_HUMAN | STX12_HUMAN | SYVC_HUMAN |
| SNX32_HUMAN | SRRT_HUMAN | STX16_HUMAN | SYWC_HUMAN |
| SNX5_HUMAN | SRR_HUMAN | STX17_HUMAN | SYYC_HUMAN |
| SNX6_HUMAN | SRS11_HUMAN | STX18_HUMAN | T106B_HUMAN |
| SNX8_HUMAN | SRSF1_HUMAN | STX4_HUMAN | T22D3_HUMAN |
| SO4A1_HUMAN | SRSF2_HUMAN | STX5_HUMAN | T2AG_HUMAN |
| SOAT1_HUMAN | SRSF3_HUMAN | STX6_HUMAN | T2EB_HUMAN |
| SODC_HUMAN | SRSF4_HUMAN | STX7_HUMAN | T2FB_HUMAN |
| SON_HUMAN | SRSF5_HUMAN | STX8_HUMAN | T2H2L_HUMAN |
| SORCN_HUMAN | SRSF6_HUMAN | STXB1_HUMAN | TAB2_HUMAN |
| SP16H_HUMAN | SRSF7_HUMAN | STXB2_HUMAN | TACC3_HUMAN |
| SPA5L_HUMAN | SRSF9_HUMAN | STXB3_HUMAN | TADBP_HUMAN |
| SPAG7_HUMAN | SSA27_HUMAN | SUFU_HUMAN | TAF10_HUMAN |
| SPB6_HUMAN | SSBP_HUMAN | SUGT1_HUMAN | TAF1B_HUMAN |
| SPC24_HUMAN | SSF1_HUMAN | SUMO1_HUMAN | TAF5L_HUMAN |
| SPDLY_HUMAN | SSNA1_HUMAN | SUMO2_HUMAN | TAF7_HUMAN |
| SPEE_HUMAN | SSPN_HUMAN | SUMO3_HUMAN | TAF9B_HUMAN |
| SPF30_HUMAN | SSRA_HUMAN | SUN1_HUMAN | TAF9_HUMAN |
| SPF45_HUMAN | SSRD_HUMAN | SURF4_HUMAN | TAGL2_HUMAN |
| SPG20_HUMAN | SSRG_HUMAN | SUV91_HUMAN | TALDO_HUMAN |
| SPIT2_HUMAN | SSRP1_HUMAN | SUV92_HUMAN | TANC2_HUMAN |
| SPOP_HUMAN | SSU72_HUMAN | SUZ12_HUMAN | TAP2_HUMAN |
| SPRY7_HUMAN | ST1A1_HUMAN | SYAC_HUMAN | TARB1_HUMAN |
| SPSY_HUMAN | STABP_HUMAN | SYAP1_HUMAN | TATD1_HUMAN |
| SPT5H_HUMAN | STAG1_HUMAN | SYCC_HUMAN | TAXB1_HUMAN |
| SPT6H_HUMAN | STAG2_HUMAN | SYDC_HUMAN | TB10A_HUMAN |
| SPTA2_HUMAN | STAM1_HUMAN | SYEP_HUMAN | TB10B_HUMAN |
| SPTB2_HUMAN | STAM2_HUMAN | SYF1_HUMAN | TBA1A_HUMAN |
| SPTC1_HUMAN | STAT2_HUMAN | SYFA_HUMAN | TBA1B_HUMAN |
| SPTCS_HUMAN | STAT3_HUMAN | SYFB_HUMAN | TBA1C_HUMAN |
| SQSTM_HUMAN | STAU1_HUMAN | SYG_HUMAN | TBB2A_HUMAN |
| SR140_HUMAN | STEA3_HUMAN | SYHC_HUMAN | TBB3_HUMAN |
| SRC8_HUMAN | STIP1_HUMAN | SYIC_HUMAN | TBB4A_HUMAN |
| SREK1_HUMAN | STML2_HUMAN | SYJ2B_HUMAN | TBB4B_HUMAN |
| SRP09_HUMAN | STMN1_HUMAN | SYK_HUMAN | TBB5_HUMAN |
| SRP14_HUMAN | STPAP_HUMAN | SYLC_HUMAN | TBB6_HUMAN |
| SRP54_HUMAN | STRAP_HUMAN | SYMC_HUMAN | TBC15_HUMAN |
| SRP68_HUMAN | STRBP_HUMAN | SYMPK_HUMAN | TBC17_HUMAN |
| SRP72_HUMAN | STRN3_HUMAN | SYNC_HUMAN | TBCA_HUMAN |
| SRPK1_HUMAN | STRN4_HUMAN | SYQ_HUMAN | TBCB_HUMAN |
| TBCD4_HUMAN | TFG_HUMAN | TM7S3_HUMAN | TPC12_HUMAN |
| TBCD_HUMAN | TFR1_HUMAN | TM87A_HUMAN | TPD52_HUMAN |
| TBCE_HUMAN | TGFR1_HUMAN | TM9S3_HUMAN | TPD53_HUMAN |
| TBG1_HUMAN | TGS1_HUMAN | TM9S4_HUMAN | TPD54_HUMAN |
| TBL1R_HUMAN | THIC_HUMAN | TMCC1_HUMAN | TPIS_HUMAN |
| TBL2_HUMAN | THIO_HUMAN | TMCO1_HUMAN | TPM1_HUMAN |
| TBL3_HUMAN | THOC2_HUMAN | TMCO7_HUMAN | TPM4_HUMAN |
| TBP_HUMAN | THOC3_HUMAN | TMED4_HUMAN | TPP2_HUMAN |
| TCAL1_HUMAN | THOC4_HUMAN | TMED9_HUMAN | TPPC1_HUMAN |
| TCAL4_HUMAN | THOC6_HUMAN | TMEDA_HUMAN | TPPC3_HUMAN |
| TCAL8_HUMAN | THOP1_HUMAN | TMM31_HUMAN | TPPC4_HUMAN |
| TCEA1_HUMAN | THTM_HUMAN | TMM59_HUMAN | TPPC5_HUMAN |
| TCOF_HUMAN | THTPA_HUMAN | TMM66_HUMAN | TPPC8_HUMAN |
| TCP4_HUMAN | THUM3_HUMAN | TMOD3_HUMAN | TPR_HUMAN |
| TCPA_HUMAN | TIAR_HUMAN | TMUB1_HUMAN | TPX2_HUMAN |
| TCPB_HUMAN | TIF1A_HUMAN | TMUB2_HUMAN | TR10B_HUMAN |
| TCPD_HUMAN | TIF1B_HUMAN | TMX1_HUMAN | TR10D_HUMAN |
| TCPE_HUMAN | TIFA_HUMAN | TMX2_HUMAN | TR150_HUMAN |
| TCPG_HUMAN | TIGAR_HUMAN | TNKS1_HUMAN | TRA2A_HUMAN |
| TCPH_HUMAN | TIM10_HUMAN | TNKS2_HUMAN | TRA2B_HUMAN |
| TCPQ_HUMAN | TIM13_HUMAN | TNPO1_HUMAN | TRABD_HUMAN |
| TCPW_HUMAN | TIM50_HUMAN | TNPO2_HUMAN | TRAD1_HUMAN |
| TCPZ_HUMAN | TIM8A_HUMAN | TNPO3_HUMAN | TRAF2_HUMAN |
| TCRG1_HUMAN | TIM8B_HUMAN | TNR6_HUMAN | TRAF4_HUMAN |
| TCTP_HUMAN | TIM9_HUMAN | TOIP1_HUMAN | TRAF7_HUMAN |
| TDIF2_HUMAN | TIM_HUMAN | TOLIP_HUMAN | TRAP1_HUMAN |
| TDRKH_HUMAN | TIPIN_HUMAN | TOM1_HUMAN | TRI11_HUMAN |
| TE2IP_HUMAN | TIPRL_HUMAN | TOM20_HUMAN | TRI18_HUMAN |
| TEAN2_HUMAN | TITIN_HUMAN | TOM22_HUMAN | TRI25_HUMAN |
| TEBP_HUMAN | TKT_HUMAN | TOM34_HUMAN | TRI26_HUMAN |
| TECR_HUMAN | TLE1_HUMAN | TOM40_HUMAN | TRI27_HUMAN |
| TECT3_HUMAN | TLE3_HUMAN | TOM70_HUMAN | TRI32_HUMAN |
| TELO2_HUMAN | TLK2_HUMAN | TOM7_HUMAN | TRI33_HUMAN |
| TERA_HUMAN | TLN1_HUMAN | TOP1_HUMAN | TRI44_HUMAN |
| TES_HUMAN | TM115_HUMAN | TOP2A_HUMAN | TRI56_HUMAN |
| TF2B_HUMAN | TM165_HUMAN | TOP2B_HUMAN | TRI65_HUMAN |
| TF2H3_HUMAN | TM192_HUMAN | TOPB1_HUMAN | TRIM1_HUMAN |
| TF2H5_HUMAN | TM1L1_HUMAN | TOPK_HUMAN | TRIM4_HUMAN |
| TF3C1_HUMAN | TM1L2_HUMAN | TP4A1_HUMAN | TRIP4_HUMAN |
| TF3C3_HUMAN | TM209_HUMAN | TP4A2_HUMAN | TRIPB_HUMAN |
| TF3C4_HUMAN | TM237_HUMAN | TP4AP_HUMAN | TRIPC_HUMAN |
| TF3C5_HUMAN | TM41B_HUMAN | TPC10_HUMAN | TRM1L_HUMAN |
| TFDP1_HUMAN | TM45A_HUMAN | TPC11_HUMAN | TRM1_HUMAN |
| TRM6_HUMAN | UB2D3_HUMAN | UBP25_HUMAN | UTP18_HUMAN |
| TRRAP_HUMAN | UB2E1_HUMAN | UBP28_HUMAN | UTP23_HUMAN |
| TRUA_HUMAN | UB2G2_HUMAN | UBP2L_HUMAN | UTP6_HUMAN |
| TRXR1_HUMAN | UB2L3_HUMAN | UBP30_HUMAN | UTRO_HUMAN |
| TS101_HUMAN | UB2Q1_HUMAN | UBP33_HUMAN | UXT_HUMAN |
| TSC2_HUMAN | UB2R1_HUMAN | UBP34_HUMAN | VA0D1_HUMAN |
| TSN10_HUMAN | UB2R2_HUMAN | UBP36_HUMAN | VAMP1_HUMAN |
| TSNAX_HUMAN | UB2V1_HUMAN | UBP3_HUMAN | VAMP2_HUMAN |
| TSN_HUMAN | UB2V2_HUMAN | UBP48_HUMAN | VAMP4_HUMAN |
| TSR3_HUMAN | UBA1_HUMAN | UBP5_HUMAN | VAMP7_HUMAN |
| TSYL1_HUMAN | UBA3_HUMAN | UBP7_HUMAN | VAMP8_HUMAN |
| TSYL2_HUMAN | UBA6_HUMAN | UBQL1_HUMAN | VANG1_HUMAN |
| TTC12_HUMAN | UBAC1_HUMAN | UBQL2_HUMAN | VAPA_HUMAN |
| TTC26_HUMAN | UBAP1_HUMAN | UBR4_HUMAN | VAPB_HUMAN |
| TTC27_HUMAN | UBB_HUMAN | UBR5_HUMAN | VAS1_HUMAN |
| TTC32_HUMAN | UBC12_HUMAN | UBR7_HUMAN | VASP_HUMAN |
| TTC37_HUMAN | UBCP1_HUMAN | UBX2A_HUMAN | VAT1_HUMAN |
| TTC5_HUMAN | UBE2C_HUMAN | UBXN1_HUMAN | VATA_HUMAN |
| TTC9C_HUMAN | UBE2H_HUMAN | UBXN4_HUMAN | VATB2_HUMAN |
| TTF2_HUMAN | UBE2K_HUMAN | UBXN6_HUMAN | VATC1_HUMAN |
| TTK_HUMAN | UBE2N_HUMAN | UBXN7_HUMAN | VATF_HUMAN |
| TTL12_HUMAN | UBE2O_HUMAN | UBXN8_HUMAN | VATH_HUMAN |
| TULP3_HUMAN | UBE2S_HUMAN | UCHL1_HUMAN | VCIP1_HUMAN |
| TUT4_HUMAN | UBE2T_HUMAN | UCHL5_HUMAN | VDAC1_HUMAN |
| TX264_HUMAN | UBE3A_HUMAN | UCK2_HUMAN | VDAC2_HUMAN |
| TXD17_HUMAN | UBE3C_HUMAN | UCRIL_HUMAN | VDAC3_HUMAN |
| TXLNA_HUMAN | UBE4A_HUMAN | UEVLD_HUMAN | VIGLN_HUMAN |
| TXN4A_HUMAN | UBE4B_HUMAN | UFC1_HUMAN | VIME_HUMAN |
| TXN4B_HUMAN | UBF1_HUMAN | UFD1_HUMAN | VINC_HUMAN |
| TXND9_HUMAN | UBFD1_HUMAN | UHRF1_HUMAN | VIR_HUMAN |
| TXNIP_HUMAN | UBL4A_HUMAN | UIMC1_HUMAN | VP13A_HUMAN |
| TXNL1_HUMAN | UBL5_HUMAN | UK114_HUMAN | VP13C_HUMAN |
| TYDP2_HUMAN | UBL7_HUMAN | ULA1_HUMAN | VP13D_HUMAN |
| TYSY_HUMAN | UBP10_HUMAN | ULK3_HUMAN | VP26A_HUMAN |
| TYW1_HUMAN | UBP11_HUMAN | UMPS_HUMAN | VP33A_HUMAN |
| TYY1_HUMAN | UBP13_HUMAN | UN45A_HUMAN | VP33B_HUMAN |
| U2AF1_HUMAN | UBP14_HUMAN | UNC5C_HUMAN | VPP1_HUMAN |
| U2AF2_HUMAN | UBP16_HUMAN | UPK3L_HUMAN | VPP2_HUMAN |
| U520_HUMAN | UBP19_HUMAN | URB2_HUMAN | VPS16_HUMAN |
| U5S1_HUMAN | UBP1_HUMAN | USMG5_HUMAN | VPS29_HUMAN |
| UACA_HUMAN | UBP20_HUMAN | USO1_HUMAN | VPS35_HUMAN |
| UAP1_HUMAN | UBP22_HUMAN | USP9X_HUMAN | VPS36_HUMAN |
| UB2D1_HUMAN | UBP24_HUMAN | UTP15_HUMAN | VPS39_HUMAN |
| VPS45_HUMAN | XPO7_HUMAN | ZMAT2_HUMAN | ##PYGL_HUMAN |
| VPS4A_HUMAN | XPOT_HUMAN | ZMYM1_HUMAN | ##RL6_HUMAN |
| VPS4B_HUMAN | XPP1_HUMAN | ZMYM2_HUMAN | ##SMC1A_HUMAN |
| VRK1_HUMAN | XRCC1_HUMAN | ZMYM3_HUMAN | ##TCPQ_HUMAN |
| VRK3_HUMAN | XRCC4_HUMAN | ZN207_HUMAN | ##TITIN_HUMAN |
| VTA1_HUMAN | XRCC5_HUMAN | ZN264_HUMAN | ##TXND3_HUMAN |
| WAC_HUMAN | XRCC6_HUMAN | ZN281_HUMAN | |
| WAP53_HUMAN | XRN2_HUMAN | ZN326_HUMAN | |
| WASH1_HUMAN | XRP2_HUMAN | ZN330_HUMAN | |
| WBP11_HUMAN | YAF2_HUMAN | ZN346_HUMAN | |
| WBP2_HUMAN | YAP1_HUMAN | ZN451_HUMAN | |
| WBS22_HUMAN | YBOX1_HUMAN | ZN460_HUMAN | |
| WDHD1_HUMAN | YETS4_HUMAN | ZN503_HUMAN | |
| WDR11_HUMAN | YI017_HUMAN | ZN598_HUMAN | |
| WDR12_HUMAN | YIPF3_HUMAN | ZN622_HUMAN | |
| WDR1_HUMAN | YKT6_HUMAN | ZN638_HUMAN | |
| WDR26_HUMAN | YMEL1_HUMAN | ZN711_HUMAN | |
| WDR36_HUMAN | YTHD1_HUMAN | ZN768_HUMAN | |
| WDR41_HUMAN | YTHD2_HUMAN | ZNF24_HUMAN | |
| WDR43_HUMAN | Z280C_HUMAN | ZNT1_HUMAN | |
| WDR44_HUMAN | Z3H7A_HUMAN | ZO1_HUMAN | |
| WDR48_HUMAN | ZBT10_HUMAN | ZO2_HUMAN | |
| WDR59_HUMAN | ZC11A_HUMAN | ZPR1_HUMAN | |
| WDR61_HUMAN | ZC3HE_HUMAN | ZRAB2_HUMAN | |
| WDR67_HUMAN | ZC3HF_HUMAN | ZSWM6_HUMAN | |
| WDR6_HUMAN | ZCCHV_HUMAN | ZUFSP_HUMAN | |
| WDR74_HUMAN | ZCH10_HUMAN | ZW10_HUMAN | |
| WDR75_HUMAN | ZCH12_HUMAN | ZWILC_HUMAN | |
| WDR82_HUMAN | ZCHC2_HUMAN | ZWINT_HUMAN | |
| WDR85_HUMAN | ZCHC3_HUMAN | ZYX_HUMAN | |
| WDTC1_HUMAN | ZCHC8_HUMAN | ZZEF1_HUMAN | |
| WIZ_HUMAN | ZDH13_HUMAN | ##AHNK2_HUMAN | |
| WLS_HUMAN | ZEB1_HUMAN | ##AHNK_HUMAN | |
| WPB5_HUMAN | ZF106_HUMAN | ##BAP31_HUMAN | |
| WRB_HUMAN | ZF161_HUMAN | ##CENPF_HUMAN | |
| WRIP1_HUMAN | ZFAN5_HUMAN | ##CLH1_HUMAN | |
| WRP73_HUMAN | ZFAN6_HUMAN | ##CNTRL_HUMAN | |
| WWP1_HUMAN | ZFN2B_HUMAN | ##ENOA_HUMAN | |
| XIAP_HUMAN | ZFR_HUMAN | ##FAS_HUMAN | |
| XPC_HUMAN | ZFX_HUMAN | ##HUWE1_HUMAN | |
| XPO1_HUMAN | ZFY16_HUMAN | ##MCM7_HUMAN | |
| XPO2_HUMAN | ZFY19_HUMAN | ##NBN_HUMAN | |
| XPO5_HUMAN | ZKSC1_HUMAN | ##PRKDC_HUMAN | |

What is claimed is:

1. A method of increasing mitophagy in a cell comprising contacting the cell with an inhibitor of USP30.

2. A method of increasing mitochondrial ubiquitination in a cell comprising contacting the cell with an inhibitor of USP30.

3. A method of increasing ubiquitination of at least one, at least two, at least three, at least four, at least five, at least six, at least seven, at least eight, at least nine, at least ten, at least eleven, at least twelve, at least thirteen, or fourteen proteins selected from Tom20, MIRO, MUL1, ASNS, FKBP8, TOM70, MAT2B, PRDX3, IDE, VDAC1, VDAC2, VDAC3, IP05, PSD13, UBP13, and PTH2 in a cell comprising contacting the cell with an inhibitor of USP30.

4. The method of claim 3, wherein the method comprises increasing ubiquitination of at least one, at least two, or three amino acids selected from K56, K61, and K68 of Tom 20.

5. The method of claim 3, wherein the method comprises increasing ubiquitination of at least one, at least two, at least three, at least four, at least five, at least six, at least seven, or eight amino acids selected from K153, K187, K330, K427, K512, K535, K567, and K572 of MIRO.

6. The method of claim 3, wherein the method comprises increasing ubiquitination of at least one, at least two, or three amino acids selected from K273, K299, and K52 of MUL1.

7. The method of claim 3, wherein the method comprises increasing ubiquitination of at least one, at least two, at least three, at least four, at least five, at least six, at least seven, at least eight, or nine amino acids selected from K147, K168, K176, K221, K244, K275, K478, K504, and K556 of ASNS.

8. The method of claim 3, wherein the method comprises increasing ubiquitination of at least one, at least two, at least three, at least four, at least five, at least six, at least seven, or eight amino acids selected from K249, K271, K273, K284, K307, K317, K334, and K340 of FKBP8.

9. The method of claim 3, wherein the method comprises increasing ubiquitination of at least one, at least two, at least three, at least four, at least five, at least six, at least seven, at least eight, at least nine, or at least ten amino acids selected from K78, K120, K123, K126, K129, K148, K168, K170, K178, K185, K204, K230, K233, K245, K275, K278, K312, K326, K349, K359, K441, K463, K470, K471, K494, K501, K524, K536, K563, K570, K599, K600, and K604 of TOM70.

10. The method of claim 3, wherein the method comprises increasing ubiquitination of at least one, at least two, at least three, or four amino acids selected from K209, K245, K316, and K326 of MAT2B.

11. The method of claim 3, wherein the method comprises increasing ubiquitination of at least one, at least two, at least three, at least four, or five amino acids selected from K83, K91, K166, K241, and K253 of PRDX3.

12. The method of claim 3, wherein the method comprises increasing ubiquitination of at least one, at least two, at least three, at least four, at least five, or six amino acids selected from K558, K657, K854, K884, K929, and K933 of IDE.

13. The method of claim 3, wherein the method comprises increasing ubiquitination of at least one, at least two, at least three, at least four, at least five, at least six, or seven amino acids selected from K20, K53, K61, K109, K110, K266, and K274 of VDAC1.

14. The method of claim 3, wherein the method comprises increasing ubiquitination of at least one, at least two, at least three, at least four, at least five, or six amino acids selected from K31, K64, K120, K121, K277, and K285 of VDAC2.

15. The method of claim 3, wherein the method comprises increasing ubiquitination of at least one, at least two, at least three, at least four, at least five, at least six, at least seven, or eight amino acids selected from K20, K53, K61, K109, K110, K163, K266, and K274 of VDAC3.

16. The method of claim 3, wherein the method comprises increasing ubiquitination of at at least one, at least two, at least three, at least four, at least five, at least six, at least seven, at least eight, at least nine, or at least ten amino acids selected from K238, K353, K436, K437, K548, K556, K613, K678, K690, K705, K775, and K806 of IP05.

17. The method of claim 3, wherein the method comprises increasing ubiquitination of at least one, at least two, at least three, at least four, at least five, at least six, at least seven, at least eight, at least nine, or at least ten amino acids selected from K2, K32, K99, K115, K122, K132, K161, K186, K313, K321, K347, K350, and K361 of PSD13.

18. The method of claim 3, wherein the method comprises increasing ubiquitination of at least one, at least two, at least three, at least four, at least five, at least six, at least seven, at least eight, at least nine, or at least ten amino acids selected from K18, K190, K259, K326, K328, K401, K405, K414, K418, K435, K586, K587, and K640 of UBP13.

19. The method of claim 3, wherein the method comprises increasing ubiquitination of at least one, at least two, at least three, at least four, at least five, at least six, at least seven, at least eight, or nine amino acids selected from 47, 76, 81, 95, 106, 119, 134, 171, 177 of PTH2.

20. The method of claim 1, wherein the cell is under oxidative stress.

21. A method of reducing oxidative stress in a cell comprising contacting the cell with an inhibitor of USP30.

22. The method of claim 1, wherein the cell comprises a pathogenic mutation in Parkin, a pathogenic mutation in PINK1, or a pathogenic mutation in Parkin and a pathogenic mutation in PINK1.

23. The method of claim 22, wherein the cell comprises a pathogenic mutation in Parkin selected from the mutations in Table 1.

24. The method of claim 22, wherein the cell comprises a pathogenic mutation in PINK1 selected from the mutations in Table 2.

25. The method of claim 1, wherein the cell is selected from a neuron, a cardiac cell, and a muscle cell.

26. The method of claim 1, wherein the cell is comprised in a subject.

27. The method of claim 1, wherein the cell is ex vivo or in vitro.

28. A method of treating a condition involving a mitochondrial defect in a subject comprising administering to the subject an effective amount of an inhibitor of USP30.

29. The method of claim 28, wherein the condition involving a mitochondrial defect is selected from a condition involving a mitophagy defect, a condition involving a mutation in mitochondrial DNA, a condition involving mitochondrial oxidative stress, a condition involving a defect in mitochondrial shape or morphology, a condition involving a defect in mitochondrial membrane potential, and a condition involving a lysosomal storage defect.

30. The method of claim 28, wherein the condition involving a mitochondrial defect is selected from a neurodegenerative disease; mitochondrial myopathy, encephalopathy, lactic acidosis, and stroke-like episodes (MELAS) syndrome; Leber's hereditary optic neuropathy (LHON); neuropathy, ataxia, retinitis pigmentosa-maternally inherited Leigh syndrome (NARP-MILS); Danon disease; ischemic heart disease leading to myocardial infarction; multiple sulfatase deficiency (MSD); mucolipidosis II (ML II); mucolipidosis III (ML III); mucolipidosis IV (ML IV); GM1-gangliosidosis (GM1); neuronal ceroid-lipofuscinoses (NCL1); Alpers disease; Barth syndrome; Beta-oxidation defects; carnitine-acyl-carnitine deficiency; carnitine deficiency; creatine deficiency syndromes; co-enzyme Q10 deficiency; complex I deficiency; complex II deficiency; complex III deficiency; complex IV deficiency; complex V deficiency; COX deficiency; chronic progressive external ophthalmoplegia syndrome (CPEO); CPT I deficiency; CPT II deficiency; glutaric aciduria type II; Kearns-Sayre syndrome; lactic acidosis; long-chain acyl-CoA dehydrongenase deficiency (LCHAD); Leigh disease or syndrome; lethal infantile cardiomyopathy (LIC); Luft disease; glutaric aciduria type II; medium-chain acyl-CoA dehydrongenase deficiency (MCAD); myoclonic epilepsy and ragged-red fiber (MERRF) syndrome; mitochondrial recessive ataxia syndrome; mitochondrial cytopathy; mitochondrial DNA depletion syndrome; myoneurogastointestinal disorder and encephalopathy; Pearson syndrome; pyruvate carboxylase deficiency; pyruvate dehydrogenase deficiency; POLG mutations; medium/short-chain 3-hydroxyacyl-CoA dehydrogenase (M/SCHAD) deficiency; and very long-chain acyl-CoA dehydrongenase (VLCAD) deficiency.

31. The method of claim 30, wherein the neurodegenerative disease is selected from Alzheimer's disease, Parkinson's disease, amyotrophic lateral sclerosis (ALS), Huntington's disease, ischemia, stroke, dementia with Lewy bodies, and frontotemporal dementia.

32. A method of treating a neurodegenerative disease in a subject comprising administering to the subject an effective amount of an inhibitor of USP30.

33. The method of claim 32, wherein the neurodegenerative disease is selected from Parkinson's disease, Alzheimer's disease, Huntington's disease, amyotrophic lateral sclerosis (ALS), ischemia, stroke, dementia with Lewy bodies, and frontotemporal dementia.

34. A method of treating Parkinson's disease in a subject comprising administering to the subject an effective amount of an inhibitor of USP30.

35. The method of claim 28, wherein the subject comprises a pathogenic mutation in Parkin, a pathogenic mutation in PINK1, or a pathogenic mutation in Parkin and a pathogenic mutation in PINK1 in at least a portion of the subject's cells.

36. The method of claim 35, wherein the pathogenic mutation in Parkin is selected from the mutations in Table 1.

37. The method of claim 35, wherein the pathogenic mutation in PINK1 is selected from the mutations in Table 2.

38. A method of treating a condition involving cells undergoing oxidative stress in a subject comprising administering to the subject an effective amount of an inhibitor of USP30.

39. The method of claim 28, wherein the inhibitor of USP30 is administered orally, intramuscularly, intravenously, intraarterially, intraperitoneally, or subcutaneously.

40. The method of claim 28, wherein the method comprises administering at least one additional therapeutic agent.

41. The method of claim 40, wherein the at least one additional therapeutic agent is selected from levodopa, a dopamine agonist, a monoamino oxygenase (MAO) B inhibitor, a catechol O-methyltransferase (COMT) inhibitor, an anticholinergic, amantadine, riluzole, a cholinesterase inhibitor, memantine, tetrabenazine, an antipsychotic, clonazepam, diazepam, an antidepressant, and an anti-convulsant.

42. The method of claim 1, wherein the inhibitor of USP30 is an inhibitor of USP30 expression.

43. The method of claim 42, wherein the inhibitor of USP30 expression is selected from an antisense oligonucleotide and a short interfering RNA (siRNA).

44. The method of claim 1, wherein the inhibitor of USP30 is an inhibitor of USP30 activity.

45. The method of claim 44, wherein the inhibitor of USP30 activity is selected from an antibody, a peptide, a peptibody, an aptamer, and a small molecule.

46. A peptide comprising the amino acid sequence:

| | |
|---|---|
| X₁X₂CX₃X₄X₅X₆X₇X₈X₉X₁₀X₁₁CX₁₂ | (SEQ ID NO: 48) |

wherein:
X₁ is selected from L, M, A, S, and V;
X₂ is selected from Y, D, E, I, L, N, and S;
X₃ is selected from F, I, and Y;
X₄ is selected from F, I, and Y;
X₅ is selected from D and E;
X₆ is selected from L, M, V, and P;
X₇ is selected from S, N, D, A, and T;
X₈ is selected from Y, D, F, N, and W;
X₉ is selected from G, D, and E;
X₁₀ is selected from Y and F;
X₁ᵢ is selected from L, V, M, Q, and W; and
X₁₂ is selected from F, L, C, V, and Y;
wherein the peptide inhibits USP30 with an 1050 of less than 10 μM.

47. The peptide of claim 46, wherein the 1050 of the peptide for at least one, at least two, or at least three peptidases selected from USP7, USP5, UCHL3, and USP2 is greater than 20 μM, greater than 30 μM, greater than 40 μM, or greater than 50 μM.

48. The peptide of claim 46, wherein the peptide comprises an amino acid sequence that is at least 80%, at least 85%, at least 90%, at least 95%, or 100% identical to an amino acid sequence selected from SEQ ID NOs: 1 to 22.

49. An antisense oligonucleotide comprising a nucleotide sequence that is at least 80%, at least 85%, at least 90%, at least 95%, or 100% complementary to a region of USP30 mRNA and/or a region of USP30 pre-mRNA, wherein the region is at least at least 10, at least 15, at least 20, at least 25, at least 30, at least 40, at least 50, at least 60, at least 70, at least 80, at least 90, or at least 100 nucleotides long.

50. An siRNA comprising a nucleotide sequence that is at least 80%, at least 85%, at least 90%, at least 95%, or 100% identical to a region of USP30 mRNA and/or a region of USP30 pre-mRNA, wherein the region is at least at least 10, at least 15, at least 20, or at least 25 nucleotides long.


SEQUENCE LISTING (49 sequences)

SEQ ID NO: 1 - protein, 16 aa, Artificial sequence
  Synthetic
PLYCFYDLTYGYLCFY

SEQ ID NO: 2 - protein, 16 aa, Artificial sequence
  Synthetic
VSRCYIFWNEMFCDVE

SEQ ID NO: 3 - protein, 16 aa, Artificial sequence
  Synthetic
SLDCFFDLSYGYLCFD

SEQ ID NO: 4 - protein, 16 aa, Artificial sequence
  Synthetic
AMYCFYDMDYGYQCLY

SEQ ID NO: 5 - protein, 16 aa, Artificial sequence
  Synthetic
TLNCYYDLDYGYLCFH

SEQ ID NO: 6 - protein, 16 aa, Artificial sequence
  Synthetic
DLYCFYDLNDGYLCFS

SEQ ID NO: 7 - protein, 16 aa, Artificial sequence
  Synthetic
SLDCFFDLNYGYLCFD

SEQ ID NO: 8 - protein, 16 aa, Artificial sequence
  Synthetic
SLYCFYDLSYGYVCLY

SEQ ID NO: 9 - protein, 16 aa, Artificial sequence
  Synthetic
QSYCFYDVDWGYLCYH

SEQ ID NO: 10 - protein, 16 aa, Artificial sequence
  Synthetic
SMLCFYDVAYGYLCFD

SEQ ID NO: 11 - protein, 16 aa, Artificial sequence
  Synthetic
SAICFYDMSYGYVCFE

SEQ ID NO: 12 - protein, 16 aa, Artificial sequence
  Synthetic
TLYCFFDMSYGYWCVD

SEQ ID NO: 13 - protein, 16 aa, Artificial sequence
  Synthetic
TMYCFYDLSDDYLCFY

SEQ ID NO: 14 - protein, 16 aa, Artificial sequence
  Synthetic
ELYCFYDLANGYLCFD

SEQ ID NO: 15 - protein, 16 aa, Artificial sequence
  Synthetic
QLDCIYELNYGYLCFD

SEQ ID NO: 16 - protein, 16 aa, Artificial sequence
  Synthetic
SRDCFIDLNFGYLYCY

SEQ ID NO: 17 - protein, 16 aa, Artificial sequence
  Synthetic
ALDCFYDLNYGYLCFD

SEQ ID NO: 18 - protein, 16 aa, Artificial sequence
  Synthetic
ALDCIFEMSYGFMCFD

SEQ ID NO: 19 - protein, 16 aa, Artificial sequence
  Synthetic
ELSCFYDLSYGYLCFY

SEQ ID NO: 20 - protein, 16 aa, Artificial sequence
  Synthetic
ALYCYYDPNYGYLCFS

SEQ ID NO: 21 - protein, 16 aa, Artificial sequence
  Synthetic
RVYCFYDLTYEYLCFI

SEQ ID NO: 22 - protein, 16 aa, Artificial sequence
  Synthetic
SLECFYDLDYGYLCFE

SEQ ID NO: 23 - protein, 12 aa, Artificial sequence
  Synthetic
RAGLSKLPDLKD

SEQ ID NO: 24 - protein, 14 aa, Artificial sequence
  Synthetic
KLPDLKDAEAVQKF

SEQ ID NO: 25 - protein, 29 aa, Artificial sequence
  Synthetic
KDAEAVQKFFLEEIQLGEELLAQGEYEKG

SEQ ID NO: 26 - protein, 517 aa, Homo sapiens
MLSSRAEAAMTAADRAIQRFLRTGAAVRYKVMKNWGVIGGIAAALAAGIYVIWGPITERKKRRKGLVPGLVNLGNTCFMNSLLQGLSACPAFIRWLEEFTSQYSRDQKEPPSHQYLSLTLLHLLKALSCQEVTDDEVLDASCLLDVLRMYRWQISSFEEQDAHELFHVITSSLEDERDRQPRVTHLFDVHSLEQQSEITPKQITCRTRGSPHPTSNHWKSQHPFHGRLTSNMVCKHCEHQSPVRFDTFDSLSLSIPAATWGHPLTLDHCLHHFISSESVRDVVCDNCTKIEAKGTLNGEKVEHQRTTFVKQLKLGKLPQCLCIHLQRLSWSSHGTPLKRHEHVQFNEFLMMDIYKYHLLGHKPSQHNPKLNKNPGPTLELQDGPGAPTPVLNQPGAPKTQIFMNGACSPSLLPTLSAPMPFPLPVVPDYSSSTYLFRLMAVVVHHGDMHSGHFVTYRRSPPSARNPLSTSNQWLWVSDDTVRKASLQEVLSSSAYLLFYERVLSRMQHQSQECKSEE

SEQ ID NO: 27 - protein, 145 aa, Homo sapiens
MVGRNSAIAAGVCGALFIGYCIYFDRKRRSDPNFKNRLRERRKKQKLAKERAGLSKLPDLKDAEAVQKFFLEEIQLGEELLAQGEYEKGVDHLTNAIAVCGQPQQLLQVLQQTLPPPVFQMLLTKLPTISQRIVSAQSLAEDDVE

SEQ ID NO: 28 - protein, 618 aa, Homo sapiens
MKKDVRILLVGEPRVGKTSLIMSLVSEEFPEEVPPRAEEITIPADVTPERVPTHIVDYSEAEQSDEQLHQEISQANVICIVYAVNNKHSIDKVTSRWIPLINERTDKDSRLPLILVGNKSDLVEYSSMETILPIMNQYTEIETCVECSAKNLKNISELFYYAQKAVLHPTGPLYCPEEKEMKPACIKALTRIFKISDQDNDGTLNDAELNFFQRICFNTPLAPQALEDVKNVVRKHISDGVADSGLTLKGFLFLHTLFIQRGRHETTWTVLRRFGYDDDLDLTPEYLFPLLKIPPDCTTELNHHAYLFLQSTFDKHDLDRDCALSPDELKDLFKVFPYIPWGPDVNNTVCTNERGWITYQGFLSQWTLTTYLDVQRCLEYLGYLGYSILTEQESQASAVTVTRDKKIDLQKKQTQRNVFRCNVIGVKNCGKSGVLQALLGRNLMRQKKIREDHKSYYAINTVYVYGQEKYLLLHDISESEFLTEAEIICDVVCLVYDVSNPKSFEYCARIFKQHFMDSRIPCLIVAAKSDLHEVKQEYSISPTDFCRKHKMPPPQAFTCNTADAPSKDIFVKLTTMAMYPHVTQADLKSSTFWLRASFGATVFAVLGFAMYKALLKQR

SEQ ID NO: 29 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 30 - protein, 581 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 31 - DNA, 3766 bp, Homo sapiens
tgcggccgcaggttccgctgtctcgggaaccgtcgtatccctcggtccggcggcggcggcggcggtagcggaggagacggtttcaggcctccggtgcggctgcaatgctgagctcccgggccgaggcggcgatgaccgcggccgacagggccatccagcgcttcctgcggaccggggcggccgtcagatataaagtcatgaagaactggggagttataggtggaattgctgctgctcttgcagcaggaatatatgttatttggggtcccattacagaaagaaagaagcgtagaaaagggcttgtgcctggccttgttaatttagggaacacctgcttcatgaactccctgctacaaggcctgtctgcctgtcctgctttcatcaggtggctggaagagttcacctcccagtactccagggatcagaaggagcccccctcacaccagtatttatccttaacactcttgcaccttctgaaagccttgtcctgccaagaagttactgatgatgaggtcttagatgcaagctgcttgttggatgtcttaagaatgtacagatggcagatctcatcatttgaagaacaggatgctcacgaattattccatgtcattacctcgtcattggaagatgagcgagaccgccagcctcgggtcacacatttgtttgatgtgcattccctggagcagcagtcagaaataactcccaaacaaattacctgccgcacaagagggtcacctcaccctacatccaatcactggaagtctcaacatccttttcatggaagactcactagtaatatggtctgcaaacactgtgaacaccagagtcctgttcgatttgatacctttgatagcctttcactaagtattccagccgccacatggggtcacccattgaccctggaccactgccttcaccacttcatctcatcagaatcagtgcgggatgttgtgtgtgacaactgtacaaagattgaagccaagggaacgttgaacggggaaaaggtggaacaccagaggaccacttttgttaaacagttaaaactagggaagctccctcagtgtctctgcatccacctacagcggctgagctggtccagccacggcacgcctctgaagcggcatgagcacgtgcagttcaatgagttcctgatgatggacatttacaagtaccacctccttggacataaacctagtcaacacaaccctaaactgaacaagaacccagggcctacactggagctgcaggatgggccgggagcccccacaccagttctgaatcagccaggggcccccaaaacacagatttttatgaatggcgcctgctccccatctttattgccaacgctgtcagcgccgatgcccttccctctcccagttgttcccgactacagctcctccacatacctcttccggctgatggcagttgtcgtccaccatggagacatgcactctggacactttgtcacttaccgacggtccccaccttctgccaggaaccctctctcaactagcaatcagtggctgtgggtctccgatgacactgtccgcaaggccagcctgcaggaggtcctgtcctccagcgcctacctgctgttctacgagcgcgtcctttccaggatgcagcaccagagccaggagtgcaagtctgaagaatgactgtgccctcctgcaaggctagagctgatggcactgtctgcactgtccaggaaaaaagtaaaactgtactgttgcgtgtgcaagcggccccactagagccttccagccttctggtgtgttctaagagcaggctccacctgggagccagccccagttcacaccaaaccaggctccctgaacagtcctgttcatgtgtgtaggtggttctgttgtgttaagaaagcattcattatgtccggagtgtctttttactcatctgatacaggtaattaaaagaactcagattcttgaagccaccgttttcatattgtaatgttaggtgttctcagaggggaggtacctttgtctaatcaacgtttccacttagatcttttatttttaataagcaggcccataaaaattgttgacaagaattaatgaaattattaaaggcaacaatttagaagaaaaagtgcctttcactttcgattgcttttgtagcacgtccattgtgaaatattccttccaggctactcaaaggatagcaagagaacaggtaaatgatgcctaaagaacaccttcctttttctatgccttttctaatctttcaattctttctatggagtaaaggctcatctgccaaatctgccccctggggaaactctttcactactttgtcagttataagtgaagagcttacttgttgcttttatcttttgtatattggactgagatgtaattacactgtattataaaactctgtgaatagccagaactgagctggatctttgcaacacctgattcctctgctctgtggaaaactttttcttacacaaggatccactgtggacggttactttcatctgtttatttattgcccatgcagagctcttaaggtttacaggtgggagcttggggctgtataaaaaaataatccctgccctgagttgacacctggcttaggaaggaagggctgactatggggctgcagtctctctgaacctcagtttcctcatttgtgaagtgaagggttagatttgatgaccaccaaagttcagcccttttcacgaaaaggagaaagcagcttttgactttttaaaaaacatataactacagctggcatctagtattgtcatgttgctctaggtccatattctgaatttattcatttccaatagcctaatacaaaaagtatatattgagcactttcttcccttttcaggtaagtctctgaatgcagcccagggccaaaggaattttgatgacacagtagtacctatgttttaagctatatttttaatttagaaaaatggataccaaattcaaaccgactcatcagaggtaagatttggaatcagacctttccaaaaggtcatctgaggtaaggctaagaccgcacttcctctgctgggggtgagctggcagacacaccaaacagtgccttggcagcagctcacagtgcaggaagcccaggtgatcactcttctgctgggcccaggctgcaccctgaggactcagtaactcactctcaacagaatattctgtgcaggctctccaggctctgggcgtcagggtgcaaggggcagcttgaactgtacggtccgtcctgcactcacccgatgcagaccttgactttgatgttgaaatgaacacacttgttttacccaagtctggtggaacaaatgcccaatcatgtgaccttaaagtgtactgcaaagctgtagctttaagtaattgctgttctgccactgcttactctgaaatctaccatcaaagaaagatagagaaaaggggctgagccttggaatatatggttataagcagatctttctttggtcagagaccagggtttgagccaaggctgtaaatgtgaacaatagctgtgcaaagccttttaacctgacttcttcattttgtaaattattatgcattaagtagcagcccaataatctgatttctagttttattttcaaagtaagtagcttcttttgggaaaaacctaagttaaactagtagttttgccataataactgctgatttatgtatttgctaaaggtacttttgtatctgctgtgtattatagcaataaaataatcattttgttagaaaaaaatcaaaaaaaaaaaaaa

SEQ ID NO: 32 - protein, 352 aa, Homo sapiens
MESGGRPSLCQFILLGTTSVVTAALYSVYRQKARVSQELKGAKKVHLGEDLKSILSEAPGKCVPYAVIEGAVRSVKETLNSQFVENCKGVIQRLTLQEHKMVWNRTTHLWNDCSKIIHQRTNTVPFDLVPHEDGVDVAVRVLKPLDSVDLGLETVYEKFHPSIQSFTDVIGHYISGERPKGIQETEEMLKVGATLTGVGELVLDNNSVRLQPPKQGMQYYLSSQDFDSLLQRQESSVRLWKVLALVFGFATCATLFFILRKQYLQRQERLRLKQMQEEFQEHEAQLLSRAKPEDRESLKSACVVCLSSFKSCVFLECGHVCSCTECYRALPEPKKCPICRQAITRVIPLYNS

SEQ ID NO: 33 - protein, 561 aa, Homo sapiens
MCGIWALFGSDDCLSVQCLSAMKIAHRGPDAFRFENVNGYTNCCFGFHRLAVVDPLFGMQPIRVKKYPYLWLCYNGEIYNHKKMQQHFEFEYQTKVDGEIILHLYDKGGIEQTICMLDGVFAFVLLDTANKKVFLGRDTYGVRPLFKAMTEDGFLAVCSEAKGLVTLKHSATPFLKVEPFLPGHYEVLDLKPNGKVASVEMVKYHHCRDVPLHALYDNVEKLFPGFEIETVKNNLRILFNNAVKKRLMTDRRIGCLLSGGLDSSLVAATLLKQLKEAQVQYPLQTFAIGMEDSPDLLAARKVADHIGSEHYEVLFNSEEGIQALDEVIFSLETYDITTVRASVGMYLISKYIRKNTDSVVIFSGEGSDELTQGYIYFHKAPSPEKAEEESERLLRELYLFDVLRADRTTAAHGLELRVPFLDHRFSSYYLSLPPEMRIPKNGIEKHLLRETFEDSNLIPKEILWRPKEAFSDGITSVKNSWFKILQEYVEHQVDDAMMANAAQKFPFNTPKTKEGYYYRQVFERHYPGRADWLSHYWMPKWINATDPSARTLTHYKSAVKA

SEQ ID NO: 34 - protein, 412 aa, Homo sapiens
MASCAEPSEPSAPLPAGVPPLEDFEVLDGVEDAEGEEEEEEEEEEEDDLSELPPLEDMGQPPAEEAEQPGALAREFLAAMEPEPAPAPAPEEWLDILGNGLLRKKTLVPGPPGSSRPVKGQVVTVHLQTSLENGTRVQEEPELVFTLGDCDVIQALDLSVPLMDVGETAMVTADSKYCYGPQGRSPYIPPHAALCLEVTLKTAVDGPDLEMLTGQERVALANRKRECGNAHYQRADFVLAANSYDLAIKAITSSAKVDMTFEEEAQLLQLKVKCLNNLAASQLKLDHYRAALRSCSLVLEHQPDNIKALFRKGKVLAQQGEYSEAIPILRAALKLEPSNKTIHAELSKLVKKHAAQRSTETALYRKMLGNPSRLPAKCPGKGAWSIPWKWLFGATAVALGGVALSVVIAARN

SEQ ID NO: 35 - protein, 608 aa, Homo sapiens
MAASKPVEAAVVAAAVPSSGSGVGGGGTAGPGTGGLPRWQLALAVGAPLLLGAGAIYLWSRQQRRREARGRGDASGLKRNSERKTPEGRASPAPGSGHPEGPGAHLDMNSLDRAQAAKNKGNKYFKAGKYEQAIQCYTEAISLCPTEKNVDLSTFYQNRAAAFEQLQKWKEVAQDCTKAVELNPKYVKALFRRAKAHEKLDNKKECLEDVTAVCILEGFQNQQSMLLADKVLKLLGKEKAKEKYKNREPLMPSPQFIKSYFSSFTDDIISQPMLKGEKSDEDKDKEGEALEVKENSGYLKAKQYMEEENYDKIISECSKEIDAEGKYMAEALLLRATFYLLIGNANAAKPDLDKVISLKEANVKLRANALIKRGSMYMQQQQPLLSTQDFNMAADIDPQNADVYHHRGQLKILLDQVEEAVADFDECIRLRPESALAQAQKCFALYRQAYTGNNSSQIQAAMKGFEEVIKKFPRCAEGYALYAQALTDQQQFGKADEMYDKCIDLEPDNATTYVHKGLLQLQWKQDLDRGLELISKAIEIDNKCDFAYETMGTIEVQRGNMEKAIDMFNKAINLAKSEMEMAHLYSLCDAAHAQTEVAKKYGLKPPTL

SEQ ID NO: 36 - protein, 334 aa, Homo sapiens
MVGREKELSIHFVPGSCRLVEEEVNIPNRRVLVTGATGLLGRAVHKEFQQNNWHAVGCGFRRARPKFEQVNLLDSNAVHHIIHDFQPHVIVHCAAERRPDVVENQPDAASQLNVDASGNLAKEAAAVGAFLIYISSDYVFDGTNPPYREEDIPAPLNLYGKTKLDGEKAVLENNLGAAVLRIPILYGEVEKLEESAVTVMFDKVQFSNKSANMDHWQQRFPTHVKDVATVCRQLAEKRMLDPSIKGTFHWSGNEQMTKYEMACAIADAFNLPSSHLRPITDSPVLGAQRPRNAQLDCSKLETLGIGQRTPFRIGIKESLWPFLIDKRWRQTVFH

SEQ ID NO: 37 - protein, 256 aa, Homo sapiens
MAAAVGRLLRASVARHVSAIPWGISATAALRPAACGRTSLTNLLCSGSSQAKLFSTSSSCHAPAVTQHAPYFKGTAVVNGEFKDLSLDDFKGKYLVLFFYPLDFTFVCPTEIVAFSDKANEFHDVNCEVVAVSVDSHFSHLAWINTPRKNGGLGHMNIALLSDLTKQISRDYGVLLEGSGLALRGLFIIDPNGVIKHLSVNDLPVGRSVEETLRLVKAFQYVETHGEVCPANWTPDSPTIKPSPAASKEYFQKVNQ

SEQ ID NO: 38 - protein, 1019 aa, Homo sapiens [sequence omitted]

SEQ ID NO: 39 - protein, 283 aa, Homo sapiens
MAVPPTYADLGKSARDVFTKGYGFGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGHKLGLGLEFQA

SEQ ID NO: 40 - protein, 1097 aa, Homo sapiens
MAAAAAEQQQFYLLLGNLLSPDNVVRKQAEETYENIPGQSKITFLLQAIRNTTAAEEARQMAAVLLRRLLSSAFDEVYPALPSDVQTAIKSELLMIIQMETQSSMRKKVCDIAAELARNLIDEDGNNQWPEGLKFLFDSVSSQNVGLREAALHIFWNFPGIFGNQQQHYLDVIKRMLVQCMQDQEHPSIRTLSARATAAFILANEHNVALFKHFADLLPGFLQAVNDSCYQNDDSVLKSLVEIADTVPKYLRPHLEATLQLSLKLCGDTSLNNMQRQLALEVIVTLSETAAAMLRKHTNIVAQTIPQMLAMMVDLEEDEDWANADELEDDDFDSNAVAGESALDRMACGLGGKLVLPMIKEHIMQMLQNPDWKYRHAGLMALSAIGEGCHQQMEGILNEIVNFVLLFLQDPHPRVRYAACNAVGQMATDFAPGFQKKFHEKVIAALLQTMEDQGNQRVQAHAAAALINFTEDCPKSLLIPYLDNLVKHLHSIMVLKLQELIQKGTKLVLEQVVTSIASVADTAEEKFVPYYDLFMPSLKHIVENAVQKELRLLRGKTIECISLIGLAVGKEKFMQDASDVMQLLLKTQTDFNDMEDDDPQISYMISAWARMCKILGKEFQQYLPVVMGPLMKTASIKPEVALLDTQDMENMSDDDGWEFVNLGDQQSFGIKTAGLEEKSTACQMLVCYAKELKEGFVEYTEQVVKLMVPLLKFYFHDGVRVAAAESMPLLLECARVRGPEYLTQMWHFMCDALIKAIGTEPDSDVLSEIMHSFAKCIEVMGDGCLNNEHFEELGGILKAKLEEHFKNQELRQVKRQDEDYDEQVEESLQDEDDNDVYILTKVSDILHSIFSSYKEKVLPWFEQLLPLIVNLICPHRPWPDRQWGLCIFDDVIEHCSPASFKYAEYFLRPMLQYVCDNSPEVRQAAAYGLGVMAQYGGDNYRPFCTEALPLLVRVIQSADSKTKENVNATENCISAVGKIMKFKPDCVNVEEVLPHWLSWLPLHEDKEEAVQTFNYLCDLIESNHPIVLGPNNTNLPKIFSIIAEGEMHEAIKHEDPCAKRLANVVRQVQTSGGLWTECIAQLSPEQQAAIQELLNSA

SEQ ID NO: 41 - protein, 179 aa, Homo sapiens
MPSKSLVMEYLAHPSTLGLAVGVACGMCLGWSLRVCFGMLPKSKTSKTHTDTESEASILGDSGEYKMILVVRNDLKMGKGKVAAQCSHAAVSAYKQIQRRNPEMLKQWEYCGQPKVVVKAPDEETLIALLAHAKMLGLTVSLIQDAGRTQIAPGSQTVLGIGPGPADLIDKVTGHLKLY

SEQ ID NO: 42 - protein, 376 aa, Homo sapiens
MKDVPGFLQQSQNSGPGQPAVWHRLEELYTKKLWHQLTLQVLDFVQDPCFAQGDGLIKLYENFISEFEHRVNPLSLVEIILHVVRQMTDPNVALTFLEKTREKVKSSDEAVILCKTAIGALKLNIGDLQVTKETIEDVEEMLNNLPGVTSVHSRFYDLSSKYYQTIGNHASYYKDALRFLGCVDIKDLPVSEQQERAFTLGLAGLLGEGVFNFGELLMHPVLESLRNTDRQWLIDTLYAFNSGNVERFQTLKTAWGQQPDLAANEAQLLRKIQLLCLMEMTFTRPANHRQLTFEEIAKSAKITVNEVELLVMKALSVGLVKGSIDEVDKRVHMTWVQPRVLDLQQIKGMKDRLEFWCTDVKSMEMLVEHQAHDILT

SEQ ID NO: 43 - protein, 863 aa, Homo sapiens
MQRRGALFGMPGGSGGRKMAAGDIGELLVPHMPTIRVPRSGDRVYKNECAFSYDSPNSEGGLYVCMNTFLAFGREHVERHFRKTGQSVYMHLKRHVREKVRGASGGALPKRRNSKIFLDLDTDDDLNSDDYEYEDEAKLVIFPDHYEIALPNIEELPALVTIACDAVLSSKSPYRKQDPDTWENELPVSKYANNLTQLDNGVRIPPSGWKCARCDLRENLWLNLTDGSVLCGKWFFDSSGGNGHALEHYRDMGYPLAVKLGTITPDGADVYSFQEEEPVLDPHLAKHLAHFGIDMLHMHGTENGLQDNDIKLRVSEWEVIQESGTKLKPMYGPGYTGLKNLGNSCYLSSVMQAIFSIPEFQRAYVGNLPRIFDYSPLDPTQDFNTQMTKLGHGLLSGQYSKPPVKSELIEQVMKEEHKPQQNGISPRMFKAFVSKSHPEFSSNRQQDAQEFFLHLVNLVERNRIGSENPSDVFRFLVEERIQCCQTRKVRYTERVDYLMQLPVAMEAATNKDELIAYELTRREAEANRRPLPELVRAKIPFSACLQAFSEPENVDDFWSSALQAKSAGVKTSRFASFPEYLVVQIKKFTFGLDWVPKKFDVSIDMPDLLDINHLRARGLQPGEEELPDISPPIVIPDDSKDRLMNQLIDPSDIDESSVMQLAEMGFPLEACRKAVYFTGNMGAEVAFNWIIVHMEEPDFAEPLTMPGYGGAASAGASVFGASGLDNQPPEEIVAIITSMGFQRNQAIQALRATNNNLERALDWIFSHPEFEEDSDFVIEMENNANANIISEAKPEGPRVKDGSGTYELFAFISHMGTSTMSGHYICHIKKEGRWVIYNDHKVCASERPPKDLGYMYFYRRIPS

SEQ ID NO: 44 - protein, 294 aa, Homo sapiens
MATHGQTCARPMCIPPSYADLGKAARDIFNKGFGFGLVKLDVKTKSCSGVEFSTSGSSNTDTGKVTGTLETKYKWCEYGLTFTEKWNTDNTLGTEIAIEDQICQGLKLTFDTTFSPNTGKKSGKIKSSYKRECINLGCDVDFDFAGPAIHGSAVFGYEGWLAGYQMTFDSAKSKLTRNNFAVGYRTGDFQLHTNVNDGTEFGGSIYQKVCEDLDTSVNLAWTSGTNCTRFGIAAKYQLDPTASISAKVNNSSLIGVGYTQTLRPGVKLTLSALVDGKSINAGGHKVGLALELEA

SEQ ID NO: 45 - protein, 283 aa, Homo sapiens
MCNTPTYCDLGKAAKDVFNKGYGFGMVKIDLKTKSCSGVEFSTSGHAYTDTGKASGNLETKYKVCNYGLTFTQKWNTDNTLGTEISWENKLAEGLKLTLDTIFVPNTGKKSGKLKASYKRDCFSVGSNVDIDFSGPTIYGWAVLAFEGWLAGYQMSFDTAKSKLSQNNFALGYKAADFQLHTHVNDGTEFGGSIYQKVNEKIETSINLAWTAGSNNTRFGIAAKYMLDCRTSLSAKVNNASLIGLGYTQTLRPGVKLTLSALIDGKNFSAGGHKVGLGFELEA

SEQ ID NO: 46 - protein, 19 aa, Artificial sequence
  Synthetic
RLTSNMVCKHCEHQSPVRF

SEQ ID NO: 47 - protein, 15 aa, Artificial sequence
  Synthetic
RDVVCDNCTKIEAKG

SEQ ID NO: 48 - protein, 14 aa, Artificial sequence
  Synthetic
XXCXXXXXXXXXCX

SEQ ID NO: 49 - protein, 16 aa, Artificial sequence
  Synthetic
XXXCXXXXXXXXXCXX